(19) United States
(12) Patent Application Publication
(10) Pub. No.: US 2016/0186266 A1
(43) Pub. Date: Jun. 30, 2016

(54) MOLECULAR PROFILING FOR PERSONALIZED MEDICINE

(71) Applicant: CARIS LIFE SCIENCES, INC., Irving, TX (US)

(72) Inventor: Arlet Alarcon, Phoenix, AZ (US)

(73) Assignee: Carislife Sciences, Inc., Irving, TX (US)

(21) Appl. No.: 13/504,479

(22) PCT Filed: Oct. 27, 2010
(86) PCT No.: PCT/US10/54366

§ 371 Date: Oct. 15, 2012

Related U.S. Application Data
(60) Provisional application No. 61/279,970, filed on Oct. 27, 2009.
(60) Provisional application No. 61/261,709, filed on Nov. 16, 2009.
(60) Provisional application No. 61/294,440, filed on Jan. 12, 2010.
(60) Provisional application No. 61/346,862, filed on May 20, 2010.
(60) Provisional application No. 61/354,145, filed on Jun. 11, 2010.
(60) Provisional application No. 61/362,287, filed on Jul. 7, 2010.
(60) Provisional application No. 61/406,352, filed on Oct. 25, 2010.

(51) Int. Cl. C12Q 1/68; G01N 33/574

(52) U.S. Cl. CPC C12Q 1/6886; G01N 33/57415; C12Q 2600/106; C12Q 2600/156; C12Q 2600/118; C12Q 2600/158

(57) ABSTRACT

Provided herein are methods and systems of molecular profiling of diseases, such as cancer. In some embodiments, the molecular profiling can be used to identify treatments for a disease, such as treatments that were not initially identified as a treatment for the disease or not expected to be a treatment for a particular disease.

### RELATED APPLICATIONS

This application claims the benefit of U.S. provisional patent application 61/279,970, filed on Oct. 27, 2009, U.S. provisional patent application 61/261,709, filed on Nov. 16, 2009, U.S. provisional patent application 61/354,145, filed on Jun. 11, 2010, U.S. provisional patent application 61/406,352, filed on Oct. 25, 2010, U.S. provisional patent application 61/346,862, filed on May 20, 2010, and U.S. provisional patent application 61/362,287, filed on Jul. 7, 2010; all of which applications are incorporated herein by reference in their entirety.

### BACKGROUND

Disease states in patients are typically treated with treatment regimens or therapies that are selected based on clinical based criteria; that is, a treatment therapy or regimen is selected for a patient based on the determination that the patient has been diagnosed with a particular disease (which diagnosis has been made from classical diagnostic assays). Although the molecular mechanisms behind various disease states have been the subject of studies for years, the specific application of a diseased individual's molecular profile in determining treatment regimens and therapies for that individual has been disease specific and not widely pursued.

Some treatment regimens have been determined using molecular profiling in combination with clinical characterization of a patient such as observations made by a physician (such as a code from the International Classification of Diseases, for example, and the dates such codes were determined), laboratory test results, x-rays, biopsy results, statements made by the patient, and any other medical information typically relied upon by a physician to make a diagnosis in a specific disease. However, using a combination of selection material based on molecular profiling and clinical characterizations (such as the diagnosis of a particular type of cancer) to determine a treatment regimen or therapy presents a risk that an effective treatment regimen may be overlooked for a particular individual since some treatment regimens may work well for different disease states even though they are associated with treating a particular type of disease state.

Patients with refractory or metastatic cancer are of particular concern for treating physicians. The majority of patients with metastatic or refractory cancer eventually run out of treatment options or may suffer a cancer type with no real treatment options. For example, some patients have very limited options after their tumor has progressed in spite of front line, second line and sometimes third line and beyond) therapies. For these patients, molecular profiling of their cancer may provide the only viable option for prolonging life.

More particularly, additional targets or specific therapeutic agents can be identified assessment of a comprehensive number of targets or molecular findings examining molecular mechanisms, genes, gene expressed proteins, and/or combinations of such in a patient's tumor. Identifying multiple agents that can treat multiple targets or underlying mechanisms would provide cancer patients with a viable therapeutic alternative on a personalized basis so as to avoid standard therapies, which may simply not work or identify therapies that would not otherwise be considered by the treating physician.

There remains a need for better theranostic assessment of cancer victims, including molecular profiling analysis that identifies one or more individual profiles to provide more informed and effective personalized treatment options, resulting in improved patient care and enhanced treatment outcomes. The present invention provides methods and systems for identifying treatments for these individuals by molecular profiling a sample from the individual.

### SUMMARY OF THE INVENTION

The present invention provides methods and system for molecular profiling, using the results from molecular profiling to identify treatments for individuals. In some embodiments, the treatments were not identified initially as a treatment for the disease.

In an aspect, the invention provides a method of identifying a candidate treatment for a subject in need thereof, comprising: a) determining a molecular profile for the subject on a panel of gene or gene products, wherein the molecular profile comprises the results of: performing immunohistochemistry (IHC) analysis on a sample from the subject on one or more of: AR, BCRP, BRCA1, BRCA2, CAV-1, CK 14, CK 5/6, CK17, c-kit, cMET, COX2, Cyclin D1, ECAD, EGFR, ER, ERCC1, HER2, IGFR1, IGFRBP3, IGFRBP4, IGFRBP5, Ki67, MGMT, MPR1, P53, p95, PDGFR, PGP, PR, PTEN, RRM1, SPARC, TLE3, TOP2A, TOPO1, TS, and β-III tubulin; performing microarray analysis on the sample on one or more of: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPIIA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing fluorescent in-situ hybridization (FISH) analysis on the sample on at least one of cMYC, EGFR, EML4-ALK fusion, HER2, and MET; and performing DNA sequence analysis on the sample on at least one of BRAF, c-kit, EGFR, KRAS, and PIK3CA; b) comparing the molecular profile of the subject to a molecular profile of a reference to identify a comparison molecular profile; and c) identifying a treatment that is associated with the comparison molecular profile, thereby identifying the candidate treatment.

In another aspect, the invention provides a method of identifying a candidate treatment for a cancer in a subject in need thereof, comprising: a) determining a molecular profile for the subject on a panel of gene or gene products, wherein the molecular profile comprises the results of: performing an immunohistochemistry (IHC) analysis on a sample from the subject on at least the group of proteins consisting of: AR, BCRP, BRCA1, BRCA2, CAV-1, CK 14, CK 5/6, CK17, c-kit, cMET, COX2, Cyclin D1, ECAD, EGFR, ER, ERCC1, HER2, IGFR1, IGFRBP3, IGFRBP4, IGFRBP5, Ki67, MGMT, MPR1, P53, p95, PDGFR, PGP, PR, PTEN, RRM1, SPARC, TLE3, TOP2A, TOPO1, TS, and β-III tubulin; performing a microarray analysis on the sample on at least the group of genes consisting of: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing a fluorescent in-situ hybridization (FISH) analysis on the sample on at least the group of genes consisting of cMYC, EGFR, EML4-ALK fusion and HER2; performing DNA sequencing on the sample on at least the group of genes consisting of BRAF, c-kit, EGFR, KRAS, and PIK3CA; b) comparing the molecular profile of the subject to a molecular profile of a reference to identify a comparison molecular profile; and c) identifying a treatment that is associated with the comparison molecular profile, thereby identifying the candidate treatment.

In yet another aspect, the invention provides a method of identifying a candidate treatment for a subject with a breast cancer, comprising determining a molecular profile for the subject on a panel of gene or gene products, wherein the molecular profile comprises the results of: performing an immunohistochemistry (IHC) analysis on a sample from the subject on at least one of HER2, ER, PR, P53 and Ki67; performing a microarray analysis on the sample on at least one of: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing a fluorescent in-situ hybridization (FISH) analysis on the sample on at least HER2. If the cancer is HER2 positive (HER2+), the molecular profile further comprises performing IHC analysis on the sample on at least one of AR, C-Kit, MRP1, PDGFR, PGP, PTEN, SPARC, TOP2A, TS, CAV1, CK14, CK17, CK5/6, ECAD, P95, and TLE3; performing FISH analysis on the sample on cMYC and TOP2A; and performing sequence analysis on the sample on PIK3CA. If the cancer is HER2 negative (HER2−) and positive for either ER (ER+) or PR (PR+), the molecular profile further comprises performing IHC analysis on the sample on at least one of AR, C-Kit, MRP1, PDGFR, PGP, PTEN, SPARC, TOP2A, TS, CAV-1, CK14, CK17, CK 5/6, CYCLIN D1, ECAD, EGFR, P95, TLE3; and performing FISH analysis on the sample on cMYC. If the cancer is triple negative (IIER2−, ER− and PR−), the molecular profile further comprises performing IHC analysis on the sample on at least one of AR, C-Kit, MRP1, PDGFR, PGP, PTEN, SPARC, TS, TOP2A, CAV1, CK14, CK17, CK5/6, ECAD, P95, TLE3. The molecular profile of the subject is compared to a molecular profile of a reference to identify a comparison molecular profile; and a treatment is identified that is associated with the comparison molecular profile, thereby identifying the candidate treatment.

In the methods of the invention, identifying a treatment that is associated the comparison molecular profile can include correlating the comparison molecular profile with a rules database, wherein the rules database comprises a mapping of treatments whose biological activity is determined against cancer cells that have different level of, overexpress, underexpress, and/or have mutations in one or more members of the panel of gene or gene products; and identifying the treatment based on the correlating. In some embodiments, the rules database comprises one or more of the rules listed in Table 3 and/or Table 4 herein. In some embodiments, the mapping of treatments contained within the rules database is based on the efficacy of various treatments particular for a target gene or gene product.

The sample comprises a biological sample from the subject, including without limitation a bodily fluid, a tissue sample, formalin-fixed paraffin-embedded (FFPE) tissue, fresh frozen (FF) tissue, or tissue comprised in a solution that preserves nucleic acid or protein molecules. The sample may comprise cells from any tissue of the body, e.g., the cells can be selected from the group consisting of adipose, adrenal cortex, adrenal gland, adrenal gland-medulla, appendix, bladder, blood, blood vessel, hone, bone cartilage, brain, breast, cartilage, cervix, colon, colon sigmoid, dendritic cells, skeletal muscle, enodmetrium, esophagus, fallopian tube, fibroblast, gallbladder, kidney, larynx, liver, lung, lymph node, melanocytes, mesothelial lining, myoepithelial cells, osteoblasts, ovary, pancreas, parotid, prostate, rectum, salivary gland, sinus tissue, skeletal muscle, skin, small intestine, smooth muscle, stomach, synovium, joint lining tissue, tendon, testis, thymus, thyroid, uterus, and uterus corpus.

In the subject methods, the reference can be from a non-cancerous sample. In one embodiment, the reference is from the subject, e.g., normal adjacent tissue or a non-diseased sample taken at a different time course. In another embodiment, the reference is from another individual that the subject. The reference profile can derived from a plurality of reference samples. For example, the reference can be an average profile from a number of non-cancerous samples. In another embodiment, the reference comprises profiles from different individuals for different biomarkers.

In some embodiments of the invention, the molecular profiling consists of IHC. This may be the case when the sample has to pass a quality control test before certain techniques are performed. For example, the mRNA for the sample must be of high enough quality for microarray expression profiling to be performed. The quality control test can include an A260/A280 ratio or a Ct value of RT-PCR of RPL13a mRNA. In some embodiments, the quality control test comprises an A260/A280 ratio <1.5 or the RPL13a Ct value is >30.

The methods of the invention include assessment of multiple biomarkers. In some embodiments, the IHC analysis is performed on at least 5, 10 or 15 of the biomarkers listed for IHC analysis. In some embodiments, IHC is performed on substantially all of the biomarkers listed for IHC analysis. In some embodiments, the microarray analysis is performed on at least 5, 10, 15, 20, 30, 40, 50, 60, 70, or 80 of the biomarkers listed for microarray analysis. In some embodiments, microarray analysis is performed on substantially all of the listed biomarkers for microarray analysis. FISH and sequence analysis can also be performed on all of the biomarkers listed for FISH and sequence analysis, respectively.

In some embodiments, the molecular profiling further comprises IHC analysis on the sample on BCRP, ERCC1, MGMT, RRM1 and TOPO1; and FISH analysis on the sample on EGFR. For example, wherein the therapeutic history of the cancer comprises fourth line therapy or is unknown, or if the cancer is metastatic, the molecular profiling can comprise IHC analysis on the sample on BCRP, ERCC1, MGMT, RRM1 and TOPO1; and FISH analysis on the sample on EGFR. In other embodiments, the FISH or IHC analysis further comprises analysis of one or more of hENT1, cMet, P21, PARP-1, TLE3 and IGF1R. For example, wherein the cancer is HER2 negative (HER2−) and positive for ER (ER+) or PR (PR+), and the FISH or IHC analysis can further comprise analysis of one or more of hENT1, cMet, P21, PARP-1, TLE3 and IGF1R.

The panel of gene or gene products used for molecular profiling according to the subject methods can include one or more of ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKNIA, CDKNIB, CDK2, CDW52, CES2, CK 14, CK 17, CK 5/6, c-KIT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, GART, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, LYN, MET, MGMT, MLH1, MMR, MRP1, MS4A1, MSH2, MSH5, Myc, NFKB1, NFKB2, NFKBIA, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, RAF1, RARA, RRM1, RRM2, RRM2B, RXRG, SPARC, SRC, SSTR1, SSTR2, SSTR3, SSTR4, SSTR5, Survivin, TK1, TLE3, TNF, TOP1, TOP2A, TOP2B, TS, TXN, TXNRD1, TYMS, VDR, VEGF, VEGFA, VEGFC, VHL, YES1, ZAP70. The panel of gene or gene products can include one or more gene or gene product in Table 1.

The microarray analysis used according to the methods of the invention can include a low density microarray, an expression microarray, a comparative genomic hybridization (CGH) microarray, a single nucleotide polymorphism (SNP) microarray, a proteomic array and/or an antibody array. In some embodiments, the microarray analysis comprises identifying whether a gene is upregulated or downregulated relative to a reference with statistical significance. Statistical significance can be determined at a p-value of less than or equal to some threshold, e.g., 0.05, 0.01, 0.005, (1001, 0.0005, or 0.0001. The p-value can be corrected for multiple comparisons. A number of corrections for multiple comparisons are known in the art, such as Bonneferoni's correction or a modification thereof.

The IHC analysis according to the methods of the invention may also comprise a threshold. In some embodiments, IHC analysis comprises determining whether 30% or more of said sample is +2 or greater in staining intensity.

The methods of the invention can identify a prioritized list of candidate treatments. In some embodiments, prioritizing comprises ordering the treatments from higher priority to lower priority according to treatments based on microarray analysis and either IHC or FISH analysis; treatments based on IHC analysis but not microarray analysis; and treatments based on microarray analysis but not IHC analysis.

The candidate treatment identified by the methods of the invention can be one or more therapeutic agents. In some embodiments, the one or more therapeutic agents comprise 5-fluorouracil, abarelix, Alemtuzumab, aminoglutethimide, Anastrazole, aromatase inhibitors (anastrazole, letrozole), asparaginase, aspirin, ATRA, azacitidine, bevacizumab, bexarotene, Bicalutamide, bortezomib, calcitriol, capecitabine, Carboplatin, celecoxib, Cetuximab, Chemocndocrine therapy, cholecalciferol, Cisplatin, carboplatin, Cyclophosphamide, Cyclophosphamide/Vincristine, cytarabine, dasatinib, decitabine, Doxorubicin, Epirubicin, epirubicin, Erlotinib, Etoposide, exemestane, fluoropyrimidines, Flutamide, fulvestrant, Gefitinib, Gefitinib and Trastuzumab, Gemcitabine, gonadorelin, Goserelin, hydroxyurea, Imatinib, Irinotecan, Ixabepilone, Lapatinib, Letrozole, Leuprolide, liposomal doxorubicin, medroxyprogesterone, megestrol, methotrexate, mitomycin, nab-paclitaxel, octreotide, Oxaliplatin, Paclitaxel, Panitumumab, pegaspargase, pemetrexed, pentostatin, sorafenib, sunitinib, Tamoxifen, Tamoxifen-based treatment, Temozolomide, topotecan, toremifene, Trastuzumab, VBMCP/Cyclophosphamide, Vincristine, or any combination thereof. In some embodiments, the one or more therapeutic agents comprise 5FU, bevacizumab, capecitabine, cetuximab, cetuximab+gemcitabine, cetuximab+irinotecan, cyclophospohamide, diethylstibesterol, doxorubicin, erlotinib, etoposide, exemestane, fluoropyrimidines, gemcitabine, gemcitabine+etoposide, gemcitabine+pemetrexed, irinotecan, irinotecan+sorafenib, lapatinib, lapatinib+tamoxifen, letrozole, letrozole+capecitabine, mitomycin, nab-paclitaxel, nab-paclitaxel+gemcitabine, nab-paclitaxel+trastuzumab, oxaliplatin, oxaliplatin+5FU+trastuzumab, panitumumab, pemetrexed, sorafenib, sunitinib, sunitinib, sunitinib+mitomycin, tamoxifen, temozolomide, temozolomide+bevacizumab, temozolomide+sorafenib, trastuzumab, vincristine, or any combination thereof.

In some embodiments, the one or more therapeutic agents are chosen from the class of therapeutic agents identified as Anthracyclines and related substances, Anti-androgens, Anti-estrogens, Antigrowth hormones, Combination therapy, DNA methyltransferase inhibitors, Endocrine therapy—Enzyme inhibitor, Endocrine therapy—other hormone antagonists and related agents, Folic acid analogs, Gonadotropin releasing hormone analogs, Gonadotropin-releasing hormones, Monoclonal antibodies (EGFR-Targeted), Monoclonal antibodies (Her2-Targeted), Monoclonal antibodies (Multi-Targeted), Other alkylating agents, Antineoplastic agents, Cytotoxic antibiotics, Platinum compounds, Podophyllotoxin derivatives, Progestogens, Protein kinase inhibitors (EGFR-Targeted), Protein kinase inhibitors (Her2 targeted), Pyrimidine analogs, Pyrimidine analogs, Salicylic acid and derivatives, Src-family protein tyrosine kinase inhibitors, Taxanes, Vinca Alkaloids and analogs, Vitamin D and analogs, and Protein kinase inhibitors.

In some embodiments, the one or more therapeutic agents comprise one or more of 5-fluorouracil, abarelix, alemtuzumab, aminoglutethimide, anastrozole, asparaginase, aspirin, ATRA, azacitidine, bevacizumab, bexarotene, bicalutamide, calcitriol, capecitabine, carboplatin, celecoxib, cetuximab, chemotherapy, cholecalciferol, cisplatin, cytarabine, dasatinib, daunorubicin, decitabine, doxorubicin, epirubicin, erlotinib, etoposide, exemestane, flutamide, fulvestrant, gefitinib, gemcitabine, gonadorelin, goserelin, hydroxyurea, imatinib, irinotecan, lapatinib, letrozole, leuprolide, liposomal-doxorubicin, medroxyprogesterone, megestrol, megestrol acetate, methotrexate, mitomycin, nab-paclitaxel, octreotide, oxaliplatin, paclitaxel, panitumumab, pegaspargase, pemetrexed, pentostatin, sorafenib, sunitinib, tamoxifen, Taxanes, temozolomide, toremifene, trastuzumab, VBMCP, and vincristine.

The method of the invention can be performed wherein the subject has been previously treated with the candidate treatment. Alternately the subject has not previously been treated with one or more identified candidate therapeutic agents. The cancer can be a metastatic cancer. The cancer can also be a recurrent cancer. In some embodiments, the cancer is refractory to a prior treatment. The prior treatment can include the standard of care for the cancer.

The methods of the invention can be used for molecular profiling on any cancer sample of adequate quantity and quality for analysis. In some embodiments, the cancer comprises a prostate, lung, melanoma, small cell (esopha/retroperit), cholangiocarcinoma, mesothelioma, head and neck (SCC), pancreas, pancreas neuroendocrine, small cell, gastric, peritoneal pseudomyxoma, anal Canal (SCC), vagina (SCC), cervical, renal, eccrine seat adenocarinoma, salivary gland adenocarinoma, uterine soft tissue sarcoma (uterine), GIST (Gastric), or thyroid-anaplastic cancer.

In other embodiments, the cancer is a cancer of the accessory, sinuses, middle and inner ear, adrenal glands, appendix, hematopoietic system, bones and joints, spinal cord, breast, cerebellum, cervix uteri, connective and soft tissue, corpus uteri, esophagus, eye, nose, eyeball, fallopian tube, extrahepatic bile ducts, mouth, intrahepatic bile ducts, kidney, appendix-colon, larynx, lip, liver, lung and bronchus, lymph nodes, cerebral, spinal, nasal cartilage, retina, eye, oropharynx, endocrine glands, female genital, ovary, pancreas, penis and scrotum, pituitary gland, pleura, prostate gland, rectum renal pelvis, ureter, peritonem, salivary gland, skin, small intestine, stomach, testis, thymus, thyroid gland, tongue, unknown, urinary bladder, uterus, vagina, labia, or vulva.

In still other embodiments, the cancer comprises a breast, colorectal, ovarian, lung, non-small cell lung cancer, cholangiocarcinoma, mesothelioma, sweat gland, or GIST cancer.

The cancer can be a breast cancer, pancreatic cancer, cancer of the colon and/or rectum, leukemia, skin cancer, hone cancer, prostate cancer, liver cancer, lung cancer, brain cancer, cancer of the larynx, gallbladder, parathyroid, thyroid, adrenal, neural tissue, head and neck, stomach, bronchi, kidneys, basal cell carcinoma, squamous cell carcinoma of both ulcerating and papillary type, metastatic skin carcinoma, ostco sarcoma, Ewing's sarcoma, vcticulum cell sarcoma, myeloma, giant cell tumor, small-cell lung tumor, islet cell carcinoma, primary brain tumor, acute and chronic lymphocytic and granulocytic tumors, hairy-cell tumor, adenoma, hyperplasia, medullary carcinoma, pheochromocytoma, mucosal neuroma, intestinal ganglioneuroma, hyperplastic corneal nerve tumor, marfanoid habitus tumor, Wilm's tumor, seminoma, ovarian tumor, leiomyoma, cervical dysplasia and in situ carcinoma, neuroblastoma, retinoblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, and/or epidermoid carcinomas.

The methods of the invention can be used to identify a candidate therapeutic for a cancer comprising an adenocarcinoma, carcinoma, a sarcoma, a lymphoma or leukemia, a germ cell tumor, or a blastoma. The carcinoma can be epithelial neoplasms, squamous cell neoplasms, squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and/or malignant acral lentiginous melanoma. The sarcoma can include Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and/or synovialsarcoma. The lymphoma or leukemia can be chronic lymphocytic leukemia/small lymphocytic lymphoma, B-cell prolymphocytic leukemia, lymphoplasmacytic lymphoma, waldenstrom macroglobulinemia, splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/lcukcmia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and/or nodular lymphocyte-predominant Hodgkin lymphoma.

The germ cell tumor can be germinoma, dysgerminoma, seminoma, nongerminomatous germ cell tumor, embryonal carcinoma, endodermal sinus turmor, choriocarcinoma, teratoma, polyembryoma, and/or gonadoblastoma. The blastoma can be nephroblastoma, medulloblastoma, and/or retinoblastoma. Other cancers that can be assessed include labial carcinoma, larynx carcinoma, hypopharynx carcinoma, tongue carcinoma, salivary gland carcinoma, gastric carcinoma, adenocarcinoma, thyroid cancer, medullary carcinoma, papillary thyroid carcinoma, renal carcinoma, kidney parenchyma carcinoma, cervix carcinoma, uterine corpus carcinoma, endometrium carcinoma, chorion carcinoma, testis carcinoma, urinary carcinoma, melanoma, brain tumors, glioblastoma, astrocytoma, meningioma, medulloblastoma, peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and/or plasmocytoma.

In some embodiments, the cancer comprises an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

In one embodiment, the methods of the invention are used to identify a candidate treatment for a cancer of unknown primary (CUP).

The methods of the invention can be used to determine a prognosis for the cancer based on the molecular profiling comparison. The prognosis may be based on analysis of one or more of the biomarkers in Table 6 herein.

The methods of invention can provide patient benefit. In some embodiments, progression free survival (PFS) or disease free survival (DFS) for the subject is extended by selection of the candidate treatment.

In an aspect, the invention provides a method for identifying a candidate treatment for an individual with breast cancer comprising: determining an expression level or a mutation of a gene from a biological sample of said individual, wherein said gene is selected from the group consisting of: ER, PR, HER2, KT-67 and P53; and identifying the candidate treatment based on a change in expression or a mutation in said gene as compared to a reference.

In another aspect, the invention provides a method for identifying a candidate treatment for an individual with breast cancer comprising: determining an expression level or a mutation of a gene from a biological sample of said individual, wherein said gene is selected from the group consisting of: SPARC, TOP2A, TOTO1, PGP, BCRP, MRP1, PTEN, TS, ERCC1, RRM1, MGMT, c-kit, PDGFR, AR, EGFR, KRAS, BRAF, p95 and PI3K; and identifying the candidate treatment based on a change in expression or a mutation in said gene as compared to a reference.

In yet another aspect, the invention provides a method for identifying a candidate treatment for an individual with HER-2 positive breast cancer comprising: determining an expression level or a mutation of a gene from a biological sample of said individual, wherein said gene is selected from the group consisting of: TOP2A, PGP, MRP1, TS, ERCC1, BCRP, RRM1, TOPOI, TOPOII, TLE3, C-MYC, TOP2, P95, PTEN, E-Cad, HER2, and PI3K; and identifying the candidate treatment based on a change in expression or a mutation in said gene as compared to a reference.

In still another aspect, the invention provides a method for identifying a candidate treatment for an individual with triple negative breast cancer comprising: determining an expression level or a mutation of a gene from a biological sample of said individual, wherein said gene is selected from the group consisting of: AR, KRAS, BRCA1, PARP-1, SPARC MC, SPARC PC, CK 5/6, CK14, CK17, TOP2A, PGP, MRP1, TS, ERCC1, BCRP, RRM1, TOPOI, TOPOII, and TLE3; and identifying the candidate treatment the individual based on a change in expression or a mutation in said gene as compared to a reference.

In another aspect, the invention provides a method for identifying a candidate treatment for an individual with Ductal Carcinoma in Situ comprising: determining an expression level or a mutation of a gene from a biological sample of said individual, wherein said gene is selected from the group consisting of: ER, PR, HER2, Ki-67, P53, BCL2 and E-Cadherin; and identifying the candidate treatment based on a change in expression or a mutation in said gene as compared to a reference.

The expression level can be determined by analysis of mRNA levels of said gene or protein levels of said gene. The reference can be the expression level or nucleic acid sequence of the gene or gene product in a sample without cancer. The methods may further comprise determining an expression level of a second gene. Determining according to the invention can be performed using immunohistochemistry (IHC) analysis, microarray analysis, in-situ hybridization (TSH), or real-time PCR. ISH can be fluorescent in-situ hybridization (FISH). Determining an expression level of said second gene can use the same method used for said first gene. Alternately, determining an expression level of said second gene can use a different method than that used for said first gene. In some embodiments, determining an expression level of said first gene is by IHC and said second gene is by microarray. The methods may further comprise identifying a mutation, polymorphism, or deletion, or insertion in a gene. The identifying can be performed using IHC analysis, microarray analysis, ISH, PCR, real-time PCR, or sequencing. In an embodiment, the breast cancer is an invasive breast cancer. The invasive breast cancer can be HER-2 positive or triple negative breast cancer. The breast cancer may be a metastatic cancer, a refractory cancer or a relapse.

In an aspect, the invention provides a method for identifying a candidate treatment for an individual with cancer comprising: performing FISH for EGFR and/or HER2 on a biological sample from the individual; performing mutational analysis on the sample for one or more of EGFR, c-kit, BRAF and KRAS; performing IHC on the sample for one or more of TOP2A, PTEN, TS, COX2, TOPO1, ERCC1, RRM1, MPR1, SPARC, BCRP, c-kit, MGMT, PDGFR, AR, PR, ER, PGP, and HER2; and identifying the candidate treatment based on a change in expression or a mutation in said genes or gene products as compared to a reference. The reference can be the expression level or nucleic acid sequence of the gene or gene product in a sample without cancer. The reference sample can be from the individual, e.g., normal adjacent tissue or a sample collected at a different time point, or from another individual.

In another aspect, the invention provides a method for identifying a candidate treatment for an individual with breast cancer comprising: performing FISH for cMYC and/or HER2 on a biological sample from the individual; performing mutational analysis on the sample for PIK3CA; performing IHC on the sample for one or more of P53, Ki67, p95, CK 14, CK 5/6, Cyclin D1, CAV-1, CK17, EGFR, ECAD, c-kit, MGMT, PDGFR, AR, MPR1, SPARC, PTEN, TOP2A, TS, PR, ER, PGP, HER2 and TLE3; and identifying the candidate treatment based on a change in expression or a mutation in said genes or gene products as compared to a reference. The reference can be the expression level or nucleic acid sequence of the gene or gene product in a sample without cancer. The reference sample can be from the individual, e.g., normal adjacent tissue or a sample collected at a different time point, or from another individual.

In still another aspect, the invention provides a method for identifying a candidate treatment for an individual with ovarian cancer comprising: performing FISH for HER2 a biological sample from the individual; performing IHC on the sample for one or more of TOP2A, TS, PR, ER, PGP, HER2, TLE3, BRCA1, BRCA2, IGFRBP3, IGFRBP4, IGFRBP5, TOPO1, ERCC1 and RRM1; and identifying the candidate treatment based on a change in expression or a mutation in said genes or gene products as compared to a reference. The reference can be the expression level or nucleic acid sequence of the gene or gene product in a sample without cancer. The reference sample can be from the individual, e.g., normal adjacent tissue or a sample collected at a different time point, or from another individual.

In yet another aspect, the invention provides a method for identifying a candidate treatment for an individual with colorectal cancer comprising: performing sequencing for BRAF and/or KRAS on a biological sample from the individual; performing IHC on the sample for one or more of TOP2A, TS, PTEN and COX2; and identifying the candidate treatment based on a change in expression or a mutation in said genes or gene products as compared to a reference. The reference can be the expression level or nucleic acid sequence of the gene or gene product in a sample without cancer. The reference sample can be from the individual, e.g., normal adjacent tissue or a sample collected at a different time point, or from another individual.

In an aspect, the invention provides a method for identifying a candidate treatment for an individual with lung cancer comprising: performing FISH on EGFR, EML4-ALK fusion and/or MET on a biological sample from the individual; performing mutational analysis on the sample for EGFR, BRAF and/or KRAS; performing IHC on the sample for one or more of TOP2A, PTEN, COX2, TOPO1, ERCC1, RRM1, MPR1, SPARC, BCRP, β-III tubulin, IGFR1 and cMET; and identifying the candidate treatment based on a change in expression or a mutation in said genes or gene products as compared to a reference. The reference can be the expression level or nucleic acid sequence of the gene or gene product in a sample without cancer. The reference sample can be from the individual, e.g., normal adjacent tissue or a sample collected at a different time point, or from another individual.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 illustrates a block diagram of an illustrative embodiment of a system for determining individualized medical intervention for a particular disease state that utilizes molecular profiling of a patient's biological specimen that is non disease specific.

FIG. 2 is a flowchart of an illustrative embodiment of a method for determining individualized medical intervention for a particular disease state that utilizes molecular profiling of a patient's biological specimen that is non disease specific.

FIGS. 3A through 3D illustrate an illustrative patient profile report in accordance with step 80 of FIG. 2.

FIG. 4 is a flowchart of an illustrative embodiment of a method for identifying a therapeutic agent capable of interacting with a target.

FIGS. 5-14 are flowcharts and diagrams illustrating various parts of an information-based personalized medicine drug discovery system and method in accordance with the present invention.

FIGS. 15-25 are computer screen print outs associated with various components of the information-based personalized shown in FIGS. 5-14.

FIGS. 26A-26H represent a table that shows the frequency of a significant change in expression of gene expressed proteins by tumor type.

FIGS. 27A-27H represent a table that shows the frequency of a significant change in expression of certain genes by tumor type.

FIGS. 28A-28O represent a table that shows the frequency of a significant change in expression for certain gene expressed proteins by tumor type.

FIG. 29 is a table which shows biomarkers (gene expressed proteins) tagged as targets in order of frequency based on FIG. 28.

FIGS. 30A-30O represent a table that shows the frequency of a significant change in expression for certain genes by tumor type.

FIG. 31 is a table which shows genes tagged as targets in order of frequency based on FIG. 30.

FIG. 32 illustrates progression free survival (PFS) using therapy selected by molecular profiling (period B) with PFS for the most recent therapy on which the patient has just progressed (period A). If PFS(B)/PFS(A) ratio ≧1.3, then molecular profiling selected therapy was defined as having benefit for patient.

FIG. 33 is a schematic of methods for identifying treatments by molecular profiling if a target is identified.

FIG. 34 illustrates the distribution of the patients in the study as performed in Example 1.

FIG. 35 is graph depicting the results of the study with patients having PFS ratio ≧1.3 was 18/66 (27%).

FIG. 36 is a waterfall plot of all the patients for maximum % change of summed diameters of target lesions with respect to baseline diameter.

FIG. 37 illustrates the relationship between what clinician selected as what she/he would use to treat the patient before knowing what the molecular profiling results suggested. There were no matches for the 18 patients with PFS ratio ≧1.3.

FIG. 38 is a schematic of the overall survival for the 18 patients with PFS ratio ≧1.3 versus all 66 patients.

FIG. 39 illustrates a molecular profiling system that performs analysis of a cancer sample using a variety of components that measure expression levels, chromosomal aberrations and mutations. The molecular “blueprint” of the cancer is used to generate a prioritized ranking of druggable targets in tumor and their associated therapies.

FIG. 40 shows an example output of microarray profiling results and calls made using a cutoff value.

FIGS. 41A-41J illustrate an illustrative patient report based on molecular profiling.

FIGS. 42A-B illustrate a workflow chart for identifying a therapeutic for an individual having breast cancer. The workflow of FIG. 42A feeds into the workflow of FIG. 42B as indicated.

FIGS. 43A-B illustrates biomarkers used for identifying a therapeutic for an individual having breast cancer such as when following the workflow of FIG. 42. FIG. 43A illustrate a biomarker centric view of the workflow described above in different cancer settings. FIG. 43B illustrates additional biomarkers assessed depending on the criteria shown.

FIG. 44 illustrates the percentage of HER2 positive breast cancers that are likely to respond to treatment with trastuzumab (Herceptin®), which is about 30%. Characteristics of the tumor that can be identified by molecular profiling are shown as well.

FIGS. 45A-45N show an illustrative patient report based on molecular profiling.

FIG. 46 illustrates a diagram showing a biomarker centric (FIG. 46A) and therapeutic centric (FIG. 46B) approach to identifying a therapeutic agent.

### DETAILED DESCRIPTION OF THE INVENTION

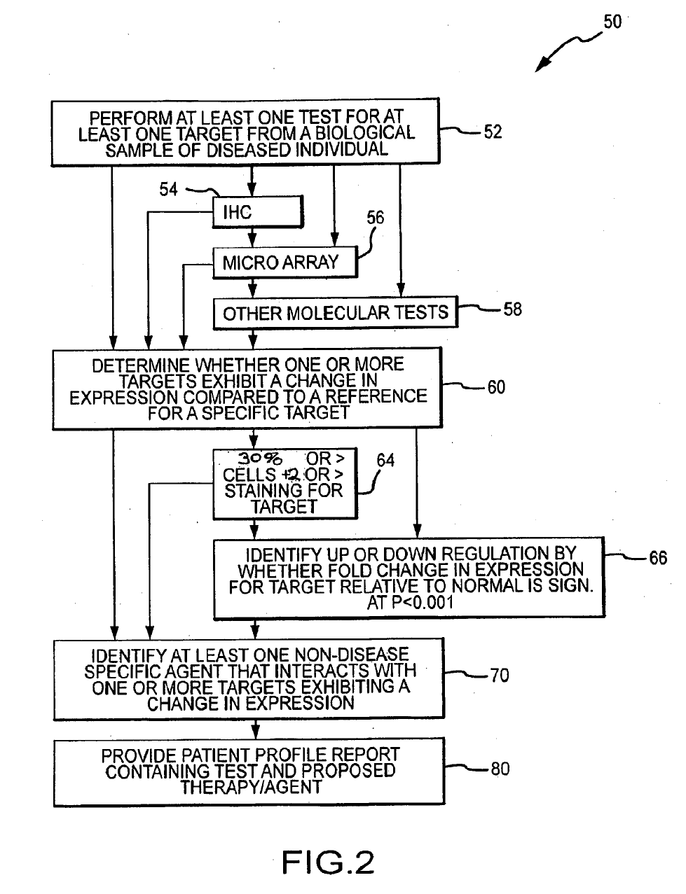

The present invention provides methods and systems for identifying therapeutic agents for use in treatments on an individualized basis by using molecular profiling. The molecular profiling approach provides a method for selecting a candidate treatment for an individual that could favorably change the clinical course for the individual with a condition or disease, such as cancer. The molecular profiling approach provides clinical benefit for individuals, such as identifying drug target(s) that provide a longer progression free survival (PFS), longer disease free survival (DFS), longer overall survival (OS) or extended lifespan. Methods and systems of the invention are directed to molecular profiling of cancer on an individual basis that can provide alternatives for treatment that may be convention or alternative to conventional treatment regimens. For example, alternative treatment regimes can be selected through molecular profiling methods of the invention where, a disease is refractory to current therapies, e.g., after a cancer has developed resistance to a standard-of-care treatment. Illustrative schemes for using molecular profiling to identify a treatment regime are shown in FIGS. 2, 39 and 42, each of which is described in further detail herein.

Molecular profiling can be performed by any known means for detecting a molecule in a biological sample. Molecular profiling comprises methods that include but are not limited to, nucleic acid sequencing, such as a DNA sequencing or mRNA sequencing; immunohistochemistry (IHC); in situ hybridization (ISH); fluorescent in situ hybridization (FISH); various types of microarray (mRNA expression arrays, protein arrays, etc); various types of sequencing (Sanger, pyrosequencing, etc); comparative genomic hybridization (CGH); NextGen sequencing; Northern blot; Southern blot; immunoassay; and any other appropriate technique to assay the presence or quantity of a biological molecule of interest. In various embodiments of the invention, any one or more of these methods can be used concurrently or subsequent to each other for assessing target genes disclosed herein.

Molecular profiling of individual samples is used to select one or more candidate treatments for a disorder in a subject, e.g., by identifying targets for drugs that may be effective for a given cancer. For example, the candidate treatment can be a treatment known to have an effect on cells that differentially express genes as identified by molecular profiling techniques, an experimental drug, a government or regulatory approved drug or any combination of such drugs, which may have been studied and approved for a particular indication that is the same as or different from the indication of the subject from whom a biological sample is obtain and molecularly profiled.

When multiple biomarker targets are revealed by assessing target genes by molecular profiling, one or more decision rules can be put in place to prioritize the selection of certain therapeutic agent for treatment of an individual on a personalized basis. Rules of the invention aide prioritizing treatment, e.g., direct results of molecular profiling, anticipated efficacy of therapeutic agent, prior history with the same or other treatments, expected side effects, availability of therapeutic agent, cost of therapeutic agent, drug-drug interactions, and other factors considered by a treating physician. Based on the recommended and prioritized therapeutic agent targets, a physician can decide on the course of treatment for a particular individual. Accordingly, molecular profiling methods and systems of the invention can select candidate treatments based on individual characteristics of diseased cells, e.g., tumor cells, and other personalized factors in a subject in need of treatment, as opposed to relying on a traditional one-size fits all approach that is conventionally used to treat individuals suffering from a disease, especially cancer. In some cases, the recommended treatments are those not typically used to treat the disease or disorder inflicting the subject. In some cases, the recommended treatments are used after standard-of-care therapies are no longer providing adequate efficacy.

Biological Entities

Nucleic acids include deoxyribonucleotides or ribonucleotides and polymers thereof in either single- or double-stranded form, or complements thereof. Nucleic acids can contain known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleic acid sequence can encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell Probes 8:91-98 (1994)). The term nucleic acid can be used interchangeably with gene, cDNA, mRNA, oligonucleotide, and polynucleotide.

A particular nucleic acid sequence may implicitly encompass the particular sequence and “splice variants” and nucleic acid sequences encoding truncated forms. Similarly, a particular protein encoded by a nucleic acid can encompass any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or created using recombinant techniques.

The terms “genetic variant” and “nucleotide variant” are used herein interchangeably to refer to changes or alterations to the reference human gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The genetic variant or nucleotide variant may occur in transcriptional regulatory regions, untranslated regions of mRNA, exons, introns, exon/intron junctions, etc. The genetic variant or nucleotide variant can potentially result in stop codons, frame shifts, deletions of amino acids, altered gene transcript splice forms or altered amino acid sequence.

An allele or gene allele comprises generally a naturally occurring gene having a reference sequence or a gene containing a specific nucleotide variant.

A haplotype refers to a combination of genetic (nucleotide) variants in a region of an mRNA or a genomic DNA on a chromosome found in an individual. Thus, a haplotype includes a number of genetically linked polymorphic variants which are typically inherited together as a unit.

As used herein, the term “amino acid variant” is used to refer to an amino acid change to a reference human protein sequence resulting from genetic variants or nucleotide variants to the reference human gene encoding the reference protein. The term “amino acid variant” is intended to encompass not only single amino acid substitutions, but also amino acid deletions, insertions, and other significant changes of amino acid sequence in the reference protein.

The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous. Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s).

The term “locus” refers to a specific position or site in a gene sequence or protein. Thus, there may be one or more contiguous nucleotides in a particular gene locus, or one or more amino acids at a particular locus in a polypeptide. Moreover, a locus may refer to a particular position in a gene where one or more nucleotides have been deleted, inserted, or inverted.

As used herein, the terms “polypeptide,” “protein,” and “peptide” are used interchangeably to refer to an amino acid chain in which the amino acid residues are linked by covalent peptide bonds. The amino acid chain can be of any length of at least two amino acids, including full-length proteins. Unless otherwise specified, polypeptide, protein, and peptide also encompass various modified forms thereof, including but not limited to glycosylated forms, phosphorylated forms, etc. A polypeptide, protein or peptide can also be referred to as a gene product.

Lists of gene and gene products that can be assayed by molecular profiling techniques are presented herein. Lists of genes may be presented in the context of molecular profiling techniques that detect a gene product (e.g., an mRNA or protein). One of skill will understand that this implies detection of the gene product of the listed genes. Similarly, lists of gene products may be presented in the context of molecular profiling techniques that detect a gene sequence or copy number. One of skill will understand that this implies detection of the gene corresponding to the gene products, including as an example DNA encoding the gene products. As will be appreciated by those skilled in the art, a “biomarker” or “marker” comprises a gene and/or gene product depending on the context.

The terms “label” and “detectable label” can refer to any composition detectable by spectroscopic, photochemical, biochemical, immunochemical, electrical, optical, chemical or similar methods. Such labels include biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. Labels can include, e.g., ligands that bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, N.Y. (1997); and in Haugland Handbook of Fluorescent Probes and Research Chemicals, a combined handbook and catalogue Published by Molecular Probes, Inc. (1996).

Detectable labels include, but are not limited to, nucleotides (labeled or unlabelled), compomers, sugars, peptides, proteins, antibodies, chemical compounds, conducting polymers, binding moieties such as biotin, mass tags, calorimetric agents, light emitting agents, chemiluminescent agents, light scattering agents, fluorescent tags, radioactive tags, charge tags (electrical or magnetic charge), volatile tags and hydrophobic tags, biomolecules (e.g., members of a binding pair antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B 12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) and the like.

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3.sup.rd Ed., W. H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art. See, e.g., Winter and Harris, Immunol. Today 14:243-246 (1993); Ward et al., Nature 341:544-546 (1989); Harlow and Lane, Antibodies, 511-52, Cold Spring Harbor Laboratory publications, New York, 1988; Hilyard et al., Protein Engineering: A practical approach (IRL Press 1992); Borrebaeck, Antibody Engineering, 2d ed. (Oxford University Press 1995); each of which is incorporated herein by reference.

Unless otherwise specified, antibodies can include both polyclonal and monoclonal antibodies. Antibodies also include genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immunol:5368, Zhu et al. (1997) Protein Sci 6:781, Hu et al. (1997) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four framework regions interrupted by three hyper-variable regions, also called complementarity-determining regions (CDRs). The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional spaces. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{II} CDR3 is located in the variable domain of the heavy chain of the antibody in which it is found, whereas a V_{L} CDR1 is the CDR1 from the variable domain of the light chain of the antibody in which it is found. References to V_{H} refer to the variable region of an immunoglobulin heavy chain of an antibody, including the heavy chain of an Fv, scFv, or Fab. References to V_{L} refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site. A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-327 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope” and “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “primer”, “probe,” and “oligonucleotide” are used herein interchangeably to refer to a relatively short nucleic acid fragment or sequence. They can comprise DNA, RNA, or a hybrid thereof, or chemically modified analog or derivatives thereof. Typically, they are single-stranded. However, they can also be double-stranded having two complementing strands which can be separated by denaturation. Normally, primers, probes and oligonucleotides have a length of from about 8 nucleotides to about 200 nucleotides, preferably from about 12 nucleotides to about 100 nucleotides, and more preferably about 18 to about 50 nucleotides. They can be labeled with detectable markers or modified using conventional manners for various molecular biological applications.

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Because a naturally existing chromosome (or a viral equivalent thereof) includes a long nucleic acid sequence, an isolated nucleic acid can be a nucleic acid molecule having only a portion of the nucleic acid sequence in the chromosome but not one or more other portions present on the same chromosome. More specifically, an isolated nucleic acid can include naturally occurring nucleic acid sequences that flank the nucleic acid in the naturally existing chromosome (or a viral equivalent thereof). An isolated nucleic acid can be substantially separated from other naturally occurring nucleic acids that are on a different chromosome of the same organism. An isolated nucleic acid can also be a composition in which the specified nucleic acid molecule is significantly enriched so as to constitute at least 10%, 20%, 30%, 40%, 50%, 60%, 70%, 80%, 90%, 95%, or at least 99% of the total nucleic acids in the composition.

An isolated nucleic acid can be a hybrid nucleic acid having the specified nucleic acid molecule covalently linked to one or more nucleic acid molecules that are not the nucleic acids naturally flanking the specified nucleic acid. For example, an isolated nucleic acid can be in a vector. In addition, the specified nucleic acid may have a nucleotide sequence that is identical to a naturally occurring nucleic acid or a modified form or mutein thereof having one or more mutations such as nucleotide substitution, deletion/insertion, inversion, and the like.

An isolated nucleic acid can be prepared from a recombinant host cell (in which the nucleic acids have been recombinantly amplified and/or expressed), or can be a chemically synthesized nucleic acid having a naturally occurring nucleotide sequence or an artificially modified form thereof.

The term “isolated polypeptide” as used herein is defined as a polypeptide molecule that is present in a form other than that found in nature. Thus, an isolated polypeptide can be a non-naturally occurring polypeptide. For example, an isolated polypeptide can be a “hybrid polypeptide.” An isolated polypeptide can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

The term “high stringency hybridization conditions,” when used in connection with nucleic acid hybridization, includes hybridization conducted overnight at 42° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, includes hybridization conducted overnight at 37° C. in a solution containing 50% formamide, 5×SSC (750 mM NaCl, 75 mM sodium citrate), 50 mM sodium phosphate, pH 7.6, S×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

For the purpose of comparing two different nucleic acid or polypeptide sequences, one sequence (test sequence) may be described to be a specific percentage identical to another sequence (comparison sequence). The percentage identity can be determined by the algorithm of Karlin and Altschul, Proc. Natl. Acad. Sci. USA, 90:5873-5877 (1993), which is incorporated into various BLAST programs. The percentage identity can be determined by the “BLAST 2 Sequences” tool, which is available at the National Center for Biotechnology Information (NCBI) website. See Tatusova and Madden, FEMS Microbiol. Lett., 174(2):247-250 (1999). For pairwisc DNA-DNA comparison, the BLASTN program is used with default parameters (e.g., Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP program can be employed using default parameters (e.g., Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST is used to compare two sequences, it aligns the sequences and yields the percent identity over defined, aligned regions. If the two sequences are aligned across their entire length, the percent identity yielded by the BLAST is the percent identity of the two sequences. If BLAST does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence. Various versions of the BLAST programs can be used to compare sequences, e.g., BLAST 2.1.2 or BLAST+ 2.2.22.

A subject or individual can be any animal which may benefit from the methods of the invention, including, e.g., humans and non-human mammals, such as primates, rodents, horses, dogs and cats. Subjects include without limitation a eukaryotic organisms, most preferably a mammal such as a primate, e.g., chimpanzee or human, cow; dog; cat; a rodent, e.g., guinea pig, rat, mouse; rabbit; or a bird; reptile; or fish. Subjects specifically intended for treatment using the methods described herein include humans. A subject may be referred to as an individual or a patient.

Treatment of a disease or individual according to the invention is an approach for obtaining beneficial or desired medical results, including clinical results, but not necessarily a cure. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment also includes prolonging survival as compared to expected survival if not receiving treatment or if receiving a different treatment. A treatment can include administration of a therapeutic agent, which can be an agent that exerts a cytotoxic, cytostatic, or immunomodulatory effect on diseased cells, e.g., cancer cells, or other cells that may promote a diseased state, e.g., activated immune cells. Therapeutic agents selected by the methods of the invention are not limited. Any therapeutic agent can be selected where a link can be made between molecular profiling and potential efficacy of the agent. Therapeutic agents include without limitation drugs, small molecules, protein therapies, antibody therapies, viral therapies, gene therapies, and the like. Cancer treatments or therapies include apoptosis-mediated and non-apoptosis mediated cancer therapies including, without limitation, chemotherapy, hormonal therapy, radiotherapy, immunotherapy, and combinations thereof. Chemotherapeutic agents comprise therapeutic agents and combinations of therapeutic agents that treat, cancer cells, e.g., by killing those cells. Examples of different types of chemotherapeutic drugs include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids).

A biomarker refers generally to a molecule, including a gene or product thereof, nucleic acid, protein, carbohydrate structure, or glycolipid, characteristics of which can be detected in a tissue or cell to provide information that is predictive, diagnostic, prognostic and/or theranostic for sensitivity or resistance to candidate treatment.

Biological Samples

A sample as used herein includes any relevant biological sample that can be used for molecular profiling, e.g., sections of tissues such as biopsy or tissue removed during surgical or other procedures, bodily fluids, autopsy samples, and frozen sections taken for histological purposes. Such samples include blood and blood fractions or products (e.g., serum, huffy coat, plasma, platelets, red blood cells, and the like), sputum, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. A sample may be processed according to techniques understood by those in the art. A sample can be without limitation fresh, frozen or fixed cells or tissue. In some embodiments, a sample comprises formalin-fixed paraffin-embedded (FFPE) tissue, fresh tissue or fresh frozen (FF) tissue. A sample can comprise cultured cells, including primary or immortalized cell lines derived from a subject sample. A sample can also refer to an extract from a sample from a subject. For example, a sample can comprise DNA, RNA or protein extracted from a tissue or a bodily fluid. Many techniques and commercial kits are available for such purposes. The fresh sample from the individual can be treated with an agent to preserve RNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample. A sample is typically obtained from a subject.

A biopsy comprises the process of removing a tissue sample for diagnostic or prognostic evaluation, and to the tissue specimen itself. Any biopsy technique known in the art can be applied to the molecular profiling methods of the present invention. The biopsy technique applied can depend on the tissue type to be evaluated (e.g., colon, prostate, kidney, bladder, lymph node, liver, bone marrow, blood cell, lung, breast, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Molecular profiling can use a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Standard molecular biology techniques known in the art and not specifically described are generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) can be carried out generally as in PCR Protocols: A Guide to Methods and Applications, Academic Press, San Diego, Calif. (1990).

Gene Expression Profiling

The methods and systems of the invention comprise expression profiling, which includes assessing differential expression of one or more target genes disclosed herein. Differential expression can include overexpression and/or underexpression of a biological product, e.g., a gene, mRNA or protein, compared to a control (or a reference). The control can include similar cells to the sample but without the disease (e.g., expression profiles obtained from samples from healthy individuals). A control can be a previously determined level that is indicative of a drug target efficacy associated with the particular disease and the particular drug target. The control can be derived from the same patient, e.g., a normal adjacent portion of the same organ as the diseased cells, the control can be derived from healthy tissues from other patients, or previously determined thresholds that are indicative of a disease responding or not-responding to a particular drug target. The control can also be a control found in the same sample, e.g. a housekeeping gene or a product thereof (e.g., mRNA or protein). For example, a control nucleic acid can be one which is known not to differ depending on the cancerous or non-cancerous state of the cell. The expression level of a control nucleic acid can be used to normalize signal levels in the test and reference populations. Illustrative control genes include, but are not limited to, e.g., β-actin, glyceraldehyde 3-phosphate dehydrogenase and ribosomal protein P1. Multiple controls or types of controls can be used. The source of differential expression can vary. For example, a gene copy number may be increased in a cell, thereby resulting in increased expression of the gene. Alternately, transcription of the gene may be modified, e.g., by chromatin remodeling, differential methylation, differential expression or activity of transcription factors, etc. Translation may also be modified, e.g., by differential expression of factors that degrade mRNA, translate mRNA, or silence translation, e.g., microRNAs or siRNAs. In some embodiments, differential expression comprises differential activity. For example, a protein may carry a mutation that increases the activity of the protein, such as constitutive activation, thereby contributing to a diseased state. Molecular profiling that reveals changes in activity can be used to guide treatment selection.

Methods of gene expression profiling include methods based on hybridization analysis of polynucleotides, and methods based on sequencing of polynucleotides. Commonly used methods known in the art for the quantification of mRNA expression in a sample include northern blotting and in situ hybridization (Parker & Barnes (1999) Methods in Molecular Biology 106:247-283); RNAse protection assays (Hod (1992) Biotechniques 13:852-854); and reverse transcription polymerase chain reaction (RT-PCR) (Weis et al. (1992) Trends in Genetics 8:263-264). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Reverse Transcriptase PCR (RT-PCR)

RT-PCR can be used to determine RNA levels, e.g., mRNA or miRNA levels, of the biomarkers of the invention. RT-PCR can be used to compare such RNA levels of the biomarkers of the invention in different sample populations, in normal and tumor tissues, with or without drug treatment, to characterize patterns of gene expression, to discriminate between closely related RNAs, and to analyze RNA structure.

The first step is the isolation of RNA, e.g., mRNA, from a sample. The starting material can be total RNA isolated from human tumors or tumor cell lines, and corresponding normal tissues or cell lines, respectively. Thus RNA can be isolated from a sample, e.g., tumor cells or tumor cell lines, and compared with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using purification kit, buffer set and protease from commercial manufacturers, such as Qiagen, according to the manufacturer's instructions (QIAGEN Inc., Valencia, Calif.). For example, total RNA from cells in culture can be isolated using Qiagen RNeasy mini-columns. Numerous RNA isolation kits are commercially available and can be used in the methods of the invention.

In the alternative, the first step is the isolation of miRNA from a target sample. The starting material is typically total RNA isolated from human tumors or tumor cell lines, and corresponding normal tissues or cell lines, respectively. Thus RNA can be isolated from a variety of primary tumors or tumor cell lines, with pooled DNA from healthy donors. If the source of miRNA is a primary tumor, miRNA can be extracted, for example, from frozen or archived paraffin-embedded and fixed (e.g. formalin-fixed) tissue samples.

General methods for miRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Numerous RNA isolation kits are commercially available and can be used in the methods of the invention.

Whether the RNA comprises mRNA, miRNA or other types of RNA, gene expression profiling by RT-PCR can include reverse transcription of the RNA template into cDNA, followed by amplification in a PCR reaction. Commonly used reverse transcriptases include, but are not limited to, avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif., USA), or LightCycler (Roche Molecular Biochemicals, Mannheim, Germany). In one specific embodiment, the 5′ nuclease procedure is run on a real-time quantitative PCR device such as the ART PRISM 7700 Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optic cables for all 96 wells, and detected at the CCD. The system includes software for running the instrument and for analyzing the data.

TaqMan data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

Real time quantitative PCR (also quantitative real time polymerase chain reaction, QRT-PCR or Q-PCR) is a more recent variation of the RT-PCR technique. Q-PCR can measure PCR product accumulation through a dual-labeled fluorogenic probe (i.e., TaqMan probe). Real time PCR is compatible both with quantitative competitive PCR, where internal competitor for each target sequence is used for normalization, and with quantitative comparative PCR using a normalization gene contained within the sample, or a housekeeping gene for RT-PCR. See, e.g. Held et al. (1996) Genome Research 6:986-994.

Protein-based detection techniques are also useful for molecular profiling, especially when the nucleotide variant causes amino acid substitutions or deletions or insertions or frame shift that affect the protein primary, secondary or tertiary structure. To detect the amino acid variations, protein sequencing techniques may be used. For example, a protein or fragment thereof corresponding to a gene can be synthesized by recombinant expression using a DNA fragment isolated from an individual to be tested. Preferably, a cDNA fragment of no more than 100 to 150 base pairs encompassing the polymorphic locus to be determined is used. The amino acid sequence of the peptide can then be determined by conventional protein sequencing methods. Alternatively, the HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected is analyzed. See Gatlin et al., Anal. Chem., 72:757-763 (2000).

Microarray

The biomarkers of the invention can also be identified, confirmed, and/or measured using the microarray technique. Thus, the expression profile biomarkers can be measured in cancer samples using microarray technology. In this method, polynucleotide sequences of interest are plated, or arrayed, on a microchip substrate. The arrayed sequences are then hybridized with specific DNA probes from cells or tissues of interest. The source of mRNA can be total RNA isolated from a sample, e.g., human tumors or tumor cell lines and corresponding normal tissues or cell lines. Thus RNA can be isolated from a variety of primary tumors or tumor cell lines. If the source of mRNA is a primary tumor, niRNA can be extracted, for example, from frozen or archived paraffin-embedded and fixed (e.g. formalin-fixed) tissue samples, which are routinely prepared and preserved in everyday clinical practice.

The expression profile of biomarkers can be measured in either fresh or paraffin-embedded tumor tissue, or body fluids using microarray technology. In this method, polynucleotide sequences of interest are plated, or arrayed, on a microchip substrate. The arrayed sequences are then hybridized with specific DNA probes from cells or tissues of interest. As with the RT-PCR method, the source of miRNA typically is total RNA isolated from human tumors or tumor cell lines, including body fluids, such as serum, urine, tears, and exosomes and corresponding normal tissues or cell lines. Thus RNA can be isolated from a variety of sources. If the source of miRNA is a primary tumor, miRNA can be extracted, for example, from frozen tissue samples, which are routinely prepared and preserved in everyday clinical practice.

Also known as biochip, DNA chip, or gene array, cDNA microarray technology allows for identification of gene expression levels in a biologic sample. cDNAs or oligonucleotides, each representing a given gene, are immobilized on a substrate, e.g., a small chip, bead or nylon membrane, tagged, and serve as probes that will indicate whether they are expressed in biologic samples of interest. The simultaneous expression of thousands of genes can be monitored simultaneously.

In a specific embodiment of the microarray technique, PCR amplified inserts of cDNA clones are applied to a substrate in a dense array. In one aspect, at least 100, 200, 300, 400, 500, 600, 700, 800, 900, 1,000, 1,500, 2,000, 3000, 4000, 5000, 6000, 7000, 8000, 9000, 10,000, 15,000, 20,000, 25,000, 30,000, 35,000, 40,000, 45,000 or at least 50,000 nucleotide sequences are applied to the substrate. Each sequence can correspond to a different gene, or multiple sequences can be arrayed per gene. The microarrayed genes, immobilized on the microchip, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al. (1996) Proc. Natl. Acad. Sci. USA 93(2):106-149). Microarray analysis can be performed by commercially available equipment following manufacturer's protocols, including without limitation the Affymetrix GeneChip technology (Affymetrix, Santa Clara, Calif.), Agilent (Agilent Technologies, Inc., Santa Clara, Calif.), or Illumina (Illumina, Inc., San Diego, Calif.) microarray technology.

The development of microarray methods for large-scale analysis of gene expression makes it possible to search systematically for molecular markers of cancer classification and outcome prediction in a variety of tumor types.

In some embodiments, the Agilent Whole Human Genome Microarray Kit (Agilent Technologies, Inc., Santa Clara, Calif.). The system can analyze more than 41,000 unique human genes and transcripts represented, all with public domain annotations. The system is used according to the manufacturer's instructions.

In some embodiments, the Illumina Whole Genome DASL assay (Illumina Inc., San Diego, Calif.) is used. The system offers a method to simultaneously profile over 24,000 transcripts from minimal RNA input, from both fresh frozen (FF) and formalin-fixed paraffin embedded (FFPE) tissue sources, in a high throughput fashion.

Microarray expression analysis comprises identifying whether a gene or gene product is up-regulated or down-regulated relative to a reference. The identification can be performed using a statistical test to determine statistical significance of any differential expression observed. In some embodiments, statistical significance is determined using a parametric statistical test. The parametric statistical test can comprise, for example, a fractional factorial design, analysis of variance (ANOVA), a t-test, least squares, a Pearson correlation, simple linear regression, nonlinear regression, multiple linear regression, or multiple nonlinear regression. Alternatively, the parametric statistical test can comprise a one-way analysis of variance, two-way analysis of variance, or repeated measures analysis of variance. In other embodiments, statistical significance is determined using a nonparametric statistical test. Examples include, but are not limited to, a Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than about 0.05, 0.01, 0.005, 0.001, 0.0005, or 0.0001. Although the microarray systems used in the methods of the invention may assay thousands of transcripts, data analysis need only be performed on the transcripts of interest, thereby reducing the problem of multiple comparisons inherent in performing multiple statistical tests. The p-values can also be corrected for multiple comparisons, e.g., using a Bonferroni correction, a modification thereof, or other technique known to those in the art, e.g., the Hochberg correction, Holm-Bonferroni correction, Sidak correction, or Dunnett's correction. The degree of differential expression can also be taken into account. For example, a gene can be considered as differentially expressed when the fold-change in expression compared to control level is at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold different in the sample versus the control. The differential expression takes into account both overexpression and underexpression. A gene or gene product can be considered up or down-regulated if the differential expression meets a statistical threshold, a fold-change threshold, or both. For example, the criteria for identifying differential expression can comprise both a p-value of 0.001 and fold change of at least 1.5-fold (up or down). One of skill will understand that such statistical and threshold measures can be adapted to determine differential expression by any molecular profiling technique disclosed herein.

Various methods of the invention make use of many types of microarrays that detect the presence and potentially the amount of biological entities in a sample. Arrays typically contain addressable moieties that can detect the presence of the entity in the sample, e.g., via a binding event. Microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). DNA arrays typically comprise addressable nucleotide sequences that can bind to sequences present in a sample. MicroRNA arrays, e.g., the MMChips array from the University of Louisville or commercial systems from Agilent, can be used to detect microRNAs. Protein microarrays can be used to identify protein-protein interactions, including without limitation identifying substrates of protein kinases, transcription factor protein-activation, or to identify the targets of biologically active small molecules. Protein arrays may comprise an array of different protein molecules, commonly antibodies, or nucleotide sequences that bind to proteins of interest. Antibody microarrays comprise antibodies spotted onto the protein chip that are used as capture molecules to detect proteins or other biological materials from a sample, e.g., from cell or tissue lysate solutions. For example, antibody arrays can be used to detect biomarkers from bodily fluids, e.g., serum or urine, for diagnostic applications. Tissue microarrays comprise separate tissue cores assembled in array fashion to allow multiplex histological analysis. Cellular microarrays, also called transfection microarrays, comprise various capture agents, such as antibodies, proteins, or lipids, which can interact with cells to facilitate their capture on addressable locations. Chemical compound microarrays comprise arrays of chemical compounds and can be used to detect protein or other biological materials that bind the compounds. Carbohydrate arrays (glycoarrays) comprise arrays of carbohydrates and can detect, e.g., protein that bind sugar moieties. One of skill will appreciate that similar technologies or improvements can be used according to the methods of the invention.

Gene Expression Analysis by Massively Parallel Signature Sequencing (MPSS)

This method, described by Brenner et al. (2000) Nature Biotechnology 18:630-634, is a sequencing approach that combines non-gel-based signature sequencing with in vitro cloning of millions of templates on separate microbeads. First, a microbead library of DNA templates is constructed by in vitro cloning. This is followed by the assembly of a planar array of the template-containing microbeads in a flow cell at a high density. The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a cDNA library.

MPSS data has many uses. The expression levels of nearly all transcripts can be quantitatively determined; the abundance of signatures is representative of the expression level of the gene in the analyzed tissue. Quantitative methods for the analysis of tag frequencies and detection of differences among libraries have been published and incorporated into public databases for SAGE™ data and are applicable to MPSS data. The availability of complete genome sequences permits the direct comparison of signatures to genomic sequences and further extends the utility of MPSS data. Because the targets for MPSS analysis are not pre-selected (like on a microarray), MPSS data can characterize the full complexity of transcriptomes. This is analogous to sequencing millions of ESTs at once, and genomic sequence data can be used so that the source of the MPSS signature can be readily identified by computational means.

Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (e.g., about 10-14 hp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, e.g. Velculescu et al. (1995) Science 270:484-487; and Velculescu et al. (1997) Cell 88:243-51.

DNA Copy Number Profiling

Any method capable of determining a DNA copy number profile of a particular sample can be used for molecular profiling according to the invention as long as the resolution is sufficient to identify the biomarkers of the invention. The skilled artisan is aware of and capable of using a number of different platforms for assessing whole genome copy number changes at a resolution sufficient to identify the copy number of the one or more biomarkers of the invention. Some of the platforms and techniques are described in the embodiments below.

In some embodiments, the copy number profile analysis involves amplification of whole genome DNA by a whole genome amplification method. The whole genome amplification method can use a strand displacing polymerase and random primers.

In some aspects of these embodiments, the copy number profile analysis involves hybridization of whole genome amplified DNA with a high density array. In a more specific aspect, the high density array has 5,000 or more different probes. In another specific aspect, the high density array has 5,000, 10,000, 20,000, 50,000, 100,000, 200,000, 300,000, 400,000, 500,000, 600,000, 700,000, 800,000, 900,000, or 1,000,000 or more different probes. In another specific aspect, each of the different probes on the array is an oligonucleotide having from about 15 to 200 bases in length. In another specific aspect, each of the different probes on the array is an oligonucleotide having from about 15 to 200, 15 to 150, 15 to 100, 15 to 75, 15 to 60, or 20 to 55 bases in length.

In some embodiments, a microarray is employed to aid in determining the copy number profile for a sample, e.g., cells from a tumor. Microarrays typically comprise a plurality of oligomers (e.g., DNA or RNA polynucleotides or oligonucleotides, or other polymers), synthesized or deposited on a substrate (e.g., glass support) in an array pattern. The support-bound oligomers are “probes”, which function to hybridize or bind with a sample material (e.g., nucleic acids prepared or obtained from the tumor samples), in hybridization experiments. The reverse situation can also be applied: the sample can be hound to the microarray substrate and the oligomer probes are in solution for the hybridization. In use, the array surface is contacted with one or more targets under conditions that promote specific, high-affinity binding of the target to one or more of the probes. In some configurations, the sample nucleic acid is labeled with a detectable label, such as a fluorescent tag, so that the hybridized sample and probes are detectable with scanning equipment. DNA array technology offers the potential of using a multitude (e.g., hundreds of thousands) of different oligonucleotides to analyze DNA copy number profiles. In some embodiments, the substrates used for arrays are surface-derivatized glass or silica, or polymer membrane surfaces (see e.g., in Z. Guo, et al., Nucleic Acids Res, 22, 5456-65 (1994); U. Maskos, E. M. Southern, Nucleic Acids Res, 20, 1679-84 (1992), and E. M. Southern, et al., Nucleic Acids Res, 22, 1368-73 (1994), each incorporated by reference herein). Modification of surfaces of array substrates can be accomplished by many techniques. For example, siliceous or metal oxide surfaces can be derivatized with bifunctional silanes, i.e., silanes having a first functional group enabling covalent binding to the surface (e.g., Si-halogen or Si-alkoxy group, as in —SiCl₃ or —Si(OCH₃)₃, respectively) and a second functional group that can impart the desired chemical and/or physical modifications to the surface to covalently or non-covalently attach ligands and/or the polymers or monomers for the biological probe array. Silylated derivatizations and other surface derivatizations that are known in the art (see for example U.S. Pat. No. 5,624,711 to Sundberg, U.S. Pat. No. 5,266,222 to Willis, and U.S. Pat. No. 5,137,765 to Farnsworth, each incorporated by reference herein). Other processes for preparing arrays are described in U.S. Pat. No. 6,649,348, to Bass et. al., assigned to Agilent Corp., which disclose DNA arrays created by in situ synthesis methods.

Polymer array synthesis is also described extensively in the literature including in the following: WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, 5,412,087, 6,147,205, 6,262,216, 6,310,189, 5,889,165, and 5,959,098 in PCT Applications Nos. PCT/US99/00730 (International Publication No. WO 99/36760) and PCT/US01/04285 (International Publication No. WO 01/58593), which are all incorporated herein by reference in their entirety for all purposes.

Nucleic acid arrays that are useful in the present invention include, but are not limited to, those that are commercially available from Affymetrix (Santa Clara, Calif.) under the brand name GeneChip™. Example arrays are shown on the website at affymetrix.com. Another microarray supplier is Illumina, Inc., of San Diego, Calif. with example arrays shown on their website at illumina.com.

In some embodiments, the inventive methods provide for sample preparation. Depending on the microarray and experiment to be performed, sample nucleic acid can be prepared in a number of ways by methods known to the skilled artisan. In some aspects of the invention, prior to or concurrent with genotyping (analysis of copy number profiles), the sample may be amplified any number of mechanisms. The most common amplification procedure used involves PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. In some embodiments, the sample may be amplified on the array (e.g., U.S. Pat. No. 6,300,070 which is incorporated herein by reference)

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), U.S. Pat. No. 9/910,292 (U.S. Patent Application Publication 20030082543), and Ser. No. 10/013,598.

Methods for conducting polynucleotide hybridization assays are well developed in the art. Hybridization assay procedures and conditions used in the methods of the invention will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S. 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The methods of the invention may also involve signal detection of hybridization between ligands in after (and/or during) hybridization. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

Immuno-Based Assays

Protein-based detection molecular profiling techniques include immunoaffinity assays based on antibodies selectively immunoreactive with mutant gene encoded protein according to the present invention. These techniques include without limitation immunoprecipitation, Western blot analysis, molecular binding assays, enzyme-linked immunosorbent assay (ELISA), enzyme-linked immunofiltration assay (ELISA), fluorescence activated cell sorting (FACS) and the like. For example, an optional method of detecting the expression of a biomarker in a sample comprises contacting the sample with an antibody against the biomarker, or an immunoreactive fragment of the antibody thereof, or a recombinant protein containing an antigen binding region of an antibody against the biomarker; and then detecting the binding of the biomarker in the sample. Methods for producing such antibodies are known in the art. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well-known antibody-based techniques can also be used including, e.g., ELISA, radioimmunoassay (RIA), immunoradiometric assays (IRMA) and immunoenzymatic assays (IEMA), including sandwich assays using monoclonal or polyclonal antibodies. See, e.g., U.S. Pat. Nos. 4,376,110 and 4,486,530, both of which are incorporated herein by reference.

In alternative methods, the sample may be contacted with an antibody specific for a biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labelled antibody to a target biomarker.

A number of variations of the sandwich assay technique exist, and all are intended to be encompassed by the present invention. Briefly, in a typical forward assay, an unlabelled antibody is immobilized on a solid substrate, and the sample to be tested brought into contact with the hound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labelled antibody are added simultaneously to the hound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

Immunohistochemistry (IHC)

IHC is a process of localizing antigens (e.g., proteins) in cells of a tissue binding antibodies specifically to antigens in the tissues. The antigen-binding antibody can be conjugated or fused to a tag that allows its detection, e.g., via visualization. In some embodiments, the tag is an enzyme that can catalyze a color-producing reaction, such as alkaline phosphatase or horseradish peroxidase. The enzyme can be fused to the antibody or non-covalently bound, e.g., using a biotin-avadin system. Alternatively, the antibody can be tagged with a fluorophore, such as fluorescein, rhodamine, DyLight Fluor or Alexa Fluor. The antigen-binding antibody can be directly tagged or it can itself be recognized by a detection antibody that carries the tag. Using IIIC, one or more proteins may be detected. The expression of a gene product can be related to its staining intensity compared to control levels. In some embodiments, the gene product is considered differentially expressed if its staining varies at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold in the sample versus the control.

IHC comprises the application of antigen-antibody interactions to histochemical techniques. In an illustrative example, a tissue section is mounted on a slide and is incubated with antibodies (polyclonal or monoclonal) specific to the antigen (primary reaction). The antigen-antibody signal is then amplified using a second antibody conjugated to a complex of peroxidase antiperoxidase (PAP), avidin-biotin-peroxidase (ABC) or avidin-biotin alkaline phosphatase. In the presence of substrate and chromogen, the enzyme forms a colored deposit at the sites of antibody-antigen binding. Immunofluorescence is an alternate approach to visualize antigens. In this technique, the primary antigen-antibody signal is amplified using a second antibody conjugated to a fluorochrome. On UV light absorption, the fluorochrome emits its own light at a longer wavelength (fluorescence), thus allowing localization of antibody-antigen complexes.

Epigenetic Status

Molecular profiling methods according to the invention also comprise measuring epigenetic change, i.e., modification in a gene caused by an epigenetic mechanism, such as a change in methylation status or histone acetylation. Frequently, the epigenetic change will result in an alteration in the levels of expression of the gene which may be detected (at the RNA or protein level as appropriate) as an indication of the epigenetic change. Often the epigenetic change results in silencing or down regulation of the gene, referred to as “epigenetic silencing.” The most frequently investigated epigenetic change in the methods of the invention involves determining the DNA methylation status of a gene, where an increased level of methylation is typically associated with the relevant cancer (since it may cause down regulation of gene expression). Aberrant methylation, which may be referred to as hypermethylation, of the gene or genes can be detected. Typically, the methylation status is determined in suitable CpG islands which are often found in the promoter region of the gene(s). The term “methylation,” “methylation state” or “methylation status” may refers to the presence or absence of 5-methylcytosine at one or a plurality of CpG dinucleotides within a DNA sequence. CpG dinucleotides are typically concentrated in the promoter regions and exons of human genes.

Diminished gene expression can be assessed in terms of DNA methylation status or in terms of expression levels as determined by the methylation status of the gene. One method to detect epigenetic silencing is to determine that a gene which is expressed in normal cells is less expressed or not expressed in tumor cells. Accordingly, the invention provides for a method of molecular profiling comprising detecting epigenetic silencing.

Various assay procedures to directly detect methylation are known in the art, and can be used in conjunction with the present invention. These assays rely onto two distinct approaches: bisulphite conversion based approaches and non-bisulphite based approaches. Non-bisulphite based methods for analysis of DNA methylation rely on the inability of methylation-sensitive enzymes to cleave methylation cytosines in their restriction. The bisulphite conversion relies on treatment of DNA samples with sodium bisulphite which converts unmethylated cytosine to uracil, while methylated cytosines are maintained (Furuichi Y, Wataya Y, Hayatsu H, Ukita T. Biochem Biophys Res Commun. 1970 Dec. 9; 41(5):1185-91). This conversion results in a change in the sequence of the original DNA. Methods to detect such changes include MS AP-PCR (Methylation-Sensitive Arbitrarily-Primed Polymerase Chain Reaction), a technology that allows for a global scan of the genome using CG-rich primers to focus on the regions most likely to contain CpG dinucleotides, and described by Gonzalgo et al., Cancer Research 57:594-599, 1997; MethyLight™, which refers to the art-recognized fluorescence-based real-time PCR technique described by Eads et al., Cancer Res. 59:2302-2306, 1999; the HeavyMethyl™ assay, in the embodiment thereof implemented herein, is an assay, wherein methylation specific blocking probes (also referred to herein as blockers) covering CpG positions between, or covered by the amplification primers enable methylation-specific selective amplification of a nucleic acid sample; HeavyMethyl™ MethyLight™ is a variation of the MethyLight™ assay wherein the MethyLight™ assay is combined with methylation specific blocking probes covering CpG positions between the amplification primers; Ms-SNuPE (Methylation-sensitive Single Nucleotide Primer Extension) is an assay described by Gonzalgo & Jones, Nucleic Acids Res. 25:2529-2531, 1997; MSP (Methylation-specific PCR) is a methylation assay described by Herman et al. Proc. Natl. Acad. Sci. USA 93:9821-9826, 1996, and by U.S. Pat. No. 5,786,146; COBRA (Combined Bisulfite Restriction Analysis) is a methylation assay described by Xiong & Laird, Nucleic Acids Res. 25:2532-2534, 1997; MCA (Methylated CpG Island Amplification) is a methylation assay described by Toyota et al., Cancer Res. 59:2307-12, 1999, and in WO 00/26401A1.

Other techniques for DNA methylation analysis include sequencing, methylation-specific PCR (MS-PCR), melting curve methylation-specific PCR (McMS-PCR), MLPA with or without bisulfite treatment, QAMA, MSRE-PCR, MethyLight, ConLight-MSP, bisulfite conversion-specific methylation-specific PCR (BS-MSP), COBRA (which relies upon use of restriction enzymes to reveal methylation dependent sequence differences in PCR products of sodium bisulfite-treated DNA), methylation-sensitive single-nucleotide primer extension conformation (MS-SNuPE), methylation-sensitive single-strand conformation analysis (MS-SSCA), Melting curve combined bisulfite restriction analysis (McCOBRA), PyroMethA, HeavyMethyl, MALDI-TOF, MassARRAY, Quantitative analysis of methylated alleles (QAMA), enzymatic regional methylation assay (ERMA), QBSUPT, MethylQuant, Quantitative PCR sequencing and oligonucleotide-based microarray systems, Pyrosequencing, Meth-DOP-PCR. A review of some useful techniques is provided in Nucleic acids research, 1998, Vol. 26, No. 10, 2255-2264; Nature Reviews, 2003, Vol. 3, 253-266; Oral Oncology, 2006, Vol. 42, 5-13, which references are incorporated herein in their entirety. Any of these techniques may be utilized in accordance with the present invention, as appropriate. Other techniques are described in U.S. Patent Publications 20100144836; and 20100184027, which applications are incorporated herein by reference in their entirety.

Through the activity of various acetylases and deacetylylases the DNA binding function of histone proteins is tightly regulated. Furthermore, histone acetylation and histone deactelyation have been linked with malignant progression. See Nature, 429: 457-63, 2004. Methods to analyze hi stone acetylation are described in U.S. Patent Publications 20100144543 and 20100151468, which applications are incorporated herein by reference in their entirety.

Sequence Analysis

Molecular profiling according to the present invention comprises methods for genotyping one or more biomarkers by determining whether an individual has one or more nucleotide variants (or amino acid variants) in one or more of the genes or gene products. Genotyping one or more genes according to the methods of the invention in some embodiments, can provide more evidence for selecting a treatment.

The biomarkers of the invention can be analyzed by any method useful for determining alterations in nucleic acids or the proteins they encode. According to one embodiment, the ordinary skilled artisan can analyze the one or more genes for mutations including deletion mutants, insertion mutants, frame shift mutants, nonsense mutants, missense mutant, and splice mutants.

Nucleic acid used for analysis of the one or more genes can be isolated from cells in the sample according to standard methodologies (Sambrook et al., 1989). The nucleic acid, for example, may be genomic DNA or fractionated or whole cell RNA, or miRNA acquired from exosomes or cell surfaces. Where RNA is used, it may be desired to convert the RNA to a complementary DNA. In one embodiment, the RNA is whole cell RNA; in another, it is poly-A RNA; in another, it is exosomal RNA. Normally, the nucleic acid is amplified. Depending on the format of the assay for analyzing the one or more genes, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects are known to occur in the biomarkers of the invention. Alterations include without limitation deletions, insertions, point mutations, and duplications. Point mutations can be silent or can result in stop codons, frame shift mutations or amino acid substitutions. Mutations in and outside the coding region of the one or more genes may occur and can be analyzed according to the methods of the invention. The target site of a nucleic acid of interest can include the region wherein the sequence varies. Examples include, but are not limited to, polymorphisms which exist in different forms such as single nucleotide variations, nucleotide repeats, multibase deletion (more than one nucleotide deleted from the consensus sequence), multibase insertion (more than one nucleotide inserted from the consensus sequence), microsatellite repeats (small numbers of nucleotide repeats with a typical 5-1000 repeat units), di-nucleotide repeats, tri-nucleotide repeats, sequence rearrangements (including translocation and duplication), chimeric sequence (two sequences from different gene origins are fused together), and the like. Among sequence polymorphisms, the most frequent polymorphisms in the human genome are single-base variations, also called single-nucleotide polymorphisms (SNPs). SNPs are abundant, stable and widely distributed across the genome.

Molecular profiling includes methods for haplotyping one or more genes. The haplotype is a set of genetic determinants located on a single chromosome and it typically contains a particular combination of alleles (all the alternative sequences of a gene) in a region of a chromosome. In other words, the haplotype is phased sequence information on individual chromosomes. Very often, phased SNPs on a chromosome define a haplotype. A combination of haplotypes on chromosomes can determine a genetic profile of a cell. It is the haplotype that determines a linkage between a specific genetic marker and a disease mutation. Haplotyping can be done by any methods known in the art. Common methods of scoring SNPs include hybridization microarray or direct gel sequencing, reviewed in Landgren et al., Genome Research, 8:769-776, 1998. For example, only one copy of one or more genes can be isolated from an individual and the nucleotide at each of the variant positions is determined. Alternatively, an allele specific PCR or a similar method can be used to amplify only one copy of the one or more genes in an individual, and the SNPs at the variant positions of the present invention are determined. The Clark method known in the art can also be employed for haplotyping. A high throughput molecular haplotyping method is also disclosed in Tost et al., Nucleic Acids Res., 30(19):e96 (2002), which is incorporated herein by reference.

Thus, additional variant(s) that are in linkage disequilibrium with the variants and/or haplotypes of the present invention can be identified by a haplotyping method known in the art, as will be apparent to a skilled artisan in the field of genetics and haplotyping. The additional variants that are in linkage disequilibrium with a variant or haplotype of the present invention can also be useful in the various applications as described below.

For purposes of genotyping and haplotyping, both genomic DNA and mRNA/cDNA can be used, and both are herein referred to generically as “gene.”

Numerous techniques for detecting nucleotide variants are known in the art and can all be used for the method of this invention. The techniques can be protein-based or nucleic acid-based. In either case, the techniques used must be sufficiently sensitive so as to accurately detect the small nucleotide or amino acid variations. Very often, a probe is utilized which is labeled with a detectable marker. Unless otherwise specified in a particular technique described below, any suitable marker known in the art can be used, including but not limited to, radioactive isotopes, fluorescent compounds, biotin which is detectable using streptavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a nucleic acid-based detection method, target DNA sample, i.e., a sample containing genomic DNA, cDNA, mRNA and/or miRNA, corresponding to the one or more genes must be obtained from the individual to be tested. Any tissue or cell sample containing the genomic DNA, miRNA, mRNA, and/or cDNA (or a portion thereof) corresponding to the one or more genes can be used. For this purpose, a tissue sample containing cell nucleus and thus genomic DNA can be obtained from the individual. Blood samples can also be useful except that only white blood cells and other lymphocytes have cell nucleus, while red blood cells are without a nucleus and contain only mRNA or miRNA. Nevertheless, miRNA and mRNA are also useful as either can be analyzed for the presence of nucleotide variants in its sequence or serve as template for cDNA synthesis. The tissue or cell samples can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of a particular nucleotide variant, sequencing of the target genomic DNA or cDNA, particularly the region encompassing the nucleotide variant locus to be detected. Various sequencing techniques are generally known and widely used in the art including the Sanger method and Gilbert chemical method. The pyrosequencing method monitors DNA synthesis in real time using a luminometric detection system. Pyrosequencing has been shown to be effective in analyzing genetic polymorphisms such as single-nucleotide polymorphisms and can also be used in the present invention. See Nordstrom et al., Biotechnol. Appl. Biochem., 31(2):107-112 (2000); Ahmadian et al., Anal. Biochem., 280:103-110 (2000).

Nucleic acid variants can be detected by a suitable detection process. Non limiting examples of methods of detection, quantification, sequencing and the like are; mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), microsequencing methods (e.g., a modification of primer extension methodology), ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), direct DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension (e.g., microarray sequence determination methods), Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization methods (e.g., hybridization using at least one probe, hybridization using at least one fluorescently labeled probe, and the like), conventional dot blot analyses, single strand conformational polymorphism analysis (SSCP, e.g., U.S. Pat. Nos. 5,891,625 and 6,013,499; Orita et al., Proc. Natl. Acad. Sci. U.S.A. 86: 27776-2770 (1989)), denaturing gradient gel electrophoresis (DGGE), heteroduplex analysis, mismatch cleavage detection, and techniques described in Sheffield et al., Proc. Natl. Acad. Sci. USA 49: 699-706 (1991), White et al., Genomics 12: 301-306 (1992), Grompe et al., Proc. Natl. Acad. Sci. USA 86: 5855-5892 (1989), and Grompe, Nature Genetics 5: 111-117 (1993), cloning and sequencing, electrophoresis, the use of hybridization probes and quantitative real time polymerase chain reaction (QRT-PCR), digital PCR, nanopore sequencing, chips and combinations thereof. The detection and quantification of alleles or paralogs can be carried out using the “closed-tube” methods described in U.S. patent application Ser. No. 11/950,395, filed on Dec. 4, 2007. In some embodiments the amount of a nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

The term “sequence analysis” as used herein refers to determining a nucleotide sequence, e.g., that of an amplification product. The entire sequence or a partial sequence of a polynucleotide, e.g., DNA or mRNA, can be determined, and the determined nucleotide sequence can be referred to as a “read” or “sequence read.” For example, linear amplification products may be analyzed directly without further amplification in some embodiments (e.g., by using single-molecule sequencing methodology). In certain embodiments, linear amplification products may be subject to further amplification and then analyzed (e.g., using sequencing by ligation or pyrosequencing methodology). Reads may be subject to different types of sequence analysis. Any suitable sequencing method can be utilized to detect, and determine the amount of, nucleotide sequence species, amplified nucleic acid species, or detectable products generated from the foregoing. Examples of certain sequencing methods are described hereafter.

A sequence analysis apparatus or sequence analysis component(s) includes an apparatus, and one or more components used in conjunction with such apparatus, that can be used by a person of ordinary skill to determine a nucleotide sequence resulting from processes described herein (e.g., linear and/or exponential amplification products). Examples of sequencing platforms include, without limitation, the 454 platform (Roche) (Margulies, M. et al. 2005 Nature 437, 376-380), Illumina Genomic Analyzer (or Solexa platform) or SOLID System (Applied Biosystems) or the Helicos True Single Molecule DNA sequencing technology (Harris T D et al. 2008 Science, 320, 106-109), the single molecule, real-time (SMRT™) technology of Pacific Biosciences, and nanopore sequencing (Soni G V and Meller A. 2007 Clin Chem 53: 1996-2001). Such platforms allow sequencing of many nucleic acid molecules isolated from a specimen at high orders of multiplexing in a parallel manner (Dear Brief Funct Genomic Proteomic 2003; 1: 397-416). Each of these platforms allows sequencing of clonally expanded or non-amplified single molecules of nucleic acid fragments. Certain platforms involve, for example, sequencing by ligation of dye-modified probes (including cyclic ligation and cleavage), pyrosequencing, and single-molecule sequencing. Nucleotide sequence species, amplification nucleic acid species and detectable products generated there from can be analyzed by such sequence analysis platforms.

Sequencing by ligation is a nucleic acid sequencing method that relics on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label, e.g., at least 1, 2, 3, 4, or 5 fluorescent labels.

Sequencing by ligation generally involves the following steps. Clonal bead populations can be prepared in emulsion microreactors containing target nucleic acid template sequences, amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Target nucleic acids may be immobilized to a solid support, hybridized with a sequencing primer, incubated with DNA polymerase, ATP sulfurylase, luciferase, apyrase, adenosine 5′ phosphosulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination. The amount of light generated is proportional to the number of bases added. Accordingly, the sequence downstream of the sequencing primer can be determined. An illustrative system for pyrosequencing involves the following steps: ligating an adaptor nucleic acid to a nucleic acid under investigation and hybridizing the resulting nucleic acid to a bead; amplifying a nucleotide sequence in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)).

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair” in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a target nucleic acid sequence to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslaysky et al., PNAS 100(7): 3960-3964 (2003)). Such a system can be used to directly sequence amplification products (linearly or exponentially amplified products) generated by processes described herein. In some embodiments the amplification products can be hybridized to a primer that contains sequences complementary to immobilized capture sequences present on a solid support, a bead or glass slide for example. Hybridization of the primer-amplification product complexes with the immobilized capture sequences, immobilizes amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting target nucleic acid and solid support under conditions in which a single molecule of sample nucleic acid hybridizes to a single molecule of a solid support. Such conditions can include providing the solid support molecules and a single molecule of target nucleic acid in a “microreactor.” Such conditions also can include providing a mixture in which the target nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a target nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons can be performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis can be facilitated by the use of sequence analysis apparatus and components described above.

Primer extension polymorphism detection methods, also referred to herein as “microsequencing” methods, typically are carried out by hybridizing a complementary oligonucleotide to a nucleic acid carrying the polymorphic site. In these methods, the oligonucleotide typically hybridizes adjacent to the polymorphic site. The term “adjacent” as used in reference to “microsequencing” methods, refers to the 3′ end of the extension oligonucleotide being sometimes 1 nucleotide from the 5′ end of the polymorphic site, often 2 or 3, and at times 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, often 1, 2, or 3 nucleotides, and the number and/or type of nucleotides that are added to the extension oligonucleotide determine which polymorphic variant or variants are present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. The extension products can be detected in any manner, such as by fluorescence methods (see, e.g., Chen & Kwok, Nucleic Acids Research 25: 347-353 (1997) and Chen et al., Proc. Natl. Acad. Sci. USA 94/20: 10756-10761 (1997)) or by mass spectrometric methods (e.g., MALDI-TOF mass spectrometry) and other methods described herein. Oligonucleotide extension methods using mass spectrometry are described, for example, in U.S. Pat. Nos. 5,547,835; 5,605,798; 5,691,141; 5,849,542; 5,869,242; 5,928,906; 6,043,031; 6,194,144; and 6,258,538.

Microsequencing detection methods often incorporate an amplification process that proceeds the extension step. The amplification process typically amplifies a region from a nucleic acid sample that comprises the polymorphic site. Amplification can be carried out utilizing methods described above, or for example using a pair of oligonucleotide primers in a polymerase chain reaction (PCR), in which one oligonucleotide primer typically is complementary to a region 3′ of the polymorphism and the other typically is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GeneAmp™ Systems available from Applied Biosystems.

Other appropriate sequencing methods include multiplex polony sequencing (as described in Shendure et al., Accurate Multiplex Polony Sequencing of an Evolved Bacterial Genome, Sciencexpress, Aug. 4, 2005, pg 1 available at www.sciencexpress.org/4 Aug. 2005/Page1/10.1126/science. 1117389, incorporated herein by reference), which employs immobilized microbeads, and sequencing in microfabricated picoliter reactors (as described in Margulies et al., Genome Sequencing in Microfabricated High-Density Picolitre Reactors, Nature, August 2005, available at www.nature.com/nature (published online 31 Jul. 2005, doi:10.1038/nature03959, incorporated herein by reference).

Whole genome sequencing may also be utilized for discriminating alleles of RNA transcripts, in some embodiments. Examples of whole genome sequencing methods include, but are not limited to, nanopore-based sequencing methods, sequencing by synthesis and sequencing by ligation, as described above.

Nucleic acid variants can also be detected using standard electrophoretic techniques. Although the detection step can sometimes be preceded by an amplification step, amplification is not required in the embodiments described herein. Examples of methods for detection and quantification of a nucleic acid using electrophoretic techniques can be found in the art. A non-limiting example comprises running a sample (e.g., mixed nucleic acid sample isolated from maternal serum, or amplification nucleic acid species, for example) in an agarose or polyacrylamide gel. The gel may be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which may then be compared to the control based on the intensity of the hand, thus detecting and quantifying the target sequence of interest. In some embodiments, restriction enzymes capable of distinguishing between maternal and paternal alleles may be used to detect and quantify target nucleic acid species. In certain embodiments, oligonucleotide probes specific to a sequence of interest are used to detect the presence of the target sequence of interest. The oligonucleotides can also be used to indicate the amount of the target nucleic acid molecules in comparison to the standard control, based on the intensity of signal imparted by the probe.

Sequence-specific probe hybridization can be used to detect a particular nucleic acid in a mixture or mixed population comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch. A number of hybridization formats are known in the art, which include but are not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

Hybridization complexes can be detected by techniques known in the art. Nucleic acid probes capable of specifically hybridizing to a target nucleic acid (e.g., mRNA or DNA) can be labeled by any suitable method, and the labeled probe used to detect the presence of hybridized nucleic acids. One commonly used method of detection is autoradiography, using probes labeled with ³H, ¹²⁵I, ³⁵S, ¹⁴C, ³²P, ³³P, or the like. The choice of radioactive isotope depends on research preferences due to ease of synthesis, stability, and half-lives of the selected isotopes. Other labels include compounds (e.g., biotin and digoxigenin), which bind to antiligands or antibodies labeled with fluorophores, chemiluminescent agents, and enzymes. In some embodiments, probes can be conjugated directly with labels such as fluorophores, chemiluminescent agents or enzymes. The choice of label depends on sensitivity required, ease of conjugation with the probe, stability requirements, and available instrumentation.

Alternatively, the restriction fragment length polymorphism (RFLP) and AFLP method may be used for molecular profiling. If a nucleotide variant in the target DNA corresponding to the one or more genes results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998).

The presence or absence of a nucleotide variant at a particular locus in the one or more genes of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA, mRNA or miRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., IIum. Mutat., 7:346-354 (1996); Chen et al., Genome Res., 10:549-547 (2000).

Another set of techniques useful in the present invention is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in the one or more genes, two oligonucleotides can be synthesized, one having the sequence just 5′ upstream from the locus with its 3′ end nucleotide being identical to the nucleotide in the variant locus of the particular gene, the other having a nucleotide sequence matching the sequence immediately 3′ downstream from the locus in the gene. The oligonucleotides can be labeled for the purpose of detection. Upon hybridizing to the target gene under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to a gene allele having a particular gene variant at a particular locus but not to other alleles can be designed by methods known in the art. The probes can have a length of, e.g., from 10 to about 50 nucleotide bases. The target DNA and the oligonucleotide probe can be contacted with each other under conditions sufficiently stringent such that the nucleotide variant can be distinguished from the wild-type gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in an RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art. See e.g., Roberts et al., Nucleic Acids Res., 25:3377-3378 (1997).

In the mutS assay, a probe can be prepared matching the gene sequence surrounding the locus at which the presence or absence of a mutation is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques which can be useful in detecting mutations or nucleotide variants in the present invention. For example, the “sunrise probes” or “molecular beacons” use the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpin-shaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the gene spanning the variant locus of interest and to differentially hybridize with different alleles. The two ends of the probe are labeled with a quenching fluorophore and a reporter fluorophore, respectively. The TaqMan probe is incorporated into a PCR reaction for the amplification of a target gene region containing the locus of interest using Taq polymerase. As Taq polymerase exhibits 5′-3′ exonuclease activity but has no 3′-5′ exonuclease activity, if the TaqMan probe is annealed to the target DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present invention can also employ a chemiluminescence-based technique. For example, an oligonucleotide probe can be designed to hybridize to either the wild-type or a variant gene locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in the gene in accordance with the present invention can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Mass spectrometry can be used for molecular profiling according to the invention. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present invention. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations. The microchip technologies combined with computerized analysis tools allow fast screening in a large scale. The adaptation of the microchip technologies to the present invention will be apparent to a person of skill in the art apprised of the present disclosure. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

As is apparent from the above survey of the suitable detection techniques, it may or may not be necessary to amplify the target DNA, i.e., the gene, cDNA, mRNA, miRNA, or a portion thereof to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2^{nd} ed., Cold Spring Harbor Laboratory, Cold Spring Harbor, N.Y., 1989. However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theor. Biol., 187:273-284 (1997).

The Invader™ assay is another technique for detecting single nucleotide variations that can be used for molecular profiling according to the invention. The Invader™ assay uses a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader™ system utilizes two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, Sniper™, a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. Sec Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations among fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995).

In addition, the allele-specific oligonucleotides (ASO) can also be used in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a nucleotide variant in the one or more gene of a particular individual should be apparent to a skilled artisan apprised of this disclosure.

Accordingly, the presence or absence of one or more genes nucleotide variant or amino acid variant in an individual can be determined using any of the detection methods described above.

Typically, once the presence or absence of one or more gene nucleotide variants or amino acid variants is determined, physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of a nucleotide variant of the present invention in the individual tested can be embodied in descriptive statements, diagrams, photographs, charts, images or any other visual forms. For example, images of gel electrophoresis of PCR products can be used in explaining the results. Diagrams showing where a variant occurs in an individual's gene are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a nucleotide variant or amino acid variant in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present invention also encompasses a method for producing a transmittable form of information on the genotype of the two or more suspected cancer samples from an individual. The method comprises the steps of (1) determining the genotype of the DNA from the samples according to methods of the present invention; and (2) embodying the result of the determining step in a transmittable form. The transmittable form is the product of the production method.

In Situ Hybridization

In situ hybridization assays are well known and are generally described in Angcrer et al., Methods Enzymol. 152:649-660 (1987). In an in situ hybridization assay, cells, e.g., from a biopsy, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters. FISH (fluorescence in situ hybridization) uses fluorescent probes that bind to only those parts of a sequence with which they show a high degree of sequence similarity.

In situ hybridization can be used to detect specific gene sequences in tissue sections or cell preparations by hybridizing the complementary strand of a nucleotide probe to the sequence of interest. Fluorescent in situ hybridization (FISH) uses a fluorescent probe to increase the sensitivity of in situ hybridization.

FISH is a cytogenetic technique used to detect and localize specific polynucleotide sequences in cells. For example, FISH can be used to detect DNA sequences on chromosomes. FISH can also be used to detect and localize specific RNAs, e.g., mRNAs, within tissue samples. In FISH uses fluorescent probes that bind to specific nucleotide sequences to which they show a high degree of sequence similarity. Fluorescence microscopy can be used to find out whether and where the fluorescent probes are hound. In addition to detecting specific nucleotide sequences, e.g., translocations, fusion, breaks, duplications and other chromosomal abnormalities, FISH can help define the spatial-temporal patterns of specific gene copy number and/or gene expression within cells and tissues.

Various types of FISH probes can be used to detect chromosome translocations. Dual color, single fusion probes can be useful in detecting cells possessing a specific chromosomal translocation. The DNA probe hybridization targets are located on one side of each of the two genetic breakpoints. “Extra signal” probes can reduce the frequency of normal cells exhibiting an abnormal FISH pattern due to the random co-localization of probe signals in a normal nucleus. One large probe spans one breakpoint, while the other probe flanks the breakpoint on the other gene. Dual color, break apart probes are useful in cases where there may be multiple translocation partners associated with a known genetic breakpoint. This labeling scheme features two differently colored probes that hybridize to targets on opposite sides of a breakpoint in one gene. Dual color, dual fusion probes can reduce the number of normal nuclei exhibiting abnormal signal patterns. The probe offers advantages in detecting low levels of nuclei possessing a simple balanced translocation. Large probes span two breakpoints on different chromosomes. Such probes are available as Vysis probes from Abbott Laboratories, Abbott Park, Ill.

Comparative Genomic Hybridization (CGH) comprises a molecular cytogenetic method of screening tumor samples for genetic changes showing characteristic patterns for copy number changes at chromosomal and subchromosomal levels. Alterations in patterns can be classified as DNA gains and losses. CGH employs the kinetics of in situ hybridization to compare the copy numbers of different DNA or RNA sequences from a sample, or the copy numbers of different DNA or RNA sequences in one sample to the copy numbers of the substantially identical sequences in another sample. In many useful applications of CGH, the DNA or RNA is isolated from a subject cell or cell population. The comparisons can be qualitative or quantitative. Procedures are described that permit determination of the absolute copy numbers of DNA sequences throughout the genome of a cell or cell population if the absolute copy number is known or determined for one or several sequences. The different sequences are discriminated from each other by the different locations of their binding sites when hybridized to a reference genome, usually metaphase chromosomes but in certain cases interphase nuclei. The copy number information originates from comparisons of the intensities of the hybridization signals among the different locations on the reference genome. The methods, techniques and applications of CGH are known, such as described in U.S. Pat. No. 6,335,167, and in U.S. App. Ser. No. 60/804,818, the relevant parts of which are herein incorporated by reference.

In an embodiment, CGH used to compare nucleic acids between diseased and healthy tissues. The method comprises isolating DNA from disease tissues (e.g., tumors) and reference tissues (e.g., healthy tissue) and labeling each with a different “color” or fluor. The two samples are mixed and hybridized to normal metaphase chromosomes. In the case of array or matrix CGH, the hybridization mixing is done on a slide with thousands of DNA probes. A variety of detection system can be used that basically determine the color ratio along the chromosomes to determine DNA regions that might be gained or lost in the diseased samples as compared to the reference.

Data and Analysis

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet as shown in U.S. Ser. Nos. 10/197,621, 10/063,559 (U.S. Publication Number 20020183936), Ser. Nos. 10/065,856, 10/065,868, 10/328,818, 10/328,872, 10/423,403, and 60/482,389. For example, one or more molecular profiling techniques can be performed in one location, e.g., a city, state, country or continent, and the results can be transmitted to a different city, state, country or continent. Treatment selection can then be made in whole or in part in the second location. The methods of the invention comprise transmittal of information between different locations.

Molecular Profiling for Treatment Selection

The methods of the invention provide a candidate treatment selection for a subject in need thereof. Molecular profiling can be used to identify one or more candidate therapeutic agents for an individual suffering from a condition in which one or more of the biomarkers disclosed herein are targets for treatment. For example, the method can identify one or more chemotherapy treatments for a cancer. In an aspect, the invention provides a method comprising: performing an immunohistochemistry (IHC) analysis on a sample from the subject to determine an IHC expression profile on at least five proteins; performing a microarray analysis on the sample to determine a microarray expression profile on at least ten genes; performing a fluorescent in-situ hybridization (FISH) analysis on the sample to determine a FISH mutation profile on at least one gene; performing DNA sequencing on the sample to determine a sequencing mutation profile on at least one gene; and comparing the IHC expression profile, microarray expression profile, FISH mutation profile and sequencing mutation profile against a rules database, wherein the rules database comprises a mapping of treatments whose biological activity is known against diseased cells that: i) overexpress or underexpress one or more proteins included in the IHC expression profile; ii) overexpress or underexpress one or more genes included in the microarray expression profile; iii) have zero or more mutations in one or more genes included in the FISH mutation profile; and/or iv) have zero or more mutations in one or more genes included in the sequencing mutation profile; and identifying the treatment if the comparison against the rules database indicates that the treatment should have biological activity against the diseased cells; and the comparison against the rules database does not contraindicate the treatment for treating the diseased cells. The disease can be a cancer. The molecular profiling steps can be performed in any order. In some embodiments, not all of the molecular profiling steps are performed. As a non-limiting example, microarray analysis is not performed if the sample quality does not meet a threshold value, as described herein. In another example, sequencing is performed only if FISH analysis meets a threshold value. Any relevant biomarker can be assessed using one or more of the molecular profiling techniques described herein or known in the art. The marker need only have some direct or indirect association with a treatment to be useful.

Molecular profiling comprises the profiling of at least one gene (or gene product) for each assay technique that is performed. Different numbers of genes can be assayed with different techniques. Any marker disclosed herein that is associated directly or indirectly with a target therapeutic can be assessed. For example, any “druggable target” comprising a target that can be modulated with a therapeutic agent such as a small molecule, is a candidate for inclusion in the molecular profiling methods of the invention. The molecular profiling can be based on either the gene, e.g., DNA sequence, and/or gene product, e.g., mRNA or protein. Such nucleic acid and/or polypeptide can be profiled as applicable as to presence or absence, level or amount, activity, mutation, sequence, haplotype, rearrangement, copy number, or other measurable characteristic. In some embodiments, a single gene and/or one or more corresponding gene products is assayed by more than one molecular profiling technique. A gene or gene product (also referred to herein as “marker” or “biomarker”), e.g., an mRNA or protein, is assessed using applicable techniques (e.g., to assess DNA, RNA, protein), including without limitation FISH, microarray, IHC, sequencing or immunoassay. Therefore, any of the markers disclosed herein can be assayed by a single molecular profiling technique or by multiple methods disclosed herein (e.g., a single marker is profiled by one or more of IHC, FISH, sequencing, microarray, etc.). In some embodiments, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or at least about 100 genes or gene products are profiled by at least one technique, a plurality of techniques, or using a combination of FISH, microarray, IHC, and sequencing. In some embodiments, at least about 100, 200, 300, 400, 500, 600, 700, 800, 900, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, or at least 50,000 genes or gene products are profiled using various techniques. The number of markers assayed can depend on the technique used. For example, microarray and massively parallel sequencing lend themselves to high throughput analysis. Because molecular profiling queries molecular characteristics of the tumor itself, this approach provides information on therapies that might not otherwise be considered based on the lineage of the tumor.

In some embodiments, a sample from a subject in need thereof is profiled using methods which include but are not limited to IHC expression profiling, microarray expression profiling, FISH mutation profiling, and/or sequencing mutation profiling (such as by PCR, RT-PCR, pyrosequencing) for one or more of the following: ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKN1A, CDKN1B, CDK2, CDW52, CES2, CK 14, CK 17, CK 5/6, c-KTT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, DART, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, INN, MET, MGMT, MLH1, MMR, MRP1, MS4A1, MSH2, MSH5, Myc, NFKB1, NFKB2, NFKBIA, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, RAF1, RARA, RRM1, RRM2, RRM2B, RXRB, RXRG, SPARC, SRC, SSTR1, SSTR2, SSTR3, SSTR4, SSTR5, Survivin, TK1, TLE3, TNF, TOP1, TOP2A, TOP2B, TS, TXN, TXNRD1, TYMS, VDR, VEGF, VEGFA, VEGFC, VHL, YES1, ZAP70.

Table 1 provides a listing of gene and corresponding protein symbols and names of many of the molecular profiling targets that are analyzed according to the methods of the invention. As understood by those of skill in the art, genes and proteins have developed a number of alternative names in the scientific literature. Thus, the listing in Table 1 comprises an illustrative but not exhaustive compilation. A further listing of gene aliases and descriptions can be found using a variety of online databases, including GeneCards® (www.genecards.org), HUGO Gene Nomenclature (www.genenames.org), Entrez Gene (www.ncbi.nlm.nih.gov/entrez/query.fcgi?db=gene), UniProtKB/Swiss-Prot (www.uniprot.org), UniProtKB/TrEMBL (www.uniprot.org), OMIM (www.ncbi.nlm.nih.gov/entrez/query.fcgi?db=OMIM), GeneLoc (genecards.weizmann.ac.il/geneloc/), and Ensembl (www.ensembl.org). Generally, gene symbols and names below correspond to those approved by HUGO, and protein names are those recommended by UniProtKB/Swiss-Prot. Common alternatives are provided as well. Where a protein name indicates a precursor, the mature protein is also implied. Throughout the application, gene and protein symbols may be used interchangeably and the meaning can be derived from context, e.g., FISH is used to analyze nucleic acids whereas IHC is used to analyze protein.

TABLE 1
Gene and Protein Names

| Gene | | Protein | |
|---|---|---|---|
| Symbol | Gene Name | Symbol | Protein Name |
| ABCB1, | ATP-binding cassette, sub-family B | ABCB1, | Multidrug resistance protein 1; |
| PGP | (MDR/TAP), member 1 | MDR1, | P-glycoprotein |
| | | PGP | |
| ABCC1, | ATP-binding cassette, sub-family C | MRP1, | Multidrug resistance-associated |
| MRP1 | (CFTR/MRP), member 1 | ABCC1 | protein 1 |
| ABCG2, | ATP-binding cassette, sub-family G | ABCG2 | ATP-binding cassette sub-family G |
| BCRP | (WHITE), member 2 | | member 2 |
| ACE2 | angiotensin I converting enzyme | ACE2 | Angiotensin-converting enzyme 2 |
| | (peptidyl-dipeptidase A) 2 | | precursor |
| ADA | adenosine deaminase | ADA | Adenosine deaminase |
| ADH1C | alcohol dehydrogenase 1C (class I), | ADH1G | Alcohol dehydrogenase 1C |
| | gamma polypeptide | | |
| ADII4 | alcohol dehydrogenase 4 (class II), | ADII4 | Alcohol dehydrogenase 4 |
| | pi polypeptide | | |
| AGT | angiotensinogen (serpin peptidase | ANGT, | Angiotensinogen precursor |
| | inhibitor, clade A, member 8) | AGT | |
| ALK | anaplastic lymphoma receptor | ALK | ALK tyrosine kinase receptor |
| | tyrosine kinase | | precursor |
| AR | androgen receptor | AR | Androgen receptor |
| AREG | amphiregulin | AREG | Amphiregulin precursor |
| ASNS | asparagine synthetase | ASNS | Asparagine synthetase [glutamine- |
| | | | hydrolyzing] |
| BCL2 | B-cell CLL/lymphoma 2 | BCL2 | Apoptosis regulator Bcl-2 |
| BDCA1, | CD1c molecule | CD1C | T-cell surface glycoprotein CD1c |
| CD1C | | | precursor |
| BIRC5 | baculoviral IAP repeat-containing 5 | BIRC5, | Baculoviral IAP repeat-containing |
| | | Survivin | protein 5; Survivin |
| BRAF | v-raf murine sarcoma viral | B-RAF, | Serine/threonine-protein kinase B-raf |
| | oncogene homolog B1 | BRAF | |
| BRCA1 | breast cancer 1, early onset | BRCA1 | Breast cancer type 1 susceptibility |
| | | | protein |
| BRCA2 | breast cancer 2, early onset | BRCA2 | Breast cancer type 2 susceptibility |
| | protein | | |
| CA2 | carbonic anhydrase II | CA2 | Carbonic anhydrase 2 |
| CAV1 | caveolin 1, caveolae protein, | CAV1 | Caveolin-1 |
| | 22kDa | | |
| CCND1 | cyclin D1 | CCND1, | G1/S-specific cyclin-D1 |
| | | Cyclin D1, | |
| | | BCL-1 | |
| CD20, | membrane-spanning 4-domains, | CD20 | B-lymphocyte antigen CD20 |
| MS4A1 | subfamily A, member 1 | | |
| CD25, | interleukin 2 receptor, alpha | CD25 | Interleukin-2 receptor subunit alpha |
| IL2RA | | | precursor |
| CD33 | CD33 molecule | CD33 | Myeloid cell surface antigen CD33 |
| | | | precursor |
| CD52, | CD52 molecule | CD52 | CAMPATH-1 antigen precursor |
| CDW52 | | | |
| CDA | cytidine deaminase | CDA | Cytidine deaminase |
| CDH1, | cadherin 1, type 1, E-cadherin | E-Cad | Cadherin-1 precursor (E-cadherin) |
| ECAD | (epithelial) | | |
| CDK2 | cyclin-dependent kinase 2 | CDK2 | Cell division protein kinase 2 |
| CDKN1A, | cyclin-dependent kinase inhibitor | CDKN1A, | Cyclin-dependent kinase inhibitor 1 |
| P21 | 1A (p21, Cip1) | p21 | |
| CDKN1B | cyclin-dependent kinase inhibitor | CDKN1B, | Cyclin-dependent kinase inhibitor 1B |
| | 1B (p27, Kip1) | p27 | |
| CDKN2A, | cyclin-dependent kinase inhibitor | CD21A, | Cyclin-dependent kinase inhibitor 2A, |
| P16 | 2A (melanoma, p16, inhibits | p16 | isoforms 1/2/3 |
| | CDK4) | | |
| CES2 | carboxylesterase 2 (intestine, liver) | CES2, | Carboxylesterase 2 precursor |
| | | EST2 | |
| CK 5/6 | cytokeratin 5/cytokeratin 6 | CK 5/6 | Keratin, type II cytoskeletal 5; Keratin, |
| | | | type II cytoskeletal 6 |
| CK14, | keratin 14 | CK14 | Keratin, type I cytoskeletal 14 |
| KRT14 | | | |
| CK17, | keratin 17 | CK17 | Keratin, type I cytoskeletal 17 |
| KRT17 | | | |
| COX2, | prostaglandin-endoperoxide | COX-2, | Prostaglandin G/H synthase 2 |
| PTGS2 | synthase 2 (prostaglandin G/H | PTGS2 | precursor |
| | synthase and cyclooxygenase) | | |
| DCK | deoxycytidine kinasc | DCK | Deoxycytidine kinasc |
| DHFR | dihydrofolate reductase | DHFR | Dihydrofolate reductase |
| DNMT1 | DNA (cytosine-5-)- | DNMT1 | DNA (cytosine-5)-methyltransferase 1 |
| | methyltransferase 1 | | |
| DNMT3A | DNA (cytosine-5-)- | DNMT3A | DNA (cytosine-5)-methyltransferase |
| | methyltransferase 3 alpha | | 3A |
| DNMT3B | DNA (cytosine-5-)- | DNMT3B | DNA (cytosine-5)-methyltransferase |
| | methyltransferase 3 beta | | 3B |
| ECGF1, | thymidine phosphorylase | TYMP, | Thymidine phosphorylase precursor |
| TYMP | | PD-ECGF, | |
| | | ECDF1 | |
| EGFR, | epidermal growth factor receptor | EGFR, | Epidermal growth factor receptor |
| ERBB1, | (erythroblastic leukemia viral | ERBB1, | precursor |
| HER1 | (v-erb-b) oncogene homolog, avian) | HER1 | |
| EML4 | echinoderm microtubule associated | EMLA | Echinoderm microtubule-associated |
| | protein like 4 | | protein-like 4 |
| EPHA2 | EPH receptor A2 | EPHA2 | Ephrin type-A receptor 2 precursor |
| ER, ESR1 | estrogen receptor 1 | ER, ESR1 | Estrogen receptor |
| ERBB2, | v-erb-b2 erythroblastic leukemia | ERBB2, | Receptor tyrosine-protein kinase |
| HER2/NEU | viral oncogene homolog 2, | HER2, | erbB-2 precursor |
| | neuro/glioblastoma derived | HER-2/neu | |
| | oncogene homolog (avian) | | |
| ERCC1 | excision repair cross- | ERCC1 | DNA excision repair protein ERCC-1 |
| | complementing rodent repair | | |
| | deficiency, complementation group | | |
| | 1 (includes overlapping antisense | | |
| | sequence) | | |
| ERCC3 | excision repair cross- | ERCC3 | TFIIH basal transcription factor |
| | complementing rodent repair | | complex helicase XPB subunit |
| | deficiency, complementation group | | |
| | 3 (xeroderma pigmentosum group B | | |
| | complementing) | | |
| EREG | Epiregulin | EREG | Proepiregulin precursor |
| FLT1 | fms-related tyrosine kinase 1 | FLT-1, | Vascular endothelial growth factor |
| | (vascular endothelial growth | VEGFR1 | receptor 1 precursor |
| | factor/vascular permeability factor | | |
| | receptor) | | |
| FOLR1 | folate receptor 1 (adult) | FOLR1 | Folate receptor alpha precursor |
| FOLR2 | folate receptor 2 (fetal) | FOLR2 | Folate receptor beta precursor |
| FSHB | follicle stimulating hormone, beta | FSHB | Follitropin subunit beta precursor |
| | polypeptide | | |
| FSHPRH1, | centromere protein I | FSHPRH1, | Centromere protein I |
| CENP1 | | CENP1 | |
| FSHR | follicle stimulating hormone | FSHR | Follicle-stimulating hormone receptor |
| | receptor | | precursor |
| FYN | FYN oncogene related to SRC, | FYN | Tyrosine-protein kinase Fyn |
| | FGR, YES | | |
| GART | phosphoribosylglycinamide | GART, | Trifunctional purine biosynthetic |
| | formyltransferase, | PUR2 | protein adenosine-3 |
| | phosphoribosylglycinamide | | |
| | synthetase, | | |
| | phosphoribosylaminoimidazole | | |
| | synthetase | | |
| GNRII1 | gonadotropin-releasing hormone 1 | GNRII1, | Progonadoliberin-1 precursor |
| | (luteinizing-releasing hormone) | GON1 | |
| GNRHR1, | gonadotropin-releasing hormone | GNRHR1 | Gonadotropin-releasing hormone |
| GNRHR | receptor | | receptor |
| GSTP1 | glutathione S-transferase pi 1 | GSTP1 | Glutathione S-transferase P |
| HCK | hemopoietic cell kinase | HCK | Tyrosine-protein kinase HCK |
| HDAC1 | histone deacetylase 1 | HDAC1 | Histone deacetylase 1 |
| HGF | hepatocyte growth factor | HGF | Hepatocyte growth factor precursor |
| | (hepapoietin A; scatter factor) | | |
| HIF1A | hypoxia inducible factor 1, alpha | HIF1A | Hypoxia-inducible factor 1-alpha |
| | subunit (basic helix-loop-helix | | |
| | transcription factor) | | |
| HIG1, | HIG1 hypoxia inducible domain | HIG1, | HIG1 domain family member IA |
| HIGD1A, | family, member 1A | HIGD1A, | |
| HIG1A | | HIG1A | |
| HSP90AA1, | heat shock protein 90kDa alpha | HSP90, | Heal shock protein HSP 90-alpha |
| HSP90, | (cytosolic), class A member 1 | HSP90A | |
| HSPCA | | | |
| IGF1R | insulin-like growth factor 1 receptor | IGF-1R | Insulin-like growth factor 1 receptor |
| | | | precursor |
| IGFBP3, | insulin-like growth factor binding | IGFBP-3, | Insulin-like growth factor-binding |
| IGFRBP3 | protein 3 | IBP-3 | protein 3 precursor |
| IGFBP4, | insulin-like growth factor binding | IGFBP-4, | Insulin-like growth factor-binding |
| IGFRBP4 | protein 4 | IBP-4 | protein 4 precursor |
| IGFBP5, | insulin-like growth factor binding | IGFBP-5, | Insulin-like growth factor-binding |
| IGFRBP5 | protein 5 | IBP-5 | protein 5 precursor |
| IL13RA1 | interleukin 13 receptor, alpha 1 | IL-13RA1 | Interleukin-13 receptor subunit alpha-1 |
| | | | precursor |
| KDR | kinase insert domain receptor (a | KDR, | Vascular endothelial growth factor |
| | type III receptor tyrosine kinase) | VEGFR2 | receptor 2 precursor |
| KIT, | v-kit Hardy-Zuckerman 4 feline | KIT, c-KIT | Mast/stem cell growth factor receptor |
| c-KIT | sarcoma viral oncogene homolog | | precursor |
| KRAS | v-Ki-ras2 Kirsten rat sarcoma viral | K-RAS | GTPase KRas precursor |
| | oncogene homolog | | |
| LCK | lymphocyte-specific protein | LCK | Tyrosine-protein kinase Lck |
| | tyrosine kinase | | |
| LTB | lymphotoxin beta (TNF | LTB, | Lymphotoxin-beta |
| | superfamily, member 3) | TNF3 | |
| LTBR | lymphotoxin beta receptor (TNFR | LTBR, | Tumor necrosis factor receptor |
| | superfamily, member 3) | LTBR3, | superfamily member 3 precursor |
| | | TNFR | |
| LYN | v-yes-1 Yamaguchi sarcoma viral | LYN | Tyrosine-protein kinase Lyn |
| | related oncogene homolog | | |
| MET, | met proto-oncogene (hepatocyte | MET, | Hepatocyte growth factor receptor |
| c-MET | growth factor receptor) | c-MET | precursor |
| MGMT | O-6-methylguanine-DNA | MGMT | Methylated-DNA--protein-cysteine |
| | methyltransferase | | methyltransferase |
| MKI67, | antigen identified by monoclonal | Ki67, | Antigen KI-67 |
| KI67 | antibody Ki-67 | Ki-67 | |
| MLH1 | mutL homolog 1, colon cancer, | MLH1 | DNA mismatch repair protein Mlh1 |
| | nonpolyposis type 2 (E. coli) | | |
| MMR | mismatch repair (refers to MLH1, | | |
| | MSH2, MSH5) | | |
| MSH2 | mutS homolog 2, colon cancer, | MSH2 | DNA mismatch repair protein Msh2 |
| | nonpolyposis type 1 (E. coli) | | |
| MSH5 | mutS homolog 5 (E. coli) | MSH5, | MutS protein homolog 5 |
| | | hMSH5 | |
| MYC, | v-myc myelocytomatosis viral | MYC, | Myc proto-oncogene protein |
| c-MYC | oncogene homolog (avian) | c-MYC | |
| NBN, P95 | nibrin | NBN, p95 | Nibrin |
| NDGR1 | N-myc downstream regulated 1 | NDGR1 | Protein NDGR1 |
| NFKB1 | nuclear factor of kappa light | NFKB1 | Nuclear factor NF-kappa-B p105 |
| | polypeptide gene enhancer in | | subunit |
| | B-cells 1 | | |
| NFKB2 | nuclear factor of kappa light | NFKB2 | Nuclear factor NF-kappa-B p100 |
| | polypeptide gene enhancer in | | subunit |
| | B-cells 2 (p49/p100) | | |
| NFKBIA | nuclear factor of kappa light | NFKBIA | NF-kappa-B inhibitor alpha |
| | polypeptide gene enhancer in | | |
| | B-cells inhibitor, alpha | | |
| ODC1 | ornithine decarboxylase 1 | ODC | Ornithine decarboxylase |
| OGFR | opioid growth factor receptor | OGFR | Opioid growth factor receptor |
| PARP1 | poly (ADP-ribose) polymerase 1 | PARP-1 | Poly [ADP-ribose] polymerase 1 |
| PDGFC | platelet derived growth factor C | PDGF-C, | Platelet-derived growth factor C |
| | | VEGF-E | precursor |
| PDGFR | platelet-derived growth factor | PDGFR | Platelet-derived growth factor receptor |
| | receptor | | |
| PDGFRA | platelet-derived growth factor | PDGFRA, | Alpha-type platelet-derived growth |
| | receptor, alpha polypeptide | PDGFR2, | factor receptor precursor |
| | | CD140 A | |
| PDGFRB | platelet-derived growth factor | PDGFRB, | Beta-type platelet-derived growth |
| | receptor, beta polypeptide | PDGFR, | factor receptor precursor |
| | | PDGFR1, | |
| | | CD140 B | |
| PIK3CA | phosphoinositide-3-kinase, | PI3K | phosphoinositide-3-kinase, catalytic, |
| | catalytic, alpha polypeptide | subunit | alpha polypeptide |
| | | p110α | |
| PSMD9, | proteasome (prosome, macropain) | p27 | 26S proteasome non-ATPase |
| P27 | 26S subunit, non-ATPase, 9 | | regulatory subunit 9 |
| PTEN | phosphatase and tensin homolog | | |
| RRM1 | ribonucleotide reductase M1 | RRM1, | Ribonucleoside-diphosphate reductase |
| | | RR1 | large subunit |
| RRM2 | ribonucleotide reductase M2 | RRM2, | Ribonucleoside-diphosphate reductase |
| | | RR2M, | subunit M2 |
| | | RR2 | |
| RRM2B | ribonucleotide reductase M2 B | RRM2B, | Ribonucleoside-diphosphate reductase |
| | (TP53 inducible) | P53R2 | subunit M2 B |
| RXRB | retinoid X receptor, beta | RXRB | Retinoic acid receptor RXR-beta |
| RXRG | retinoid X receptor, gamma | RXRG, | Retinoic acid receptor RXR-gamma |
| | | RXRC | |
| SLC29A1 | solute carrier family 29 (nucleoside | ENT-1 | Equilibrative nucleoside transporter 1 |
| | transporters), member 1 | | |
| SPARC | secreted protein, acidic, cysteine-rich | SPARC | SPARC precursor; Osteonectin |
| | (osteonectin) | | |
| SRC | v-src sarcoma (Schmidt-Ruppin A-2) | SRC | Proto-oncogene tyrosine-protein kinase |
| | viral oncogene homolog (avian) | | Src |
| SSTR1 | somatostatin receptor 1 | SSTR1, | Somatostatin receptor type 1 |
| | | SSR1, | |
| | | SS1R | |
| SSTR2 | somatostatin receptor 2 | SSTR2, | Somatostatin receptor type 2 |
| | | SSR2, | |
| | | SS2R | |
| SSTR3 | somatostatin receptor 3 | SSTR3, | Somatostatin receptor type 3 |
| | | SSR3, | |
| | | SS3R | |
| SSTR4 | somatostatin receptor 4 | SSTR4, | Somatostatin receptor type 4 |
| | | SSR4, | |
| | | SS4R | |
| SSTR5 | somatostatin receptor 5 | SSTR5, | Somatostatin receptor type 5 |
| | | SSR5, | |
| | | SS5R | |
| TK1 | thymidine kinase 1, soluble | TK1, KITH | Thymidine kinase, cytosolic |
| TLE3 | transducin-like enhancer of split 3 | TLE3 | Transducin-like enhancer protein 3 |
| | (E(sp1) homolog, Drosophila) | | |
| TNF | tumor necrosis factor (TNF | TNF, | Tumor necrosis factor precursor |
| | superfamily, member 2) | TNF-alpha, | |
| | | TNF-a | |
| TOP1, | topoisomerase (DNA) I | TOP1, | DNA topoisomerase 1 |
| TOPO1 | | TOPO1 | |
| TOP2A, | topoisomerase (DNA) II alpha | TOP2A, | DNA topoisomerase 2-alpha; |
| TOPO2A | 170kDa | TOP2, | Topoisomerase II alpha |
| | | TOPO2A | |
| TOP2B, | topoisomerase (DNA) II beta | TOP2B, | DNA topoisomerase 2-beta; |
| TOPO2B | 180kDa | TOPO2B | Topoisomerase II beta |
| TP53 | tumor protein p53 | p53 | Cellular tumor antigen p53 |
| TUBB3 | tubulin, beta 3 | Beta III | Tubulin beta-3 chain |
| | | tubulin, | |
| | | TUBB3, | |
| | | TUBB4 | |
| TXN | thioredoxin | TXN, | Thioredoxin |
| | | TRX, | |
| | | TRX-1 | |
| TXNRD1 | thioredoxin reductase 1 | TXNRD1, | Thioredoxin reductase 1, cytoplasmic; |
| | | TXNR | Oxidoreductase |
| TYMS, | thymidylate synthetase | TYMS, TS | Thymidylate synthase |
| TS | | | |
| VDR | vitamin D (1,25-dihydroxyvitamin | VDR | Vitamin D3 receptor |
| | D3) receptor | | |
| VEGFA, | vascular endothelial growth | VEGF-A, | Vascular endothelial growth factor A |
| VEGF | factor A | VEGF | precursor |
| VEGFC | vascular endothelial growth | VEGF-C | Vascular endothelial growth factor C |
| | factor C | | precursor |
| VHL | von Hippel-Lindau tumor | VHL | Von Hippel-Lindau disease tumor |
| | suppressor | | suppressor |
| YES1 | v-yes-1 Yamaguchi sarcoma viral | YES1, Yes, | Proto-oncogene tyrosine-protein kinase |
| | oncogene homolog 1 | p61-Yes | Yes |
| ZAP70 | zeta-chain (TCR) associated protein | ZAP-70 | Tyrosine-protein kinase ZAP-70 |
| | kinase 70kDa | | |

In some embodiments, additional molecular profiling methods are performed. These can include without limitation PCR, RT-PCR, Q-PCR, SAGE, MPSS, immunoassays and other techniques to assess biological systems described herein or known to those of skill in the art. The choice of genes and gene products to be assayed can be updated over time as new treatments and new drug targets are identified. Once the expression or mutation of a biomarker is correlated with a treatment option, it can be assessed by molecular profiling. One of skill will appreciate that such molecular profiling is not limited to those techniques disclosed herein but comprises any methodology conventional for assessing nucleic acid or protein levels, sequence information, or both. The methods of the invention can also take advantage of any improvements to current methods or new molecular profiling techniques developed in the future. In some embodiments, a gene or gene product is assessed by a single molecular profiling technique. In other embodiments, a gene and/or gene product is assessed by multiple molecular profiling techniques. In a non-limiting example, a gene sequence can be assayed by one or more of FISH and pyrosequencing analysis, the mRNA gene product can be assayed by one or more of RT-PCR and microarray, and the protein gene product can be assayed by one or more of IHC and immunoassay. One of skill will appreciate that any combination of biomarkers and molecular profiling techniques that will benefit disease treatment are contemplated by the invention.

Genes and gene products that are known to play a role in cancer and can be assayed by any of the molecular profiling techniques of the invention include without limitation 2AR, A DISINTEGRIN, ACTIVATOR OF THYROID AND RETINOIC ACID RECEPTOR (ACTR), ADAM 11, ADIPOGENESIS INHIBITORY FACTOR (ADIF), ALPHA 6 INTEGRIN SUBUNIT, ALPHA V INTEGRIN SUBUNIT, ALPHA-CATENIN, AMPLIFIED IN BREAST CANCER 1 (AIB1), AMPLIFIED IN BREAST CANCER 3 (AIB3), AMPLIFIED IN BREAST CANCER 4 (AIB4), AMYLOID PRECURSOR PROTEIN SECRETASE (APPS), AP-2 GAMMA, APPS, ATP-BINDING CASSETTE TRANSPORTER (ABCT), PLACENTA-SPECIFIC (ABCP), ATP-BINDING CASSETTE SUBFAMILY C MEMBER (ABCC1), BAG-1, BASIGIN (BSG), BCEI, B-CELL DIFFERENTIATION FACTOR (BCDF), B-CELL LEUKEMIA 2 (BCL-2), B-CELL STIMULATORY FACTOR-2 (BSF-2), BCL-1, BCL-2-ASSOCIATED X PROTEIN (BAX), BCRP, BETA 1 INTEGRIN SUBUNIT, BETA 3 INTEGRIN SUBUNIT, BETA 5 INTEGRIN SUBUNIT, BETA-2 INTERFERON, BETA-CATENIN, BETA-CATENIN, BONE SIALOPROTEIN (BSP), BREAST CANCER ESTROGEN-INDUCIBLE SEQUENCE (BCEI), BREAST CANCER RESISTANCE PROTEIN (BCRP), BREAST CANCER TYPE 1 (BRCA1), BREAST CANCER TYPE 2 (BRCA2), BREAST CARCINOMA AMPLIFIED SEQUENCE 2 (BCAS2), CADHERIN, EPITHELIAL CADHERIN-11, CADHERIN-ASSOCIATED PROTEIN, CALCITONIN RECEPTOR (CTR), CALCIUM PLACENTAL PROTEIN (CAPL), CALCYCLIN, CALLA, CAMS, CAPL, CARCINOEMBRYONIC ANTIGEN (CEA), CATENIN, ALPHA 1, CATHEPSIN B, CATHEPSIN D, CATHEPSIN K, CATHEPSIN L2, CATHEPSIN 0, CATHEPSIN 01, CATIIEPSIN V, CD10, CD146, CD147, CD24, CD29, CD44, CD51, CD54, CD61, CD66e, CD82, CD87, CD9, CEA, CELLULAR RETINOL-BINDING PROTEIN 1 (CRBP1), c-ERBB-2, CK7, CK8, CK18, CK19, CK20, CLAUDIN-7, c-MET, COLLAGENASE, FIBROBLAST, COLLAGENASE, INTERSTITIAL, COLLAGENASE-3, COMMON ACUTE LYMPHOCYTIC LEUKEMIA ANTIGEN (CALLA), CONNEXIN 26 (Cx26), CONNEXIN 43 (Cx43), CORTACTIN, COX-2, CTLA-8, CTR, CTSD, CYCLIN D1, CYCLOOXYGENASE-2, CYTOKERATIN 18, CYTOKERATIN 19, CYTOKERATIN 8, CYTOTOXIC T-LYMPHOCYTE-ASSOCIATED SERINE ESTERASE 8 (CTLA-8), DIFFERENTIATION-INHIBITING ACTIVITY (DIA), DNA AMPLIFIED IN MAMMARY CARCINOMA 1 (DAM1), DNA TOPOISOMERASE II ALPHA, DR-NM23, E-CADHERIN, EMMPRIN, EMS1, ENDOTHELIAL CELL GROWTH FACTOR (ECGR), PLATELET-DERIVED (PD-ECGF), ENKEPHALINASE, EPIDERMAI, GROWTH FACTOR RECEPTOR (EGFR), EPISIALIN, EPITHELIAL MEMBRANE ANTIGEN (EMA), ER-ALPHA, ERBB2, ERBB4, ER-BETA, ERF-1, ERYTHROID-POTENTIATING ACTIVITY (EPA), ESR1, ESTROGEN RECEPTOR-ALPHA, ESTROGEN RECEPTOR-BETA, ETS-1, EXTRACELLULAR MATRIX METALLOPROTEINASE INDUCER (EMMPRIN), FIBRONECTIN RECEPTOR, BETA POLYPEPTIDE (FNRB), FIBRONECTIN RECEPTOR BETA SUBUNIT (FNRB), FLK-1, GA15.3, GA733.2, GALECTIN-3, GAMMA-CATENIN, GAP JUNCTION PROTEIN (26 kDa), GAP JUNCTION PROTEIN (43 kDa), GAP JUNCTION PROTEIN ALPHA-1 (GJA1), GAP JUNCTION PROTEIN BETA-2 (GJB2), GCP1, GELATINASE A, GELATINASE B, GELATINASE (72 kDa), GELATINASE (92 kDa), GLIOSTATIN, GLUCOCORTICOID RECEPTOR INTERACTING PROTEIN 1 (GRIP1), GLUTATHIONE S-TRANSFERASE p, GM-CSF, GRANULOCYTE CHEMOTACTIC PROTEIN 1 (GCP1), GRANULOCYTE-MACROPHAGE-COLONY STIMULATING FACTOR, GROWTH FACTOR RECEPTOR BOUND-7 (GRB-7), GSTp, HAP, HEAT-SHOCK COGNATE PROTEIN 70 (HSC70), HEAT-STABLE ANTIGEN, HEPATOCYTE GROWTH FACTOR (HGF), HEPATOCYTE GROWTH FACTOR RECEPTOR (HGFR), HEPATOCYTE-STIMULATING FACTOR III (HSF III), HER-2, HER2/NEU, HERMES ANTIGEN, HET, HHM, HUMORAL HYPERCALCEMIA OF MALIGNANCY (HHM), ICERE-1, INT-1, INTERCELLULAR ADHESION MOLECULE-1 (ICAM-1), INTERFERON-GAMMA-INDUCING FACTOR (IGIF), INTERLEUKIN-1 ALPHA (IL-1A), INTERLEUKIN-1 BETA (IL-1B), INTERLEUKIN-11 (IL-11), INTERLEUKIN-17 (IL-17), INTERLEUKIN-18 (IL-18), INTERLEUKIN-6 (IL-6), INTERLEUKIN-8 (IL-8), INVERSELY CORRELATED WITH ESTROGEN RECEPTOR EXPRESSION-1 (ICERE-1), KAIl, KDR, KERATIN 8, KERATIN 18, KERATIN 19, KISS-1, LEUKEMIA INHIBITORY FACTOR (LIF), LIF, LOST IN INFLAMMATORY BREAST CANCER (LIBC), LOT (“LOST ON TRANSFORMATION”), LYMPHOCYTE HOMING RECEPTOR, MACROPHAGE-COLONY STIMULATING FACTOR, MAGE-3, MAMMAGLOBIN, MASPIN, MC56, M-CSF, MDC, MDNCF, MDR, MELANOMA CELL ADHESION MOLECULE (MCAM), MEMBRANE METALLOENDOPEPTIDASE (MME), MEMBRANE-ASSOCIATED NEIJTRAL ENDOPEPTIDASE (NEP), CYSTEINE-RICH PROTEIN (MDC), METASTASIN (MTS-1), MLN64, MMP1, MMP2, MMP3, MMP1, MMP9, MMP11, MMP13, MMP14, MMP15, MMP16, MMP17, MOESIN, MONOCYTE ARGININE-SERPIN, MONOCYTE-DERIVED NEUTROPHIL CHEMOTACTIC FACTOR, MONOCYTE-DERIVED PLASMINOGEN ACTIVATOR INHIBITOR, MTS-1, MUC-1, MUC18, MUCIN LIKE CANCER ASSOCIATED ANTIGEN (MCA), MUCIN, MUC-1, MULTIDRUG RESISTANCE PROTEIN 1 (MDR, MDR1), MULTIDRUG RESISTANCE RELATED PROTEIN-1 (MRP, MRP-1), N-CADHERIN, NEP, NEU, NEUTRAL ENDOPEPTIDASE, NEUTROPHIL-ACTIVATING PEPTIDE 1 (NAP1), NM23-H1, NM23-H2, NME1, NME2, NUCLEAR RECEPTOR COACTIVATOR-1 (NCoA-1), NUCLEAR RECEPTOR COACTIVATOR-2 (NCoA-2), NUCLEAR RECEPTOR COACTIVATOR-3 (NCoA-3), NUCLEOSIDE DIPHOSPHATE KINASE A (NDPKA), NUCLEOSIDE DIPHOSPHATE KINASE B (NDPKB), ONCOSTATIN M (OSM), ORNITHINE DECARBOXYLASE (ODC), OSTEOCLAST DIFFERENTIATION FACTOR (ODF), OSTEOCLAST DIFFERENTIATION FACTOR RECEPTOR (ODFR), OSTEONECTIN (OSN, ON), OSTEOPONTIN (OPN), OXYTOCIN RECEPTOR (OXTR), p27/kipl, p300/CRP COINTEGRATOR ASSOCIATE PROTEIN (p/CIP), p53, p9Ka, PAI-1, PAI-2, PARATHYROID ADENOMATOSIS 1 (PRAD1), PARATHYROID HORMONE-LIKE HORMONE (PTHLH), PARATHYROID HORMONE-RELATED PEPTIDE (PTHrP), P-CADHERIN, PD-ECGF, PDGF, PEANUT-REACTIVE URINARY MUCIN (PUM), P-GLYCOPROTEIN (P-GP), PGP-1, PHGS-2, PHS-2, PIP, PLAKOGLOBIN, PLASMINOGEN ACTIVATOR INHIBITOR (TYPE 1), PLASMINOGEN ACTIVATOR INHIBITOR (TYPE 2), PLASMINOGEN ACTIVATOR (TISSUE-TYPE), PLASMINOGEN ACTIVATOR (UROKINASE-TYPE), PLATELET GLYCOPROTEIN IIIc (GP3A), PLAU, PLEOMORPHIC ADENOMA GENE-LIKE 1 (PLAGL1), POLYMORPHIC EPITHELIAL MUCIN (PEM), PRAD1, PROGESTERONE RECEPTOR (PgR), PROGESTERONE RESISTANCE, PROSTAGLANDIN ENDOPEROXIDE SYNTHASE-2, PROSTAGLANDIN G/H SYNTHASE-2, PROSTAGLANDIN H SYNTHASE-2, pS2, PS6K, PSORIASIN, PTHLH, PTHrP, RAD51, RAD52, RAD54, RAP46, RECEPTOR-ASSOCIATED COACTIVATOR 3 (RAC3), REPRESSOR OF ESTROGEN RECEPTOR ACTIVITY (REA), S100A4, S100A6, S100A7, S6K, SART-1, SCAFFOLD ATTACHMENT FACTOR B (SAF-B), SCATTER FACTOR (SF), SECRETED PHOSPHOPROTEIN-1 (SPP-1), SECRETED PROTEIN, ACIDIC AND RICH IN CYSTEINE (SPARC), STANNICALCIN, STEROID RECEPTOR COACTIVATOR-1 (SRC-1), STEROID RECEPTOR COACTIVATOR-2 (SRC-2), STEROID RECEPTOR COACTIVATOR-3 (SRC-3), STEROID RECEPTOR RNA ACTIVATOR (SRA), STROMELYSIN-1, STROMELYSIN-3, TENASCIN-C (TN-C), TESTES-SPECIFIC PROTEASE 50, THROMBOSPONDIN I, THROMBOSPONDIN II, THYMIDINE PHOSPHORYLASE (TP), THYROID HORMONE RECEPTOR ACTIVATOR MOLECULE 1 (TRAM-1), TIGHT JUNCTION PROTEIN 1 (TJP1), TIMP1, TIMP2, TIMP3, TIMP4, TISSIJE-TYPE PLASMINOGEN ACTIVATOR, TN-C, TP53, tPA, TRANSCRIPTIONAL INTERMEDIARY FACTOR 2 (TIF2), TREFOIL FACTOR 1 (TFF1), TSG101, TSP-1, TSP1, TSP-2, TSP2, TSP50, TUMOR CELL COLLAGENASE STIMULATING FACTOR (TCSF), TUMOR-ASSOCIATED EPITHELIAL MUCIN, uPA, uPAR, UROKINASE, UROKINASE-TYPE PLASMINOGEN ACTIVATOR, UROKINASE-TYPE PLASMINOGEN ACTIVATOR RECEPTOR (uPAR), UVOMORULIN, VASCULAR ENDOTHELIAL GROWTH FACTOR, VASCULAR ENDOTHELIAL GROWTH FACTOR RECEPTOR-2 (VEGFR2), VASCULAR ENDOTHELIAL GROWTH FACTOR-A, VASCULAR PERMEABILITY FACTOR, VEGFR2, VERY LATE T-CELL ANTIGEN BETA (VLA-BETA), VIMENTIN, VITRONECTIN RECEPTOR ALPHA POLYPEPTIDE (VNRA), VITRONECTIN RECEPTOR, VON WILLEBRAND FACTOR, VPF, VWF, WNT-1, ZAC, 70-1, and ZONULA OCCLUDENS-1.

The gene products used for IHC expression profiling include without limitation one or more of AR, BCRP, CD52, c-kit, ER, ERCC1, Her2/neu, MGMT, MRP1, PDGFR, PGP, PR, PTEN, RRM1, SPARC, TOP2A, TOPO1, and TS. In some embodiments, IHC analysis includes one or more of c-Met, EML4-ALK fusion, hENT-1, IGF-1R, MMR, p16, p21, p27, PARP-1, PI3K, and TLE3. IHC profiling of EGFR can also be performed. IHC is also used to detect or test for various gene products, including without limitation one or more of the following: EGFR, SPARC, C-kit, ER, PR, Androgen receptor, PGP, RRM1, TOPO1, BRCP1, MRP1, MGMT, PDGFR, DCK, ERCC1, Thymidylate synthase, Her2/neu, or TOPO2A. In some embodiments, IHC is used to detect on or more of the following proteins, including without limitation: ADA, AR, ASNA, BCL2, BRCA2, c-Met, CD33, CDW52, CES2, DNMT1, EGFR, EML4-ALK fusion, ERBB2, ERCC3, ESR1, FOLR2, GART, GSTP1, HDAC1, hENT-1, HIF1A, HSPCA, IGF-1R, IL2RA, KIT, MLH1, MMR, MS4A1, MASH2, NFKB2, NFKBIA, OGFR, p16, p21, p27, PARP-1, PI3K, PDGFC, PDGFRA, PDGFRB, PGR, POLA, PTEN, PTGS2, RAF1, RARA, RXRB, SPARC, SSTR1, TK1, TLE3, TNF, TOP1, TOP2A, TOP2B, TXNRD1, TYMS, VDR, VEGF, VHL, or ZAP70. The proteins can be detected by IHC using monoclonal or polyclonal antibodies. In sone embodiments, both are used. As an illustrative example, SPARC can be detected by anti-SPARC monoclonal (SPARC mono, SPARC m) and/or anti-SPARC polyclonal (SPARC poly, SPARC p) antibodies.

In some embodiments, IHC analysis according to the methods of the invention includes one or more of AR, c-Kit, CAV-1, CK 5/6, CK14, CK17, ECAD, ER, Her2/Neu, Ki67, MRP1, P53, PDGFR, PGP, PR, PTEN, SPARC, TLE3 and TS. All of these genes can be examined. As indicated by initial results of IHC or other molecular profiling methods as described herein, additional IHC assayscan be performed. In one embodiment, the additional IHC comprises that of p95, or p95, Cyclin D1 and EGFR. IHC can also be performed on IGFRBP3, IGFRBP4, IGFRBP5, or other forms of IGFRBP (e.g., IGFRBP1, IGFRBP2, IGFRBP6, IGFRBP7). In another embodiment, the additional IHC comprises that of one or more of BCRP, ERCC1, MGMT, P95, RRM1, TOP2A, and TOP1. In still another embodiment, the additional IHC comprises that of one or more of BCRP, Cyclin D1, EGFR, ERCC1, MGMT, P95, RRM1, TOP2A, and TOP1. All of these additional genes can be examined. The additional IHC can be selected on the basis of molecular characteristics of the tumor so that IHC is only performed where it is likely to indicate a candidate therapy for treating the cancer. As described herein, the molecular characteristics of the tumor determined can be determined by IHC combined with one or more of FISH, DNA microarray and mutation analysis.

Microarray expression profiling can be used to simultaneously measure the expression of one or more genes or gene products, including without limitation ABCC1, ABCG2, ADA, ALK, AR, ASNS, BCL2, BIRC5, BRCA1, BRCA2, CD33, CD52, CDA, CES2, DCK, DHFR, DNMT1, DNMT3A, DNMT3B, ECGF1, EGFR, EML4, EPHA2, ERBB2, ERCC1, ERCC3, ESR1, FLT1, FOLR2, FYN, GART, GNRH1, GSTP1, HCK, HDAC1, hENT-1, HIF1A, HSP90AA1, IGF-1R, IL2RA, HSP90AA1, KDR, KIT, LCK, LYN, MET, MGMT, MLH1, MMR, MS4A1, MSH2, NFKB1, NFKB2, OGFR, PDGFC, PDGFRA, PDGFRB, p16, p21, p27, PARP-1, PGR, PI3K, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TLE3, TNF, TOP1, TOP2A, TOP2B, TXNRD1, TYMS, VDR, VEGFA, VHL, YES1, and ZAP70. In some embodiments, the genes used for the microarray expression profiling comprise one or more of: EGFR, SPARC, C-kit, ER, PR, Androgen receptor, PGP, RRM1, TOPO1, BRCP1, MRP1, MGMT, PDGFR, DCK, ERCC1, Thymidylate synthase, Her2/neu, TOPO2A, ADA, AR, ASNA, BCL2, BRCA2, CD33, CDW52, CES2, DNMT1, EGFR, ERBB2, ERCC3, ESR1, FOLR2, GART, GSTP1, HDAC1, HIF1A, HSPCA, IL2RA, KIT, MLH1, MS4A1, MASH2, NFKB2, NFKBIA, OGFR, PDGFC, PDGFRA, PDGFRB, PGR, POLA, PTEN, PTGS2, RAF1, RARA, RXRB, SPARC, SSTR1, TK1, TNF, TOP1, TOP2A, TOP2B, TXNRD1, TYMS, VDR, VEGF, VHL, and ZAP70. The microarray expression profiling can be performed using a low density microarray, an expression microarray, a comparative genomic hybridization (CGH) microarray, a single nucleotide polymorphism (SNP) microarray, a proteomic array an antibody array, or other array as disclosed herein or known to those of skill in the art. In some embodiments, high throughput expression arrays are used. Such systems include without limitation commercially available systems from Agilent or Illumina, as described in more detail herein.

Microarray expression profiling can be used to simultaneously measure the expression of one or more genes or gene products, including without limitation ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VIIL, YES1, and ZAP70.

FISH mutation profiling can be used to profile one or more of EGFR and HER2. In some embodiments, FISH is used to detect or test for one or more of the following genes, including without limitation: EGFR, SPARC, C-kit, ER, PR, AR, PGP, RRM1, TOPO1, BRCP1, MRP1, MGMT, PDGFR, DCK, ERCC1, TS, HER2, or TOPO2A. In some embodiments, FISH is used to detect or test for one or more of EML4-ALK fusion and IGF-1R. In some embodiments, FISH is used to detect or test various biomarkers, including without limitation one or more of the following: ADA, AR, ASNA, BCL2, BRCA2, c-Met, CD33, CDW52, CES2, DNMT1, EGFR, EML4-ALK fusion, ERBB2, ERCC3, ESR1, FOLR2, GART, GSTP1, HDAC1, hENT-1, HIF1A, HSPCA, IGF-1R, IL2RA, KIT, MLH1, MMR, MS4A1, MASH2, NFKB2, NFKBIA, OGFR, p16, p21, p27, PARP-1, PI3K, PDGFC, PDGFRA, PDGFRB, POR, POLA, PTEN, PTGS2, RAF1, RARA, RXRII, SPARC, SSTR1, TK1, TLE3, TNF, TOP1, TOP2A, TOP2B, TXNRD1, TYMS, VDR, VEGF, VHL, or ZAP70.

In some embodiments, FISH is used to detect or test for HER2. Depending on the results of the HER2 analysis and other molecular profiling techniques, additional FISH testing may be performed. The additional FISH testing can comprise that of CMYC and/or TOP2A. For example, FISH testing may indicate that a cancer is HER2+. The cancer may be a breast cancer. HER2+ cancers may then be followed up by FISH testing for CMYC and TOP2A, whereas HER2− cancers are followed up with FISH testing for CMYC. For some cancers, e.g., triple negative breast cancer (i.e., ER−/PR−/HER2−), additional FISH testing may not be performed. The decision whether to perform additional FISH testing can be guided by whether the additional FISH testing is likely to reveal information about candidate therapies for the cancer. The additional FISH can be selected on the basis of molecular characteristics of the tumor so that FISH is only performed where it is likely to indicate a candidate therapy for treating the cancer. As described herein, the molecular characteristics of the tumor determined can be determined by one or more of IHC, FISH, DNA microarray and sequence analysis.

In some embodiments, the genes used for the mutation profiling comprise one or more of KRAS, BRAF, c-KIT and EGFR. Mutation profiling can be determined by sequencing, including Sanger sequencing, array sequencing, pyrosequencing, NextGen sequencing, etc. Sequence analysis may reveal that genes harbor activating mutations so that drugs that inhibit activity are indicated for treatment. Alternately, sequence analysis may reveal that genes harbor mutations that inhibit or eliminate activity, thereby indicating treatment for compensating therapies. In embodiments, sequence analysis comprises that of exon 9 and 11 of c-KIT. Sequencing may also be performed on EGFR-kinase domain exons 18, 19, 20, and 21. Mutations, amplifications or misregulations of EGFR or its family members are implicated in about 30% of all epithelial cancers. Sequencing can also be performed on PI3K, encoded by the PIK3CA gene. This gene is a found mutated in many cancers. Sequencing analysis can also comprise assessing mutations in one or more ABCC1, ABCG2, ADA, AR, ASNS, BCL2, BIRC5, BRCA1, BRCA2, c-Met, CD33, CD52, CDA, CES2, DCK, DHFR, DNMT1, DNMT3A, DNMT3B, ECGF1, EGFR, EPIIA2, EML4-ALK fusion, ERBB2, ERCC1, ERCC3, ESR1, FLT1, FOLR2, FYN, GART, GNRH1, GSTP1, HCK, HDAC1, hENT-1, HIF1A, HSP90AA1, IL2RA, HSP90AA1, KDR, KIT, LCK, LYN, MGMT, MLH1, MMR, MS4A1, MSH2, NFKB1, NFKB2, OGFR, p16, p21, p27, PARP-1, PI3K, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TLE3, TNF, TOP1, TOP2A, TOP2B, TXNRD1, TYMS, VDR, VEGFA, VHL, YES1, and ZAP70.

In some embodiments, mutational analysis is performed on PIK3CA. The decision whether to perform mutational analysis on PIK3CA can be guided by whether this testing is likely to reveal information about candidate therapies for the cancer. The PIK3CA mutational analysis can be selected on the basis of molecular characteristics of the tumor so that the analysis is only performed where it is likely to indicate a candidate therapy for treating the cancer. As described herein, the molecular characteristics of the tumor determined can be determined by one or more of IHC, FISH, DNA microarray and sequence analysis. In one embodiment, PIK3CA is analyzed for a HER2+ cancer. The cancer can be a breast cancer.

In a related aspect, the invention provides a method of identifying a candidate treatment for a subject in need thereof by using molecular profiling of sets of known biomarkers. For example, the method can identify a chemotherapeutic agent for an individual with a cancer. The method comprises: obtaining a sample from the subject; performing an immunohistochemistry (IHC) analysis on the sample to determine an IHC expression profile on one or more, e.g. 2, 3, 4, 5, 6, 7, 8, 9, 10 or more, of: SPARC, PGP, Her2/neu, ER, PR, c-kit, AR, CD52, PDGFR, TOP2A, TS, ERCC1, RRM1, BCRP, TOPO1, PTEN, MGMT, MRP1, c-Met, EML4-ALK fusion, hENT-1, IGF-1R, MMR, p16, p21, p27, PARP-1, PI3K, and TLE3; performing a microarray analysis on the sample to determine a microarray expression profile on one or more, e.g. 2, 3, 4, 5, 6, 7, 8, 9, 10 or more, of: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGF-1R, IL2RA, HSP90AA1, KDR, KIT, LCK, LYN, MGMT, MLH1, MS4A1, MSH2, NFKB1, NFKB2, OGFR, PARP1, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing a fluorescent in-situ hybridization (FISH) analysis on the sample to determine a FISH mutation profile on at least one of EGFR, HER2, EML4-ALK fusion and IGF-1R; performing DNA sequencing on the sample to determine a sequencing mutation profile on at least one of KRAS, BRAF, c-KIT, PI3K (PIK3CA) and EGFR; and comparing the IHC expression profile, microarray expression profile, FISH mutation profile and sequencing mutation profile against a rules database, wherein the rules database comprises a mapping of treatments whose biological activity is known against diseased cells that: i) overexpress or underexpress one or more proteins included in the MC expression profile; ii) overexpress or underexpress one or more genes included in the microarray expression profile; iii) have zero or more mutations in one or more genes included in the FISH mutation profile; and/or iv) have zero or more mutations in one or more genes included in the sequencing mutation profile; and identifying the treatment if the comparison against the rules database indicates that the treatment should have biological activity against the disease; and the comparison against the rules database does not contraindicate the treatment for treating the disease. The disease can be a cancer. The molecular profiling steps can be performed in any order. In some embodiments, not all of the molecular profiling steps are performed. As a non-limiting example, microarray analysis is not performed if the sample quality does not meet a threshold value, as described herein. In some embodiments, the IHC expression profiling is performed on at least 20%, 30%, 40%, 50%, 60%, 70%, 80%, 90%, or 95% of the gene products above. In some embodiments, the microarray expression profiling is performed on at least 20%, 30%, 40%, 50%, 60%, 70%, 80%, 90%, or 95% of the genes listed above.

In a related aspect, the invention provides a method of identifying a candidate treatment for a subject in need thereof by using molecular profiling of defined sets of known biomarkers. For example, the method can identify a chemotherapeutic agent for an individual with a cancer. The method comprises: obtaining a sample from the subject, wherein the sample comprises formalin-fixed paraffin-embedded (FFPE) tissue or fresh frozen tissue, and wherein the sample comprises cancer cells; performing an immunohistochemistry (IHC) analysis on the sample to determine an IHC expression profile on at least: SPARC, PGP, Her2/neu, ER, PR, c-kit, AR, CD52, PDGFR, TOP2A, TS, ERCC1, RRM1, BCRP, TOPO1, PTEN, MGMT, MRP1, c-Met, EML4-ALK fusion, hENT-1, IGF-1R, MMR, p16, p21, p27, PARP-1, PI3K, and TLE3; performing a microarray analysis on the sample to determine a microarray expression profile on at least: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGF-1R, IL2RA, HSP90AA1, KDR, KIT, LCK, LYN, MGMT, MLH1, MS4A1, MSH2, NFKB1, NFKB2, OGFR, PARP1, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing a fluorescent in-situ hybridization (FISH) analysis on the sample to determine a FISH mutation profile on at least one of EGFR, HER2, EML4-ALK fusion and IGF-1R; performing DNA sequencing on the sample to determine a sequencing mutation profile on at least KRAS, BRAF, c-KIT, PI3K (PIK3CA) and EGFR. The IHC expression profile, microarray expression profile, FISH mutation profile and sequencing mutation profile are compared against a rules database, wherein the rules database comprises a mapping of treatments whose biological activity is known against diseased cells that: i) overexpress or underexpress one or more proteins included in the IHC expression profile; ii) overexpress or underexpress one or more genes included in the microarray expression profile; iii) have zero or more mutations in one or more genes included in the FISH mutation profile; or iv) have zero or more mutations in one or more genes included in the sequencing mutation profile; and identifying the treatment if the comparison against the rules database indicates that the treatment should have biological activity against the disease; and the comparison against the rules database does not contraindicate the treatment for treating the disease. The disease can be a cancer. The molecular profiling steps can be performed in any order. In some embodiments, not all of the molecular profiling steps are performed. As a non-limiting example, microarray analysis is not performed if the sample quality does not meet a threshold value, as described herein. In some embodiments, the biological material is mRNA and the quality control test comprises a A260/A280 ratio and/or a Ct value of RT-PCR using a housekeeping gene, e.g., RPL13a. In embodiments, the mRNA does not pass the quality control test if the A260/A280 ratio <1.5 or the RPL13a Ct value is >30. In that case, microarray analysis may not be performed. Alternately, microarray results may be attenuated, e.g., given a lower priority as compared to the results of other molecular profiling techniques.

In some embodiments, molecular profiling is always performed on certain genes or gene products, whereas the profiling of other genes or gene products is optional. For example, IHC expression profiling may be performed on at least SPARC, TOP2A and/or PTEN. Similarly, microarray expression profiling may be performed on at least CD52. In other embodiments, genes in addition to those listed above are used to identify a treatment. For example, the group of genes used for the IHC expression profiling can further comprise DCK, EGFR, BRCA1, CK 14, CK 17, CK 5/6, E-Cadherin, p95, PARP-1, SPARC and TLE3. In some embodiments, the group of genes used for the IHC expression profiling further comprises Cox-2 and/or Ki-67. In some embodiments, HSPCA is assayed by microarray analysis. In some embodiments, FISH mutation is performed on c-Myc and TOP2A. In some embodiments, sequencing is performed on PI3K.

The methods of the invention can be used in any setting wherein differential expression or mutation analysis have been linked to efficacy of various treatments. In some embodiments, the methods are used to identify candidate treatments for a subject having a cancer. Under these conditions, the sample used for molecular profiling preferably comprises cancer cells. The percentage of cancer in a sample can be determined by methods known to those of skill in the art, e.g., using pathology techniques. Cancer cells can also be enriched from a sample, e.g., using microdissection techniques or the like. A sample may be required to have a certain threshold of cancer cells before it is used for molecular profiling. The threshold can be at least about 5, 10, 20, 30, 40, 50, 60, 70, 80, 90 or 95% cancer cells. The threshold can depend on the analysis method. For example, a technique that reveals expression in individual cells may require a lower threshold that a technique that used a sample extracted from a mixture of different cells. In some embodiments, the diseased sample is compared to a normal sample taken from the same patient, e.g., adjacent but non-cancer tissue.

In embodiments, the methods of the invention are used detect gene fusions, such as those listed in Table 2. A fusion gene is a hybrid gene created by the juxtaposition of two previously separate genes. This can occur by chromosomal translocation or inversion, deletion or via trans-splicing. The resulting fusion gene can cause abnormal temporal and spatial expression of genes, leading to abnormal expression of cell growth factors, angiogenesis factors, tumor promoters or other factors contributing to the neoplastic transformation of the cell and the creation of a tumor. For example, such fusion genes can be oncogenic due to the juxtaposition of: 1) a strong promoter region of one gene next to the coding region of a cell growth factor, tumor promoter or other gene promoting oncogenesis leading to elevated gene expression, or 2) due to the fusion of coding regions of two different genes, giving rise to a chimeric gene and thus a chimeric protein with abnormal activity. Fusion genes are characteristic of many cancers, such as those listed in Table 2. Once a therapeutic intervention is associated with a fusion, the presence of that fusion in any type of cancer identifies the therapeutic intervention as a candidate therapy for treating the cancer.

TABLE 2
Fusion Genes and Associated Cancers

| 5′ Upstream | 3′ downstream | |
|---|---|---|
| Fusion Gene | Fusion Gene | |
| Partner | Partner | Cancer Lineage |
| ACSL3 | ETV1 | Prostate cancer |
| AKAP9 | BRAF | Papillary thyroid carcinoma |
| Alpha | TFEB | Renal cell carcinoma |
| ARHGAP20 | BRWD3 | B-cell chronic lymphocytic |
| | | leukemia (B-CLL) |
| ASPSCR1 | TFE3 | Renal-cell carcinoma |
| ATIC | ALK | Anaplastic large cell lymphoma (ALCL) |
| BCL11B | TLX3 | T-cell acute lymphoblastic/lymphocytic |
| | | leukemia (T-ALL) |
| BCL3 | MYC | B-cell chronic lymphocytic leukemia |
| | | (B-CLL) |
| BCL7A | MYC | B-cell chronic lymphocytic leukemia |
| | | (B-CLL) |
| BCR | ABL1 | Chronic myelogenous leukemia (CML) |
| BCR | FGFR1 | CML-like Myeloproliferative disorder |
| | | (MPD) |
| BCR | JAK2 | Chronic myelogenous leukemia (CML) |
| BCR | PDGFRA | Atypical CML |
| BIRC3 | MALT1 | B-cell non Hodgkin lymphoma, |
| | | MALT-lymphomas |
| BRD4 | NUT | Poorly differentiated epithelial carcinoma |
| | | (Aggressive midline carcinoma) |
| BRWD3 | ARIIGAP20 | B-cell chronic lymphocytic leukemia |
| | | (B-CLL) |
| BTG1 | MYC | B-cell chronic lymphocytic leukemia |
| | | (B-CLL) |
| CARS | ALK | Inflammatory myofibroblastic tumor |
| CANT1 | ETV4 | Prostate cancer |
| CBFB | MYH11 | Acute myelogenous leukemia (AML) |
| CCDC6 | PDGFRB | Philadelphia chr negative |
| | | Myeloproliferative disorder (MPD) |
| CCDC6 | RET | Papillary thyroid carcinoma |
| CCNDI | FSTL3 | Chronic myelogenous leukemia (CML) |
| CD74 | ROS1 | Non small cell lung carcinoma (NSCLC) |
| CDH11 | USP6 | Aneurysmal bone cyst |
| CDK6 | EV1I | Myeloid leukemia |
| CDK6 | MLL | Acute lymphoblastic/lymphocytic |
| | | leukemia (ALL) |
| CDK6 | TLX3 | Acute lymphoblastic/lymphocytic |
| | | leukemia (ALL) |
| CEP110 | FGFR1 | Myeloproliferative disorder |
| | | (Myeloproliferative disorder (MPD)) |
| CHCHD7 | PLAG1 | Pleomorphic salivary gland adenomas |
| | | (PA) (Head and Neck) |
| CHIC2 | ETV6 | Acute myelogenous leukemia (AML) |
| CHTA | BCL6 | Diffuse large B-cell lymphoma (DLBCL) |
| CLTC | ALK | Diffuse large B-cell lymphoma (DLBCL) |
| CLTC | TFE3 | Pediatric renal adcnocarcinoma |
| C15ORF21 | ETV1 | Prostate cancer |
| COL1A1 | PDGFB | Dermatofibrosarcoma protuberans |
| COL1A1 | USP6 | Aneurysmal bone cyst |
| COL1A2 | PLAG1 | Lipoblastoma |
| CRC1 | MAML2 | Mucoepidermoid carcinoma |
| CRTC1 | MAML2 | Mucoepidermoid carcinomas, |
| | | Warthin's tumor |
| CRTC3 | MAML2 | Mucoepidermoid carcinoma |
| CTNNB1 | PLAG1 | Pleomorphic salivary gland adenomas |
| | | (PA) (Head and Neck) |
| DDX5 | ETV4 | Prostate cancer |
| EIF4A2 | BCL6 | Non-Hodgkin lymphoma (NHL) |
| EML1 | ABL1 | T-cell acute lymphoblastic/lymphocytic |
| | | leukemia (T-ALL) |
| EML4 | ALK | Non small cell lung carcinoma (NSCLC) |
| EPC1 | PHF1 | Endometrial stromal sarcoma |
| ERC1 | RET | Papillary thyroid carcinoma |
| ETV6 | ABL1 | Chronic myelogenous leukemia |
| | | (CML), Acute myelogenous |
| | | leukemia (AML), Acute |
| | | lymphoblastic/lymphocytic leukemia |
| | | (ALL) |
| ETV6 | ABL2 | T-cell acute lymphoblastic/lymphocytic |
| | | leukemia (T-ALL), Acute |
| | | myelogenous leukemia (AML) |
| ETV6 | ACSL6 | Polycythemia vera |
| ETV6 | ARNT | Acute myelogenous leukemia (AML) |
| ETV6 | CDX2 | Acute myelogenous leukemia (AML) |
| ETV6 | EVI1 | Chronic myelogenous leukemia (CML) |
| ETV6 | FGFR3 | Peripheral T-cell lymphoma |
| ETV6 | FLT3 | ALL, Myeloproliferative disorder (MPD) |
| ETV6 | HLXB9 | Acute myelogenous leukemia (AML) |
| ETV6 | JAK2 | Philadelphia chr negative |
| | | Myeloproliferative disorder |
| | | (MPD), B cell malignancies |
| ETV6 | MDS2 | Myelodisplastic syndrome |
| ETV6 | MN1 | Chronic myelogenous leukemia (CML) |
| ETV6 | NTRK3 | Secretory breast cancer |
| ETV6 | PDGFRB | Chronic myelomonocytic leukemia |
| | | (CMML) |
| ETV6 | PER1 | Acute myelogenous leukemia (AML) |
| ETV6 | RUNX1 | Acute lymphoblastic/lymphocytic |
| | | leukemia (ALL) |
| ETV6 | SYK | Myelodisplastic syndrome |
| ETV6 | TCBA1 | Chronic myelogenous leukemia (CML) |
| ETV6 | TTL | Acute lymphoblastic/lymphocytic |
| | | leukemia (ALL) |
| EWSR1 | ATF1 | Soft tissue sarcoma |
| EWSR1 | DDIT3 | Myxoid liposarcoma |
| EWSR1 | ERG | Ewing sarcomas |
| EWSR1 | ETV 1 | Ewing sarcomas |
| EWSR1 | ETV4 | Ewing sarcomas |
| EWSR1 | FEV | Ewing sarcomas |
| EWSR1 | FLI1 | Ewing sarcomas |
| EWSR1 | NR4A3 | Malignant tumor of soft tissue origin |
| EWSR1 | POU5F1 | Undifferentiated bone tumor |
| EWSR1 | TEC | Ewing sarcomas |
| EWSR1 | WT1 | Soft tissue sarcoma |
| EWSR1 | ZNF278 | Small round cell sarcoma |
| EWSR1 | ZNF384 | Acute lymphoblastic leukemia |
| FGFR1OP | FGFR1 | Stem-cell myeloproliferative disorder |
| | | characterized by myeloid hyperplasia, |
| | | T -cell lymphoblastic leukemia/ |
| | | lymphoma and peripheral blood |
| | | eosinophilia, and it generally progresses |
| | | to acute myeloid leukemia; |
| FGFR1OP2 | FGFR1 | Myeloproliferative disorder (MPD) |
| | | is characterized by myeloid hyperplasia, |
| | | eosinophilia and T-cell or |
| | | B -cell lymphoblastic lymphoma |
| FHIT | HMGA2 | Pleomorphic salivary gland adenomas |
| | | (PA) (Head and Neck) |
| FIP1L1 | PDGFRA | Hypereosinophilia |
| FLT3 | ETV6 | Hypereosinophilia |
| FLJ35294 | ETV1 | Prostate cancer |
| FUS | ATF1 | Angiomatoid fibrous histiocytoma (AFH) |
| FUS | CREB3L1 | Fibromyxoid sarcoma |
| FUS | CREB3L2 | Low-grade fibromyxoid sarcoma (LGFMS) |
| FUS | DDIT3 | Myxoid liposarcoma |
| FUS | DDIT3 | The Myxoid/Round Cell Liposarcoma |
| FUS | ERG | Ewing sarcomas |
| GAPDH | BCL6 | B-cell non Hodgkin lymphoma (B-NHL), |
| | | Diffuse large B-cell lymphoma |
| | | (DLBCL) |
| GOLGA5 | RET | Papillary thyroid carcinoma |
| GOPC | ROS1 | Glioblastoma |
| HAS2 | PLAG1 | Lipoblastoma |
| HERV | ETV1 | Prostate cancer |
| HIP1 | PDGFRB | Chronic myelomonocytic leukemia |
| | | (CMML) |
| HIST1H4I | BCL6 | B-cell Non-Hodgkin lymphoma (B-NHL) |
| HMGA1 | LAMA4 | Pulmonary chondroid hamartoma |
| HMGA2 | CCNB1IP1 | Benign mesenchymal tumors |
| HMGA2 | COX6C | Uterine leiomyoma |
| HMGA2 | CXCR7 | Lipoma |
| HMGA2 | PHIT | Pleomorphic salivary gland adenomas |
| | | (PA) (Head and Neck) |
| HMGA2 | LHFP | Solitary lipomas |
| HMGA2 | LPP | Lipoma, parosteal lipoma, and pulmonary |
| | | chondroid hamartoma |
| HMGA2 | NFIB | Pleomorphic salivary gland adenomas |
| | | (PA) (Head and Neck) |
| HMGA2 | RAD51L1 | Uterine leiomyomata |
| HNRPA2B1 | ETV1 | Prostate cancer |
| HOOK3 | RET | Papillary thyroid carcinoma |
| HRH4 | RET | Papillary thyroid carcinoma |
| HSP90AA1 | BCL6 | B cell Non-Hodgkin lymphoma (B-NHL) |
| HSP90AB1 | BCL6 | B-cell tumors |
| IGH | MYC | Burkitt's lymphoma |
| IKZF1 | BCL6 | Diffuse large B-cell lymphoma (DLBCL) |
| IL2 | TNFRSF17 | T-cell acute lymphoblastic |
| | | leukemia (T-ALL) |
| IL21R | BCL6 | Diffuse large B-cell lymphoma (DLBCL) |
| ITK | SYK | Unspecified peripheral T-cell lymphoma |
| JAZF1 | PHF1 | Endometrial stromal sarcomas |
| JAZF1 | SUZ12 | endometrial stromal tumors and |
| | | endometrial stromal sarcoma |
| KIAA1509 | PDGFRA | Chronic eosinophilic leukemia (CEL) |
| KIAA1618 | ALK | Anaplastic large-cell lymphoma (ALCL) |
| KLK2 | ETV4 | Prostate cancer |
| KTN1 | RET | Papillary thyroid carcinoma |
| LCP1 | BCL6 | Non Hodgkin follicular, Burkitt lymphomas |
| LIFR | PLAG1 | Pleomorphic salivary gland adenomas |
| | | (PA) (Head and Neck) |
| MALAT1 | TFEB | Pediatric renal neoplasm |
| MEF2D | DAZAP1 | Acute myelogenous leukemia (AML) |
| MLL | ABI1 | acute non lymphoblastic leukemia |
| MLL | AFF1 | Acute lymphoblastic/lymphocytic |
| | | leukemia (ALL), Acute myelogenous |
| | | leukemia (AML) |
| MLL | AFF3 | Acute lymphoblastic/lymphocytic |
| | | leukemia (ALL) |
| MLL | AFF4 | Acute lymphoblastic/lymphocytic |
| | | leukemia (ALL) |
| MLL | ARHGAP26 | Acute monocytic leukemia (Acute |
| | | myelogenous leukemia (AML) |
| | | (M5b) |
| MLL | ARHGEF12 | Acute myelogenous leukemia (AML) |
| MLL | CASC5 | Acute myelogenous leukemia (AML) |
| MLL | CBL | Acute myelogenous leukemia (AML) |
| MLL | CLP1 | Monoblastic leukemia |
| MLL | CREBBP | Acute myelogenous leukemia (AML) |
| MLL | CXXC6 | Acute lymphoblastic/lymphocytic |
| | | leukemia (ALL) |
| MLL | DAB2IP | Acute myelogenous leukemia (AML) |
| MLL | ELL | Acute myelogenous leukemia (AML) |
| MLL | EP300 | Acute myelogenous leukemia (AML) |
| MLL | EPS15 | Acute myelogenous leukemia (AML) |
| MLL | FNBP1 | Acute myelogenous leukemia (AML) |
| MLL | FOXO3A | Acute myelogenous leukemia (AML) |
| MLL | GAS7 | Acute lymphoblastic/lymphocytic |
| | | leukemia (ALL) |
| MLL | GMPS | Acute myelogenous leukemia (AML) |
| MLL | GPHN | Acute myelogenous leukemia (AML) |
| MLL | LASP1 | Infant acute myeloid leukemia Acute |
| | | myelogenous leukemia (AML)-M4 |
| MLL | LPP | Secondary acute leukemia |
| MLL | MAPRE1 | Pro-B acute lymphoblastic leukemia |
| MLL | MLL | Acute myeloid and lymphoid leukemia |
| MLL | MLLT1 | Acute myelogenous leukemia (AML) |
| MLL | MLLT10 | Pediatric acute megakaryoblastic |
| | | leukemia AND acute monoblastic |
| | | leukemia |
| MLL | MLLT11 | Acute myelogenous leukemia (AML) |
| MLL | MLLT3 | Acute myelogenous leukemia (AML) |
| MLL | MLLT4 | M4/M5 ANLL |
| MLL | MLLT6 | Acute myelogenous leukemia (AML) |
| MLL | MLLT7 | Acute leukemias |
| MLL | MYO1F | Acute myelogenous leukemia (AML) |
| MLL | PICALM | Acute myelogenous leukemia (AML) |
| MLL | RARA | M5 acute non lymphocytic |
| | | leukemia (ANLL) |
| MLL | SEPT11 | Chronic neutrophilic leukemia |
| MLL | SEPT2 | Acute myelogenous leukemia (AML), |
| | | therapy-related myelodysplastic |
| | | syndrome |
| MLL | SEPT5 | De novo acute non lymphocytic leukemia |
| MLL | SEPT6 | Acute myelogenous leukemia (AML) |
| MLL | SEPT9 | Myeloid neoplasia |
| MLL | SH3GL1 | Acute leukemia |
| MLL | SORBS2 | Acute myelogenous leukemia (AML) |
| MLL | ZFYVE19 | Acute myelogenous leukemia (AML) |
| MSI2 | HOXA9 | Chronic myelogenous leukemia (CML) |
| MSN | ALK | Anaplastic large cell lymphoma (ALCL) |
| MYC | BCL7A | High-grade B cell Non-Hodgkin |
| | | lymphoma (NHL) |
| MYC | BTG1 | B-cell chronic lymphocytic |
| | | leukemia (B-CLL) |
| MYH9 | ALK | Anaplastic large cell lymphoma (ALCL) |
| MYST3 | ASXL2 | Therapy-related myelodysplastic syndrome |
| MYST3 | CREBBP | Acute myelogenous leukemia (AML) |
| MYST3 | EP300 | Acute myelomonocytic or monocytic |
| | | leukemia (M4 or M5 Acute myelogenous |
| | | leukemia (AML)) |
| MYST3 | NCOA2 | Acute leukemia |
| MYST4 | CREBBP | Acute myelogenous leukemia (AML) |
| NACA | BCL6 | Non-Hodgkin lymphoma (NHL) |
| NCOA4 | RET | Papillary thyroid carcinoma |
| NIN | PDGFRB | Chronic myeloproliferative disorder with |
| | | eosinophilia |
| NONO | TFE3 | Renal cell carcinoma |
| NPM1 | ALK | Anaplastic large-cell lymphomas (ALCL) |
| NPM1 | MLF1 | Acute myelogenous leukemia (AML) |
| NPM1 | RARA | Acute promyelocytic leukemia (APML) |
| NUMA1 | RARA | Atypical M3 acute non lymphoblastic |
| | | leukemia (ANLL) |
| NUP214 | ABL1 | T-cell acute lymphoblastic/lymphocytic |
| | | leukemia (T-ALL) |
| NUP214 | DEK | Acute myelogenous leukemia (AML) and |
| | | myelodysplastic syndrome |
| NUP214 | SET | Acute undifferentiated leukemia (AUL) |
| NUP98 | ADD3 | T-cell acute lymphoblastic leukemia with |
| | | biphenotypic characteristics (T/myeloid) |
| NUP98 | CCDC28A | Acute megakaryoblastic leukemia, |
| | | AND T cell acute lymphoblastic |
| | | leukemia (T-ALL) |
| NUP98 | DDX10 | De novo or secondary myeloid malignancies |
| NUP98 | HOXA11 | Juvenile myelomonocytic leukemia (JMML) |
| NUP98 | HOXA13 | Acute myelogenous leukemia (AML) |
| NUP98 | HOXA9 | Acute myelogenous leukemia (AML) |
| NUP98 | HOXC11 | Acute myelogenous leukemia (AML) |
| NUP98 | HOXC13 | Acute myelogenous leukemia (AML) |
| NUP98 | HOXD11 | Acute myelomonocytic leukemia |
| NUP98 | HOXD13 | Acute myelogenous leukemia (AML) |
| NUP98 | JARID1A | Acute leukemia |
| NUP98 | NSD1 | Childhood acute myelogenous |
| | | leukemia (AML) |
| NUP98 | PRRX1 | M2-ANLL, Non Hodgkin |
| | | lymphoma (NHL) |
| NUP98 | PRRX2 | Acute myelogenous leukemia (AML) |
| NUP98 | PSIP1 | Acute non lymphoblastic leukemia |
| NUP98 | RAP1GDS1 | T acute lymphoblastic leukemia |
| NUP98 | TOP1 | Acute myelogenous leukemia (AML) |
| NUP98 | WHSC1L1 | Acute myelogenous leukemia (AML) |
| NUT | BRD4 | Midline carcinoma |
| OMD | USP6 | Aneurysmal bone cyst |
| PAX3 | FOXO1 | Rhabdomyosarcoma |
| PAX5 | ETV6 | Acute lymphoblastic/lymphocytic |
| | | leukemia (ALL) |
| PAX7 | FOXO1 | Alveolar rhabdomyosarcomas |
| PAX8 | PPARy | Follicular thyroid carcinoma |
| PCM1 | JAK2 | Myeloproliferative disorder (MPD) |
| | | and acute erythroid leukemia |
| PCM1 | RET | Papillary thyroid carcinoma |
| PDE4DIP | PDGFRB | Chronic eosinophilic leukemia (CEL) |
| PICALM | MLLT10 | CML, Acute myelogenous leukemia (AML) |
| PIM1 | BCL6 | Diffuse large B-cell lymphoma (DLBCL) |
| PML | RARA | Acute promyelocytic leukemia (APML) |
| POU2AF1 | BCL6 | Non-Hodgkin lymphoma (NHL) |
| PRCC | TFE3 | Renal cell carcinoma |
| PRDM16 | EVI1 | MDS and Acute myelogenous |
| | | leukemia (AML) |
| PRKAR1A | RET | Papillary thyroid carcinoma |
| RABEPI | PDGFRB | Myeloproliferative disorder (MPD) and |
| | | Acute myelogenous leukemia (AML), |
| RANBP2 | ALK | Inflammatory myofibroblastic |
| | | tumors (IMT) |
| RBM15 | MKL1 | Acute myelogenous leukemia (AML) |
| RFG | RET | Papillary thyroid carcinoma |
| RFG9 | RET | Papillary thyroid carcinoma |
| RHOH | BCL6 | Follicular centrocytic-centroblastic |
| | | lymphoma. |
| Ria | RET | Papillary thyroid carcinoma |
| RLF | MYCL1 | Small-cell lung cancer (SCLC) |
| RPN1 | EVI1 | Acute non lymphocytic leukemia (ANLL), |
| | | Myelodysplastic syndrome |
| RUNX1 | CBFA2T3 | Myeloid malignancies. |
| RUNX1 | EVI1 | Acute myelogenous leukemia (AML), |
| | | therapy-related MDS and chronic |
| | | myeloid leukemia in blastic phase |
| RUNX1 | MDS1 | Acute myelogenous leukemia (AML), |
| | | therapy-related MDS |
| | | and chronic myeloid leukemia in |
| | | blastic phase |
| RUNX1 | RPL22 | Acute myelogenous leukemia (AML) |
| RUNX1 | RUNX1T1 | Acute myelogenous leukemia (AML) |
| RUNX1 | SH3D19 | Acute myelogenous leukemia (AML) |
| RUNX1 | USP42 | Acute myelogenous leukemia (AML) |
| RUNX1 | YTHDF2 | Acute myelogenous leukemia (AML) |
| RUNX1 | ZNF687 | Acute myelogenous leukemia (AML) |
| SEC31A | ALK | Diffuse large B-cell lymphoma (DLBCL) |
| SENP6 | TCBA1 | T-cell lymphoma |
| SFPQ | TFE3 | Renal cell carcinoma |
| SFRS3 | BCL6 | Follicular lymphoma |
| SLC5A3 | ERG | Prostate cancer |
| SLC45A3 | ETV1 | Prostate cancer |
| SLC45A3 | ETV5 | Prostate cancer |
| SPECC1 | PDGFRB | Juvenile myelomonocytic leukemia |
| SS18 | SSX1 | Synovial sarcoma |
| SS18 | SSX2 | Synovial sarcoma |
| SS18 | SSX4 | Synovial sarcoma |
| SS18L1 | SSX1 | Synovial sarcoma |
| STAT5B | RARA | Acute promyelocytic leukemia (APML) |
| TAF15 | NR4A3 | Ewing's sarcoma/primitive |
| | | neuroectodermal tumor |
| TAF15 | TEC | Ewing sarcomas |
| TAF15 | ZNF384 | Acute myelogenous leukemia (AML) |
| TAL1 | STIL | T-cell malignancies (T-ALL) |
| TCBA1 | ETV6 | Acute lymphoblastic/lymphocytic |
| | | leukemia (ALL) |
| TCEA1 | PLAG1 | Pleomorphic salivary gland adenomas |
| | | (PA) (Head and Neck) |
| TCF12 | NR4A3 | Extraskeletal myxoid chondrosarcoma |
| TCF12 | TEC | Extraskeletal myxoid chondrosarcoma |
| TCF3 | HLF | pre-B-cell acute lymphoblastic leukemia |
| TCF3 | PBX1 | Acute lymphoblastic/lymphocytic |
| | | leukemia (ALL) |
| TCF3 | TFPT | Acute lymphoblastic/lymphocytic |
| | | leukemia (ALL) |
| TFG | ALK | Anaplastic large cell lymphoma |
| | | (ALCL), Non small |
| | | cell lung carcinoma (NSCLC) |
| TFG | NR4A3 | Extraskeletal myxoid chondrosarcoma |
| TFG | NTRK1 | Papillary thyroid carcinoma |
| TFRC | BCL6 | B-cell non Hodgkin lymphoma (B-NHL), |
| | | Diffuse large B-cell lymphoma |
| | | (DLBCL) |
| THRAP3 | USP6 | Aneurysmal bone cysts |
| TIAF1 | FGFR1 | Myeloproliferative disorder (MPD) |
| TMPRSS2 | ERG | Prostate cancer |
| TMPRSS2 | ETV1 | Prostate cancer |
| TMPRSS2 | ETV4 | Prostate cancer |
| TMPRSS2 | ETV5 | Prostate cancer |
| TP53BP1 | PDGFRB | CML-like disorder associated |
| | | with eosinophilia |
| TPM3 | ALK | Anaplastic large cell lymphoma (ALCL) |
| TPM3 | NTRK1 | Papillary thyroid carcinoma |
| TPM3 | PDGFRB | Chronic eosinophilic leukemia (CEL) |
| TPM3 | TPR | Papillary thyroid carcinoma |
| TPM4 | ALK | Inflammatory Myofibroblastic Tumors |
| TPR | MET | Papillary thyroid carcinoma |
| TPR | NTRK1 | Papillary thyroid carcinoma |
| TRIM24 | FGFR1 | Myeloproliferative disorder (MPD) |
| TRIM24 | RARA | Myeloproliferative disorder (MPD) |
| TRIM24 | RET | Papillary thyroid carcinoma |
| TRIM27 | RET | Papillary thyroid carcinoma |
| TRIM33 | RET | Papillary thyroid carcinoma |
| TRIP11 | PDGFRB | Acute myelogenous leukemia (AML) |
| TTL | ETV6 | Acute lymphoblastic/lymphocytic |
| | | leukemia (ALL) |
| ZBTB16 | RARA | Acute promyelocytic leukemia (APML) |
| ZMYM2 | FGFR1 | Stem cell leukemia lymphoma |
| | | syndrome (SCLL) |

The presence of fusion genes, e.g., those described in Table 2 or elsewhere herein, can be used to guide therapeutic selection. For example, the BCR-ABL gene fusion is a characteristic molecular aberration in ˜90% of chronic myelogenous leukemia (CML) and in a subset of acute leukemias (Kurzrock et al., Annals of Internal Medicine 2003; 138:819-830). The BCR-ABL results from a translocation between chromosomes 9 and 22, commonly referred to as the Philadelphia chromosome or Philadelphia translocation. The translocation brings together the 5′ region of the BCR gene and the 3′ region of ABL1, generating a chimeric BCR-ABL1 gene, which encodes a protein with constitutively active tyrosine kinase activity (Mittleman et al., Nature Reviews Cancer 2007; 7:233-245). The aberrant tyrosine kinase activity leads to de-regulated cell signaling, cell growth and cell survival, apoptosis resistance and growth factor independence, all of which contribute to the pathophysiology of leukemia (Kurzrock et al., Annals of Internal Medicine 2003; 138:819-830). Patients with the Philadelphia chromosome are treated with imatinib and other targeted therapies. Imatinib binds to the site of the constitutive tyrosine kinase activity of the fusion protein and prevents its activity. Imatinib treatment has led to molecular responses (disappearance of BCR-ABL+ blood cells) and improved progression-free survival in BCR-ABL+ CML patients (Kantarjian et al., Clinical Cancer Research 2007; 13:1089-1097).

Another fusion gene, IGH-MYC, is a defining feature of ˜80% of Burkitt's lymphoma (Ferry et al. Oncologist 2006; 11:375-83). The causal event for this is a translocation between chromosomes 8 and 14, bringing the c-Myc oncogene adjacent to the strong promoter of the immunoglobulin heavy chain gene, causing c-myc overexpression (Mittleman et al., Nature Reviews Cancer 2007; 7:233-245). The c-myc rearrangement is a pivotal event in lymphomagenesis as it results in a perpetually proliferative state. It has wide ranging effects on progression through the cell cycle, cellular differentiation, apoptosis, and cell adhesion (Ferry et al. Oncologist 2006; 11:375-83).

A number of recurrent fusion genes have been catalogued in the Mittleman database (cgap.nci.nih.gov/Chromosomes/Mitelman). The gene fusions can be used to characterize neoplasms and cancers and guide therapy using the subject methods described herein. For example, TMPRSS2-ERG, TMPRSS2-ETV and SLC45A3-ELK4 fusions can be detected to characterize prostate cancer; and ETV6-NTRK3 and ODZ4-NRG1 can be used to characterize breast cancer. The EML4-ALK, RLF-MYCL1, TGF-ALK, or CD74-ROS1 fusions can be used to characterize a lung cancer. The ACSL3-ETV1, C15ORF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4 fusions can be used to characterize a prostate cancer. The GOPC-ROS1 fusion can be used to characterize a brain cancer. The CHCHD7-PLAG1, CTNNB1-PLAG1, FHIT-HMGA2, HMGA2-NFIB, LIFR-PLAG1, or TCEA1-PLAG1 fusions can be used to characterize a head and neck cancer. The ALPHA-TFEB, NONO-TFE3, PRCC-TFE3, SFPQ-TFE3, CLTC-TFE3, or MALAT1-TFEB fusions can be used to characterize a renal cell carcinoma (RCC). The AKAP9-BRAF, CCDC6-RET, ERC1-RETM, GOLGA5-RET, HOOKS-RET, HRH4-RET, KTN1-RET, NCOA4-RET, PCM1-RET, PRKARA1A-RET, RFG-RET, RFG9-RET, Ria-RET, TGF-NTRK1, TPM3-NTRK1, TPM3-TPR, TPR-MET, TPR-NTRK1, TRIM24-RET, TRIM27-RET or TRIM33-RET fusions can be used to characterize a thyroid cancer and/or papillary thyroid carcinoma; and the PAX8-PPARy fusion can be analyzed to characterize a follicular thyroid cancer. Fusions that are associated with hematological malignancies include without limitation TTL-ETV6, CDK6-MLL, CDK6-TLX3, ETV6-FLT3, ETV6-RUNX1, ETV6-TTL, MLL-AFF1, MLL-AFF3, MLL-AFF4, MLL-GAS7, TCBA1-ETV6, TCF3-PBX1 or TCF3-TFPT, which are characteristic of acute lymphocytic leukemia (ALL); BCL11B-TLX3, IL2-TNFRFS17, NUP214-ABL1, NUP98-CCDC28A, TALI-STIL, or ETV6-ABL2, which are characteristic of T-cell acute lymphocytic leukemia (T-ALL); ATIC-ALK, KIAA1618-ALK, MSN-ALK, MYH9-ALK, NPM1-ALK, TGF-ALK or TPM3-ALK, which are characteristic of anaplastic large cell lymphoma (ALCL); BCR-ABL1, BCR-JAK2, ETV6-EVI1, ETV6-MN1 or ETV6-TCBA1, characteristic of chronic myelogenous leukemia (CML); CBFB-MYH11, CHIC2-ETV6, ETV6-ABL1, ETV6-ABL2, ETV6-ARNT, ETV6-CDX2, ETV6-HLXB9, ETV6-PERI, MEF2D-DAZAP1, AML-AFF1, MLL-ARHGAP26, MLL-ARHGEF12, MLL-CASC5, MLL-CBL,MLL-CREBBP, MLL-DAB21P, MLL-ELL, MLL-EP300, MLL-EPS15, MLL-FNBP1, MLL-FOXO3A, MLL-GMPS, MLL-GPHN, MLL-MLLT1, MLL-MLLT11, MLL-MLLT3, MLL-MLLT6, MIL-MYO1F, MT,L-PICALM, MLL-SEPT2, MLL-SEPT6, MLL-SORBS2, MYST3-SORBS2, MYST-CREBBP, NPM1-MLF1, NUP98-HOXA13, PRDM16-EVIL, RABEP1-PDGFRB, RUNX1-EVI1, RUNX1-MDS1, RUNX1-RPL22, RUNX1-RUNX1T1, RUNX1-SH3D19, RUNX1-USP42, RUNX1-YTHDF2, RUNX1-ZNF687, or TAF15-ZNF-384, which are characteristic of acute myeloid leukemia (AML); CCND1-FSTL3, which is characteristic of chronic lymphocytic leukemia (CLL); BCL3-MYC, MYC-RTG1, BCL7A-MYC, BRWD3-ARHGAP20 or RTG1-MYC, which are characteristic of B-cell chronic lymphocytic leukemia (B-CLL); CITTA-BCL6, CLTC-ALK, IL2IR-BCL6, PIM1-BCL6, TFCR-BCL6, IKZFl-BCL6 or SEC31A-ALK, which are characteristic of diffuse large B-cell lymphomas (DLBCL); FLIP1-PDGFRA, FLT3-ETV6, KIAA1509-PDGFRA, PDE4DIP-PDGFRB, NIN-PDGFRB, TP53BP1-PDGFRB, or TPM3-PDGFRB, which are characteristic of hyper eosinophilia/chronic eosinophilia; and IGH-MYC or LCP1-BCL6, which are characteristic of Burkitt's lymphoma. One of skill will understand that additional fusions, including those yet to be identified to date, can be used to guide treatment once their presence is associated with a therapeutic intervention.

The fusion genes and gene products can be detected using one or more techniques described herein. In some embodiments, the sequence of the gene or corresponding mRNA is determined, e.g., using Sanger sequencing, NextGen sequencing, pyrosequencing, DNA microarrays, etc. Chromosomal abnormalities can be assessed using FISH or PCR techniques, among others. For example, a break apart probe can be used for FISH detection of ALK fusions such as EML4-ALK, KIF5B-ALK and/or TFG-ALK. As an alternate, PCR can be used to amplify the fusion product, wherein amplification or lack thereof indicates the presence or absence of the fusion, respectively. In some embodiments, the fusion protein fusion is detected. Appropriate methods for protein analysis include without limitation mass spectroscopy, electrophoresis (e.g., 2D gel electrophoresis or SDS-PAGE) or antibody related techniques, including immunoassay, protein array or immunohistochemistry. The techniques can be combined. As a non-limiting example, indication of an ALK fusion by FISH can be confirmed for ALK expression using IHC, or vice versa.

Treatment Selection

The systems and methods allow identification of one or more therapeutic targets whose projected efficacy can be linked to therapeutic efficacy, ultimately based on the molecular profiling. Illustrative schemes for using molecular profiling to identify a treatment regime are shown in FIGS. 2, 39 and 42, each of which is described in further detail herein. The invention comprises use of molecular profiling results to suggest associations with treatment responses. In an embodiment, the appropriate biomarkers for molecular profiling are selected on the basis of the subject's tumor type. These suggested biomarkers can be used to modify a default list of biomarkers. In other embodiments, the molecular profiling is independent of the source material. In some embodiments, rules are used to provide the suggested chemotherapy treatments based on the molecular profiling test results. In an embodiment, the rules are generated from abstracts of the peer reviewed clinical oncology literature. Expert opinion rules can be used but are optional. In an embodiment, clinical citations are assessed for their relevance to the methods of the invention using a hierarchy derived from the evidence grading system used by the United States Preventive Services Taskforce. The “best evidence” can be used as the basis for a rule. The simplest rules are constructed in the format of “if biomarker positive then treatment option one, else treatment option two.” Treatment options comprise no treatment with a specific drug, treatment with a specific drug or treatment with a combination of drugs. In some embodiments, more complex rules are constructed that involve the interaction of two or more biomarkers. In such cases, the more complex interactions are typically supported by clinical studies that analyze the interaction between the biomarkers included in the rule. Finally, a report can be generated that describes the association of the chemotherapy response and the biomarker and a summary statement of the best evidence supporting the treatments selected. Ultimately, the treating physician will decide on the best course of treatment.

As a non-limiting example, molecular profiling might reveal that the EGFR gene is amplified or overexpressed, thus indicating selection of a treatment that can block EGFR activity, such as the monoclonal antibody inhibitors cetuximab and panitumumab, or small molecule kinase inhibitors effective in patients with activating mutations in EGFR such as gefitinib, erlotinib, and lapatinib. Other anti-EGFR monoclonal antibodies in clinical development include zalutumumab, nimotuzumab, and matuzumab. The candidate treatment selected can depend on the setting revealed by molecular profiling. For example, kinase inhibitors are often prescribed with EGFR is found to have activating mutations. Continuing with the illustrative embodiment, molecular profiling may also reveal that some or all of these treatments are likely to be less effective. For example, patients taking gefitinib or erlotinib eventually develop drug resistance mutations in EGFR. Accordingly, the presence of a drug resistance mutation would contraindicate selection of the small molecule kinase inhibitors. One of skill will appreciate that this example can be expanded to guide the selection of other candidate treatments that act against genes or gene products whose differential expression is revealed by molecular profiling. Similarly, candidate agents known to be effective against diseased cells carrying certain nucleic acid variants can be selected if molecular profiling reveals such variants.

As another example, consider the drug imatinib, currently marketed by Novartis as Gleevec in the US in the form of imatinib mesylate. Imatinib is a 2-phenylaminopyrimidine derivative that functions as a specific inhibitor of a number of tyrosine kinase enzymes. It occupies the tyrosine kinase active site, leading to a decrease in kinase activity. Imatinib has been shown to block the activity of Abelson cytoplasmic tyrosine kinase (ABL), c-Kit and the platelet-derived growth factor receptor (PDGFR). Thus, imatinib can be indicated as a candidate therapeutic for a cancer determined by molecular profiling to overexpress ABL, c-KIT or PDGFR. Imatinib can be indicated as a candidate therapeutic for a cancer determined by molecular profiling to have mutations in ABL, c-KIT or PDGFR that alter their activity, e.g., constitutive kinase activity of ABLs caused by the BCR-ABL mutation. As an inhibitor of PDGFR, imatinib mesylate appears to have utility in the treatment of a variety of dermatological diseases.

Cancer therapies that can be identified as candidate treatments by the methods of the invention include without limitation: 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguaninc, Abraxanc, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, 0225, Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate Dexamethasone Sodium Phosphate, Dexasone, Dexrazoxane, DHAD, DIC, Diodex Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, Gemzar, Gleevec™, Gliadel® Wafer, GM-CSF, Goserelin, Granulocyte-Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin®, Idarubicin, Ifex®, IFN-alpha, Ifosfamide, IL-11, IL-2, Imalinib mesylate, Imidazole Carboxamide, Interferon alfa, Interferon Alfa-2b (PEG Conjugate), Interleukin-2, Interleukin-11, Intron A® (interferon alfa-2b), Iressa®. Irinotecan, Isotretinoin, Ixabepilone, Ixempra™, Kidrolase (t), Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-hound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®, and any appropriate combinations thereof.

The candidate treatments identified according to the subject methods can be chosen from the class of therapeutic agents identified as Anthracyclines and related substances, Anti-androgens, Anti-estrogens, Antigrowth hormones (e.g., Somatostatin analogs), Combination therapy (e.g., vincristine, bcnu, melphalan, cyclophosphamide, prednisone (VBMCP)), DNA methyltransferase inhibitors, Endocrine therapy-Enzyme inhibitor, Endocrine therapy—other hormone antagonists and related agents, Folic acid analogs (e.g., methotrexate), Folic acid analogs (e.g., pemetrexed), Gonadotropin releasing hormone analogs, Gonadotropin-releasing hormones, Monoclonal antibodies (EGFR-Targeted—e.g., panitumumab, cetuximab), Monoclonal antibodies (Her2-Targeted—e.g., trastuzumab), Monoclonal antibodies (Multi-Targeted—e.g., alemtuzumab), Other alkylating agents, Other antincoplastic agents (e.g., asparaginase), Other antineoplastic agents (e.g., ATRA), Other antineoplastic agents (e.g., bexarotene), Other antineoplastic agents (e.g., celecoxib), Other antineoplastic agents (e.g., gemcitabine), Other antineoplastic agents (e.g., hydroxyurea), Other antineoplastic agents (e.g., irinotecan, topotecan), Other antineoplastic agents (e.g., pentostatin), Other cytotoxic antibiotics, Platinum compounds, Podophyllotoxin derivatives (e.g., etoposide), Progestogens, Protein kinase inhibitors (EGFR-Targeted), Protein kinase inhibitors (Her2 targeted therapy—e.g., lapatinib), Pyrimidine analogs (e.g., cytarabine), Pyrimidine analogs (e.g., fluoropyrimidines), Salicylic acid and derivatives (e.g., aspirin), Src-family protein tyrosine kinase inhibitors (e.g., dasatinib), Taxanes, Taxanes (e.g., nab-paclitaxel), Vinca Alkaloids and analogs, Vitamin D and analogs, Monoclonal antibodies (Multi-Targeted—e.g., bevacizumab), Protein kinase inhibitors (e.g., imatinib, sorafenib, sunitinib).

In some embodiments, the candidate treatments identified according to the subject methods are chosen from at least the groups of treatments consisting of 5-fluorouracil, abarelix, alemtuzumab, aminoglutethimide, anastrozole, asparaginase, aspirin, ATRA, azacitidine, bevacizumab, bexarotene, bicalutamide, calcitriol, capecitabine, carboplatin, celecoxib, cetuximab, chemotherapy, cholecalciferol, cisplatin, cytarabine, dasatinib, daunorubicin, decitabine, doxorubicin, epirubicin, erlotinib, etoposide, exemestane, flutamide, fulvestrant, gefitinib, gemcitabine, gonadorelin, goserelin, hydroxyurea, imatinib, irinotecan, lapatinib, letrozole, leuprolide, liposomal-doxorubicin, medroxyprogesterone, megestrol, megestrol acetate, methotrexate, mitomycin, nab-paclitaxel, octreotide, oxaliplatin, paclitaxel, panitumumab, pegaspargase, pemetrexed, pentostatin, sorafenib, sunitinib, tamoxifen, Taxanes, temozolomide, toremifene, trastuzumab, VBMCP, and vincristine.

Rules Engine

In sone embodiments, a database is created that maps treatments and molecular profiling results. The treatment information can include the projected efficacy of a therapeutic agent against cells having certain attributes that can be measured by molecular profiling. The molecular profiling can include differential expression or mutations in certain genes, proteins, or other biological molecules of interest. Through the mapping, the results of the molecular profiling can be compared against the database to select treatments. The database can include both positive and negative mappings between treatments and molecular profiling results. In some embodiments, the mapping is created by reviewing the literature for links between biological agents and therapeutic agents. For example, a journal article, patent publication or patent application publication, scientific presentation, etc can be reviewed for potential mappings. The mapping can include results of in vivo, e.g., animal studies or clinical trials, or in vitro experiments, e.g., cell culture. Any mappings that are found can be entered into the database, e.g., cytotoxic effects of a therapeutic agent against cells expressing a gene or protein. In this manner, the database can be continuously updated. It will be appreciated that the methods of the invention are updated as well.

The rules for the mappings can contain a variety of supplemental information. In some embodiments, the database contains prioritization criteria. For example, a treatment with more projected efficacy in a given setting can be preferred over a treatment projected to have lesser efficacy. A mapping derived from a certain setting, e.g., a clinical trial, may be prioritized over a mapping derived from another setting, e.g., cell culture experiments. A treatment with strong literature support may be prioritized over a treatment supported by more preliminary results. A treatment generally applied to the type of disease in question, e.g., cancer of a certain tissue origin, may be prioritized over a treatment that is not indicated for that particular disease. Mappings can include both positive and negative correlations between a treatment and a molecular profiling result. In a non-limiting example, one mapping might suggest use of a kinase inhibitor like erlotinib against a tumor having an activating mutation in EGFR, whereas another mapping might suggest against that treatment if the EGFR also has a drug resistance mutation. Similarly, a treatment might be indicated as effective in cells that overexpress a certain gene or protein but indicated as not effective if the gene or protein is underexpressed.

The selection of a candidate treatment for an individual can be based on molecular profiling results from any one or more of the methods described. Alternatively, selection of a candidate treatment for an individual can be based on molecular profiling results from more than one of the methods described. For example, selection of treatment for an individual can be based on molecular profiling results from FISH alone, IHC alone, or microarray analysis alone. In other embodiments, selection of treatment for an individual can be based on molecular profiling results from IHC, FISH, and microarray analysis; IHC and FISH; IHC and microarray analysis, or FISH and microarray analysis. Selection of treatment for an individual can also be based on molecular profiling results from sequencing or other methods of mutation detection. Molecular profiling results may include mutation analysis along with one or more methods, such as IHC, immunoassay, and/or microarray analysis. Different combinations and sequential results can be used. For example, treatment can be prioritized according the results obtained by molecular profiling. In an embodiment, the prioritization is based on the following algorithm: 1) IHC/FISH and microarray indicates same target as a first priority; 2) IHC positive result alone next priority; or 3) microarray positive result alone as last priority. Sequencing can also be used to guide selection. In some embodiments, sequencing reveals a drug resistance mutation so that the effected drug is not selected even if techniques including IHC, microarray and/or FISII indicate differential expression of the target molecule. Any such contraindication, e.g., differential expression or mutation of another gene or gene product may override selection of a treatment.

An illustrative listing of microarray expression results versus predicted treatments is presented in Table 3. As disclosed herein, molecular profiling is performed to determine whether a gene or gene product is differentially expressed in a sample as compared to a control. The expression status of the gene or gene product is used to select agents that are predicted to be efficacious or not. For example, Table 3 shows that overexpression of the ADA gene or protein points to pentostatin as a possible treatment. On the other hand, underexpression of the ADA gene or protein implicates resistance to cytarabine, suggesting that cytarabine is not an optimal treatment.

TABLE 3
Molecular Profiling Results and Predicted Treatments

| | | Candidate | Possible |
|---|---|---|---|
| Gene Name | Expression Status | Agent(s) | Resistance |
| ADA | Overexpressed | pentostatin | |
| ADA | Underexpressed | | cytarabine |
| AR | Overexpressed | abarelix, | |
| | | bicalutamide, | |
| | | flutamide, | |
| | | gonadorelin, | |
| | | goserelin, | |
| | | leuprolide | |
| ASNS | Underexpressed | asparaginase, | |
| | | pegaspargase | |
| BCRP | Overexpressed | | cisplatin, |
| (ABCG2) | | | carboplatin, |
| | | | irinotecan, |
| | | | topotecan |
| BRCA1 | Underexpressed | mitomycin | |
| BRCA2 | Underexpressed | mitomycin | |
| CD52 | Overexpressed | alemtuzumab | |
| CDA | Overexpressed | | cytarabine |
| CES2 | Overexpressed | irinotecan | |
| c-kit | Overexpressed | sorafenib, | |
| | | sunitinib, | |
| | | imatinib | |
| COX-2 | Overexpressed | celecoxib | |
| DCK | Overexpressed | gemcitabine | cytarabine |
| DHFR | Underexpressed | methotrexate, | |
| | | pemetrexed | |
| DHFR | Overexpressed | | methotrexate |
| DNMT | Overexpressed | azacitidine, | |
| | | decitabine | |
| DNMT3A | Overexpressed | azacitidine, | |
| | | decitabine | |
| DNMT3B | Overexpressed | azacitidine, | |
| | | decitabine | |
| EGFR | Overexpressed | erlotinib, | |
| | | gefitinib, | |
| | | cetuximab, | |
| | | panitumumab | |
| EML4-ALK | Overexpressed (present) | crizotinib | |
| EPHA2 | Overexpressed | dasatinib | |
| ER | Overexpressed | anastrazole, | |
| | | exemestane, | |
| | | fulvestrant, | |
| | | letrozole, | |
| | | megestrol, | |
| | | tamoxifen, | |
| | | medroxy- | |
| | | progesterone, | |
| | | toremifene, | |
| | | amino- | |
| | | glutethimide | |
| ERCC1 | Overexpressed | | carboplatin, |
| | | | cisplatin |
| GART | Underexpressed | pemetrexed | |
| HER-2 | Overexpressed | trastuzumab, | |
| (ERBB2) | | lapatinib | |
| HIF-1α | Overexpressed | sorafenib, | |
| | | sunitinib, | |
| | | bevacizumab | |
| IκB-α | Overexpressed | bortezomib | |
| MGMT | Underexpressed | temozolomide | |
| MGMT | Overexpressed | | temozolomide |
| MRP1 | Overexpressed | | etoposide, |
| (ABCC1) | | | paclitaxel, |
| | | | docetaxel, |
| | | | vinblastine, |
| | | | vinorelbine, |
| | | | topotecan, |
| | | | teniposide |
| P-gp | Overexpressed | | doxorubicin, |
| (ABCB1) | | | etoposide, |
| | | | epirubicin, |
| | | | paclitaxel, |
| | | | docetaxel, |
| | | | vinblastine, |
| | | | vinorelbine, |
| | | | topotecan, |
| | | | teniposide, |
| | | | liposomal |
| | | | doxorubicin |
| PDGFR-α | Overexpressed | sorafenib, | |
| | | sunitinib, | |
| | | imatinib | |
| PDGFR-β | Overexpressed | sorafenib, | |
| | | sunitinib, | |
| | | imatinib | |
| PR | Overexpressed | exemestane, | |
| | | fulvestrant, | |
| | | gonadorelin, | |
| | | goserelin, | |
| | | medroxy- | |
| | | progesterone, | |
| | | megestrol, | |
| | | tamoxifen, | |
| | | toremifene | |
| RARA | Overexpressed | ATRA | |
| RRM1 | Underexpressed | gemcitabine, | |
| | | hydroxyurea | |
| RRM2 | Underexpressed | gemcitabine, | |
| | | hydroxyurea | |
| RRM2B | Underexpressed | gemcitabine, | |
| | | hydroxyurea | |
| RXR-α | Overexpressed | bexarotene | |
| RXR-β | Overexpressed | bexarotene | |
| SPARC | Overexpressed | nab-paclitaxel | |
| SRC | Overexpressed | dasatinib | |
| SSTR2 | Overexpressed | octreotide | |
| SSTR5 | Overexpressed | octreotide | |
| TOPO I | Overexpressed | irinotecan, | |
| | | topotecan | |
| TOPO IIα | Overexpressed | doxorubicin, | |
| | | epirubicin, | |
| | | liposomal- | |
| | | doxorubicin | |
| TOPO IIβ | Overexpressed | doxorubicin, | |
| | | epirubicin, | |
| | | liposomal- | |
| | | doxorubicin | |
| TS | Underexpressed | capecitabine, | |
| | | 5-fluorouracil, | |
| | | pemetrexed | |
| TS | Overexpressed | | capecitabine, |
| | | | 5-fluorouracil |
| VDR | Overexpressed | calcitriol, | |
| | | cholecalciferol | |
| VEGFR1 | Overexpressed | sorafenib, | |
| (Flt1) | | sunitinib, | |
| | | bevacizumab | |
| VEGFR2 | Overexpressed | sorafenib, | |
| | | sunitinib, | |
| | | bevacizumab | |
| VHL | Underexpressed | sorafenib, | |
| | | sunitinib | |

Table 4 presents an illustrative rules summary for treatment selection. The table is ordered by groups of related therapeutic agents. Each row describes a rule that maps the information derived from molecular profiling with an indication of benefit or lack of benefit for the therapeutic agent. Thus, the database contains a mapping of treatments whose biological activity is known against cancer cells that have alterations in certain genes or gene products, including gene copy alterations, chromosomal abnormalities, overexpression of or underexpression of one or more genes or gene products, or have various mutations. For each agent, a Lineage is presented as applicable which corresponds to a type of cancer associated with use of the agent. Agents with Benefit are listed along with a Benefit Summary Statement that describes molecular profiling information that relates to the predicted beneficial agent. Similarly, agents with Lack of Benefit are listed along with a Lack of Benefit Summary Statement that describes molecular profiling information that relates to the lack of benefit associated with the agent. Finally, the molecular profiling Criteria are shown. In the criteria, results from analysis using DNA microarray (DMA), IHC, FISH, and mutation analysis (MA) for one or more biomarkers is listed. For microarray analysis, expression can be reported as over (overexpressed) or under (underexpressed). When these criteria are met according to the application of the molecular profiling techniques to a sample, then the therapeutic agent or agents are predicted to have a benefit or lack of benefit as indicated in the corresponding row.

Lengthy table referenced here
US20160186266A1-20160630-T00001
Please refer to the end of the specification for access instructions.

The efficacy of various therapeutic agents given particular assay results, such as those in Table 4 above, is derived from reviewing, analyzing and rendering conclusions on empirical evidence, such as that is available the medical literature or other medical knowledge base. The results are used to guide the selection of certain therapeutic agents in a prioritized list for use in treatment of an individual. When molecular profiling results are obtained, e.g., differential expression or mutation of a gene or gene product, the results can be compared against the database to guide treatment selection. The set of rules in the database can be updated as new treatments and new treatment data become available. In some embodiments, the rules database is updated continuously. In some embodiments, the rules database is updated on a periodic basis. Any relevant correlative or comparative approach can be used to compare the molecular profiling results to the rules database. In one embodiment, a gene or gene product is identified as differentially expressed by molecular profiling. The rules database is queried to select entries for that gene or gene product. Treatment selection information selected from the rules database is extracted and used to select a treatment. The information, e.g., to recommend or not recommend a particular treatment, can be dependent on whether the gene or gene product is over or underexpressed, or has other abnormalities at the genetic or protein levels as compared to a reference. In some cases, multiple rules and treatments may be pulled from a database comprising the comprehensive rules set depending on the results of the molecular profiling. In some embodiments, the treatment options are presented in a prioritized list. In some embodiments, the treatment options are presented without prioritization information. In either case, an individual, e.g., the treating physician or similar caregiver may choose from the available options.

The methods described herein are used to prolong survival of a subject by providing personalized treatment. In some embodiments, the subject has been previously treated with one or more therapeutic agents to treat the disease, e.g., a cancer. The cancer may be refractory to one of these agents, e.g., by acquiring drug resistance mutations. In some embodiments, the cancer is metastatic. In some embodiments, the subject has not previously been treated with one or more therapeutic agents identified by the method. Using molecular profiling, candidate treatments can be selected regardless of the stage, anatomical location, or anatomical origin of the cancer cells.

Progression-free survival (PFS) denotes the chances of staying free of disease progression for an individual or a group of individuals suffering from a disease, e.g., a cancer, after initiating a course of treatment. It can refer to the percentage of individuals in a group whose disease is likely to remain stable (e.g., not show signs of progression) after a specified duration of time. Progression-free survival rates are an indication of the effectiveness of a particular treatment. Similarly, disease-free survival (DFS) denotes the chances of staying free of disease after initiating a particular treatment for an individual or a group of individuals suffering from a cancer. It can refer to the percentage of individuals in a group who are likely to be free of disease after a specified duration of time. Disease-free survival rates are an indication of the effectiveness of a particular treatment. Treatment strategies can be compared on the basis of the PFS or DFS that is achieved in similar groups of patients. Disease-free survival is often used with the term overall survival when cancer survival is described.

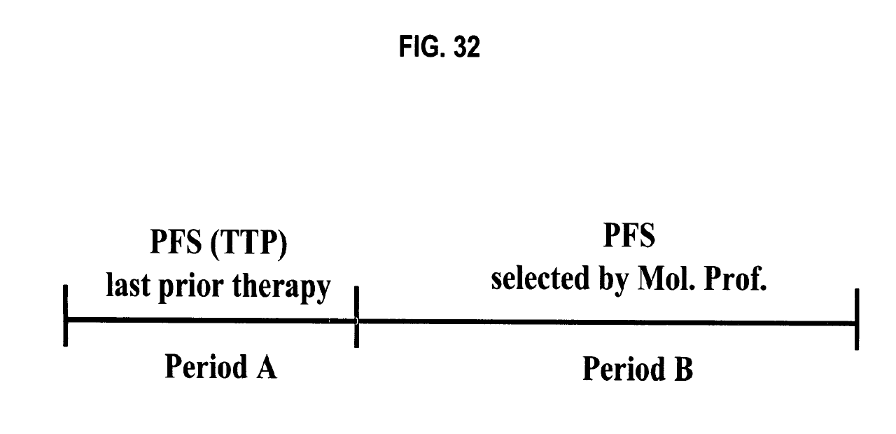

The candidate treatment selected by molecular profiling according to the invention can be compared to a non-molecular profiling selected treatment by comparing the progression free survival (PFS) using therapy selected by molecular profiling (period B) with PFS for the most recent therapy on which the patient has just progressed (period A). See FIG. 32. In one setting, a PFS(B)/PFS(A) ratio ≧1.3 was used to indicate that the molecular profiling selected therapy provides benefit for patient (Robert Temple, Clinical measurement in drug evaluation. Edited by Wu Ningano and G. T. Thicker John Wiley and Sons Ltd. 1995; Von Hoff, D. D. Clin Can Res. 4: 1079, 1999: Dhani et al. Clin Cancer Res. 15: 118-123, 2009). Other methods of comparing the treatment selected by molecular profiling to a non-molecular profiling selected treatment include determining response rate (RECIST) and percent of patients without progression or death at 4 months. The term “about” as used in the context of a numerical value for PFS means a variation of +/− ten percent (10%) relative to the numerical value. The PFS from a treatment selected by molecular profiling can be extended by at least 10%, 15%, 20%, 30%, 40%, 50%, 60%, 70%, 80%, or at least 90% as compared to a non-molecular profiling selected treatment. In some embodiments, the PFS from a treatment selected by molecular profiling can be extended by at least 100%, 150%, 200%, 300%, 400%, 500%, 600%, 700%, 800%, 900%, or at least about 1000% as compared to a non-molecular profiling selected treatment. In yet other embodiments, the PFS ratio (PFS on molecular profiling selected therapy or new treatment/PFS on prior therapy or treatment) is at least about 1.3. In yet other embodiments, the PFS ratio is at least about 1.1, 1.2, 1.3, 1.4, 1.5, 1.6, 1.7, 1.8, 1.9, or 2.0. In yet other embodiments, the PFS ratio is at least about 3, 4, 5, 6, 7, 8, 9 or 10.

Similarly, the DFS can be compared in patients whose treatment is selected with or without molecular profiling. In embodiments, DFS from a treatment selected by molecular profiling is extended by at least 10%, 15%, 20%, 30%, 40%, 50%, 60%, 70%, 80%, or at least 90% as compared to a non-molecular profiling selected treatment. In some embodiments, the DFS from a treatment selected by molecular profiling can be extended by at least 100%, 150%, 200%, 300%, 400%, 500%, 600%, 700%, 800%, 900%, or at least about 1000% as compared to a non-molecular profiling selected treatment. In yet other embodiments, the DES ratio (DFS on molecular profiling selected therapy or new treatment/DFS on prior therapy or treatment) is at least about 1.3. In yet other embodiments, the DFS ratio is at least about 1.1, 1.2, 1.3, 1.4, 1.5, 1.6, 1.7, 1.8, 1.9, or 2.0. In yet other embodiments, the DFS ratio is at least about 3, 4, 5, 6, 7, 8, 9 or 10.

In some embodiments, the candidate treatment of the invention will not increase the PFS ratio or the DFS ratio in the patient, nevertheless molecular profiling provides invaluable patient benefit. For example, in some instances no preferable treatment has been identified for the patient. In such cases, molecular profiling provides a method to identify a candidate treatment where none is currently identified. The molecular profiling may extend PFS, DFS or lifespan by at least 1 week, 2 weeks, 3 weeks, 4 weeks, 1 month, 5 weeks, 6 weeks, 7 weeks, 8 weeks, 2 months, 9 weeks, 10 weeks, 11 weeks, 12 weeks, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or 2 years. The molecular profiling may extend PFS, DFS or lifespan by at least 2½ years, 3 years, 4 years, 5 years, or more. In some embodiments, the methods of the invention improve outcome so that patient is in remission.

The effectiveness of a treatment can be monitored by other measures. A complete response (CR) comprises a complete disappearance of the disease: no disease is evident on examination, scans or other tests. A partial response (PR) refers to some disease remaining in the body, but there has been a decrease in size or number of the lesions by 30% or more. Stable disease (SD) refers to a disease that has remained relatively unchanged in size and number of lesions. Generally, less than a 50% decrease or a slight increase in size would be described as stable disease. Progressive disease (PD) means that the disease has increased in size or number on treatment. In some embodiments, molecular profiling according to the invention results in a complete response or partial response. In some embodiments, the methods of the invention result in stable disease. In some embodiments, the invention is able to achieve stable disease where non-molecular profiling results in progressive disease.

Computer Systems

Conventional data networking, application development and other functional aspects of the systems (and components of the individual operating components of the systems) may not be described in detail herein but are part of the invention. Furthermore, the connecting lines shown in the various figures contained herein are intended to represent illustrative functional relationships and/or physical couplings between the various elements. It should be noted that many alternative or additional functional relationships or physical connections may be present in a practical system.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases. Various databases used herein may include: patient data such as family history, demography and environmental data, biological sample data, prior treatment and protocol data, patient clinical data, molecular profiling data of biological samples, data on therapeutic drug agents and/or investigative drugs, a gene library, a disease library, a drug library, patient tracking data, file management data, financial management data, billing data and/or like data useful in the operation of the system. As those skilled in the art will appreciate, user computer may include an operating system (e.g., Windows NT, 95/98/2000, OS2, UNIX, Linux, Solaris, MacOS, etc.) as well as various conventional support software and drivers typically associated with computers. The computer may include any suitable personal computer, network computer, workstation, minicomputer, mainframe or the like. User computer can be in a home or medical/business environment with access to a network. In an illustrative embodiment, access is through a network or the Internet through a commercially-available web-browser software package.

As used herein, the term “network” shall include any electronic communications means which incorporates both hardware and software components of such. Communication among the parties may be accomplished through any suitable communication channels, such as, for example, a telephone network, an extranet, an intranet, Internet, point of interaction device, personal digital assistant (e.g., Palm Pilot®, Blackberry®), cellular phone, kiosk, etc.), online communications, satellite communications, off-line communications, wireless communications, transponder communications, local area network (LAN), wide area network (WAN), networked or linked devices, keyboard, mouse and/or any suitable communication or data input modality. Moreover, although the system is frequently described herein as being implemented with TCP/IP communications protocols, the system may also be implemented using IPX, Appletalk, IP-6, NetBIOS, OS1 or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAII RAY AND ERIC RAY, MASTERING HTML 4.0 (1997); and LOSHIN, TCP/IP CLEARLY EXPLAINED (1997) and DAVID GOURLEY AND BRIAN TOTTY, HTTP, THE DEFINITIVE GUIDE (2002), the contents of which are hereby incorporated by reference.

The various system components may be independently, separately or collectively suitably coupled to the network via data links which includes, for example, a connection to an Internet Service Provider (ISP) over the local loop as is typically used in connection with standard modem communication, cable modem, Dish networks, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television (ITV) network. Moreover, the system contemplates the use, sale or distribution of any goods, services or information over any network having similar functionality described herein.

As used herein, “transmit” may include sending electronic data from one system component to another over a network connection. Additionally, as used herein, “data” may include encompassing information such as commands, queries, files, data for storage, and the like in digital or any other form.

The system contemplates uses in association with web services, utility computing, pervasive and individualized computing, security and identity solutions, autonomic computing, commodity computing, mobility and wireless solutions, open source, biometrics, grid computing and/or mesh computing.

Any databases discussed herein may include relational, hierarchical, graphical, or object-oriented structure and/or any other database configurations. Common database products that may be used to implement the databases include DB2 by IBM (White Plains, N.Y.), various database products available from Oracle Corporation (Redwood Shores, Calif.), Microsoft Access or Microsoft SQL Server by Microsoft Corporation (Redmond, Wash.), or any other suitable database product. Moreover, the databases may be organized in any suitable manner, for example, as data tables or lookup tables. Each record may be a single file, a series of files, a linked series of data fields or any other data structure. Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be utilized to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed via one or more keys, numeric, alphabetical by first tuple, etc.); Binary Large Object (BLOB); stored as ungrouped data elements encoded using ISO/IEC 7816-6 data elements; stored as ungrouped data elements encoded using ISO/IEC Abstract Syntax Notation (ASN.1) as in ISO/IEC 8824 and 8825; and/or other proprietary techniques that may include fractal compression methods, image compression methods, etc.

In one illustrative embodiment, the ability to store a wide variety of information in different formats is facilitated by storing the information as a BLOB. Thus, any binary information can be stored in a storage space associated with a data set. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data by multiple and unrelated owners of the data sets. For example, a first data set which may be stored may be provided by a first party, a second data set which may be stored may be provided by an unrelated second party, and yet a third data set which may be stored, may be provided by a third party unrelated to the first and second party. Each of these three illustrative data sets may contain different information that is stored using different data storage formats and/or techniques. Further, each data set may contain subsets of data that also may be distinct from other subsets.

As stated above, in various embodiments, the data can be stored without regard to a common format. However, in one illustrative embodiment, the data set (e.g., BLOB) may be annotated in a standard manner when provided for manipulating the data. The annotation may comprise a short header, trailer, or other appropriate indicator related to each data set that is configured to convey information useful in managing the various data sets. For example, the annotation may be called a “condition header”, “header”, “trailer”, or “status”, herein, and may comprise an indication of the status of the data set or may include an identifier correlated to a specific issuer or owner of the data. Subsequent bytes of data may be used to indicate for example, the identity of the issuer or owner of the data, user, transaction/membership account identifier or the like. Each of these condition annotations are further discussed herein.

The data set annotation may also be used for other types of status information as well as various other purposes. For example, the data set annotation may include security information establishing access levels. The access levels may, for example, be configured to permit only certain individuals, levels of employees, companies, or other entities to access data sets, or to permit access to specific data sets based on the transaction, issuer or owner of data, user or the like. Furthermore, the security information may restrict/permit only certain actions such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate. The data, including the header or trailer may be received by a standalone interaction device configured to add, delete, modify, or augment the data in accordance with the header or trailer.

One skilled in the art will also appreciate that, for security reasons, any databases, systems, devices, servers or other components of the system may consist of any combination thereof at a single location or at multiple locations, wherein each database or system includes any of various suitable security features, such as firewalls, access codes, encryption, decryption, compression, decompression, and/or the like.

The computing unit of the web client may be further equipped with an Internet browser connected to the Internet or an intranet using standard dial-up, cable, DSL or any other Internet protocol known in the art. Transactions originating at a web client may pass through a firewall in order to prevent unauthorized access from users of other networks. Further, additional firewalls may be deployed between the varying components of CMS to further enhance security.

Firewall may include any hardware and/or software suitably configured to protect CMS components and/or enterprise computing resources from users of other networks. Further, a firewall may be configured to limit or restrict access to various systems and components behind the firewall for web clients connecting through a web server. Firewall may reside in varying configurations including Stateful Inspection, Proxy based and Packet Filtering among others. Firewall may be integrated within an web server or any other CMS components or may further reside as a separate entity.

The computers discussed herein may provide a suitable website or other Internet-based graphical user interface which is accessible by users. In one embodiment, the Microsoft Internet Information Server (IIS), Microsoft Transaction Server (MTS), and Microsoft SQL Server, are used in conjunction with the Microsoft operating system, Microsoft NT web server software, a Microsoft SQL Server database system, and a Microsoft Commerce Server. Additionally, components such as Access or Microsoft SQL Server, Oracle, Sybase, Informix MySQL, Interbase, etc., may be used to provide an Active Data Object (ADO) compliant database management system.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, Java applets, JavaScript, active server pages (ASP), common gateway interface scripts (CGI), extensible markup language (XML), dynamic HTML, cascading style sheets (CSS), helper applications, plug-ins, and the like. A server may include a web service that receives a request from a web server, the request including a URL (http://yahoo.com/stockquotes/ge) and an IP address (123.56.789.234). The web server retrieves the appropriate web pages and sends the data or applications for the web pages to the IP address. Web services are applications that are capable of interacting with other applications over a communications means, such as the internet. Web services are typically based on standards or protocols such as XML, XSLT, SOAP, WSDL and UDDI. Web services methods are well known in the art, and are covered in many standard texts. See, e.g., ALEX NGHIEM, IT WEB SERVICES: A ROADMAP FOR THE ENTERPRISE (2003), hereby incorporated by reference.

The web-based clinical database for the system and method of the present invention preferably has the ability to upload and store clinical data files in native formats and is searchable on any clinical parameter. The database is also scalable and may utilize an EAV data model (metadata) to enter clinical annotations from any study for easy integration with other studies. In addition, the web-based clinical database is flexible and may be XML and XSLT enabled to be able to add user customized questions dynamically. Further, the database includes exportability to CDISC ODM.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, Macromedia Cold Fusion, Microsoft Active Server Pages, Java, COBOL, assembler, PERL, Visual Basic, SQL Stored Procedures, extensible markup language (XML), with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JavaScript, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “Java Cryptography” by Jonathan Knudson, published by O'Reilly & Associates (1998); (3) “Cryptography & Network Security: Principles & Practice” by William Stallings, published by Prentice Hall; all of which are hereby incorporated by reference.

As used herein, the term “end user”, “consumer”, “customer”, “client”, “treating physician”, “hospital”, or “business” may be used interchangeably with each other, and each shall mean any person, entity, machine, hardware, software or business. Each participant is equipped with a computing device in order to interact with the system and facilitate online data access and data input. The customer has a computing unit in the form of a personal computer, although other types of computing units may be used including laptops, notebooks, hand held computers, set-top boxes, cellular telephones, touch-tone telephones and the like. The owner/operator of the system and method of the present invention has a computing unit implemented in the form of a computer-server, although other implementations are contemplated by the system including a computing center shown as a main frame computer, a mini-computer, a PC server, a network of computers located in the same of different geographic locations, or the like. Moreover, the system contemplates the use, sale or distribution of any goods, services or information over any network having similar functionality described herein.

In one illustrative embodiment, each client customer may be issued an “account” or “account number”. As used herein, the account or account number may include any device, code, number, letter, symbol, digital certificate, smart chip, digital signal, analog signal, biometric or other identifier/indicia suitably configured to allow the consumer to access, interact with or communicate with the system (e.g., one or more of an authorization/access code, personal identification number (PIN), Internet code, other identification code, and/or the like). The account number may optionally be located on or associated with a charge card, credit card, debit card, prepaid card, embossed card, smart card, magnetic stripe card, bar code card, transponder, radio frequency card or an associated account. The system may include or interface with any of the foregoing cards or devices, or a fob having a transponder and RFID reader in RF communication with the fob. Although the system may include a fob embodiment, the invention is not to be so limited. Indeed, system may include any device having a transponder which is configured to communicate with RFID reader via RF communication. Typical devices may include, for example, a key ring, tag, card, cell phone, wristwatch or any such form capable of being presented for interrogation. Moreover, the system, computing unit or device discussed herein may include a “pervasive computing device,” which may include a traditionally non-computerized device that is embedded with a computing unit. The account number may be distributed and stored in any form of plastic, electronic, magnetic, radio frequency, wireless, audio and/or optical device capable of transmitting or downloading data from itself to a second device.

As will be appreciated by one of ordinary skill in the art, the system may be embodied as a customization of an existing system, an add-on product, upgraded software, a standalone system, a distributed system, a method, a data processing system, a device for data processing, and/or a computer program product. Accordingly, the system may take the form of an entirely software embodiment, an entirely hardware embodiment, or an embodiment combining aspects of both software and hardware. Furthermore, the system may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user windows, web pages, websites, web forms, prompts, etc. Practitioners will appreciate that the illustrated steps described herein may comprise in any number of configurations including the use of windows, web pages, web forms, popup windows, prompts and the like. It should be further appreciated that the multiple steps as illustrated and described may be combined into single web pages and/or windows but have been expanded for the sake of simplicity. In other cases, steps illustrated and described as single process steps may be separated into multiple web pages and/or windows but have been combined for simplicity.

Molecular Profiling Methods

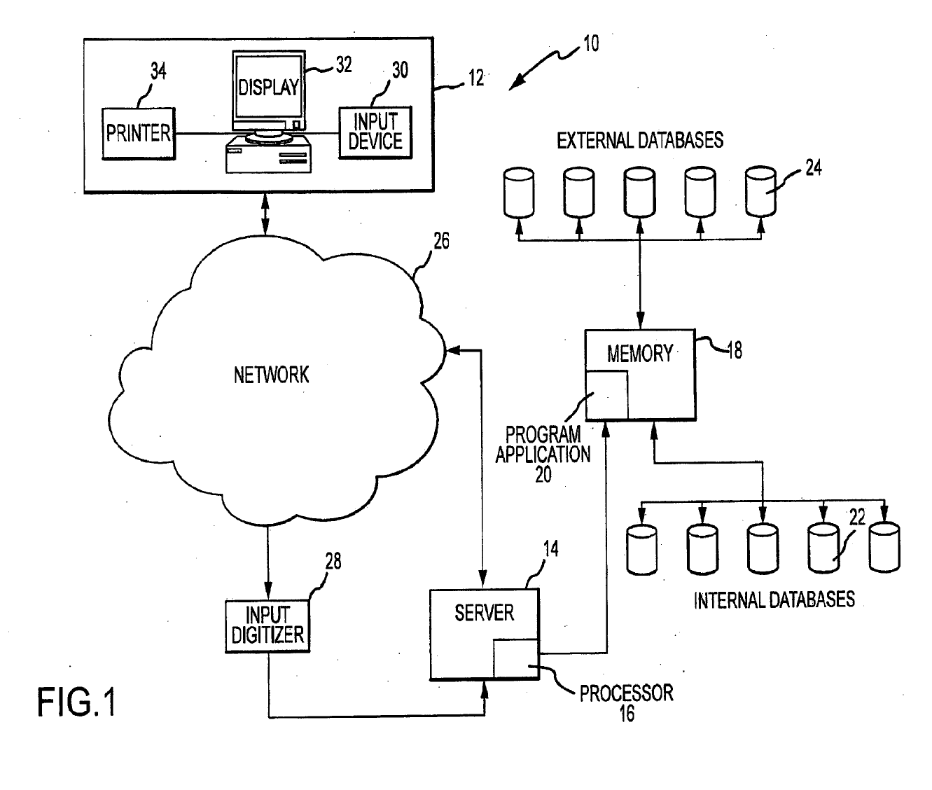

FIG. 1 illustrates a block diagram of an illustrative embodiment of a system 10 for determining individualized medical intervention for a particular disease state that utilizes molecular profiling of a patient's biological specimen. System 10 includes a user interface 12, a host server 14 including a processor 16 for processing data, a memory 18 coupled to the processor, an application program 20 stored in the memory 18 and accessible by the processor 16 for directing processing of the data by the processor 16, a plurality of internal databases 22 and external databases 24, and an interface with a wired or wireless communications network 26 (such as the Internet, for example). System 10 may also include an input digitizer 28 coupled to the processor 16 for inputting digital data from data that is received from user interface 12.

User interface 12 includes an input device 30 and a display 32 for inputting data into system 10 and for displaying information derived from the data processed by processor 16. User interface 12 may also include a printer 34 for printing the information derived from the data processed by the processor 16 such as patient reports that may include test results for targets and proposed drug therapies based on the test results.

Internal databases 22 may include, but are not limited to, patient biological sample/specimen information and tracking, clinical data, patient data, patient tracking, file management, study protocols, patient test results from molecular profiling, and billing information and tracking. External databases 24 may include, but are not limited to, drug libraries, gene libraries, disease libraries, and public and private databases such as UniGene, OMIM, GO, TIGR, GenBank, KEGG and Biocarta.

Various methods may be used in accordance with system 10. FIG. 2 shows a flowchart of an illustrative embodiment of a method 50 for determining individualized medical intervention for a particular disease state that utilizes molecular profiling of a patient's biological specimen that is non disease specific. In order to determine a medical intervention for a particular disease state using molecular profiling that is independent of disease lineage diagnosis (i.e. not single disease restricted), at least one test is performed for at least one target from a biological sample of a diseased patient in step 52. A target is defined as any molecular finding that may be obtained from molecular testing. For example, a target may include one or more genes, one or more gene expressed proteins, one or more molecular mechanisms, and/or combinations of such. For example, the expression level of a target can be determined by the analysis of mRNA levels or the target or gene, or protein levels of the gene. Tests for finding such targets may include, but are not limited, fluorescent in-situ hybridization (FISH), an in-situ hybridization (ISH), and other molecular tests known to those skilled in the art. PCR-based methods, such as real-time PCR or quantitative PCR can be used. Furthermore, microarray analysis, such as a comparative genomic hybridization (CGH) micro array, a single nucleotide polymorphism (SNP) microarray, a proteomic array, or antibody array analysis can also be used in the methods disclosed herein. In some embodiments, microarray analysis comprises identifying whether a gene is up-regulated or down-regulated relative to a reference with a significance of p≦0.001. Tests or analyses of targets can also comprise immunohistochemical (IHC) analysis. In some embodiments, IHC analysis comprises determining whether 30% or more of a sample is stained, if the staining intensity is +2 or greater, or both.

Furthermore, the methods disclosed herein also including profiling more than one target. For example, the expression of a plurality of genes can be identified. Furthermore, identification of a plurality of targets in a sample can be by one method or by various means. For example, the expression of a first gene can be determined by one method and the expression level of a second gene determined by a different method. Alternatively, the same method can be used to detect the expression level of the first and second gene. For example, the first method can be IHC and the second by microarray analysis, such as detecting the gene expression of a gene.

In some embodiments, molecular profiling can also including identifying a genetic variant, such as a mutation, polymorphism (such as a SNP), deletion, or insertion of a target. For example, identifying a SNP in a gene can be determined by microarray analysis, real-time PCR, or sequencing. Other methods disclosed herein can also be used to identify variants of one or more targets.

Accordingly, one or more of the following may be performed: an IHC analysis in step 54, a microanalysis in step 56, and other molecular tests know to those skilled in the art in step 58.

Biological samples are obtained from diseased patients by taking a biopsy of a tumor, conducting minimally invasive surgery if no recent tumor is available, obtaining a sample of the patient's blood, or a sample of any other biological fluid including, but not limited to, cell extracts, nuclear extracts, cell lysates or biological products or substances of biological origin such as excretions, blood, sera, plasma, urine, sputum, tears, feces, saliva, membrane extracts, and the like.

In step 60, a determination is made as to whether one or more of the targets that were tested for in step 52 exhibit a change in expression compared to a normal reference for that particular target. In one illustrative method of the invention, an IHC analysis may be performed in step 54 and a determination as to whether any targets from the IHC analysis exhibit a change in expression is made in step 64 by determining whether 30% or more of the biological sample cells were +2 or greater staining for the particular target. It will be understood by those skilled in the art that there will be instances where +1 or greater staining will indicate a change in expression in that staining results may vary depending on the technician performing the test and type of target being tested. In another illustrative embodiment of the invention, a micro array analysis may be performed in step 56 and a determination as to whether any targets from the micro array analysis exhibit a change in expression is made in step 66 by identifying which targets are up-regulated or down-regulated by determining whether the fold change in expression for a particular target relative to a normal tissue of origin reference is significant at p≦0.001. A change in expression may also be evidenced by an absence of one or more genes, gene expressed proteins, molecular mechanisms, or other molecular findings.

After determining which targets exhibit a change in expression in step 60, at least one non-disease specific agent is identified that interacts with each target having a changed expression in step 70. An agent may be any drug or compound having a therapeutic effect. A non-disease specific agent is a therapeutic drug or compound not previously associated with treating the patient's diagnosed disease that is capable of interacting with the target from the patient's biological sample that has exhibited a change in expression. Some of the non-disease specific agents that have been found to interact with specific targets found in different cancer patients are shown in Table 5 below.

TABLE 5
Illustrative target-drug associations

| Patients | Target(s) Found | Treatment(s) |
|---|---|---|
| Advanced Pancreatic | HER 2/neu | Herceptin ™ |
| Cancer | (IHC/Array) | |
| Advanced Pancreatic | EGFR (IHC), | Erbitux ™ |
| Cancer | HIF 1α | Rapamycin ™ |
| Advanced Ovarian Cancer | ERCC3 (Array) | Irofulvene |
| Advanced Adenoid Cystic | Vitamin D receptors, | Calcitriol ™ |
| Carcinoma | Androgen receptors | Flutamide ™ |

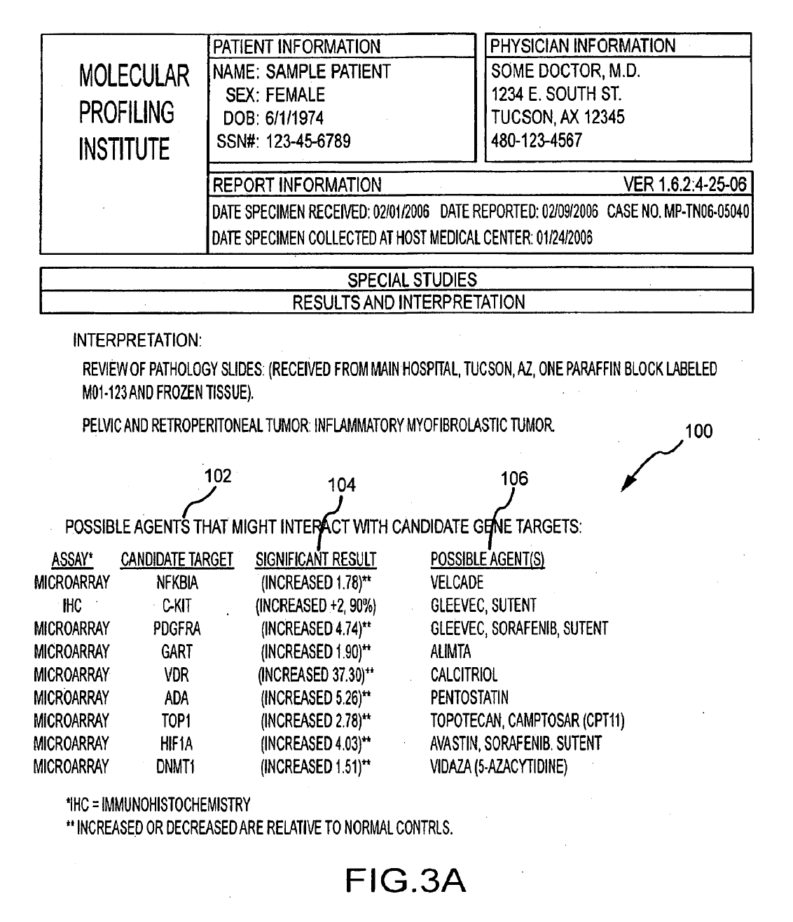

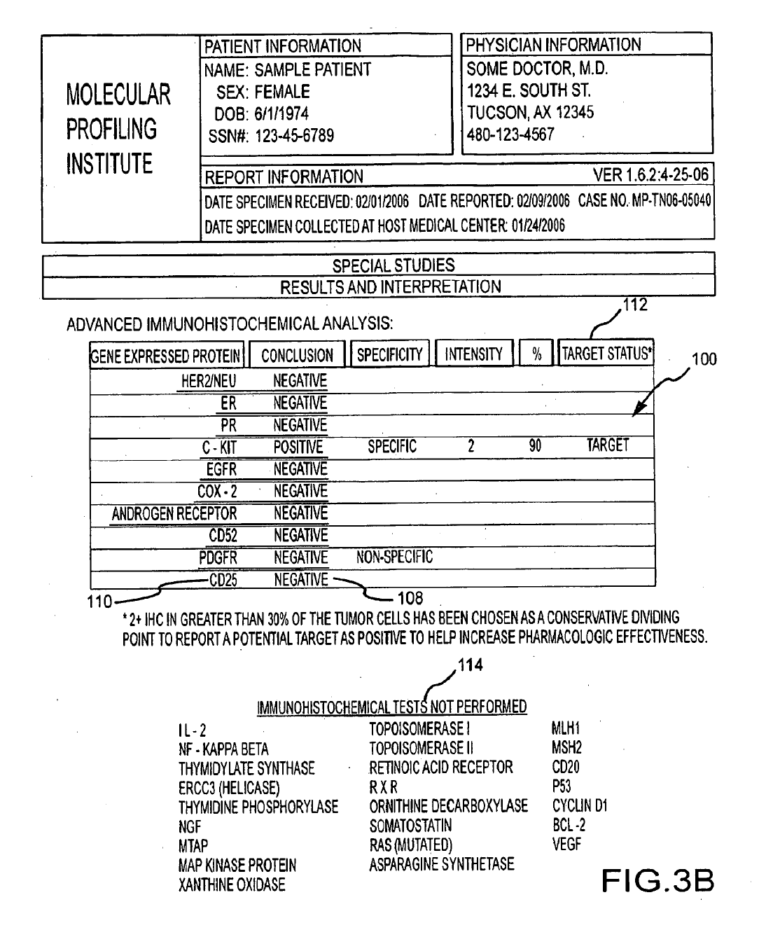

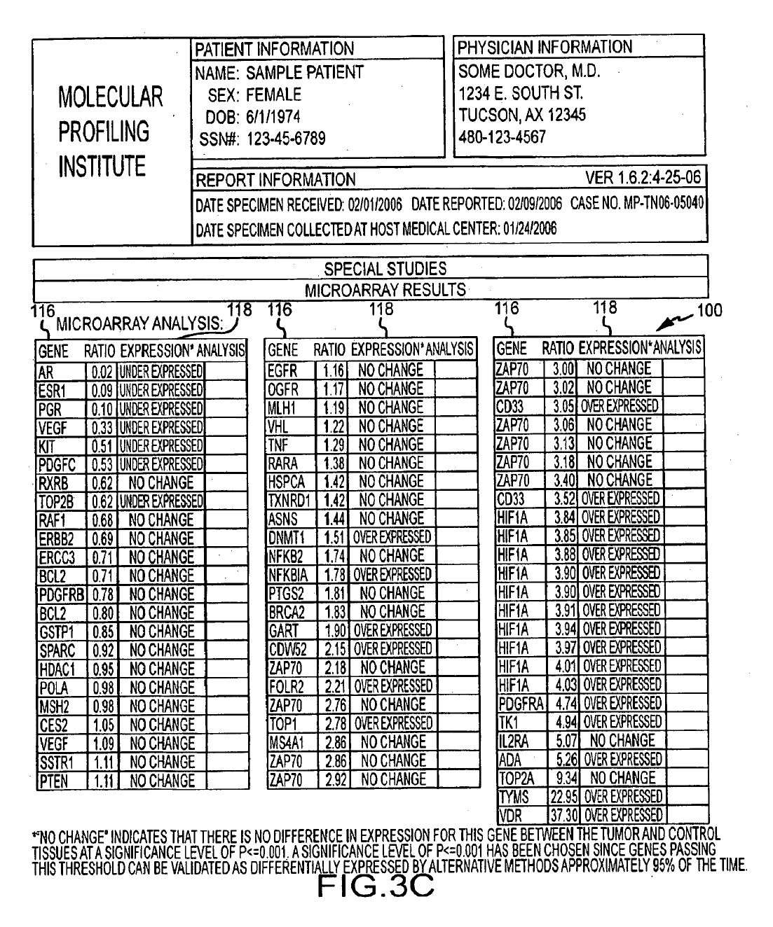

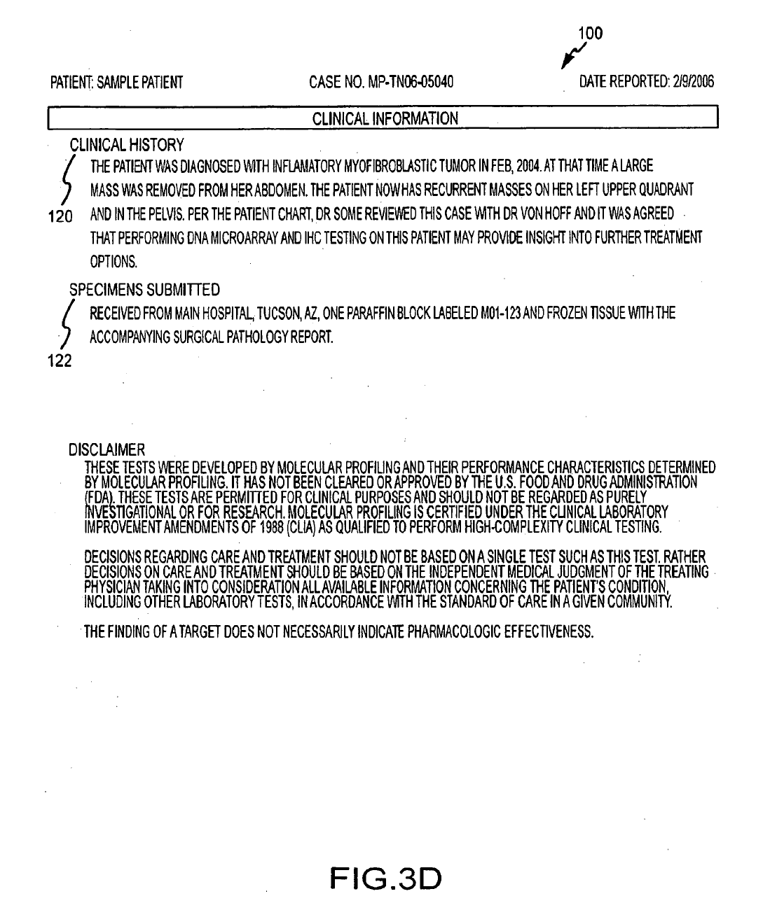

Finally, in step 80, a patient profile report may be provided which includes the patient's test results for various targets and any proposed therapies based on those results. An illustrative patient profile report 100 is shown in FIGS. 3A-3D. Patient profile report 100 shown in FIG. 3A identifies the targets tested 102, those targets tested that exhibited significant changes in expression 104, and proposed non-disease specific agents for interacting with the targets 106. Patient profile report 100 shown in FIG. 3B identifies the results 108 of immunohistochemical analysis for certain gene expressed proteins 110 and whether a gene expressed protein is a molecular target 112 by determining whether 30% or more of the tumor cells were +2 or greater staining. Report 100 also identifies immunohistochemical tests that were not performed 114. Patient profile report 100 shown in FIG. 3C identifies the genes analyzed 116 with a micro array analysis and whether the genes were under expressed or over expressed 118 compared to a reference. Finally, patient profile report 100 shown in FIG. 3D identifies the clinical history 120 of the patient and the specimens that were submitted 122 from the patient. Molecular profiling techniques can be performed anywhere, e.g., a foreign country, and the results sent by network to an appropriate party, e.g., the patient, a physician, lab or other party located remotely.

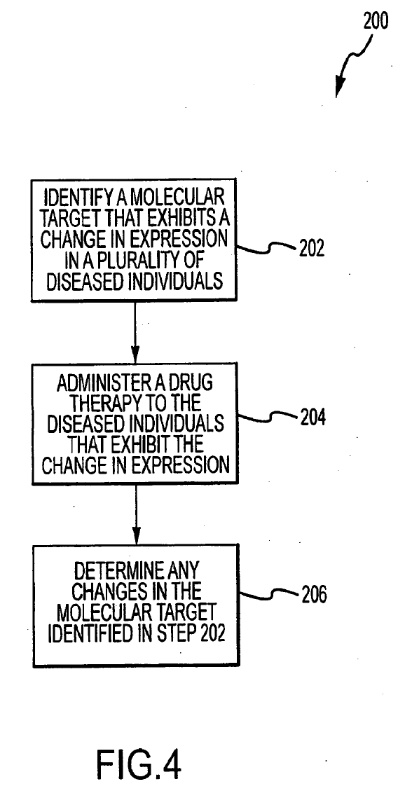

FIG. 4 shows a flowchart of an illustrative embodiment of a method 200 for identifying a drug therapy/agent capable of interacting with a target. In step 202, a molecular target is identified which exhibits a change in expression in a number of diseased individuals. Next, in step 204, a drug therapy/agent is administered to the diseased individuals. After drug therapy/agent administration, any changes in the molecular target identified in step 202 are identified in step 206 in order to determine if the drug therapy/agent administered in step 204 interacts with the molecular targets identified in step 202. If it is determined that the drug therapy/agent administered in step 204 interacts with a molecular target identified in step 202, the drug therapy/agent may be approved for treating patients exhibiting a change in expression of the identified molecular target instead of approving the drug therapy/agent for a particular disease.

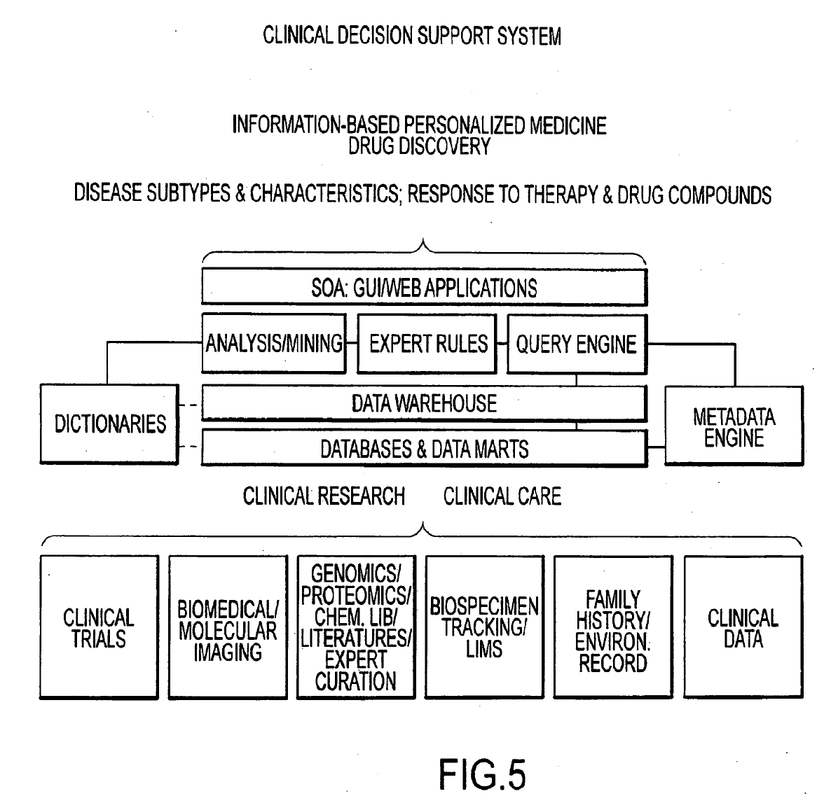

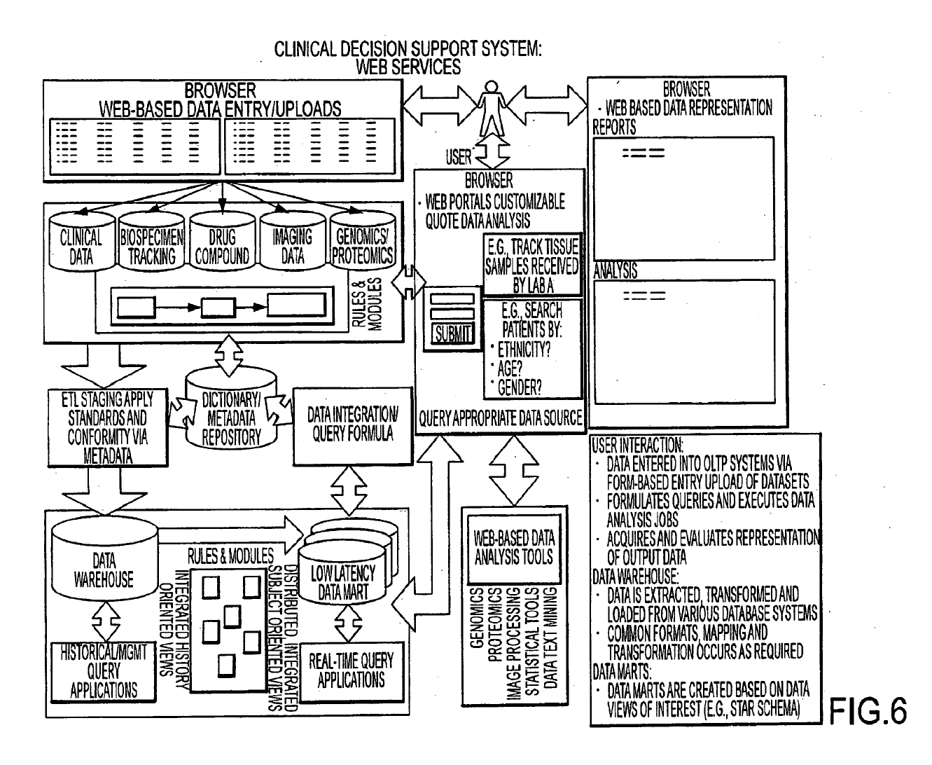

FIGS. 5-14 are flowcharts and diagrams illustrating various parts of an information-based personalized medicine drug discovery system and method in accordance with the present invention. FIG. 5 is a diagram showing an illustrative clinical decision support system of the information-based personalized medicine drug discovery system and method of the present invention. Data obtained through clinical research and clinical care such as clinical trial data, biomedical/molecular imaging data, genomics/proteomics/chemical library/literature/expert curation, biospecimen tracking/LIMS, family history/environmental records, and clinical data are collected and stored as databases and datamarts within a data warehouse. FIG. 6 is a diagram showing the flow of information through the clinical decision support system of the information-based personalized medicine drug discovery system and method of the present invention using web services. A user interacts with the system by entering data into the system via form-based entry/upload of data sets, formulating queries and executing data analysis jobs, and acquiring and evaluating representations of output data. The data warehouse in the web based system is where data is extracted, transformed, and loaded from various database systems. The data warehouse is also where common formats, mapping and transformation occurs. The web based system also includes datamarts which are created based on data views of interest.

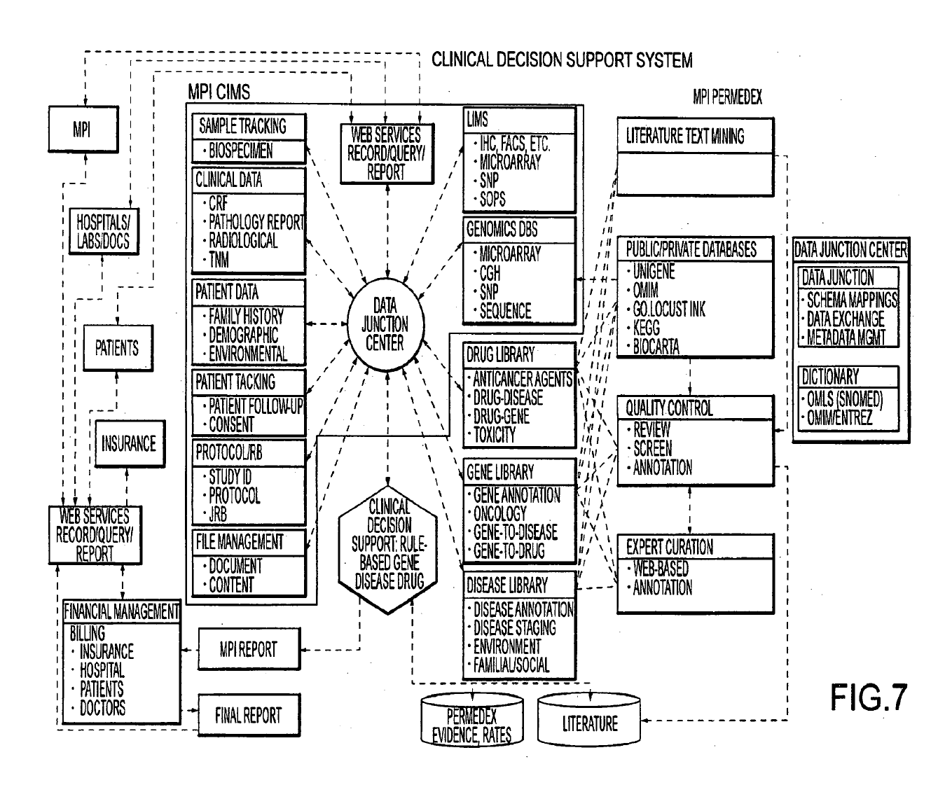

A flow chart of an illustrative clinical decision support system of the information-based personalized medicine drug discovery system and method of the present invention is shown in FIG. 7. The clinical information management system includes the laboratory information management system and the medical information contained in the data warehouses and databases includes medical information libraries, such as drug libraries, gene libraries, and disease libraries, in addition to literature text mining. Both the information management systems relating to particular patients and the medical information databases and data warehouses come together at a data junction center where diagnostic information and therapeutic options can be obtained. A financial management system may also be incorporated in the clinical decision support system of the information-based personalized medicine drug discovery system and method of the present invention.

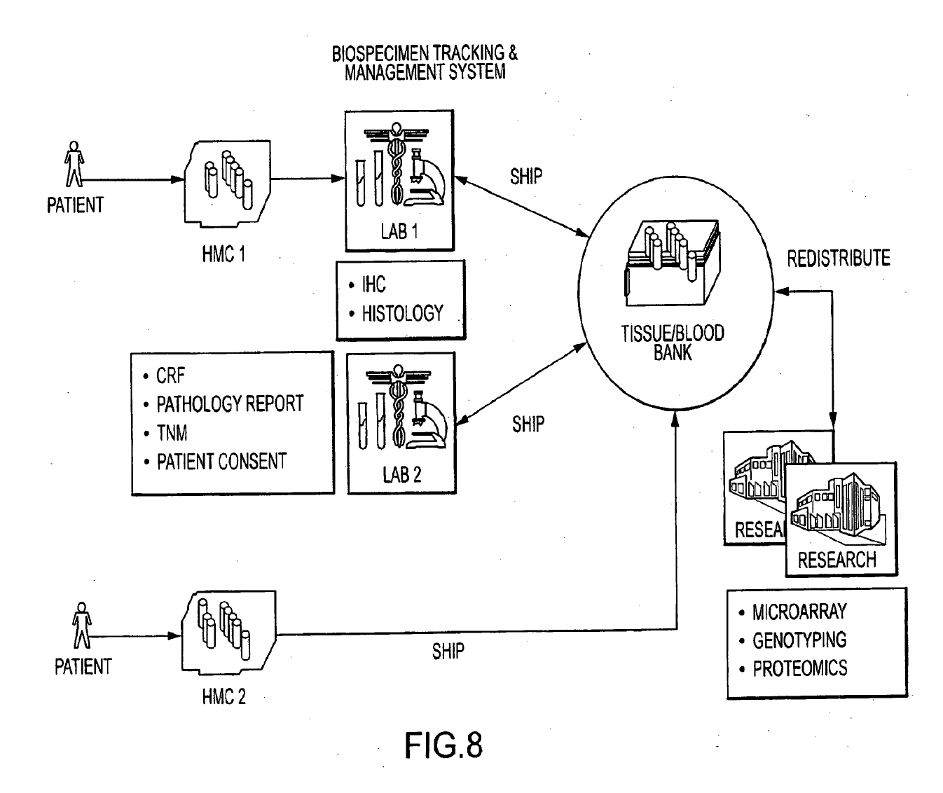

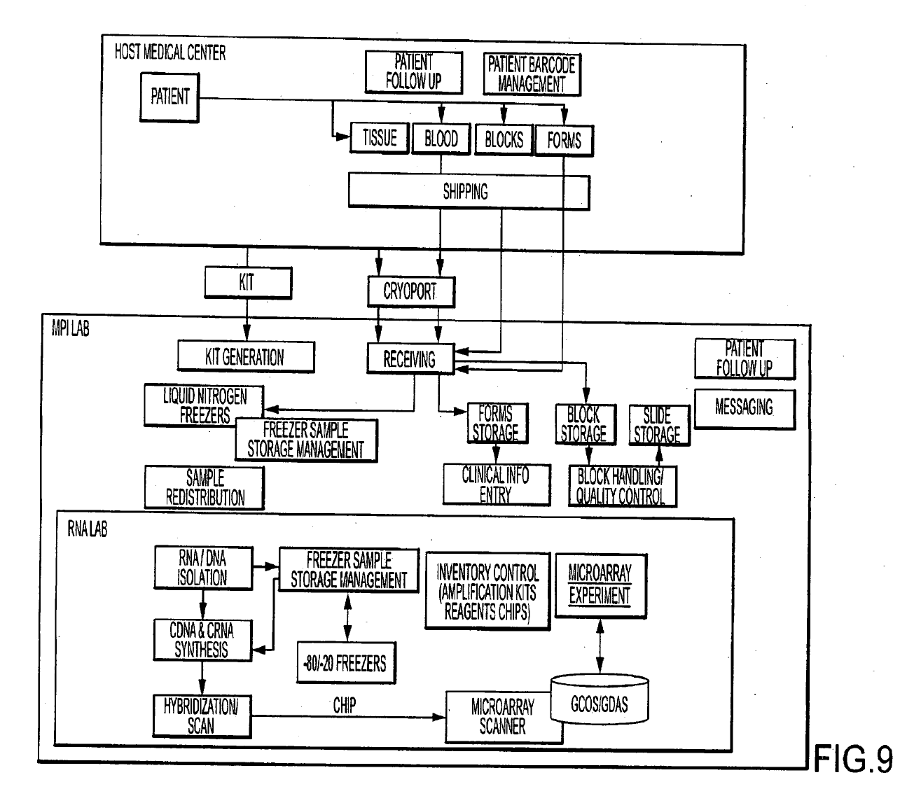

FIG. 8 is a diagram showing an illustrative biospecimen tracking and management system which may be utilized as part of the information-based personalized medicine drug discovery system and method of the present invention. FIG. 8 shows two host medical centers which forward specimens to a tissue/blood bank. The specimens may go through laboratory analysis prior to shipment. Research may also be conducted on the samples via micro array, genotyping, and proteomic analysis. This information can be redistributed to the tissue/blood bank. FIG. 9 depicts a flow chart of an illustrative biospecimen tracking and management system which may be utilized with the information-based personalized medicine drug discovery system and method of the present invention. The host medical center obtains samples from patients and then ships the patient samples to a molecular profiling laboratory which may also perform RNA and DNA isolation and analysis.

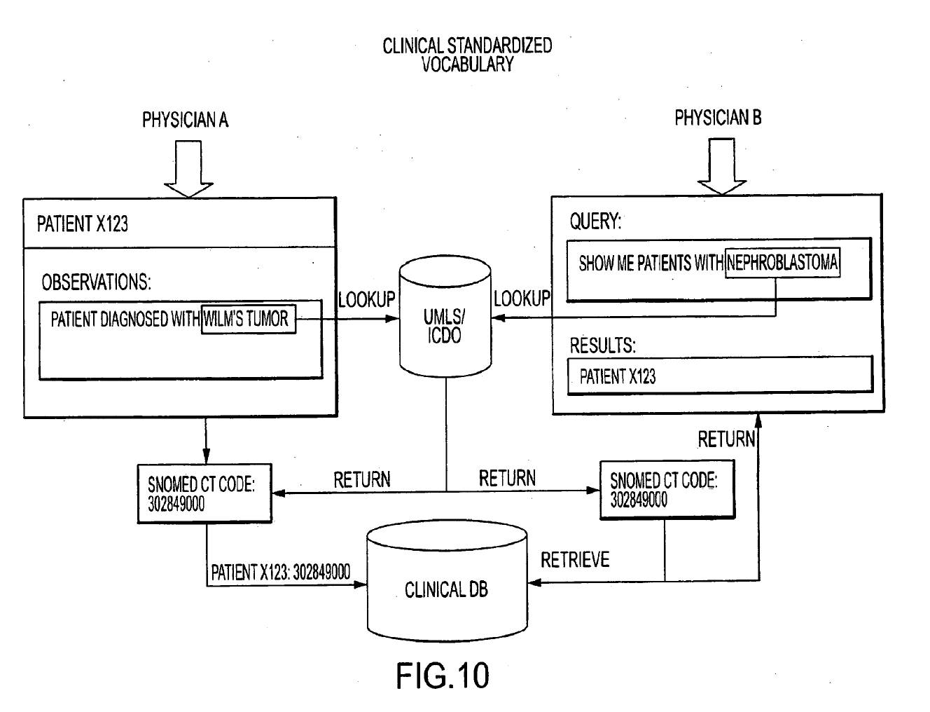

A diagram showing a method for maintaining a clinical standardized vocabulary for use with the information-based personalized medicine drug discovery system and method of the present invention is shown in FIG. 10. FIG. 10 illustrates how physician observations and patient information associated with one physician's patient may be made accessible to another physician to enable the other physician to utilize the data in making diagnostic and therapeutic decisions for their patients.

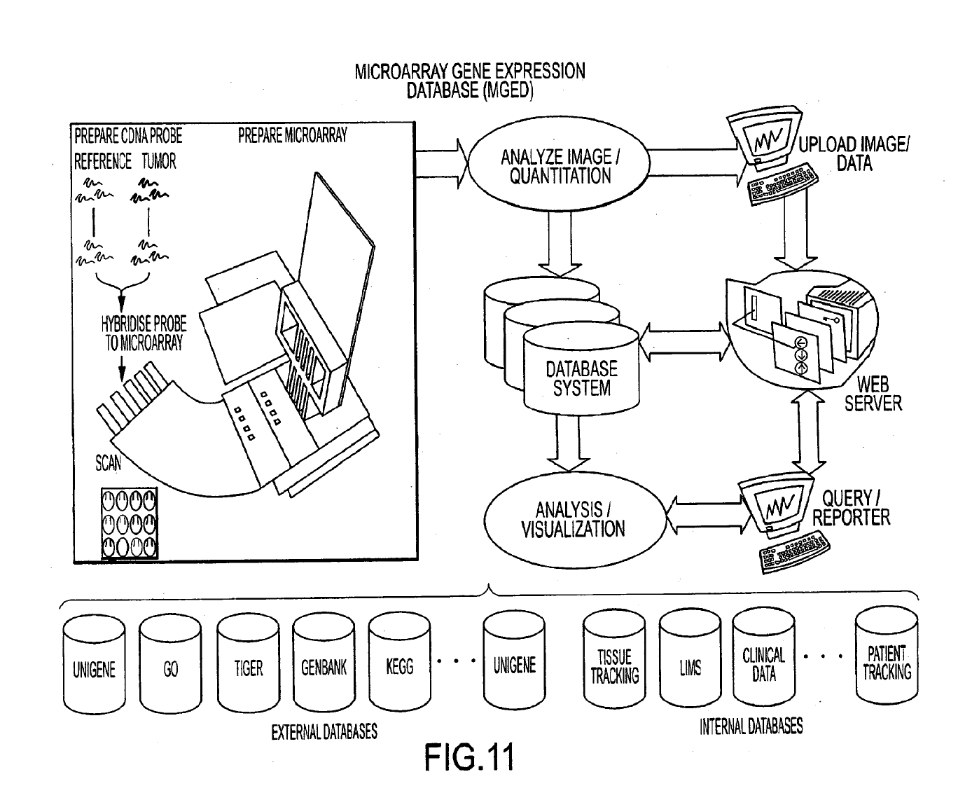

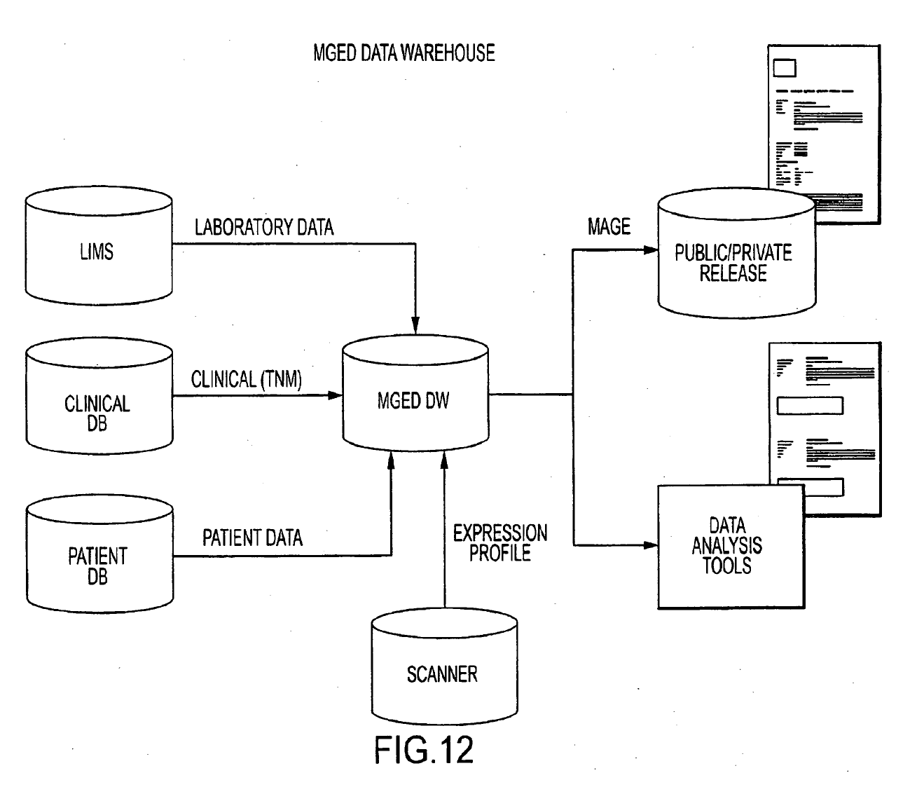

FIG. 11 shows a schematic of an illustrative microarray gene expression database which may be used as part of the information-based personalized medicine drug discovery system and method of the present invention. The micro array gene expression database includes both external databases and internal databases which can be accessed via the web based system. External databases may include, but are not limited to, UniGene, GO, TIGR, GenBank, KEGG. The internal databases may include, but are not limited to, tissue tracking, LIMS, clinical data, and patient tracking. FIG. 12 shows a diagram of an illustrative micro array gene expression database data warehouse which may be used as part of the information-based personalized medicine drug discovery system and method of the present invention. Laboratory data, clinical data, and patient data may all be housed in the micro array gene expression database data warehouse and the data may in turn be accessed by public/private release and utilized by data analysis tools.

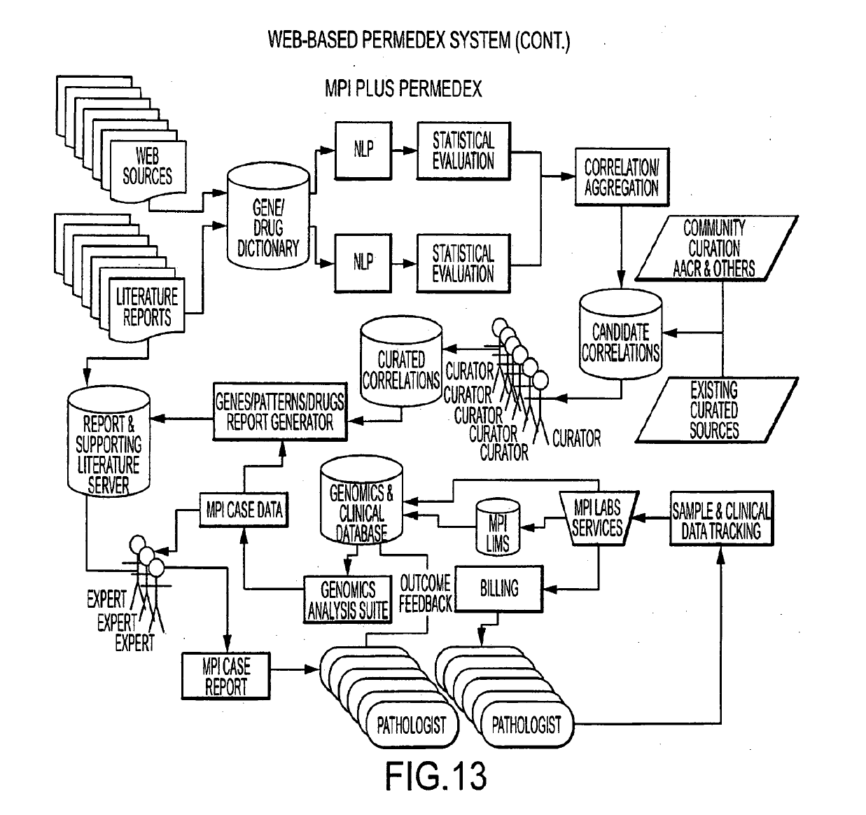

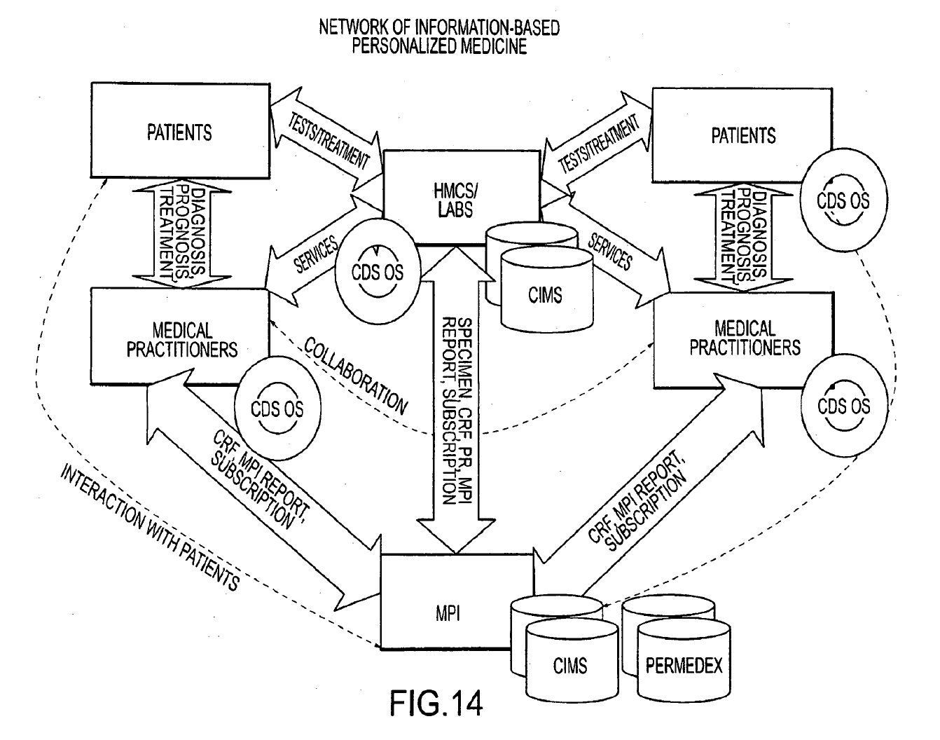

Another schematic showing the flow of information through an information-based personalized medicine drug discovery system and method of the present invention is shown in FIG. 13. Like FIG. 7, the schematic includes clinical information management, medical and literature information management, and financial management of the information-based personalized medicine drug discovery system and method of the present invention. FIG. 14 is a schematic showing an illustrative network of the information-based personalized medicine drug discovery system and method of the present invention. Patients, medical practitioners, host medical centers, and labs all share and exchange a variety of information in order to provide a patient with a proposed therapy or agent based on various identified targets.

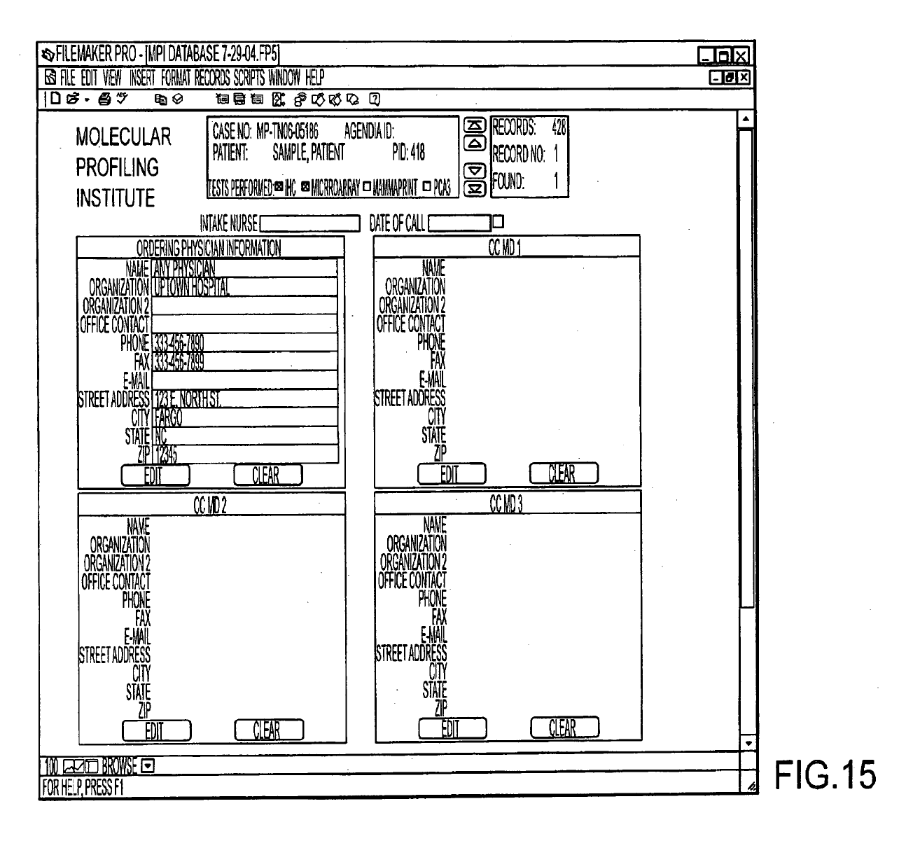

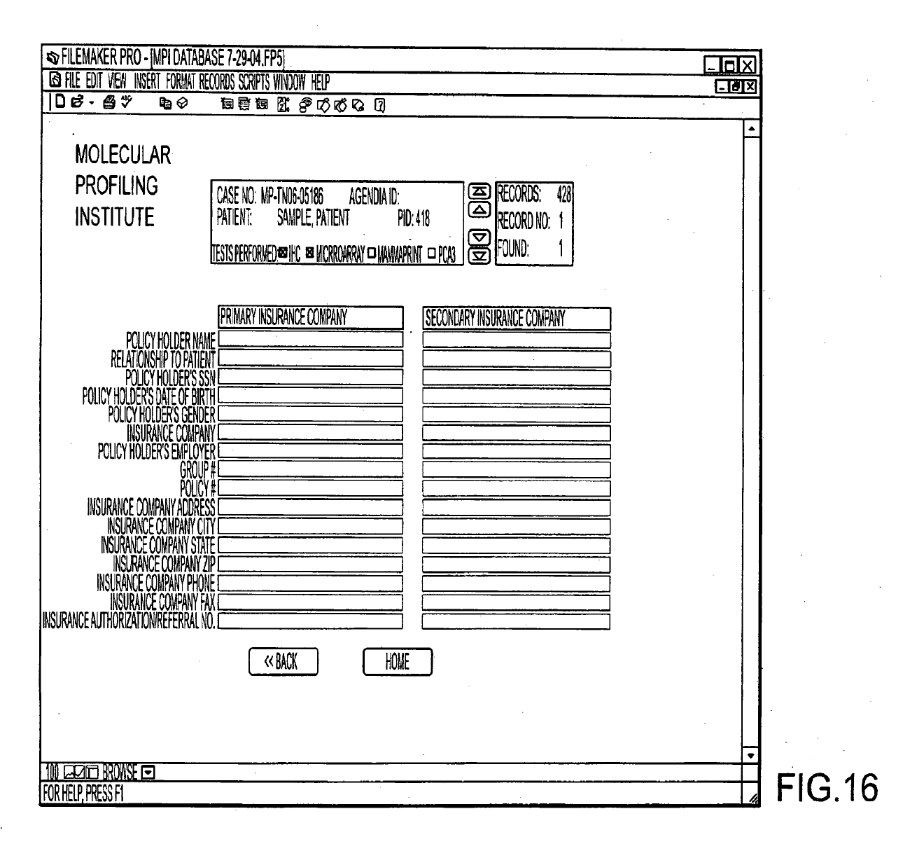

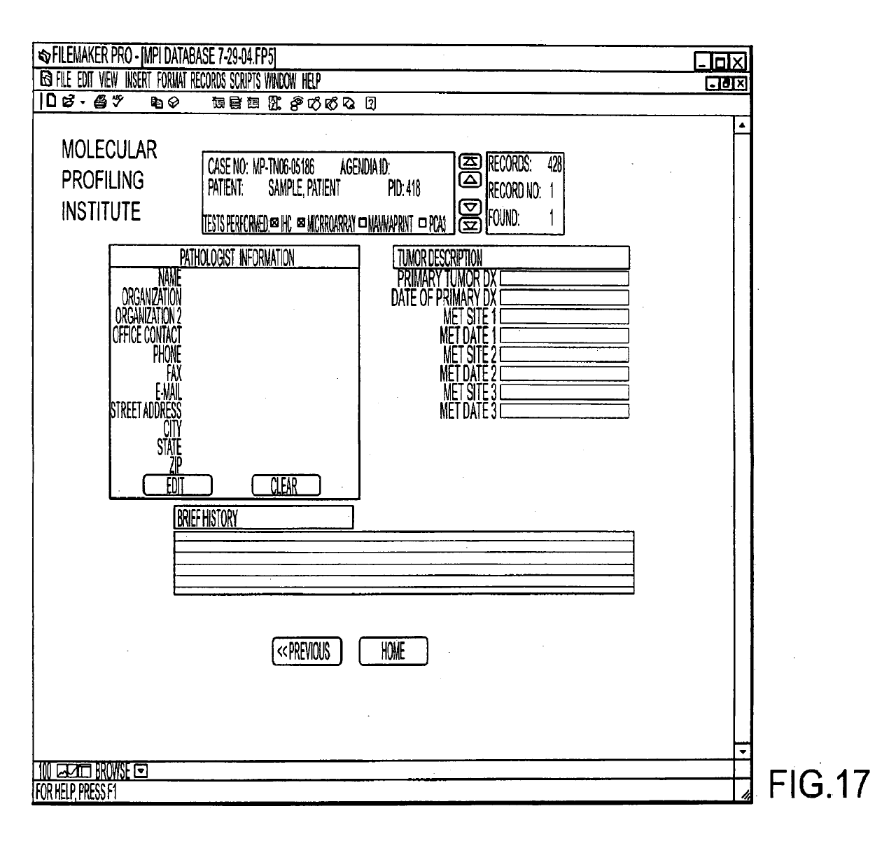

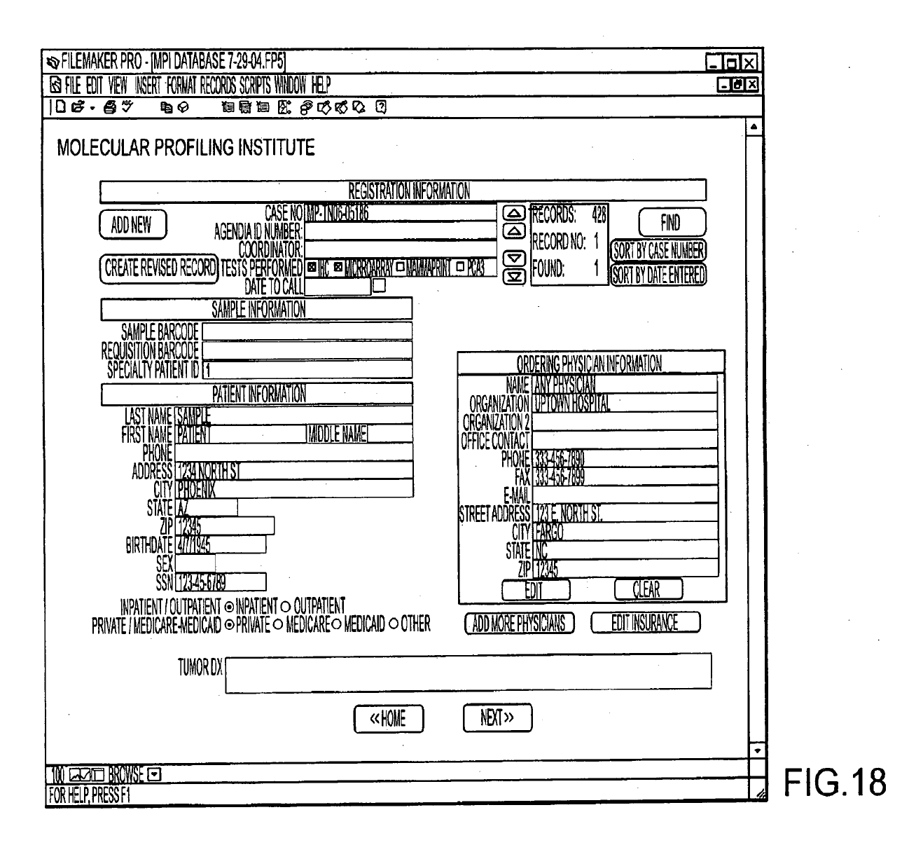

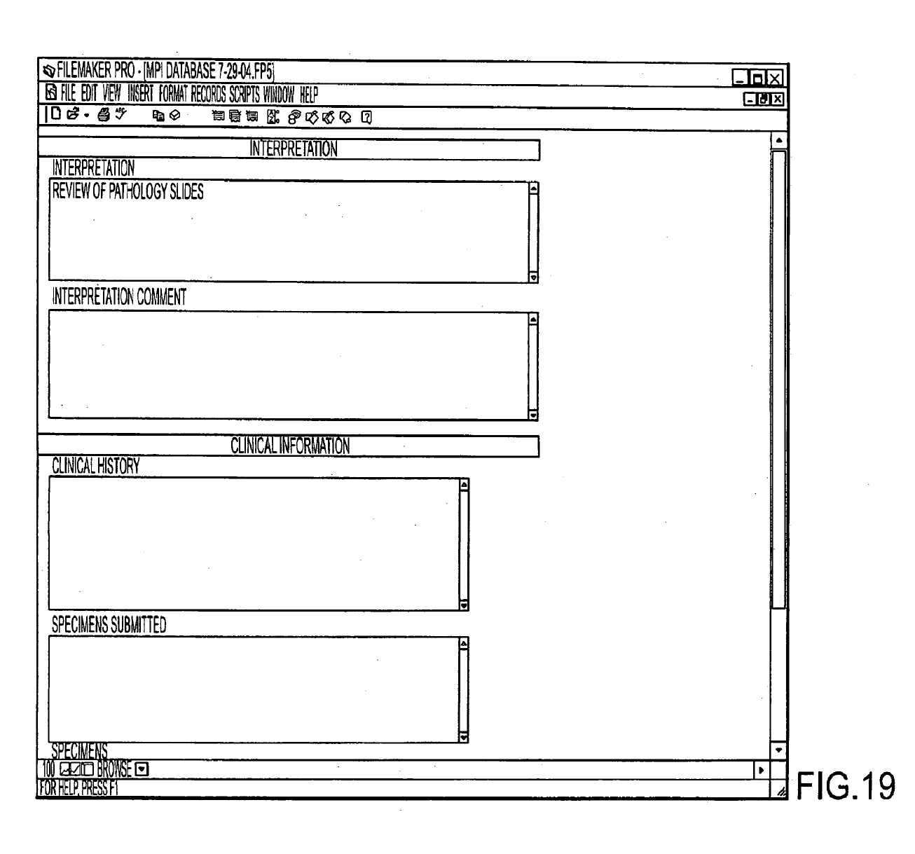

FIGS. 15-25 are computer screen print outs associated with various parts of the information-based personalized medicine drug discovery system and method shown in FIGS. 5-14. FIGS. 15 and 16 show computer screens where physician information and insurance company information is entered on behalf of a client. FIGS. 17-19 show computer screens in which information can be entered for ordering analysis and tests on patient samples.

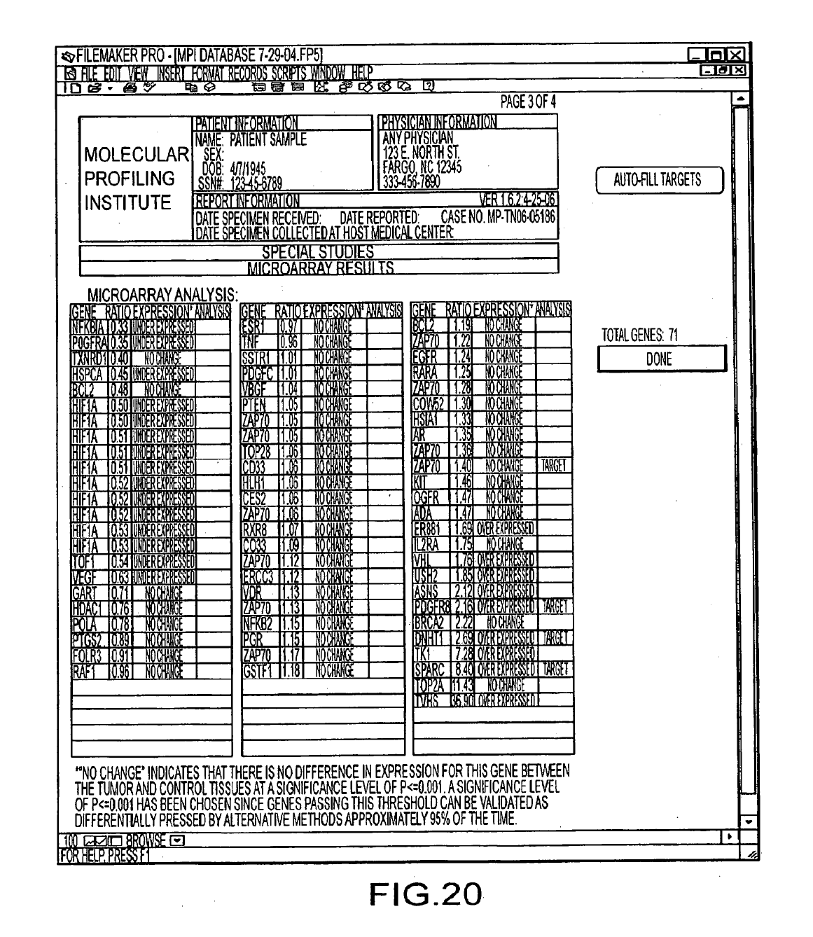

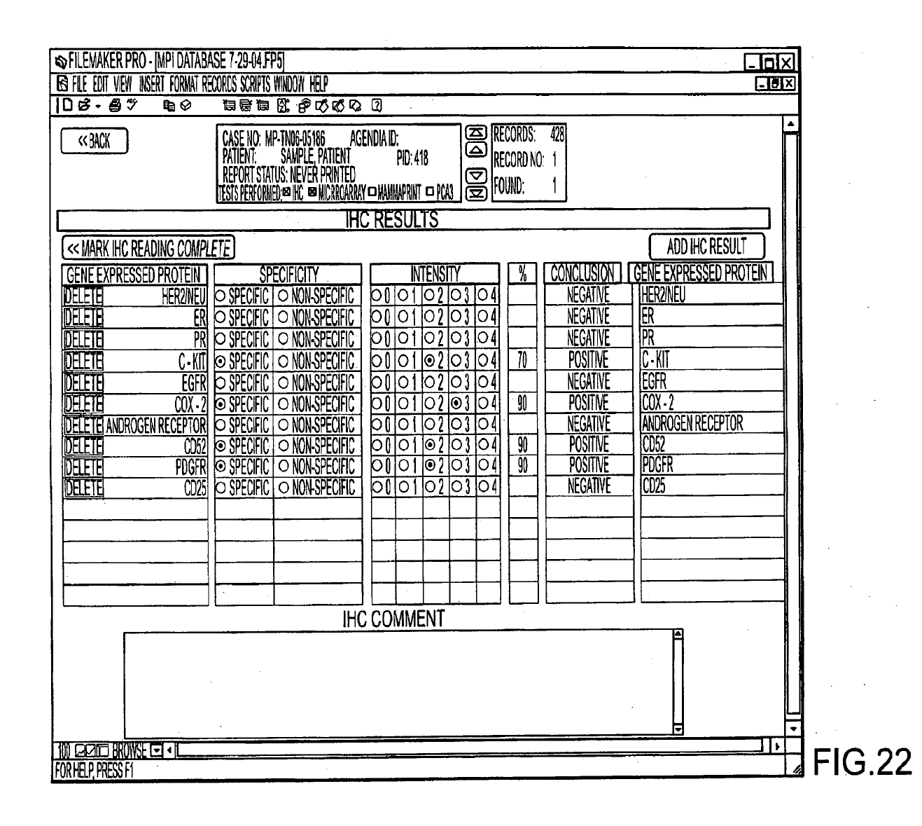

FIG. 20 is a computer screen showing micro array analysis results of specific genes tested with patient samples. This information and computer screen is similar to the information detailed in the patient profile report shown in FIG. 3C. FIG. 22 is a computer screen that shows immunohistochemistry test results for a particular patient for various genes. This information is similar to the information contained in the patient profile report shown in FIG. 3B.

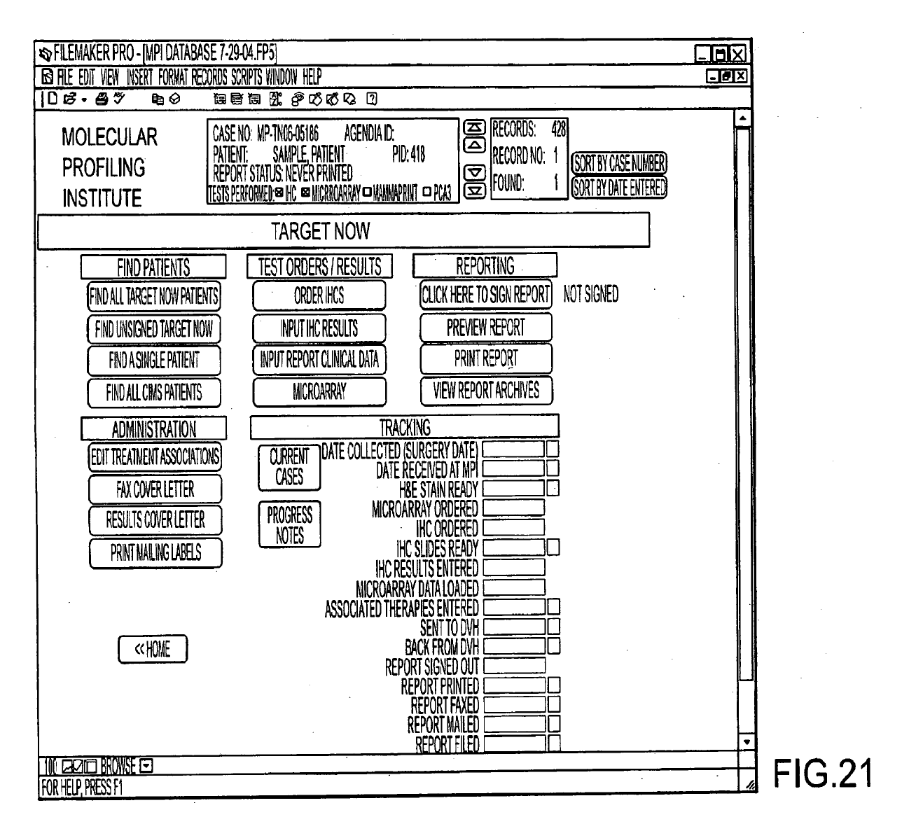

FIG. 21 is a computer screen showing selection options for finding particular patients, ordering tests and/or results, issuing patient reports, and tracking current cases/patients.

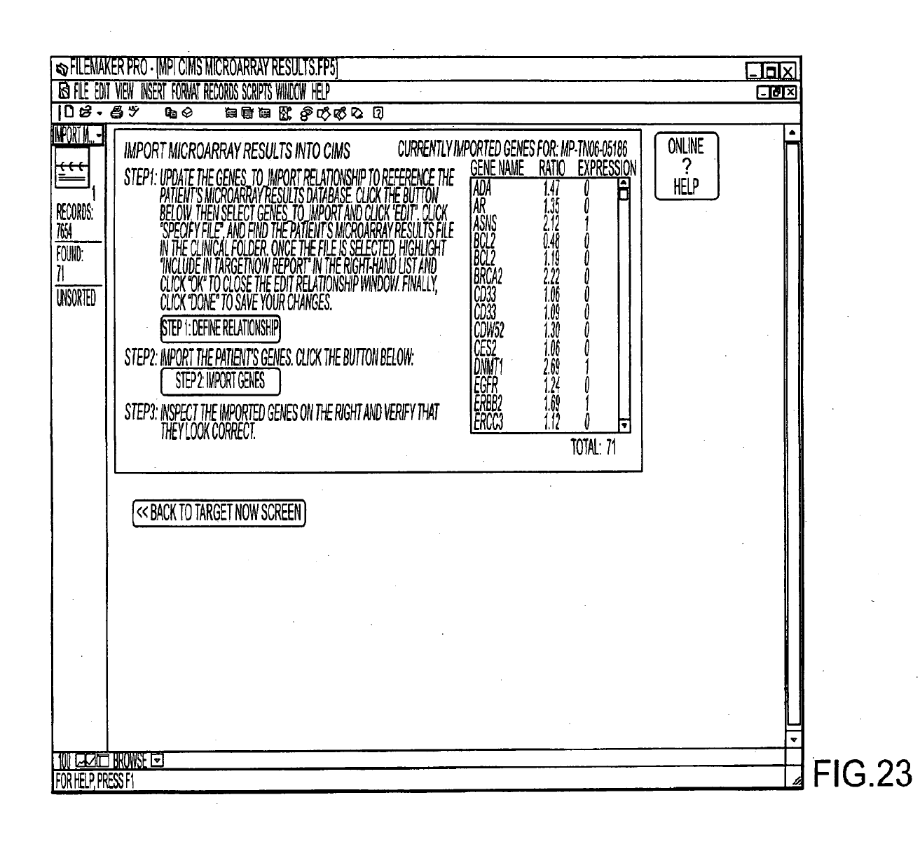

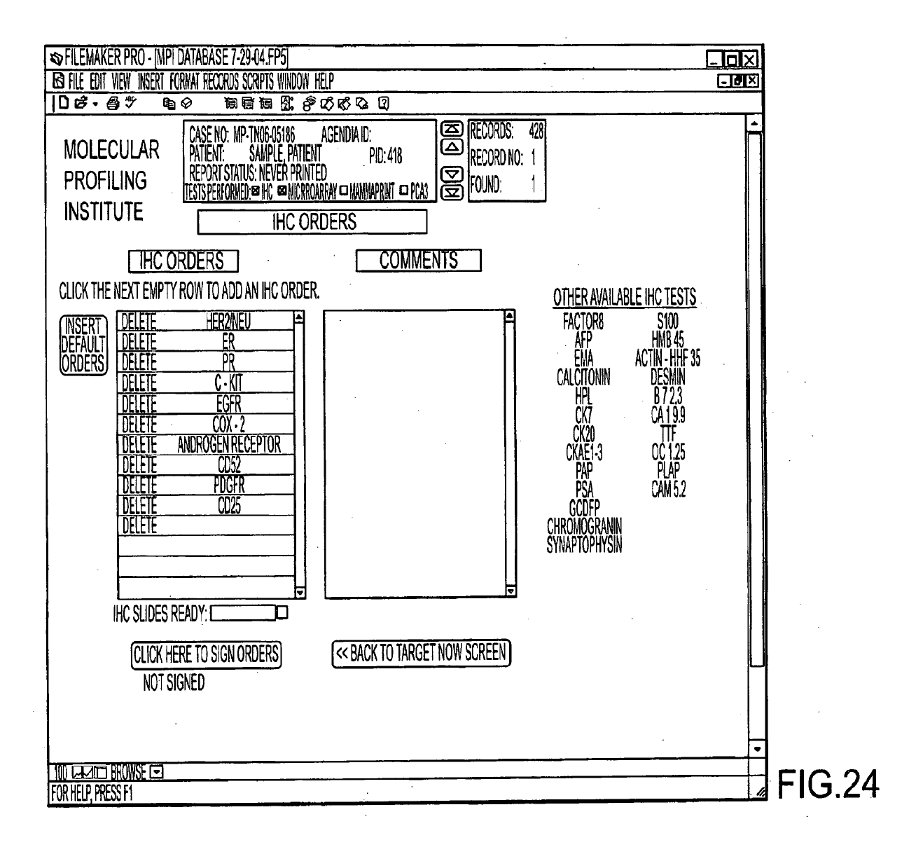

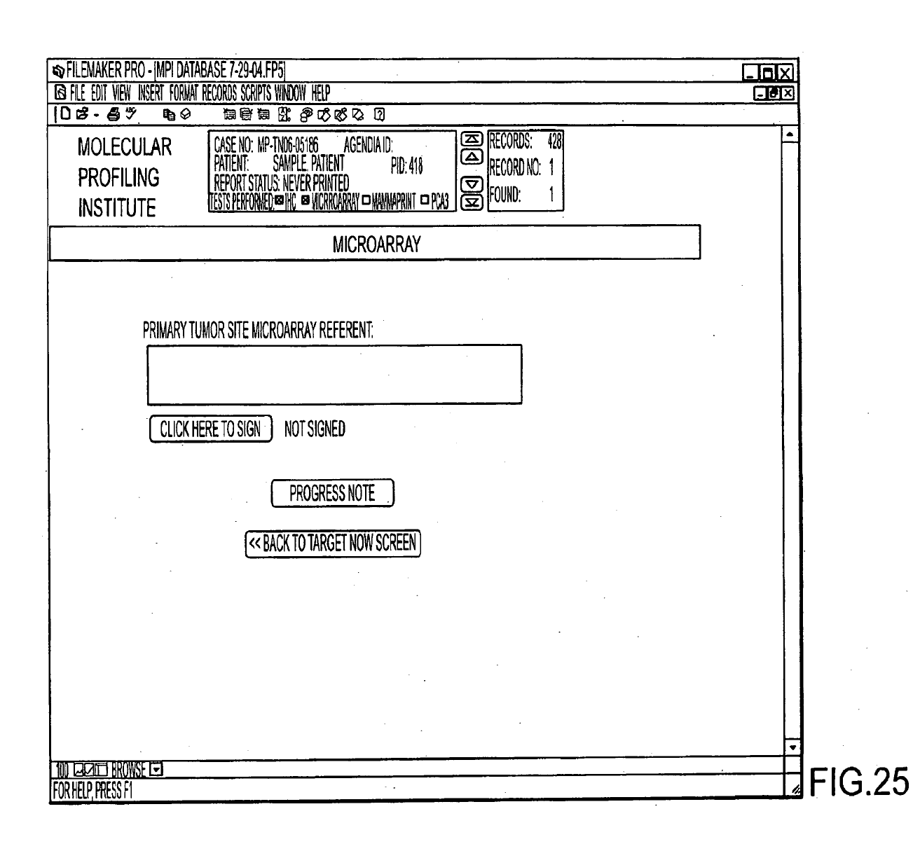

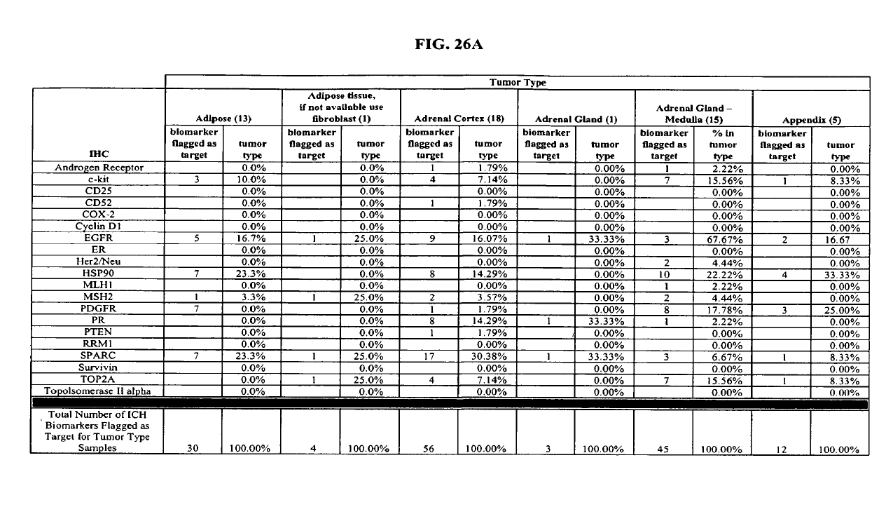

FIG. 23 is a computer screen which outlines some of the steps for creating a patient profile report as shown in FIGS. 3A through 3D. FIG. 24 shows a computer screen for ordering an immunohistochemistry test on a patient sample and FIG. 25 shows a computer screen for entering information regarding a primary tumor site for micro array analysis. It will be understood by those skilled in the art that any number and variety of computer screens may be utilized to enter the information necessary for utilizing the information-based personalized medicine drug discovery system and method of the present invention and to obtain information resulting from utilizing the information-based personalized medicine drug discovery system and method of the present invention.

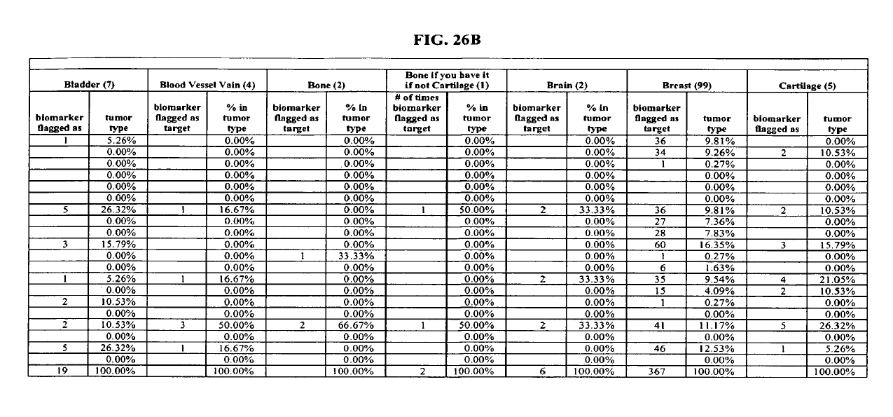

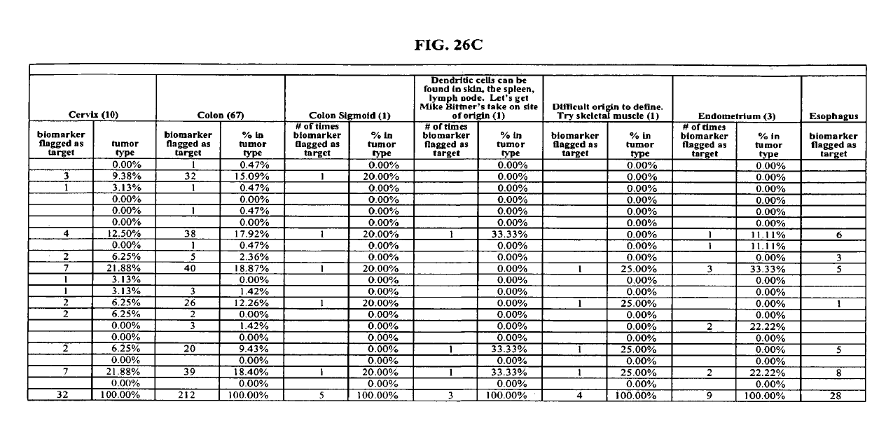

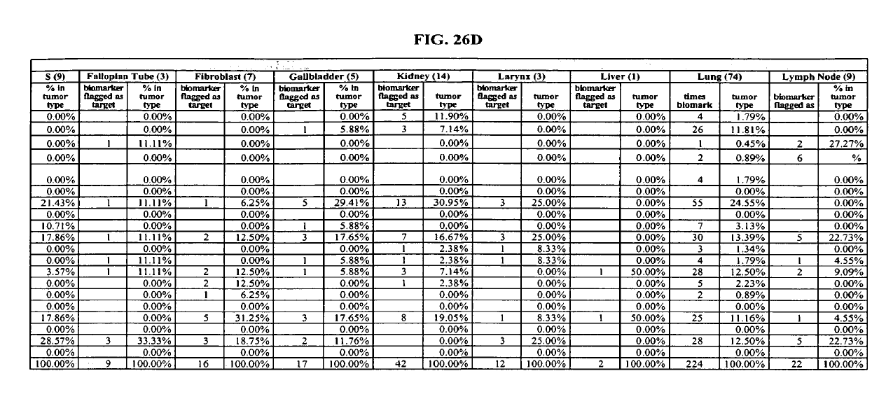

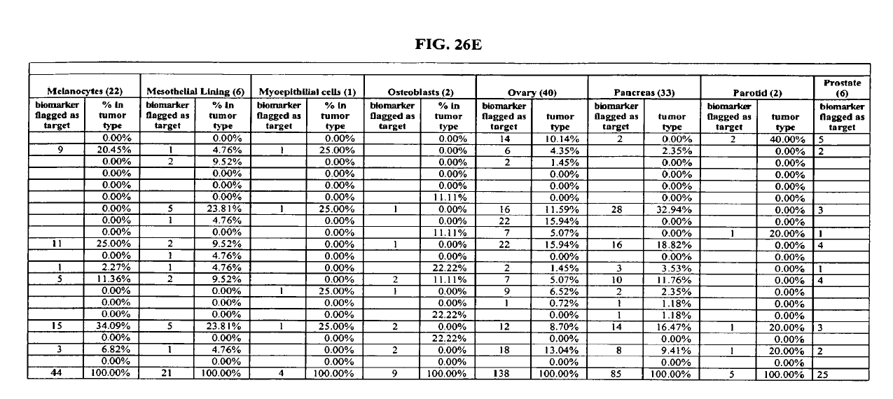

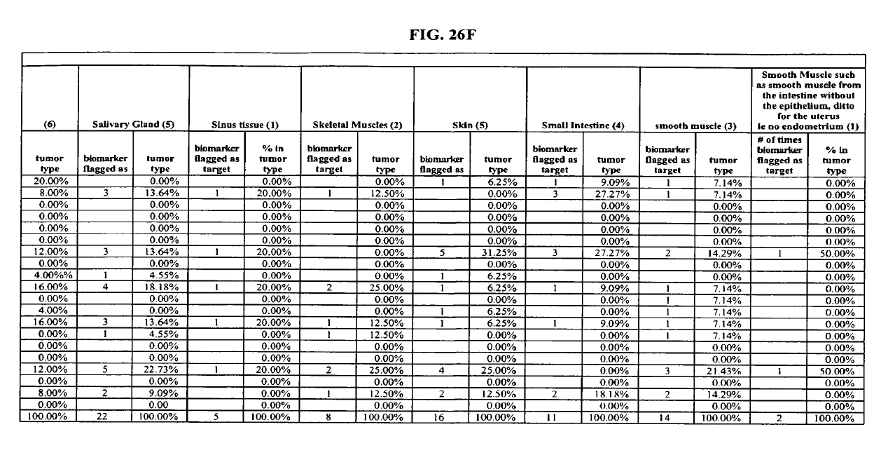

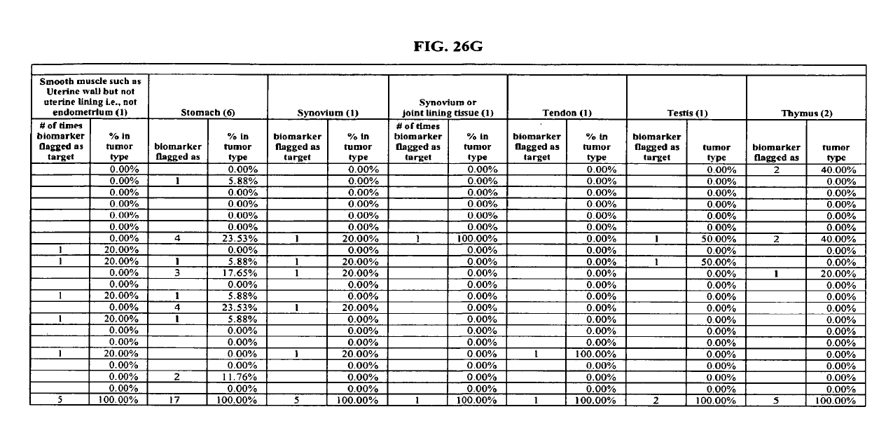

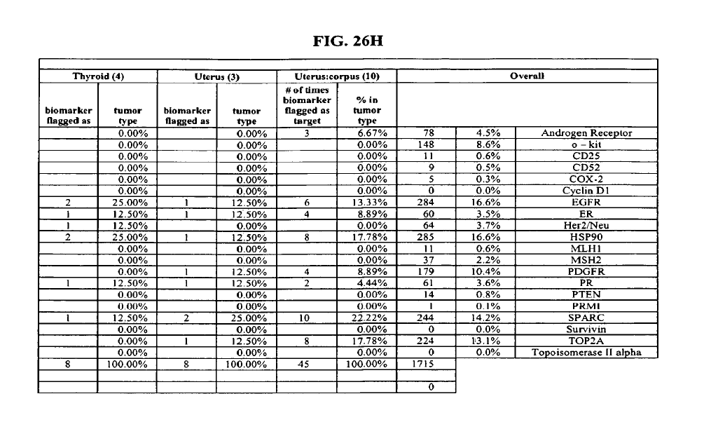

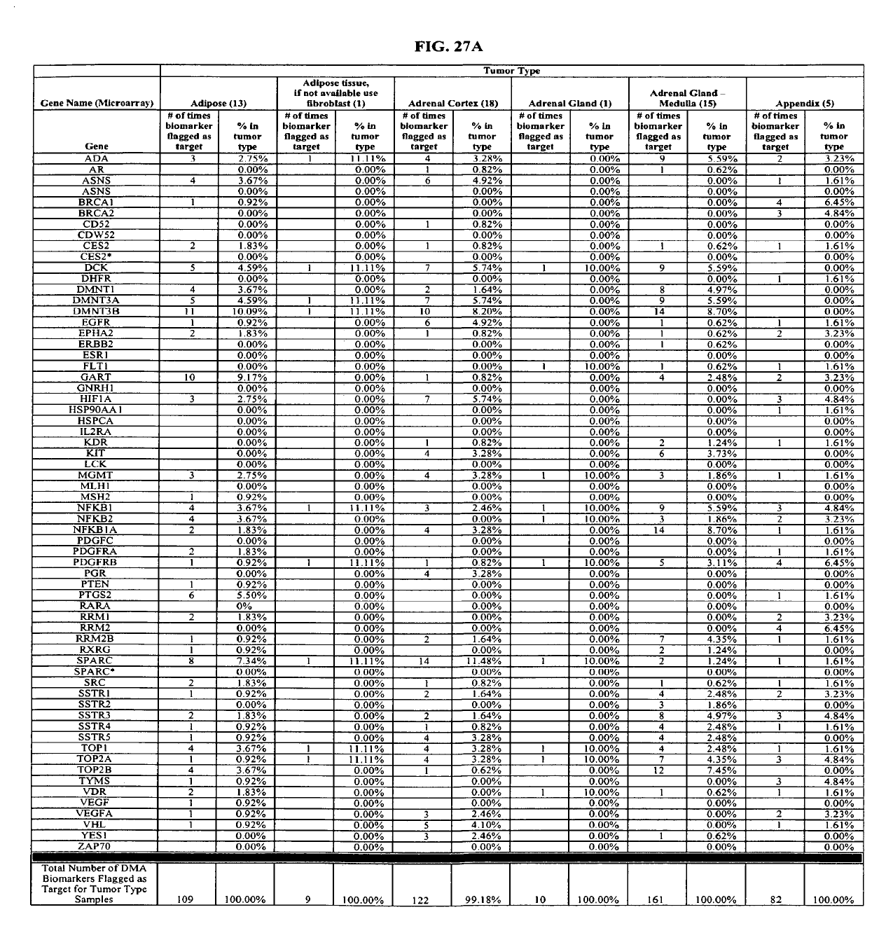

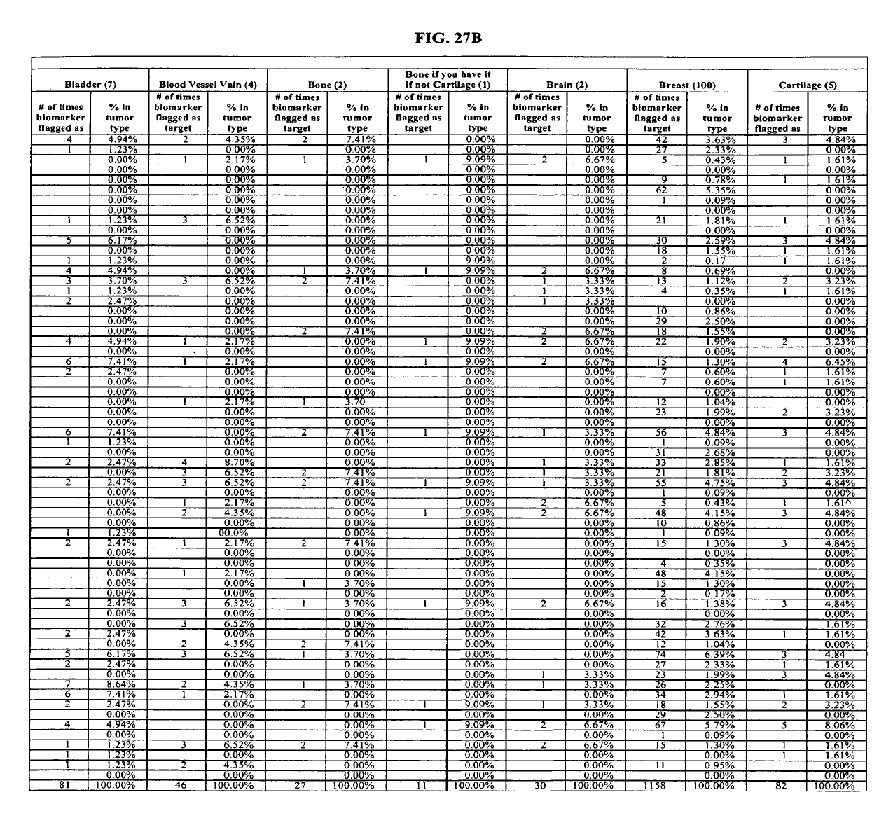

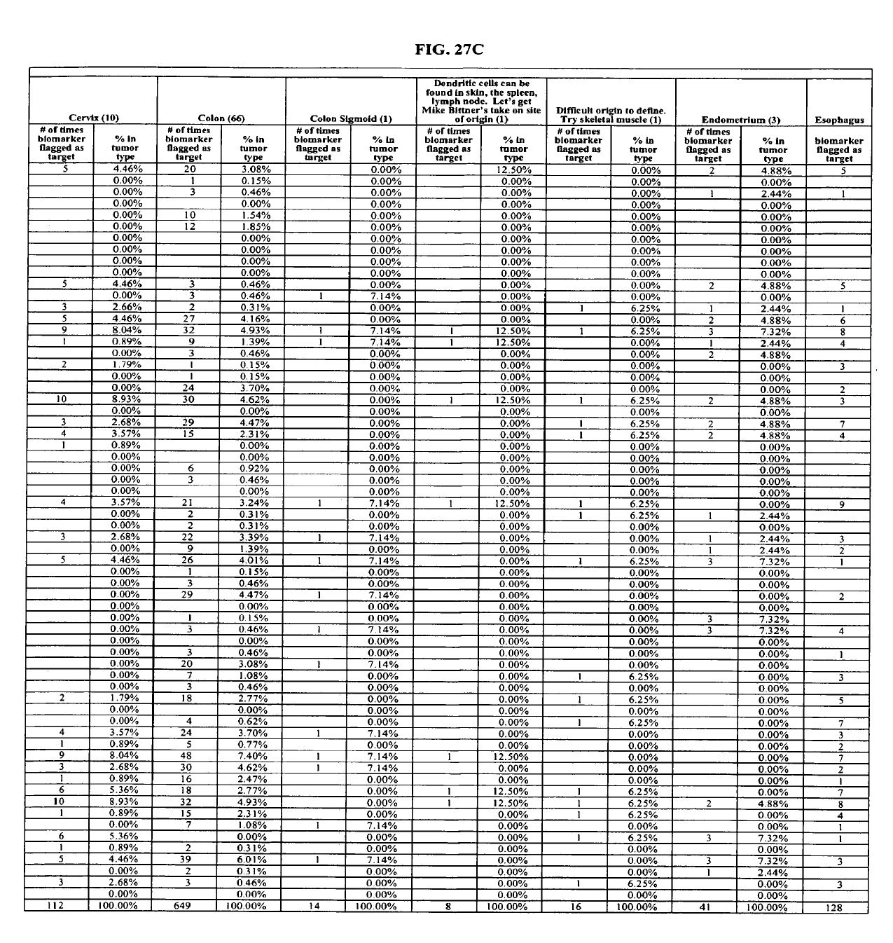

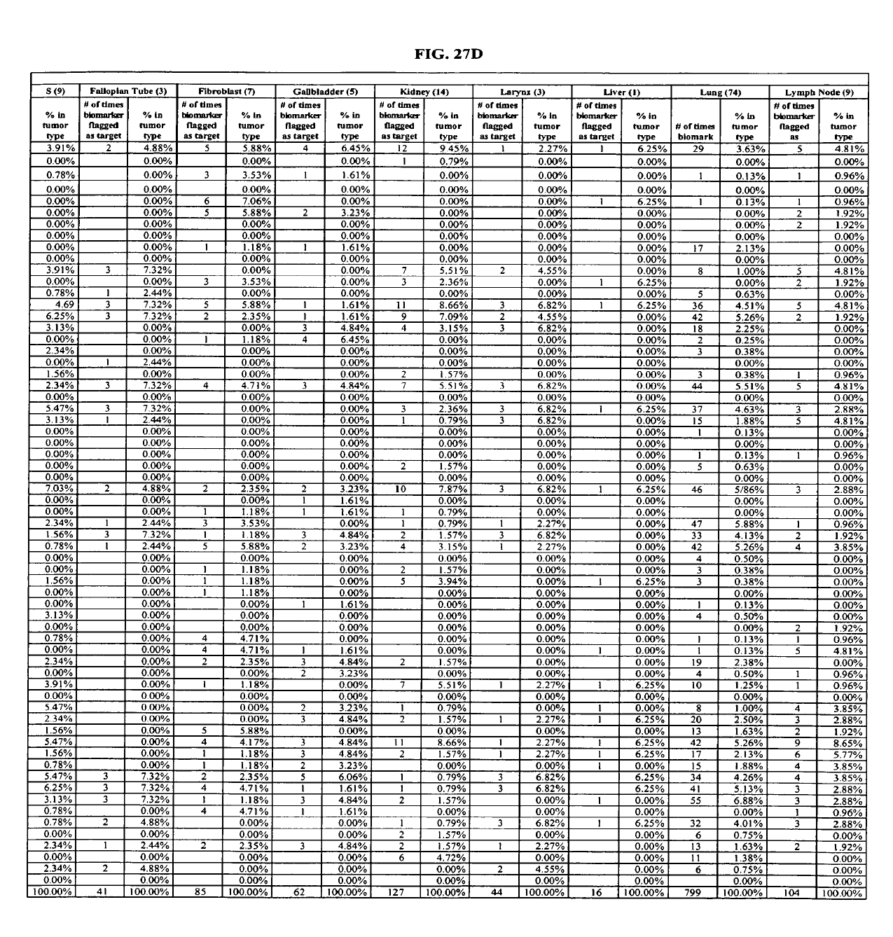

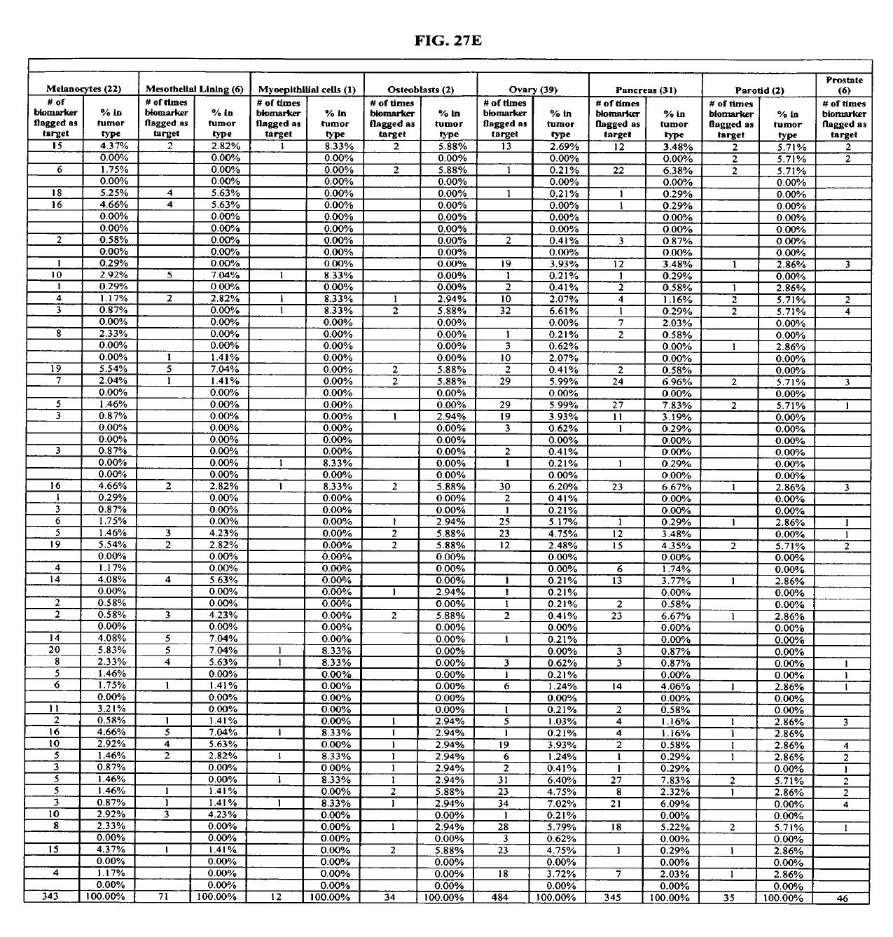

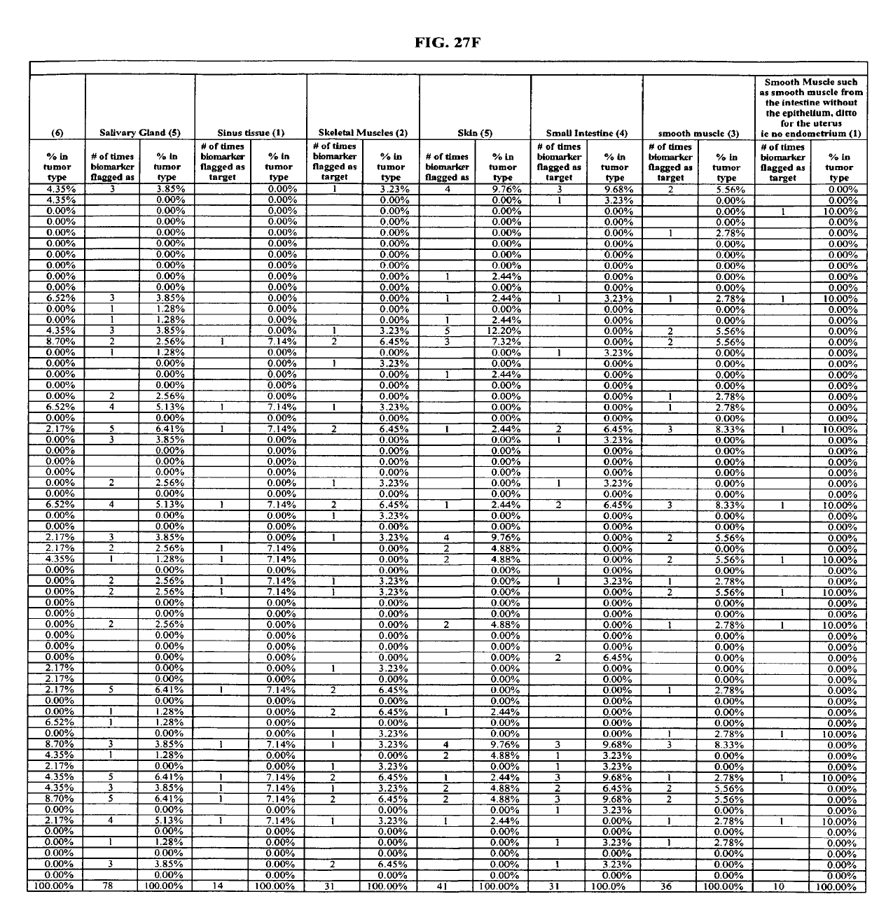

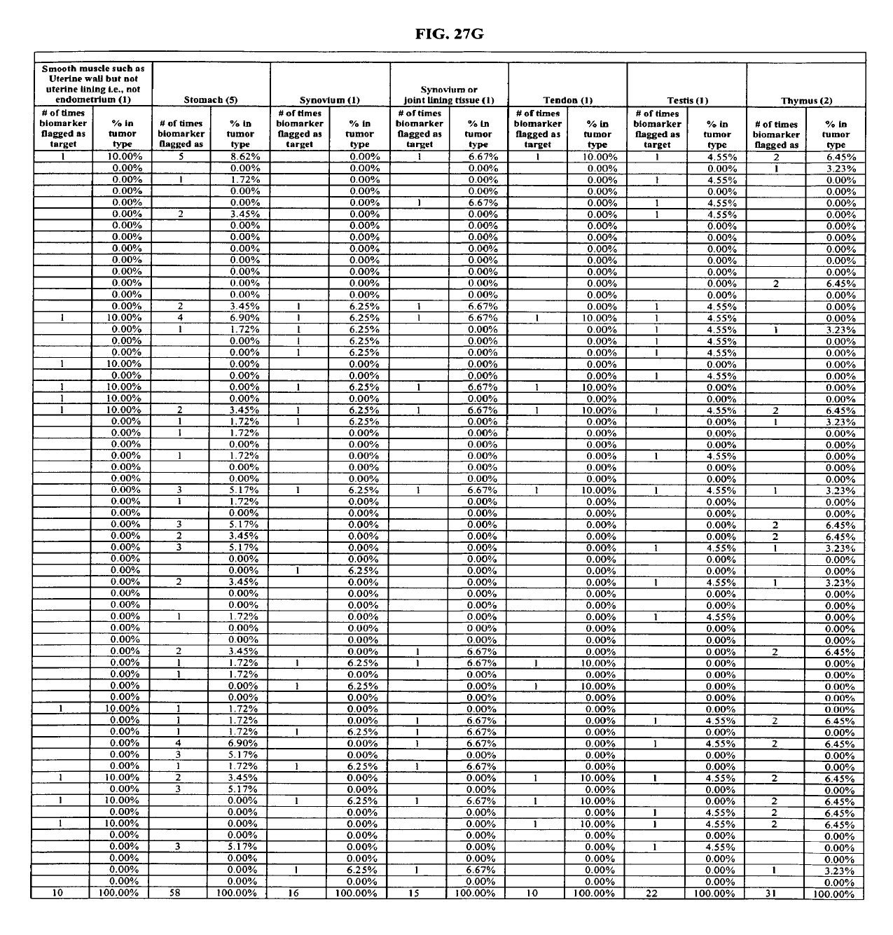

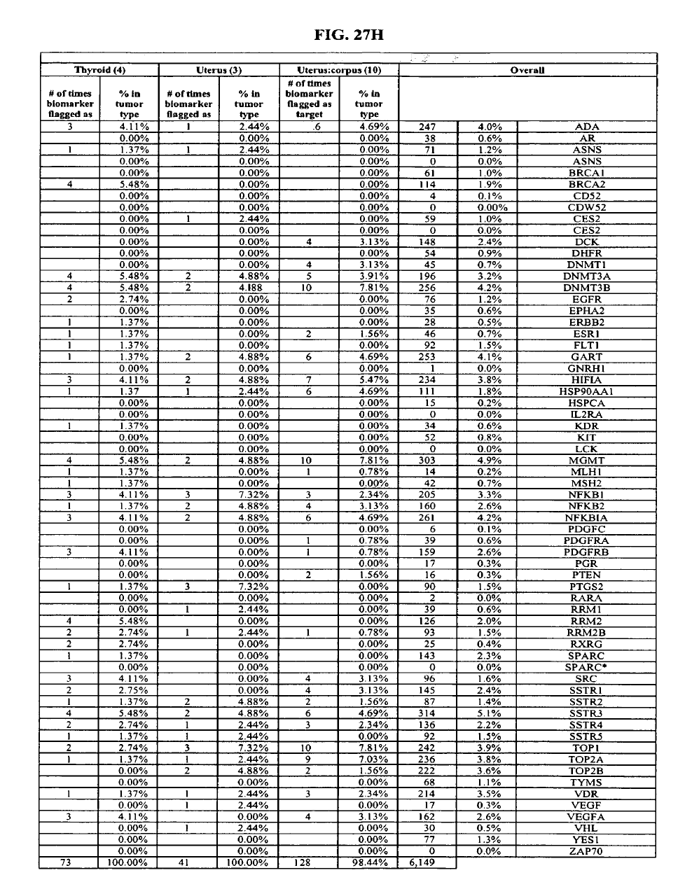

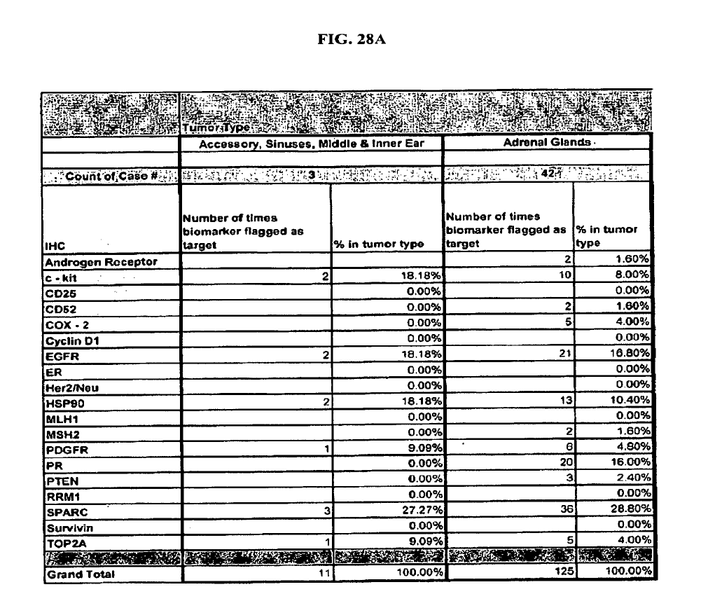

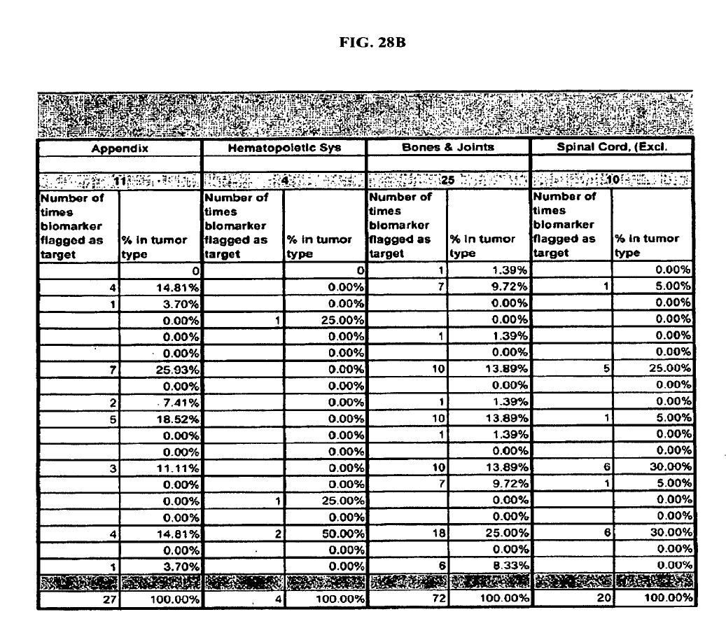

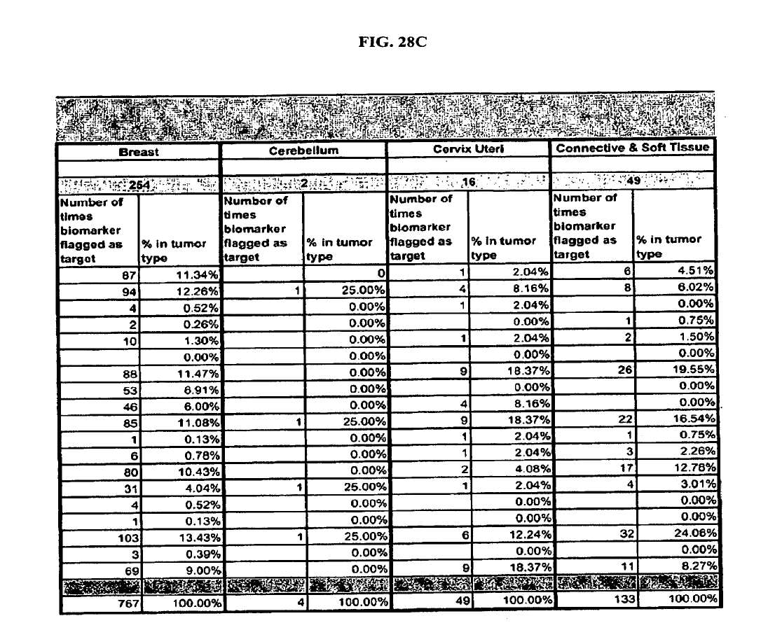

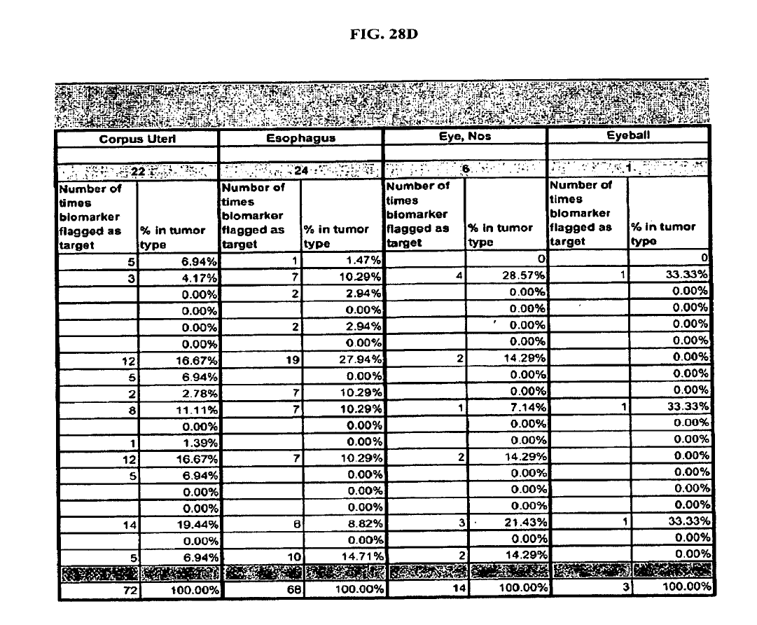

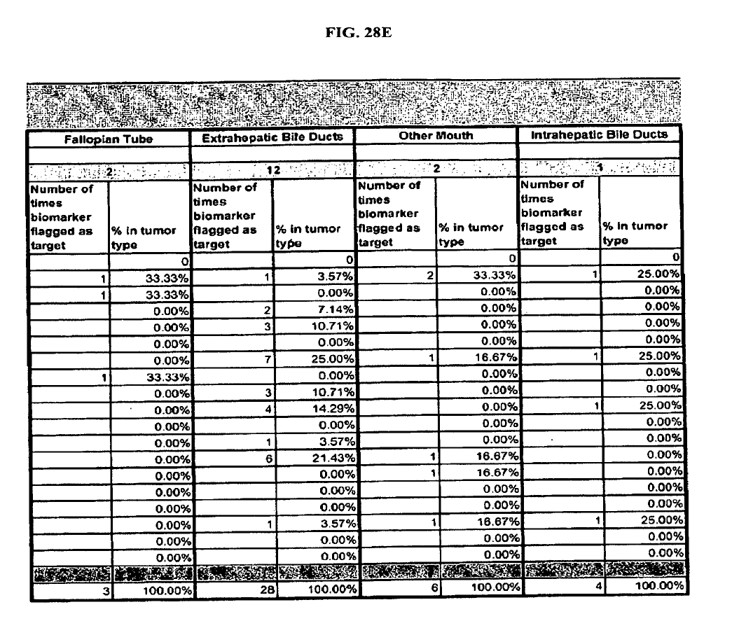

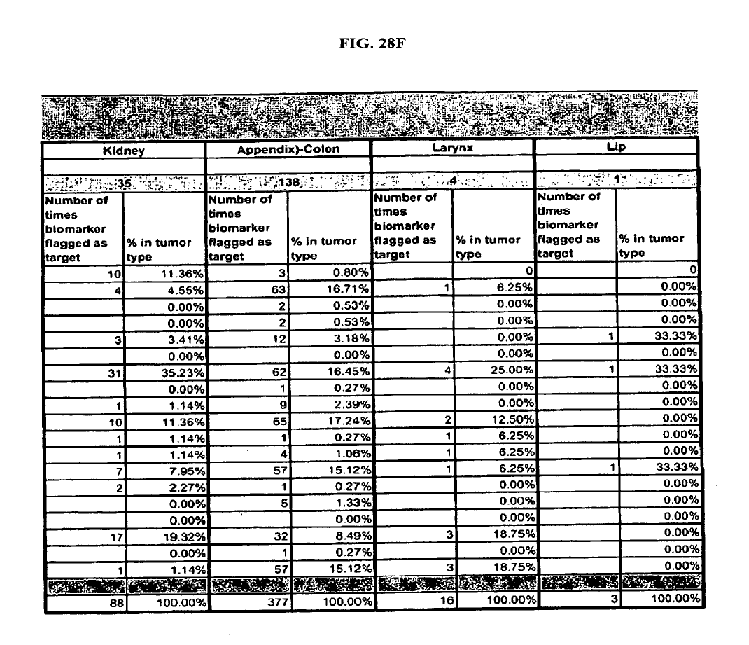

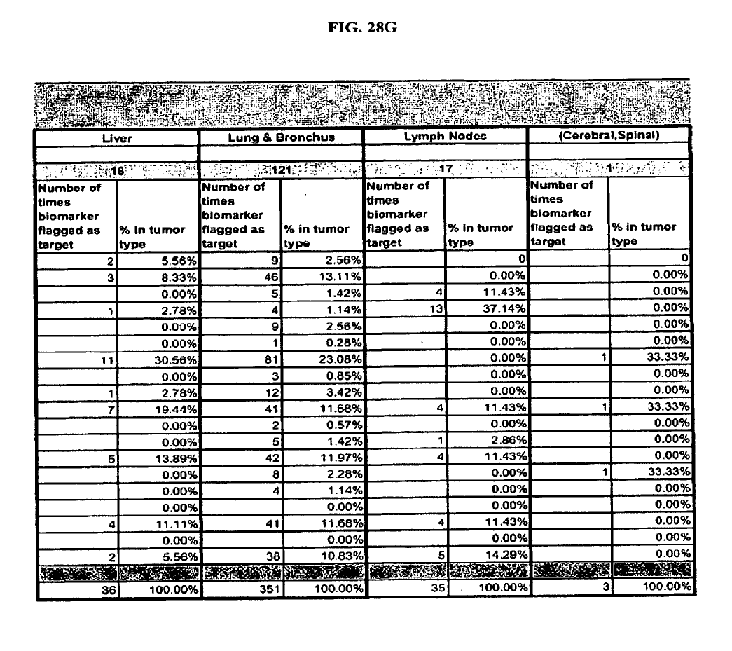

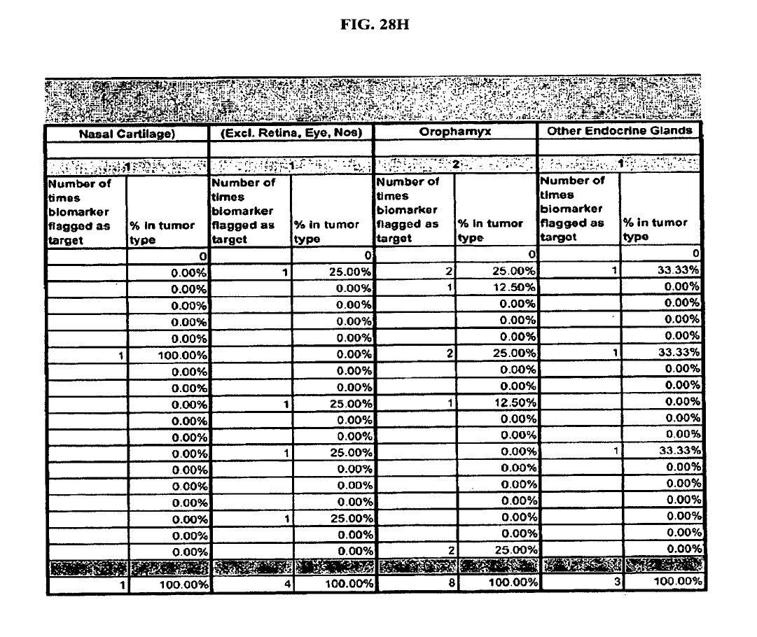

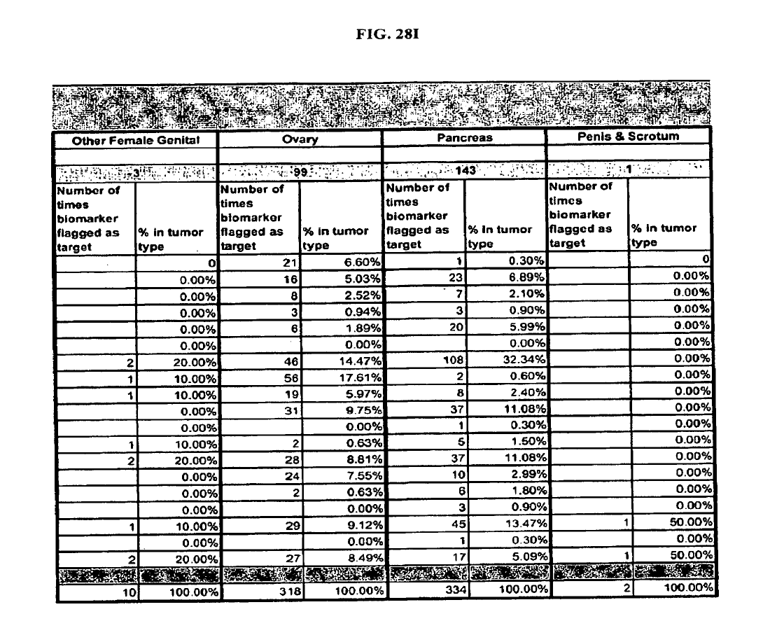

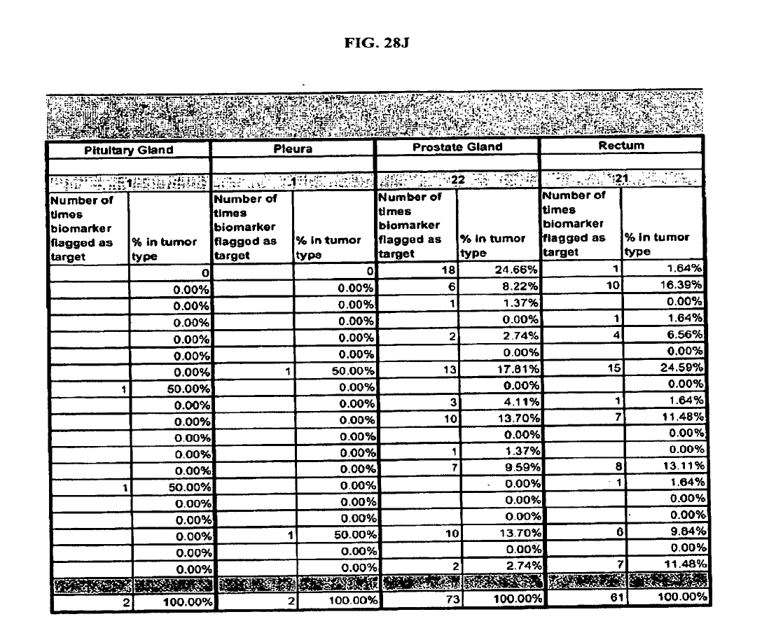

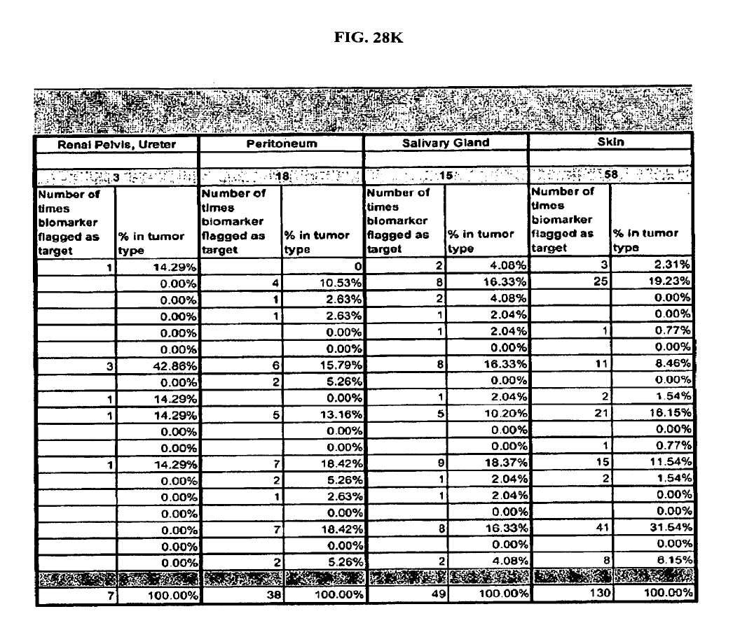

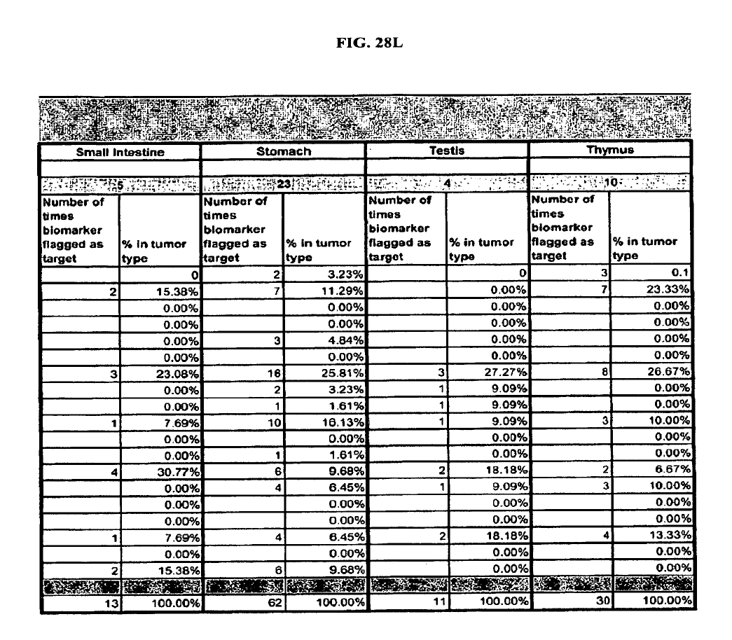

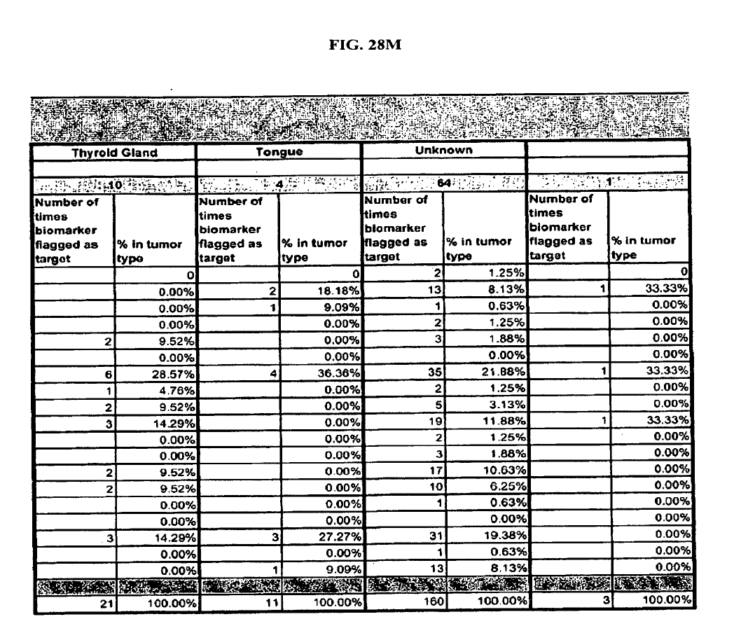

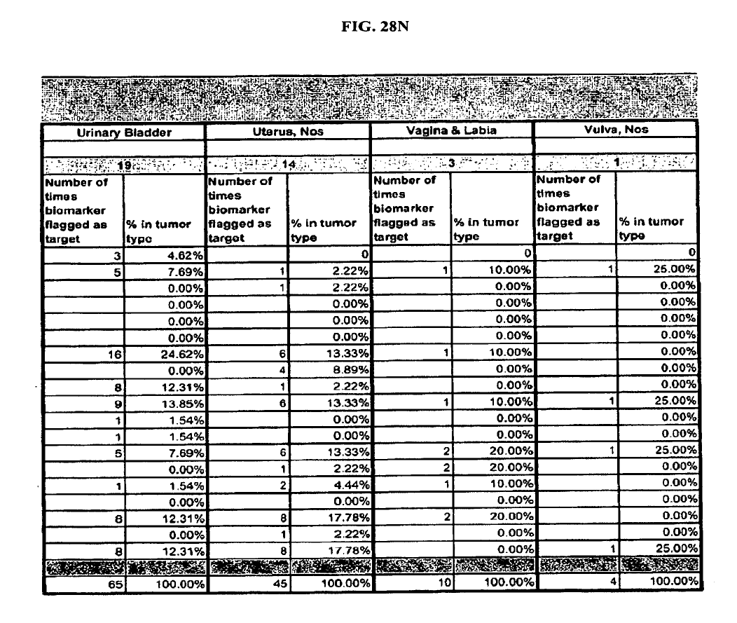

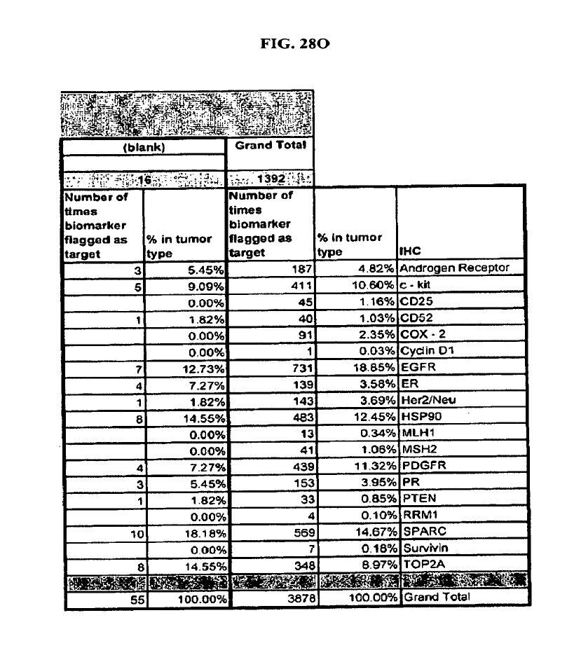

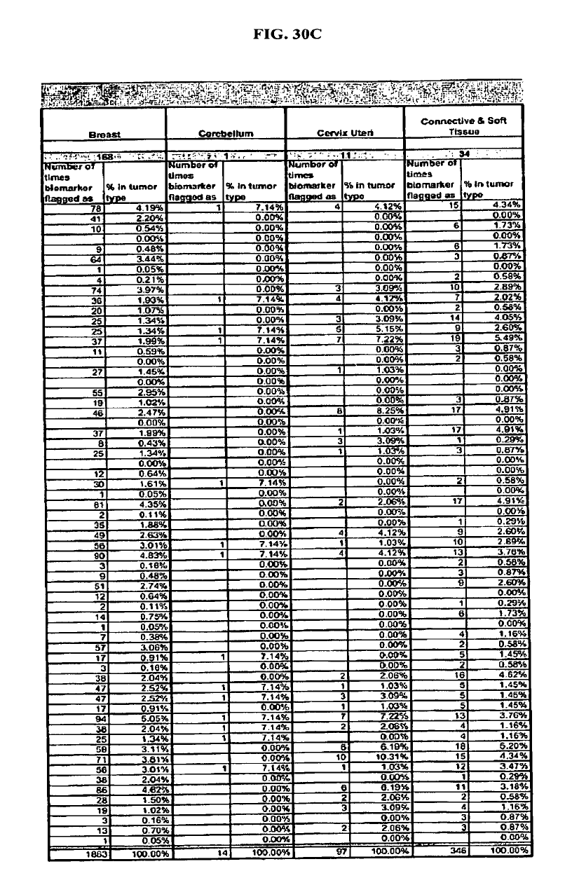

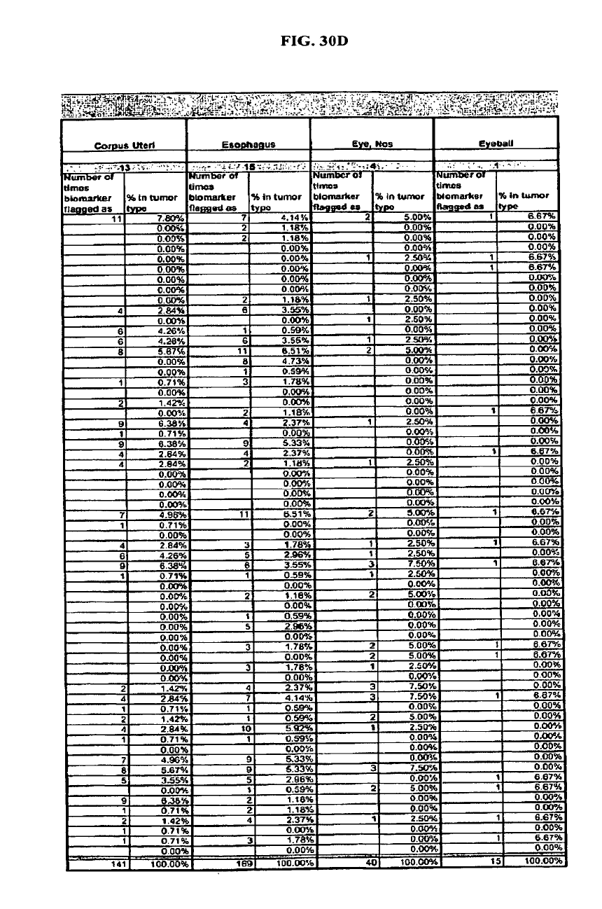

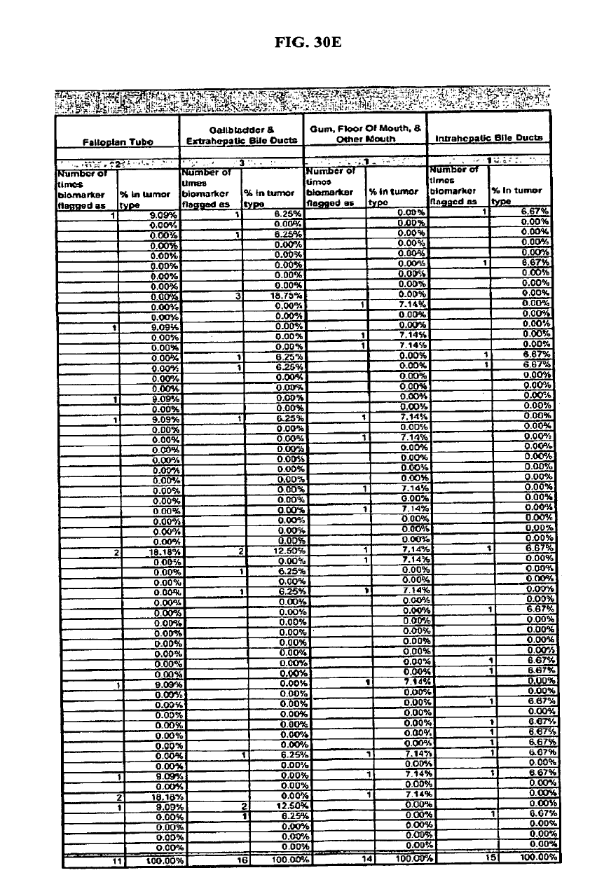

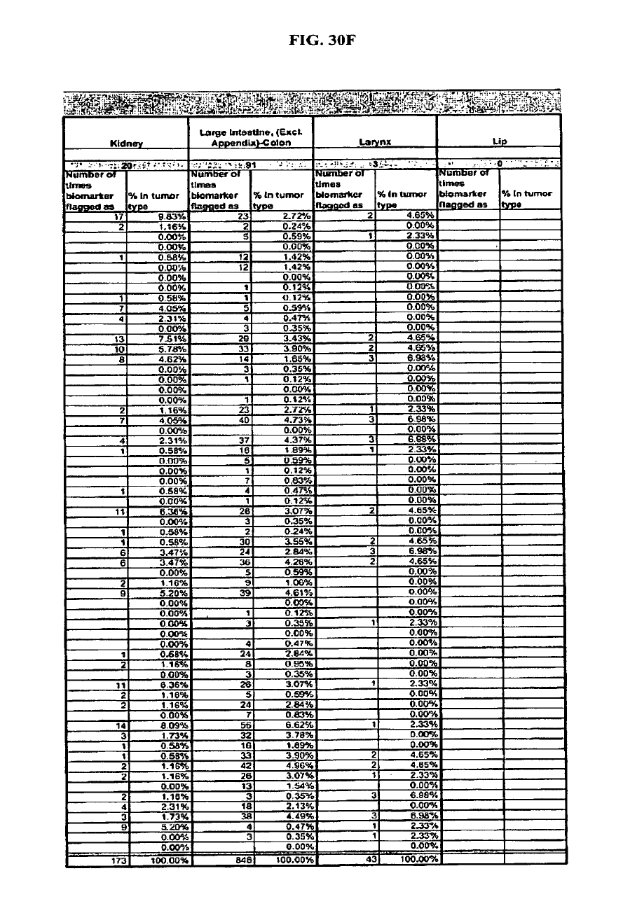

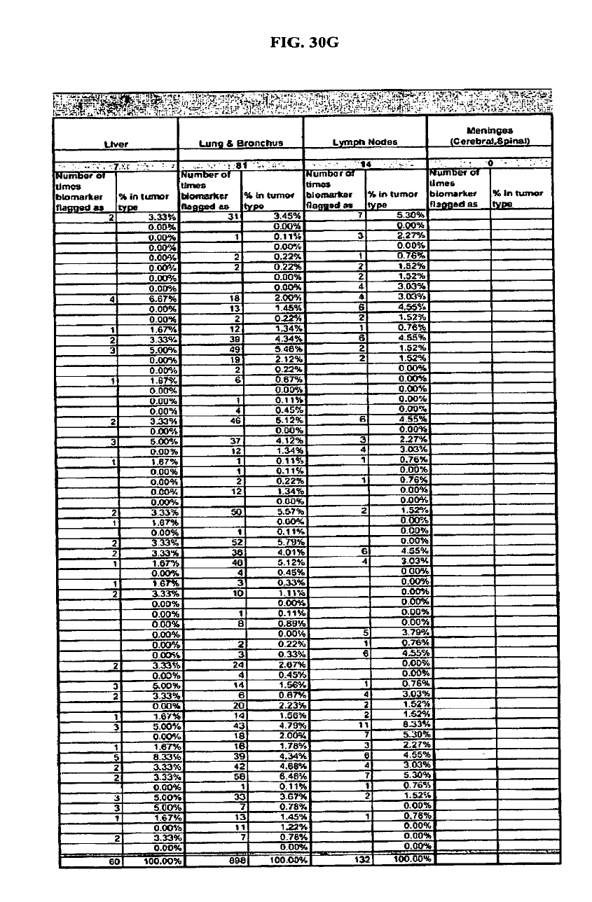

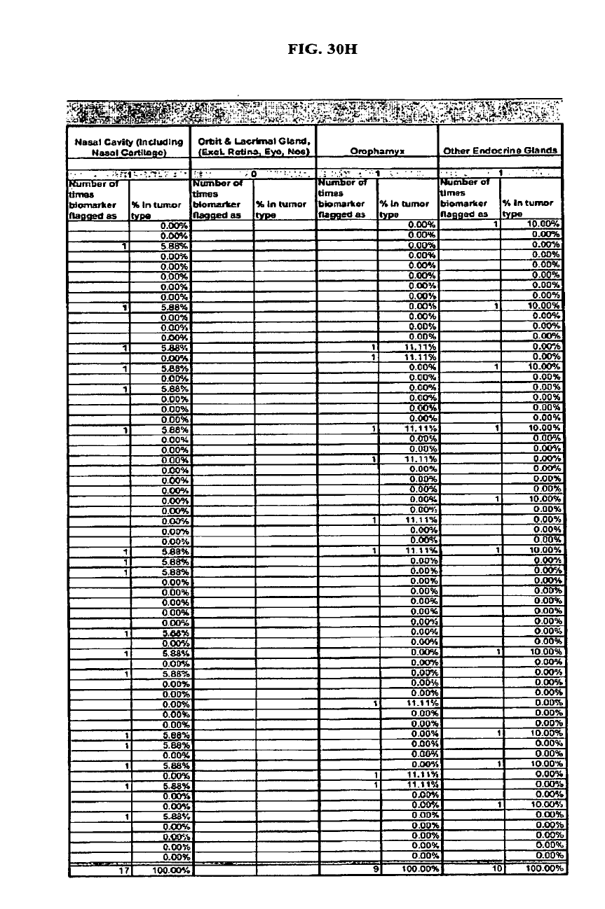

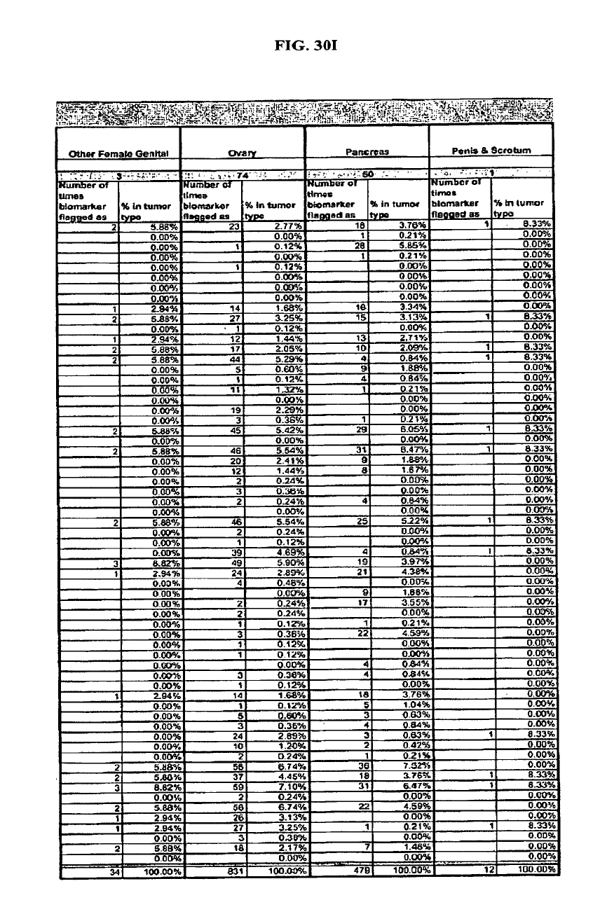

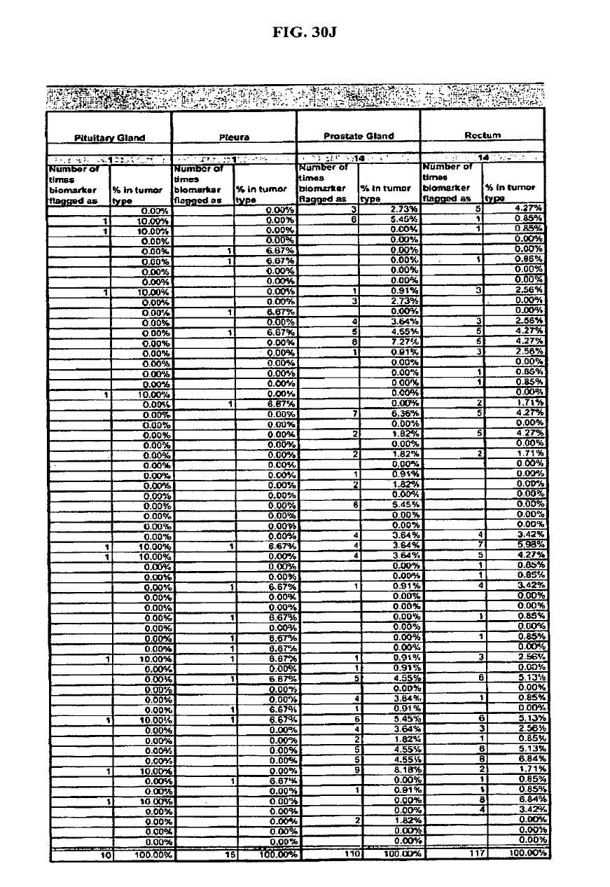

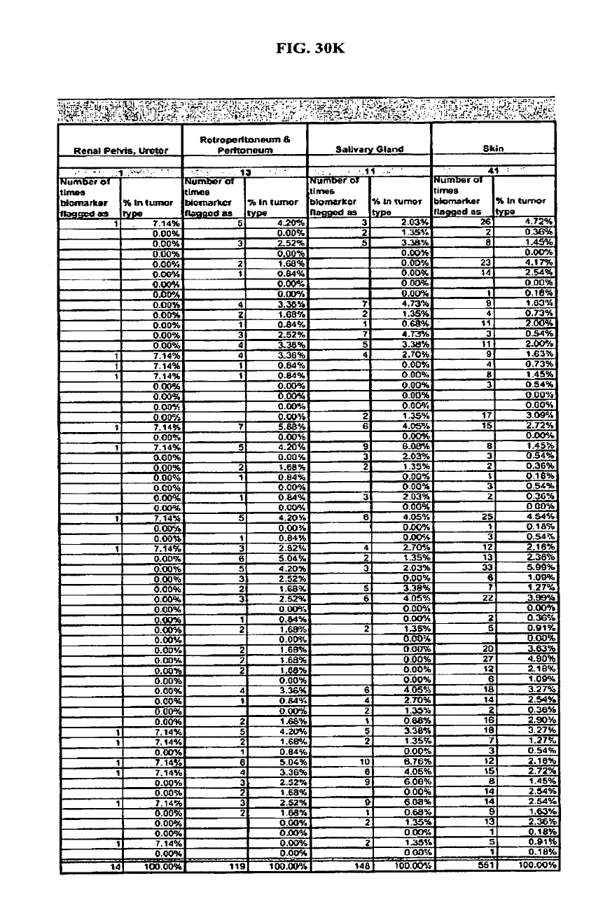

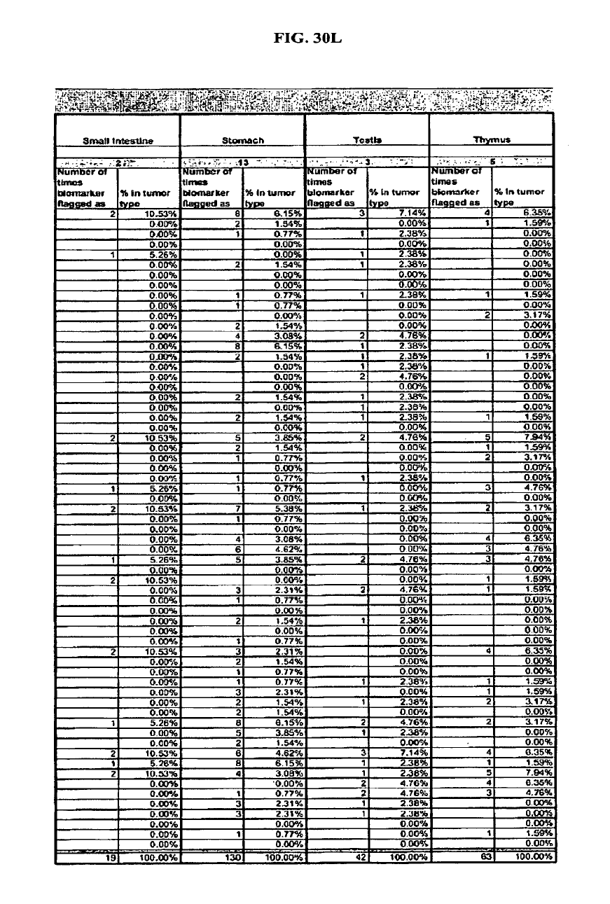

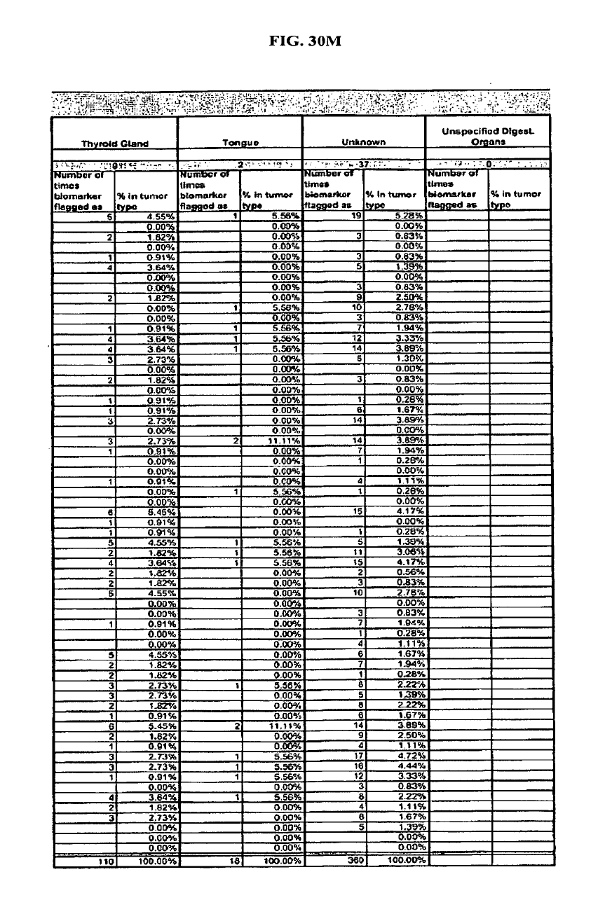

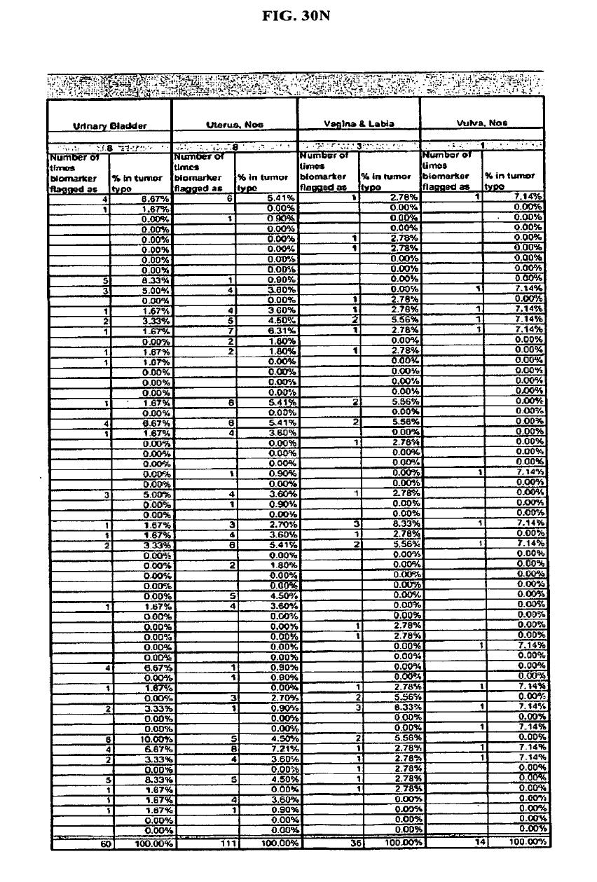

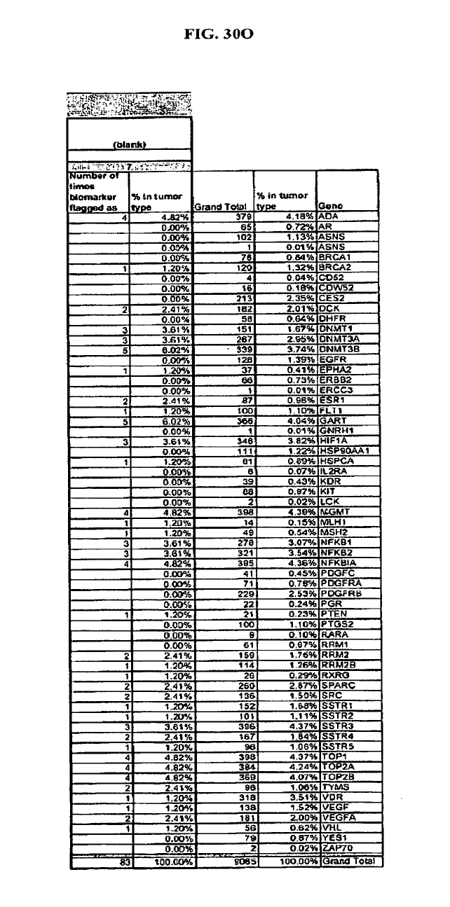

FIGS. 26-31 represent tables that show the frequency of a significant change in expression of certain genes and/or gene expressed proteins by tumor type, i.e. the number of times that a gene and/or gene expressed protein was flagged as a target by tumor type as being significantly overexpressed or underexpressed (see also Examples 1-3). The tables show the total number of times a gene and/or gene expressed protein was overexpressed or underexpressed in a particular tumor type and whether the change in expression was determined by immunohistochemistry analysis (FIG. 26, FIG. 28) or microarray analysis (FIGS. 27, 30). The tables also identify the total number of times an overexpression of any gene expressed protein occurred in a particular tumor type using immunohistochemistry and the total number of times an overexpression or underexpression of any gene occurred in a particular tumor type using gene microarray analysis.

Molecular Profiling Targets

The present invention provides methods and systems for analyzing diseased tissue using molecular profiling as previously described above. Because the methods rely on analysis of the characteristics of the tumor under analysis, the methods can be applied in for any tumor or any stage of disease, such an advanced stage of disease or a metastatic tumor of unknown origin. As described herein, a tumor or cancer sample is analyzed for molecular characteristics in order to predict or identify a candidate therapeutic treatment. The molecular characteristics can include the expression of genes or gene products, assessment of gene copy number, or mutational analysis. Any relevant determinable characteristic that can assist in prediction or identification of a candidate therapeutic can be included within the methods of the invention.

The biomarker patterns or biomarker signature sets can be determined for tumor types, diseased tissue types, or diseased cells including without limitation adipose, adrenal cortex, adrenal gland, adrenal gland-medulla, appendix, bladder, blood vessel, bone, bone cartilage, brain, breast, cartilage, cervix, colon, colon sigmoid, dendritic cells, skeletal muscle, endometrium, esophagus, fallopian tube, fibroblast, gallbladder, kidney, larynx, liver, lung, lymph node, melanocytes, mesothelial lining, myoepithelial cells, osteoblasts, ovary, pancreas, parotid, prostate, salivary gland, sinus tissue, skeletal muscle, skin, small intestine, smooth muscle, stomach, synovium, joint lining tissue, tendon, testis, thymus, thyroid, uterus, and uterus corpus.

The methods of the present invention can be used for selecting a treatment of any cancer or tumor type, including but not limited to breast cancer (including HER2+ breast cancer, HER2− breast cancer, ER/PR+, HER2− breast cancer, or triple negative breast cancer), pancreatic cancer, cancer of the colon and/or rectum, leukemia, skin cancer, bone cancer, prostate cancer, liver cancer, lung cancer, brain cancer, cancer of the larynx, gallbladder, parathyroid, thyroid, adrenal, neural tissue, head and neck, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell carcinoma, primary brain tumor, acute and chronic lymphocytic and granulocytic tumors, hairy-cell tumor, adenoma, hyperplasia, medullary carcinoma, pheochromocytoma, mucosal neuroma, intestinal ganglioneuroma, hyperplastic corneal nerve tumor, marfanoid habitus tumor, Wilm's tumor, seminoma, ovarian tumor, leiomyoma, cervical dysplasia and in situ carcinoma, neuroblastoma, retinoblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas. The cancer or tumor can comprise, without limitation, a carcinoma, a sarcoma, a lymphoma or leukemia, a germ cell tumor, a blastoma, or other cancers. Carcinomas that can be assessed using the subject methods include without limitation epithelial neoplasms, squamous cell neoplasms, squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma that can be assessed using the subject methods include without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia that can be assessed using the subject methods include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma. Germ cell tumors that can be assessed using the subject methods include without limitation germinoma, dysgerminoma, seminoma, nongerminomatous germ cell tumor, embryonal carcinoma, endodermal sinus turmor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor.

The methods of the invention can be used to determine biomarker patterns or biomarker signature sets in a number of tumor types, diseased tissue types, or diseased cells including accessory, sinuses, middle and inner car, adrenal glands, appendix, hematopoictic system, bones and joints, spinal cord, breast, cerebellum, cervix uteri, connective and soft tissue, corpus uteri, esophagus, eye, nose, eyeball, fallopian tube, extrahepatic bile ducts, other mouth, intrahepatic bile ducts, kidney, appendix-colon, larynx, lip, liver, lung and bronchus, lymph nodes, cerebral, spinal, nasal cartilage, excl. retina, eye, nos, oropharynx, other endocrine glands, other female genital, ovary, pancreas, penis and scrotum, pituitary gland, pleura, prostate gland, rectum renal pelvis, ureter, peritonem, salivary gland, skin, small intestine, stomach, testis, thymus, thyroid gland, tongue, unknown, urinary bladder, uterus, nos, vagina & labia, and vulva, nos.

In some embodiments, the molecular profiling methods are used to identify a treatment for a cancer of unknown primary (CUP). Approximately 40,000 CUP cases are reported annually in the US. Most of these are metastatic and/or poorly differentiated tumors. Because molecular profiling can identify a candidate treatment depending only upon the diseased sample, the methods of the invention can be used in the CUP setting. Moreover, molecular profiling can be used to create signatures of known tumors, which can then be used to classify a CUP and identify its origin. In an aspect, the invention provides a method of identifying the origin of a CUP, the method comprising performing molecular profiling on a panel of diseased samples to determine a panel of molecular profiles that correlate with the origin of each diseased sample, performing molecular profiling on a CUP sample, and correlating the molecular profile of the CUP sample with the molecular profiling of the panel of diseased samples, thereby identifying the origin of the CUP sample. The identification of the origin of the CUP sample can be made by matching the molecular profile of the CUP sample with the molecular profiles that correlate most closely from the panel of disease samples. The molecular profiling can use any of the techniques described herein, e.g., IHC, FISH, microarray and sequencing. The diseased samples and CUP samples can be derived from a patient sample, e.g., a biopsy sample, including a fine needle biopsy. In one embodiment, DNA microarray and IHC profiling are performed on the panel of diseased samples, DNA microarray is performed on the CUP samples, and then IHC is performed on the CUP sample for a subset of the most informative genes as indicated by the DNA microarray analysis. This approach can identify the origin of the CUP sample while avoiding the expense of performing unnecessary IHC testing. The IHC can be used to confirm the microarray findings.

The biomarker patterns or biomarker signature sets of the cancer or tumor can be used to determine a therapeutic agent or therapeutic protocol that is capable of interacting with the biomarker pattern or signature set. For example, with advanced breast cancer, immunohistochemistry analysis can be used to determine one or more gene expressed proteins that are overexpressed. Accordingly, a biomarker pattern or biomarker signature set can be identified for advanced stage breast cancer and a therapeutic agent or therapeutic protocol can be identified which is capable of interacting with the biomarker pattern or signature set.

These examples of biomarker patterns or biomarker signature sets for advanced stage breast cancer are just one example of the extensive number of biomarker patterns or biomarker signature sets for a number of advanced stage diseases or cancers that can be identified from the tables depicted in FIGS. 26-31. In addition, a number of non disease specific therapies or therapeutic protocols may be identified for treating patients with these biomarker patterns or biomarker signature sets by utilizing method steps of the present invention described above such as depicted in FIGS. 1-2 and FIGS. 5-14.

The biomarker patterns and/or biomarker signature sets disclosed in the table depicted in FIGS. 26 and 28, and the tables depicted in FIGS. 27 and 30 may be used for a number of purposes including, but not limited to, specific cancer/disease detection, specific cancer/disease treatment, and identification of new drug therapies or protocols for specific cancers/diseases. The biomarker patterns and/or biomarker signature sets disclosed in the table depicted in FIGS. 26 and 28, and the tables depicted in FIGS. 27 and 30 can also represent drug resistant expression profiles for the specific tumor type or cancer type. The biomarker patterns and/or biomarker signature sets disclosed in the table depicted in FIGS. 26 and 28, and the tables depicted in FIGS. 27 and 30 represent advanced stage drug resistant profiles.

The biomarker patterns and/or biomarker signature sets can comprise at least one biomarker. In yet other embodiments, the biomarker patterns or signature sets can comprise at least 2, 3, 4, 5, 6, 7, 8, 9, or 10 biomarkers. In some embodiments, the biomarker signature sets or biomarker patterns can comprise at least 15, 20, 30, 40, 50, or 60 biomarkers. In some embodiments, the biomarker signature sets or biomarker patterns can comprise at least 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000 or 50,000 biomarkers. Analysis of the one or more biomarkers can be by one or more methods. For example, analysis of 2 biomarkers can be performed using microarrays. Alternatively, one biomarker may be analyzed by IHC and another by microarray. Any such combinations of methods and biomarkers are contemplated herein.

The one or more biomarkers can be selected from the group consisting of, but not limited to: Her2/Neu, ER, PR, c-kit, EGFR, MLH1, MSH2, CD20, p53, Cyclin D1, bcl2, COX-2, Androgen receptor, CD52, PDGFR, AR, CD25, VEGF, HSP90, PTEN, RRM1, SPARC, Survivin, TOP2A, BCL2, HIF1A, AR, ESR1, PDGFRA, KIT, PDGFRB, CDW52, ZAP70, PGR, SPARC, GART, GSTP1, NFKBIA, MSH2, TXNRD1, HDAC1, PDGFC, PTEN, CD33, TYMS, RXRB, ADA, TNF, ERCC3, RAF1, VEGF, TOP1, TOP2A, BRCA2, TK1, FOLR2, TOP2B, MLH1, IL2RA, DNMT1, HSPCA, ERBR2, ERBB2, SSTR1, VHL, VDR, PTGS2, POLA, CES2, EGFR, OGFR, ASNS, NFKB2, RARA, MS4A1, DCK, DNMT3A, EREG, Epiregulin, FOLR1, GNRH1, GNRHR1, FSHB, FSHR, FSHPRH1, folate receptor, HGF, HIG1, IL13RA1, LTB, ODC1, PPARG, PPARGC1, Lymphotoxin Beta Receptor, Myc, Topoisomerase II, TOPO2B, TXN, VEGFC, ACE2, ADH1C, ADH4, AGT, AREG, CA2, CDK2, caveolin, NFKB1, ASNS, BDCA1, CD52, DHFR, DNMT3B, EPHA2, FLT1, HSP90AA1, KDR, LCK, MGMT, RRM1, RRM2, RRM2B, RXRG, SRC, SSTR2, SSTR3, SSTR4, SSTR5, VEGFA, or YES1.

For example, a biological sample from an individual can be analyzed to determine a biomarker pattern or biomarker signature set that comprises a biomarker such as HSP90, Survivin, RRM1, SSTRS3, DNMT3B, VEGFA, SSTR4, RRM2, SRC, RRM2B, IISP90AA1, STR2, FLT1, SSTR5, YES1, BRCA1, RRM1, DHFR, KDR, EPHA2, RXRG, or LCK. In other embodiments, the biomarker SPARC, HSP90, TOP2A, PTEN, Survivin, or RRM1 forms part of the biomarker pattern or biomarker signature set. In yet other embodiments, the biomarker MGMT, SSTRS3, DNMT3B, VEGFA, SSTR4, RRM2, SRC, RRM2B, HSP90AA1, STR2, FLT1, SSTR5, YES1, BRCA1, RRM1, DHFR, KDR, EPHA2, RXRG, CD52, or LCK is included in a biomarker pattern or biomarker signature set. In still other embodiments, the biomarker hENT1, cMet, P21, PARP-1, TLE3 or IGF1R is included in a biomarker pattern or biomarker signature set.

The expression level of HSP90, Survivin, RRM1, SSTRS3, DNMT3B, VEGFA, SSTR4, RRM2, SRC, RRM2B, IISP90AA1, STR2, FLT1, SSTR5, YES1, BRCA1, RRM1, DIIFR, KDR, EPIIA2, RXRG, or LCK can be determined and used to identify a therapeutic for an individual. The expression level of the biomarker can be used to form a biomarker pattern or biomarker signature set. Determining the expression level can be by analyzing the levels of mRNA or protein, such as by microarray analysis or IHC. In some embodiments, the expression level of a biomarker is performed by IHC, such as for SPARC, TOP2A, or PTEN, and used to identify a therapeutic for an individual. The results of the IHC can be used to form a biomarker pattern or biomarker signature set. In yet other embodiments, a biological sample from an individual or subject is analyzed for the expression level of CD52, such as by determining the mRNA expression level by methods including, but not limited to, microarray analysis. The expression level of CD52 can be used to identify a therapeutic for the individual. The expression level of CD52 can be used to form a biomarker pattern or biomarker signature set. In still other embodiments, the biomarkers hENT1, cMet, P21, PARP-1, TLE3 and/or IGF1R are assessed to identify a therapeutic for the individual.

As described herein, the molecular profiling of one or more targets can be used to determine or identify a therapeutic for an individual. For example, the expression level of one or more biomarkers can be used to determine or identify a therapeutic for an individual. The one or more biomarkers, such as those disclosed herein, can be used to form a biomarker pattern or biomarker signature set, which is used to identify a therapeutic for an individual. In some embodiments, the therapeutic identified is one that the individual has not previously been treated with. For example, a reference biomarker pattern has been established for a particular therapeutic, such that individuals with the reference biomarker pattern will be responsive to that therapeutic. An individual with a biomarker pattern that differs from the reference, for example the expression of a gene in the biomarker pattern is changed or different from that of the reference, would not be administered that therapeutic. In another example, an individual exhibiting a biomarker pattern that is the same or substantially the same as the reference is advised to be treated with that therapeutic. In some embodiments, the individual has not previously been treated with that therapeutic and thus a new therapeutic has been identified for the individual.

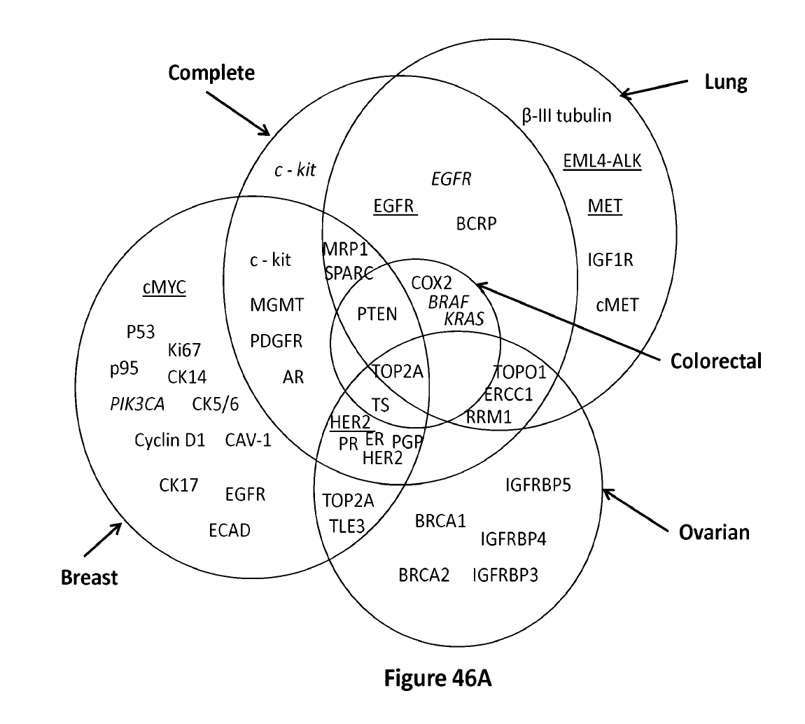

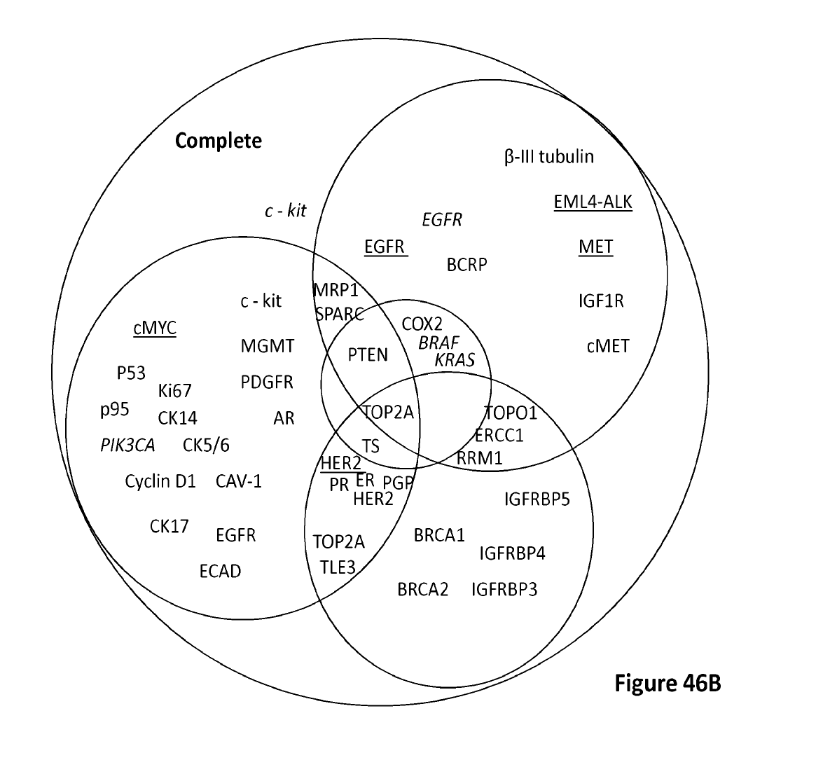

Molecular profiling according to the invention can take on a biomarker-centric or a therapeutic-centric point of view. Although the approaches are not mutually exclusive, the biomarker-centric approach focuses on sets of biomarkers that are expected to be informative for a tumor of a given tumor lineage, whereas the therapeutic-centric point approach identifies candidate therapeutics using biomarker panels that are lineage independent. In a biomarker-centric view, panels of specific biomarkers are run on different tumor types. See FIG. 46A. This approach provides a method of identifying a candidate therapeutic by collecting a sample from a subject with a cancer of known origin, and performing molecular profiling on the cancer for specific biomarkers depending on the origin of the cancer. The molecular profiling can be performed using any of the various techniques disclosed herein. As an example, FIG. 46A shows biomarker panels for breast cancer, ovarian cancer, colorectal cancer, lung cancer, and a “complete” profile to run on any cancer. In the figure, markers shown in italics are assessed using mutational analysis (e.g., sequencing approaches), marker shown underlined are analyzed by FISH, and the remainder are analyzed using IHC. DNA microarray profiling can be performed on any sample. The candidate therapeutic is selected based on the molecular profiling results according to the subject methods. An advantage to the bio-marker centric approach is only performing assays that are most likely to yield informative results. Another advantage is that this approach can focus on identifying therapeutics conventionally used to treat cancers of the specific lineage. In a therapeutic-centric approach, the biomarkers assessed are not dependent on the origin of the tumor. See FIG. 46B. This approach provides a method of identifying a candidate therapeutic by collecting a sample from a subject with a cancer, and performing molecular profiling on the cancer for a panel of biomarkers without regards to the origin of the cancer. The molecular profiling can be performed using any of the various techniques disclosed herein. As an example, in FIG. 46B, markers shown in italics are assessed using mutational analysis (e.g., sequencing approaches), marker shown underlined are analyzed by FISH, and the remainder are analyzed using IHC. DNA microarray profiling can be performed on any sample. The candidate therapeutic is selected based on the molecular profiling results according to the subject methods. An advantage to the therapeutic-marker centric approach is that the most promising therapeutics are identified only taking into account the molecular characteristics of the tumor itself. Another advantage is that the method can be preferred for a cancer of unidentified primary origin (CUP). In some embodiments, a hybrid of biomarker-centric and therapeutic-centric points of view is used to identify a candidate therapeutic. This method comprises identifying a candidate therapeutic by collecting a sample from a subject with a cancer of known origin, and performing molecular profiling on the cancer for a comprehensive panel of biomarkers, wherein a portion of the markers assessed depend on the origin of the cancer. For example, consider a breast cancer. A comprehensive biomarker panel is run on the breast cancer, e.g., the complete panel as shown in FIG. 46B, but additional sequencing analysis is performed on one or more additional markers, e.g., BRCA1 or any other marker with mutations informative for theranosis or prognosis of the breast cancer. Theranosis can be used to refer to the likely efficacy of a therapeutic treatment. Prognosis refers to the likely outcome of an illness. One of skill will appreciate that the hybrid approach can be used to identify a candidate therapeutic for any cancer having additional biomarkers that provide theranostic or prognostic information, including the cancers disclosed herein.

Methods for providing a theranosis of disease include selecting candidate therapeutics for various cancers by assessing a sample from a subject in need thereof (i.e., suffering from a particular cancer). The sample is assessed by performing an immunohistochemistry (IHC) to determine of the presence or level of: AR, BCRP, c-KIT, ER, ERCC1, IIER2, IGF1R, MET (also referred to herein as cMet), MGMT, MRP1, PDGFR, PGP, PR, PTEN, RRM1, SPARC, TOPO1, TOP2A, TS, COX-2, CK5/6, CK14, CK17, Ki67, p53, CAV-1, CYCLIN D1, EGFR, E-cadherin, p95, TLE3 or a combination thereof; performing a microarray analysis on the sample to determine a microarray expression profile on one or more (such as at least five, 10, 15, 20, 25, 30, 40, 50, 60, 70 or all) of: ABCC1, ABCG2, ADA, AR, ASNS, BCL2, BIRC5, BRCA1, BRCA2, CD33, CD52, CDA, CES2, DCK, DIIFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IL2RA, HSP90AA1, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; comparing the results obtained from the IHC and microarray analysis against a rules database, wherein the rules database comprises a mapping of candidate treatments whose biological activity is known against a cancer cell that expresses one or more proteins included in the IHC expression profile and/or expresses one or more genes included in the microarray expression profile; and determining a candidate treatment if the comparison indicates that the candidate treatment has biological activity against the cancer.

Assessment can further comprise determining a fluorescent in-situ hybridization (FISII) profile of EGFR, HER2, cMYC, TOP2A, MET, or a combination thereof, comparing the FISH profile against a rules database comprising a mapping of candidate treatments predetermined as effective against a cancer cell having a mutation profile for EGFR, HER2, cMYC, TOP2A, MET, or a combination thereof, and determining a candidate treatment if the comparison of the FISH profile against the rules database indicates that the candidate treatment has biological activity against the cancer.

As explained further herein, the FISH analysis can be performed based on the origin of the sample. This can avoid unnecessary laboratory procedures and concomitant expenses by targeting analysis of genes that are known to play a role in a particular disorder, e.g., a particular type of cancer. In an embodiment, EGFR, HER2, cMYC, and TOP2A are assessed for breast cancer. In another embodiment, EGFR and MET are assessed for lung cancer. Alternately, FISH analysis of all of EGFR, HER2, cMYC, TOP2A, MET can be performed on a sample. The complete panel may be assessed, e.g., when a sample is of unknown or mixed origin, to provide a comprehensive view of an unusual sample, or when economies of scale dictate that it is more efficient to perform FISH on the entire panel than to make individual assessments.

In an additional embodiment, the sample is assessed by performing nucleic acid sequencing on the sample to determine a presence of a mutation of KRAS, BRAF, PIK3CA (also referred to as PI3K), c-Kit, EGFR, or a combination thereof, comparing the results obtained from the sequencing against a rules database comprising a mapping of candidate treatments predetermined as effective against a cancer cell having a mutation profile for KRAS, BRAF, PIK3CA, c-Kit, EGFR, or a combination thereof; and determining a candidate treatment if the comparison of the sequencing to the mutation profile indicates that the candidate treatment has biological activity against the cancer.

As explained further herein, the nucleic acid sequencing can be performed based on the origin of the sample. This can avoid unnecessary laboratory procedures and concomitant expenses by targeting analysis of genes that are known to play a role in a particular disorder, e.g., a particular type of cancer. In an embodiment, the sequences of PIK3CA and c-KIT are assessed for breast cancer. In another embodiment, the sequences of KRAS and BRAF are assessed for GI cancers such as colorectal cancer. In still another embodiment, the sequences of KRAS, BRAF and EGFR are assessed for lung cancer. Alternately, sequencing of all of KRAS, BRAF, PIK3CA, c-Kit, EGFR can be performed on a sample. The complete panel may be sequenced, e.g., when a sample is of unknown or mixed origin, to provide a comprehensive view of an unusual sample, or when economies of scale dictate that it is more efficient to sequence the entire panel than to make individual assessments.

Pancreatic Cancer

For all stages of pancreatic cancer combined, the 1- and 5-year relative survival rates are 24% and 5% respectively. Even for those diagnosed with local disease, the 5-year survival rate is only 20%. (American Cancer Society. (2009). Cancer Facts & Figures. 2009. Atlanta: American Cancer Society. p. 19.) Target Now is a test that helps determine the status of a subject's molecular profile relevant to pancreatic cancer and delivers a single evidence-based report with individualized therapeutic guidance. Because so many pancreatic cancer patients get just one chance for chemotherapy, molecular profiling can provide the information needed to make an appropriate first choice.

Molecular profiling can be used to make informed treatment decisions for pancreatic cancer patients, including without limitation those who are eligible for systemic treatment, or have progressed on prior therapy.

Therapeutic agents that can be associated with clinical benefit or lack of clinical benefit based on biomarker status include Anti-Neoplastic Agent (gemcitabine), Platinum Analogues (cisplatin, oxaliplatin), Protein Kinase Inhibitor (erlotinib), Pyrimidine Analogues (5-fluorouracil, capecitabine), Taxane (nab-paclitaxel).

For a sample from a subject suffering from pancreatic cancer, IHC profiling can be conducted to determine the presence or level of one or more: AR, BCRP, c-KIT, ER, ERCC1, HER2, MGMT, MRP1, PDGFR, PGP, PR, PTEN, RRM1, SPARC, TOPO1, TOP2A, and TS. In some embodiments, IHC is conducted on all of these biomarkers. The IHC analysis can be combined with microarray analysis, as described further herein. The analysis can further comprise assessing EGFR, HER2 or both by FISH and/or nucleic acid sequencing of KRAS, BRAF, or both. In another embodiment, molecular profiling performed on a sample from a subject with pancreatic cancer includes the tests listed in Table 6. Based on results for one or more of the foregoing (i.e., IHC, FISH, sequencing, microarray), a treatment or therapy is selected. Based on the analysis, a likelihood of clinical benefit or lack of clinical benefit of a particular candidate treatment is determined. Illustrative treatments include without limitation an anti-neoplastic, platinum analog, protein kinase inhibitor, pyrimidine analog, or a taxane, or any combination thereof, such as gemcitabine, cisplatin, oxaliplatin, erlotinib, 5-fluorouracil, capecitabine, or nab-paclitaxel. In some embodiments, the subject assessed with pancreatic cancer is eligible for systemic treatment or has been subjected to prior therapy.

TABLE 6
Molecular Profiling for Pancreatic
Cancer: BiomarkersAssessed
IHC

| | |
|---|---|
| AR | PGP |
| BCRP | PR |
| c-KIT | PTEN |
| ER | RRM1 |
| ERCC1 | SPARC |
| HER2 | Mono |
| MGMT | SPARC Poly |
| MRP1 | TOPO1 |
| PDGFR | TOP2A |
| | TS |

FISH
EGFR (if appropriate)
HER2 (if appropriate)
Mutation Analysis
BRAF (if appropriate)
KRAS (if appropriate)
DNA Microarray
Whole genome expression array

Lung Cancer

The 1-year relative survival for lung cancer is 41%. The 5-year survival rate for all stages combined is only 15%. The 5-year survival rate is 50% for cases detected when the disease is localized, but only 16% of lung cancers are diagnosed at this early stage. Lung cancer patients often present with advanced disease, which is a major treatment challenge. Their performance status precludes using many toxic chemotherapies making initial treatment selection critical. (American Cancer Society. (2009). Cancer Facts & FIGS. 2009. Atlanta: American Cancer Society. p. 15.)

Molecular profiling results can be used to make informed treatment decisions for lung cancer patients, including without limitation those who have non-small cell lung cancer (NSCLC) with stage IV metastatic disease who have progressed through platinum combination regimens and now require select second-line therapies (and beyond), or want to guide first-line therapy for NSCLC wet stage IIIb and Stage IV disease, or have small cell lung cancer (SCLC) and have failed first line therapy, or have mesothelioma and have failed first line therapy.

Therapeutic agents that can be associated with clinical benefit or lack of clinical benefit based on biomarker status include Taxanes (paclitaxel, docetaxel, nab-paclitaxel), Vinca Alkyloids (vinblastine, vinorelbine), Anti-Neoplastic Agents (gemcitabine, mitomycin), Podophyllotoxin Derivative (etoposide), Anti-Vascular Agent (bevacizumab), Platinum Analogues (carboplatin, cisplatin), Podophyllotoxin Derivative (etoposide).

For a sample from a subject suffering from lung cancer, IHC profiling can be conducted to determine the presence or level of one or more of: AR, BCRP, c-KIT, ER, ERCC1, IGF1R, HER2, MET, MGMT, MRP1, PDGFR, PGP, PR, PTEN, RRM1, SPARC, TOPO1, TOP2A, and TS. In some embodiments, IHC is conducted on all of these biomarkers. The IHC analysis can be combined with microarray analysis. In some embodiments, the analysis further comprises nucleic acid sequencing of EGFR. The analysis can further comprise assessing one or more of EGFR, HER2 and MET by FISH and/or nucleic acid sequencing of one or more of KRAS, BRAF, and EGFR. In some embodiments, EGFR and MET are analyzed by FISH. In some embodiments, KRAS, BRAF, and EGFR are analyzed by nucleic acid sequencing. In some embodiments, molecular profiling of a lung cancer is performed to determine the presence, level or mutation in one or more of EML4-ALK, C-MET, Beta III tubulins, EGFR mutation (e.g., by FISH), PTEN, KRAS, BRAF, ERCC1, MRP1, BCRP, PGP, RRM1, TOP2A, TOPO1, and COX2. In another embodiment, molecular profiling performed on a sample from a subject with lung cancer includes the tests listed in Table 7. Based on results for one or more of the foregoing (i.e., MC, FISII, sequencing, microarray), a candidate treatment or therapy is selected. Based on the analysis, a likelihood of clinical benefit or lack of clinical benefit of a particular candidate treatment is determined. Illustrative treatments include without limitation a taxane, a vinca alkyloid, anti-neoplastic agent, podophyllotoxin derivative, anti-vascular agent, platinum analog, protein kinase inhibitor, folic acid analog, topoisomerase inhibitor, monoclonal antibody, or a or any combination thereof, such as paclitaxel, docetaxel, nab-paclitaxel, vinblastine, vinorelbine, gemcitabine, mitomycin, etoposide, bevacizumab, carboplatin, cisplatin, erlotinib, gefitinib, anthracycline, doxorubicin, pemetrexed, topotecan, irinotecan, or cetuximab. The subject may have non-small cell lung cancer (NSCLC), small cell lung cancer (SCLC), or mesothelioma. In another embodiment, the subject has NSCLC with stage IV metastatic disease and has progressed through platinum combination regimens. In yet another embodiment, the subject has NSCLC wet Stage Mb and Stage IV disease. In one embodiment, the subject has failed first line therapy.

TABLE 7
Molecular Profiling for Lung Cancer:
Biomarkers Assessed
IHC

| | |
|---|---|
| AR | PDGFR |
| BCRP | PGP |
| c-KIT | PR |
| ER | PTEN |
| ERCC1 | RRM1 |
| HER2 | SPARC Mono |
| IGF1R | SPARC Poly |
| MET | TOPO1 |
| MGMT | TOP2A |
| MRP1 | TS |

FISH
EGFR (if appropriate)
HER2 (if appropriate)
MET (if appropriate)

| | |
|---|---|
| Mutation Analysis | Mutation Analysis |
| EGFR | BRAF (if |
| | appropriate) |
| | KRAS (if |
| | appropriate) |

DNA Microarray
Whole genome expression array

Colorectal Cancer

Colorectal cancer is the second leading cause of cancer death in the United States. The 1- and 5-year relative survival for persons with colorectal cancer is 83% and 64%, respectively. The 5-year survival rate drops to 68% after cancer has spread to involve adjacent organs and lymph nodes. For persons with distant metastases, 5-year survival is 11%. The NCCN guidelines state that patients who are KRAS and BRAF mutated are not likely to respond to EGFR-inhibiting therapies and should receive alternative treatment. (American Cancer Society. (2009). Cancer Facts & FIGS. 2009. Atlanta: American Cancer Society. p. 12-13.)

Molecular profiling can be used to make informed treatment decisions for colorectal cancer patients, including without limitation those who have been treated for metastatic disease and have progressed, or have disease that is refractory to standard of care and for whom no clear treatment options exist.

Therapeutic agents that can be associated with clinical benefit or lack of clinical benefit based on biomarker status include Anti-Vascular Agent (bevacizumab), Monoclonal Antibodies (cetuximab, panitumumab), Platinum Analogue (oxaliplatin), Pyrimidine Analogues (5-fluorouracil, capecitabine), Topoisomerase Inhibitor (irinotecan).

For a sample from a subject suffering from colon cancer or colorectal cancer, IHC profiling can be conducted to determine the presence or level of one or more of: COX-2, PTEN, TOP1, TOP2A and TS. In some embodiments, IHC is conducted on all of these biomarkers. The IHC analysis can be combined with microarray analysis and/or nucleic acid sequencing of KRAS, BRAF, or both. The subject may colorectal colon cancer that is non-metastatic or treatment-naive metastatic. Alternately, the subject has colorectal cancer that is metastatic or the subject has failed prior therapy. IHC can be performed on additional biomarkers, such as one or more of: AR, BCRP, c-KIT, ER, ERCC1, HER2, MGMT, MRP1, PDGFR, PGP, PR, RRM1, and SPARC. In some embodiments, IHC is conducted on all of these additional biomarkers. The analysis can further comprise assessing HER2 by FISH. In another embodiment, molecular profiling performed on a sample from a subject with colorectal cancer includes the tests listed in Table 8. Based on results for one or more of the foregoing (i.e., IHC, FISH, sequencing, microarray), a treatment or therapy is selected. Based on the analysis, a likelihood of clinical benefit or lack of clinical benefit of a particular candidate treatment is determined. Illustrative treatments include without limitation an anti-vascular agent, monoclonal antibody, platinum analog, pyrimidine analog, topoisomerase inhibitor, or any combination thereof, such as bevacizumab, cetuximab, panitumumab, oxaliplatin, 5-fluorouracil, capecitabine, or irinotecan. The subject can be a subject that has been treated for metastatic colorectal cancer that has progressed, can be currently treated for metastatic colorectal cancer that has progressed, and/or has disease that is refractory to a standard of care. In another embodiment, the subject has no clear treatment options.

TABLE 8
Molecular Profiling for Colorectal Cancer: Biomarkers Assessed

| Non-metastatic or treatment- | | | | |
|---|---|---|---|---|
| naïve metastatic | Metastatic and failed prior therapy | | | |
| IHC | IHC | | | |
| COX-2 | AR | ERCC1 | PGP | SPARC |
| PTEN | BCRP | HER2 | PR | Mono |
| TOPO1 | c-KIT | MGMT | PTEN | SPARC |
| TS | COX-2 | MRP1 | RRM1 | Poly |
| | ER | PDGFR | | TOPO1 |
| | | | | TOP2A |
| | | | | TS |

| FISH | FISH |
|---|---|
| NA | HER2 (if appropriate) |
| Mutation Analysis | Mutation Analysis |
| BRAF | BRAF |
| KRAS | KRAS |
| DNA Microarray | DNA Microarray |
| Whole genome | Whole genome |
| expression array | expression array |

Ovarian Cancer

The 1- and 5-year relative survival of ovarian cancer patients is 75% and 46%, respectively. 5-year survival rates are 71% and 31%, for women with regional and distant disease, respectively. The 10-year relative survival rate for all stages combined is 39%. (American Cancer Society. (2009). Cancer Facts & Figures. 2009. Atlanta: American Cancer Society. p. 17-18.) Because most of these patients have recurrent disease at some point, a proactive plan for deciding treatment options based on the patient's tumor biology is an important aspect of care. ˜10% 10 year survival for patients who present with advanced stage III or IV disease. Molecular profiling can be used to make informed treatment decisions for ovarian cancer patients, including without limitation those who have metastatic disease, have progressed on platinum therapy, or have recurrent disease and have failed third line therapy.

For a sample from a subject suffering from ovarian cancer, IHC profiling can be conducted to determine the presence or level of one or more of: AR, BCRP, c-KIT, ER, ERCC1, HER2, MGMT, MRP1, PDGFR, PGP, PR, PTEN, RRM1, SPARC, TOPO1, TOP2A, and TS. In some embodiments, IHC is conducted on all of these biomarkers. In some embodiments, MC profiling for ovarian cancer is conducted to determine the presence or level of one or more of: PGP, ER, TOPO1, TOP2A, ERCC1, TS, ER, PR, RRM1, BRCA1, BRCA2, PI3KCA, IGFRBP3, IGFRBP4, IGFRBP5, HER-2 and TLE3. In some embodiments, IHC is conducted on all of these biomarkers. The IHC analysis can be combined with microarray analysis. The analysis can further comprise assessing EGFR, HER2, or both by FISH and/or nucleic acid sequencing of KRAS, BRAF, or both. Based on results for one or more of the foregoing (i.e., IHC, FISH, sequencing, microarray), a treatment or therapy is selected. Based on the analysis, a likelihood of clinical benefit or lack of clinical benefit of a particular candidate treatment is determined. Illustrative treatments include without limitation an anti-neoplastic, topoisomerase inhibitor, anthracycline, pyrimidine analog, vinca alkaloid, podophyllotoxin derivative, taxane, anti-vascular agent, platinum analog, anti-estrogen therapy, aromatase inhibitor, folic acid analog, selective estrogen receptor modulator, gonadotropin releasing hormone analog or any combination thereof, such as topotecan, irinotecan, gemcitabine, liposomal doxorubicin, capecitabine, vinblastine, vinorelbine, vincristine, etoposide, paclitaxel, docetaxel, bevacizumab, carboplatin, cisplatin, oxaliplatin, tamoxifen, fulvestrant, anastrozole, letrozole, megestrol, pemetrexed, tamoxifen, or leuprolide. In one embodiment, the subject has metastatic ovarian cancer, has progressed on platinum therapy, or has recurrent disease and has failed third line therapy.

In some embodiments, IHC profiling for a sample from a subject suffering from ovarian cancer is conducted to determine the presence or level of one or more of: ER, HER2, Ki67, p53, PGP, PR, and TS. In some embodiments, IHC is conducted on all of these biomarkers. The IHC analysis can be combined with microarray analysis and/or assessing HER2 by fluorescent in-situ hybridization (FISH). In another embodiment, molecular profiling performed on a sample from a subject with ovarian cancer includes the tests listed in Table 9.

TABLE 9
Molecular Profiling for Ovarian
Cancer: Biomarkers Assessed
IHC

| | |
|---|---|
| AR | MRP1 |
| BCRA1 | PDGFR |
| BRCA2 | PI3KCA |
| BCRP | PGP |
| c-KIT | PR |
| ER | PTEN |
| ERCC1 | RRM1 |
| HER2 | SPARC Mono |
| IGFRBP3 | SPARC Poly |
| IGFRBP4 | TLE3 |
| IGFRBP5 | TOPO1 |
| MGMT | TOP2A |
| | TS |

FISH
EGFR (if appropriate)
HER2 (if appropriate)
Mutation Analysis
BRAF (if appropriate)
KRAS (if appropriate)
DNA Microarray
Whole genome expression array

Therapeutic agents that can be associated with clinical benefit or lack of clinical benefit based on biomarker status include without limitation Topoisomerase Inhibitors (topotecan, irinotecan), Anti-Neoplastic Agent (gemcitabine), Anthracycline (liposomal doxorubicin), Prymidine Analog (capecitabine), Vinca Alkaloids (vinblastine, vinorelbine, vincristine), Podophyllotoxin Derivative (etoposide), Taxanes (paclitaxel, docetaxel), Anti-Vascular Agent (bevacizumab), Platinum Analogues (carboplatin, cisplatin, oxaliplatin), Anti-Estrogen Therapy (tamoxifen, fulvestrant), Aromatase Inhibitors (anastrozole, letrozole, megestrol), Folic Acid Analogue (pemetrexed), Selective Estrogen Receptor Modulator (tamoxifen), Gonadotropin Releasing Hormone Analogue (leuprolide).

Breast Cancer

Breast cancer is the second most common type of cancer after lung cancer, and the fifth most common cause of cancer deaths. Although breast cancer is 100-fold more prevalent in women, both sexes can be afflicted with the disease. Breast cancer usually starts in the breast, e.g., in the inner lining of the milk ducts or lobules. Various types of breast cancer are characterized by stage, aggressiveness and genetic events. Treatments include surgery (e.g., mastectomy), drugs (hormone therapy and chemotherapy, and radiation. 10 year survival ranges from 10 to 98%. Non-invasive (or “in situ”) breast cancers are confined to ducts or lobules but can become invasive. Ductal carcinoma in situ (DCIS) is the most common type of non-invasive breast cancer. Invasive (or infiltrating) cancers have started to break through normal breast tissue barriers and invade surrounding areas. Invasive cancers can be very serious.

Some breast cancers require the hormones estrogen and progesterone to proliferate and express receptors for those hormones, e.g., the estrogen receptor (ER) and progesterone receptor (PR). Such cancers can be treated with therapeutic agents that inhibit this process, e.g., tamoxifen, an antagonist of the estrogen receptor in breast tissue, and aromatase inhibitors, which block the synthesis of estrogen. Interfering with estrogen synthesis can damage the ovaries and lead to infertility. Breast cancers without hormone receptors, those that spread to the lymph nodes, or have other risk factors, may be treated more aggressively. “CA” therapy comprises a cocktail of cyclophosphamide and doxorubicin (Adriamycin®), which damage DNA. “CAT” therapy further includes a taxane drug, such as docetaxel, which attacks microtubules. ‘CMF” therapy comprises cyclophosphamide, methotrexate, and fluorouracil. All of these chemotherapeutic agents can cause serious side effects by affecting normal cells. The HER2 gene (also known as HER2/ncu and ErbB2 gene) is amplified in 20-30% of early-stage breast cancers. Trastuzumab (Herceptin™) is a monoclonal antibody that interferes with the HER2/neu receptor, thereby inhibiting cancer cell growth. Breast cancers that don't overexpress HER2 don't receive benefit from such treatment. Trastuzumab can be highly effective, but 70% of HER2 positive tumors don't respond to treatment and others may eventually develop resistance. Trastuzumab can also cause heart damage. Radiation therapy can be used but also causes heart problems. The methods of the invention can be used to identify treatment regimens including the above standard drugs and non-standard drugs for treatment of breast cancer.

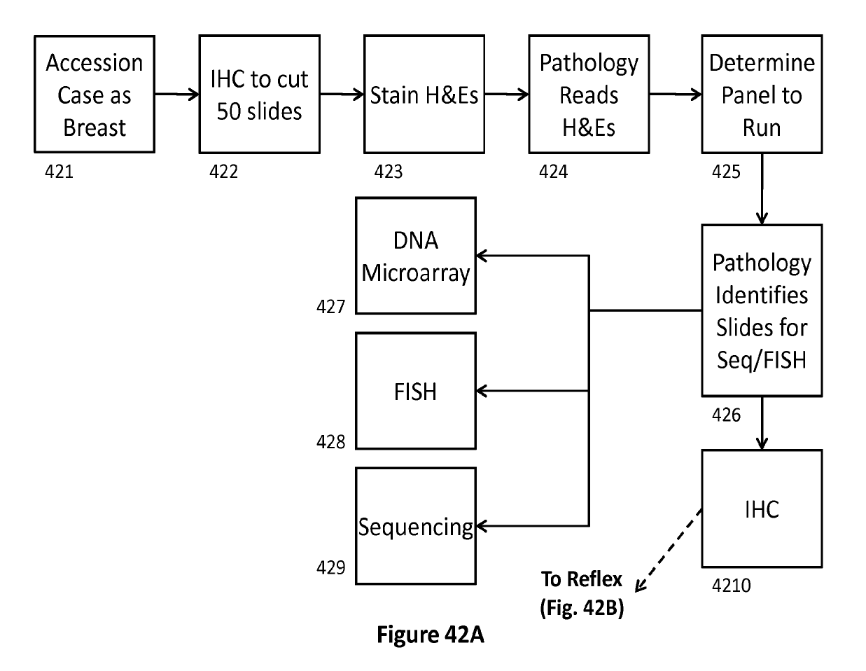

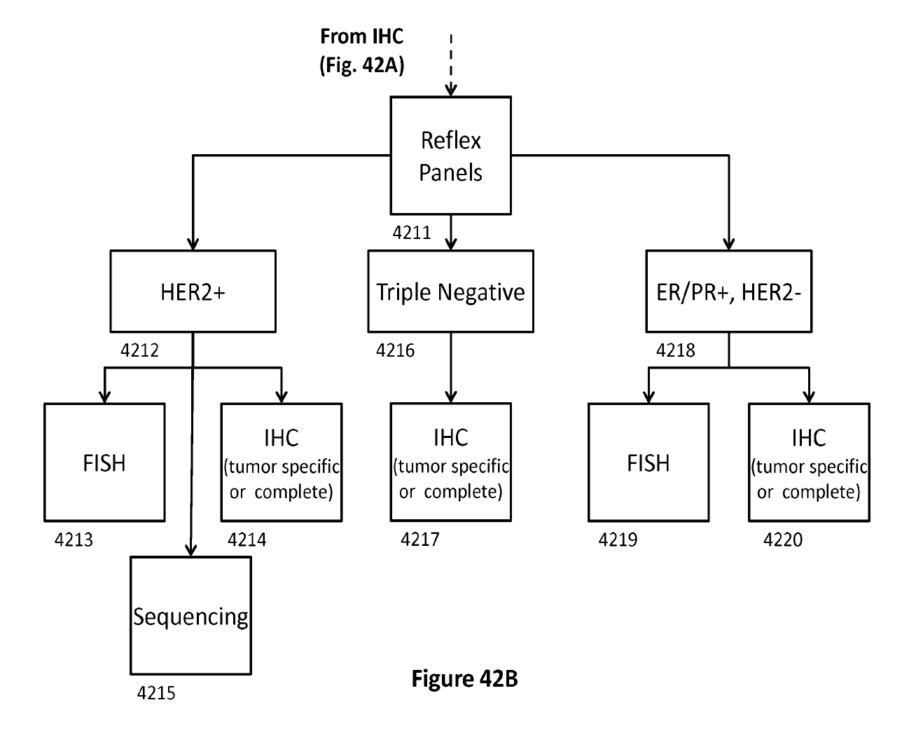

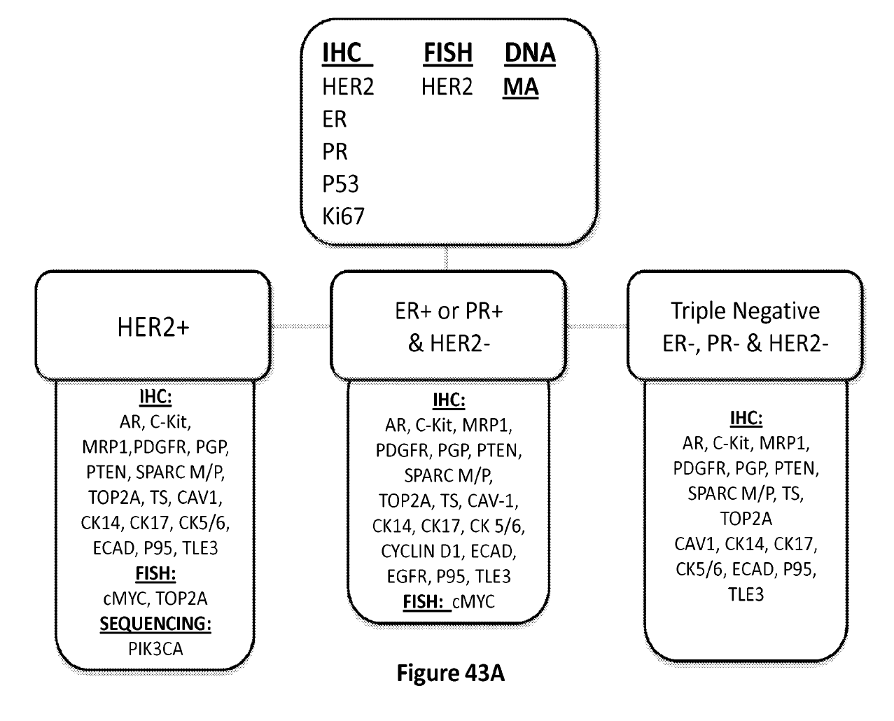

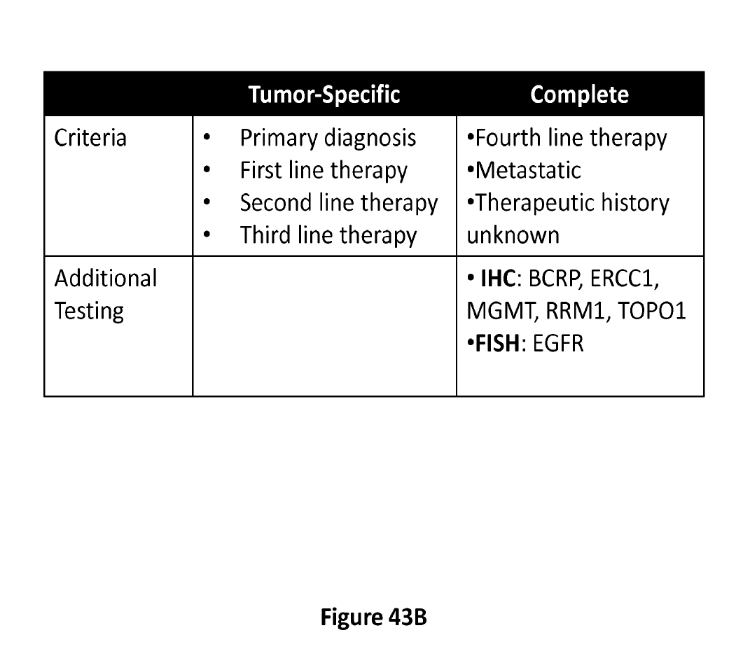

The subject methods can be used to identify a candidate treatment for a subject suffering from breast cancer comprising a triple-receptor negative breast cancer. Triple negative breast cancer includes breast cancer that expresses little to no ER or PR, and does not exhibit overexpression and/or gene amplification of HER2/neu. See, e.g., Dawood S, Broglio K, Esteva F J, Yang W, Kau S W, Islam R, Albarracin C, Yu T K, Green M, Hortobagyi G N, Gonzalez-Angulo A M. Survival among women with triple receptor-negative breast cancer and brain metastases. Ann Oncol. 2009 April; 20(4):621-7. Epub 2009 Jan. 15. Illustrative diagrams for identifying candidate treatments according to the invention are shown in FIGS. 42 and 43. FIG. 42 shows a flow diagram and FIG. 43 shows biomarkers that can be assessed. The subject may have metastatic breast cancer and completed a first, second, or third line of therapy. IHC profiling can be conducted on a sample from the subject to determine the presence or level of one or more of: AR, CK5/6, CK14, CK17, ER, HER2, Ki67, MRP1, P53, PGP, PR, SPARC and TS. In some embodiments, IHC is conducted on all of these biomarkers. The IHC analysis can be combined with microarray analysis and/or assessment of HER2 by fluorescent in-situ hybridization (FISH). The IHC analysis can determine the presence or level of additional biomarkers, such as one or more of: BCRP, c-KIT, ERCC1, MGMT, PDGFR, PTEN, RRM1, and TOP2A. In some embodiments, IHC is conducted on all of these additional biomarkers. The subject may have completed a fourth line of therapy or beyond. Based on results for one or more of the foregoing (i.e., IHC, FISH, sequencing, microarray), a treatment or therapy is selected. Based on the analysis, a likelihood of clinical benefit or lack of clinical benefit of a particular candidate treatment is determined. Illustrative treatments include without limitation an anthracycline, taxane, platinum analog, anti-neoplastic agent, camptothecin, pyrimidine analog, vinca alkaloid, gonatropin releasing hormone analog, anti-androgen, or any combination thereof, such as doxorubicin, liposomal doxorubicin, epirubicin, paclitaxel, docetaxel, nab-paclitaxel, carboplatin, cisplatin, gemcitabine, irinotecan, capecitabine, 5-fluorouracil, vinblastine, vinorelbine, goserelin, leuprolide, bicalutamide, or flutamide.

The subject methods can be used to identify a candidate treatment for a subject suffering from breast cancer that is hormone-receptor-positive and HER2 negative (ER+ and/or PR+, and HER2−). Illustrative diagrams for identifying candidate treatments according to the invention are shown in FIGS. 42 and 43. FIG. 42 shows a flow diagram and FIG. 43 shows biomarkers that can be assessed. The subject's HER2 status may have changed. The subject may have metastatic breast cancer and completed a first, second, or third line of therapy. MC profiling can be conducted on a sample from the subject to determine the presence or level of one or more of: CAV-1, c-KIT, CYCLIN D1, EGFR, ER, HER2, Ki67, p53, PR, PDGFR, PGP, PTEN and TS. In some embodiments, IHC is conducted on all of these biomarkers. The IHC analysis can be combined with microarray analysis and/or assessment of HER2, cMYC, or both, by fluorescent in-situ hybridization (FISH). The MC analysis can determine the presence or level of additional biomarkers, such as one or more of: AR, ERCC1, MGMT, MRP1, RRM1, SPARC, TOP1, and TOP2A. In some embodiments, IHC is conducted on all of these additional biomarkers. The subject may have completed a fourth line of therapy or beyond. Based on results for one or more of the foregoing (i.e., IHC, FISH, sequencing, microarray), a treatment or therapy is selected. Based on the analysis, a likelihood of clinical benefit or lack of clinical benefit of a particular candidate treatment is determined. Illustrative treatments include without limitation a monoclonal antibody, protein kinase inhibitor, anthracycline, taxane, platinum analog, anti-neoplastic agent, camptothecin, anti-estrogen therapy, armatase inhibitor, pyrimidine analogue, vinca alkaloid, gonatropin releasing hormone analogue, anti-androgen, folic acid analog, selective estrogen receptor modulator, or any combination thereof, such as trastuzumab, lapatinib, doxorubicin, liposomal doxorubicin, epirubicin, paclitaxel, docetaxel, nab-paclitaxel, carboplatin, cisplatin, gemcitabine, irinotecan, fulvestrant, anastrozole, exemestane, letrozole, capecitabine, 5-fluorouracil, vinblastine, vinorelbine, leuprolide, bicalutamide, flutamide, goserelin, methotrexate, tamoxifen, or toremifene.

The subject methods can be used to identify a candidate treatment for a subject suffering from breast cancer that is HER2 positive (HER2+). Illustrative diagrams for identifying candidate treatments according to the invention are shown in FIGS. 42 and 43. FIG. 42 shows a flow diagram and FIG. 43 shows biomarkers that can be assessed. The subject's HER2 status may have changed or has progressed on trastuzumab. The subject may have metastatic breast cancer and completed a first, second, or third line of therapy. IHC profiling can be conducted on a sample from the subject to determine the presence or level of one or more of: E-cadherin, ER, HER2, Ki67, MRP1, p53, p95, PGP, PR, PTEN, TLE3 and TS. In some embodiments, IHC is conducted on all of these biomarkers. The IHC analysis can be combined with microarray analysis, fluorescent in-situ hybridization (FISH) assessment of HER2, cMYC, TOP2A, or a combination, and sequencing of PIK3CA. The IHC analysis can determine the presence or level of additional biomarkers, such as one or more of: AR, BCRP, c-KIT, ERCC1, MGMT, PDGFR, RRM1, SPARC, TOP1, and TOP2A. In some embodiments, IHC is conducted on all of these additional biomarkers. In some embodiments, the subject has completed a fourth line of therapy or beyond. Based on results for one or more of the foregoing (i.e., IHC, FISH, sequencing, microarray), a treatment or therapy is selected. Based on the analysis, a likelihood of clinical benefit or lack of clinical benefit of a particular candidate treatment is determined. Illustrative treatments include without limitation a monoclonal antibody, protein kinase inhibitor, anthracycline, taxane, platinum analog, anti-neoplastic agent, camptothecin, anti-estrogen therapy, armatase inhibitor, pyrimidine analogue, vinca alkaloid, gonatropin releasing hormone analogue, anti-androgen, folic acid analog, selective estrogen receptor modulator, or any combination thereof, such as trastuzumab, lapatinib, doxorubicin, liposomal doxorubicin, epirubicin, paclitaxel, docetaxel, nab-paclitaxel, carboplatin, cisplatin, gemcitabine, irinotecan, fulvestrant, anastrozole, exemestane, letrozole, capecitabine, 5-fluorouracil, vinblastine, vinorelbine, leuprolide, bicalutamide, flutamide, goserelin, methotrexate, tamoxifen, or toremifene.

In one aspect, the invention provides a method for identifying a therapeutic for an individual with breast cancer comprising: a) determining an expression level or a mutation of a gene from a biological sample of said individual, wherein said gene is selected from the group consisting of: ER, PR, HER2, Ki-67 and P53; and b) identifying a therapeutic for treating the individual based on a change in expression or a mutation as compared to a reference. The expression level or mutation can be determined by, e.g., IHC, FISH, microarray, sequencing, real-time PCR or other methods as disclosed herein. The results can be used to subtype the breast cancer, e.g., according to receptor status or drug resistance status. In some embodiments, the breast cancer comprises an Invasive Breast Cancer. In some embodiments, the breast cancer is IIer-2 positive. IIer-2 expression can be determined by FISH and/or IIIC. In some embodiments, the breast cancer comprises a triple negative breast cancer. The cancer can also be metastatic. In some embodiments, the breast cancer is negative for at least one of ER, PR, or Her2. In some embodiments, the breast cancer is negative for at least two of ER, PR, or Her2. In other embodiments, the breast cancer is negative for at least one of ER, PR, or Her-2, and positive for at least one of ER, PR, or Her2. In some embodiments, the breast cancer is negative for at least two of ER, PR, or IIer2, e.g. ER-negative, PR-negative, and Her-2 positive; or ER-positive, PR-negative, and Her2 negative; or ER-negative, PR-positive, and Her2 negative. In one embodiment, the breast cancer is an ER and/or PR+, HER2− breast cancer. The subtype of the breast cancer can be further used to identify or refine a therapeutic.

In one embodiment, the breast cancer is Her-2 positive. About 20-30% of breast cancers are HER2 positive. In HER2+ breast cancer, the cancer cells have an abnormally high number of HER2 genes per cell. When this happens, an abundance of HER2 protein appears on the surface of these cancer cells. Of these, about 30% respond to trastuzumab therapy. The response may be dependent on loss of PTEN, PI3 Kinase mutations, p95HER2 expression, and/or IGF-1R expression. p95HER2 refers to a truncated form of the HER2 receptor. In one embodiment, HER-2 status is determined by FISH and/or IHC. In some embodiments, the invention provides a method of determining a therapeutic treatment for an individual having HER-2 positive breast cancer comprising: a) determining an expression level of a gene and/or a mutation in a gene from a biological sample of said individual, wherein said gene is selected from the group consisting of: HER2, PTEN, PI-3 kinase, IGF-IR and p95HER2; and b) identifying a therapeutic based on said mutation or wherein said gene exhibits a change in expression as compared to a reference. Some of the individuals will respond to lapatinib or trastuzumab. In some embodiments, loss of PTEN, mutation in PI-3 Kinase, over expression of IGF-1R or over expression p95HER2 indicates decreased probability of response to trastuzumab and can favor treatment with lapatinib. In some embodiments, the panel for identifying a therapeutic for an individual having HER2 breast cancer comprises analysis of expression and/or mutation of HER2, PTEN, IGF-1R and p95HER2, PI-3 Kinase, or a combination thereof. In some embodiments, the panel comprises TOP2A, PGP, MRP1, TS, ERCC1, BCRP, RRM1, TOPOI, TOPOII, TLE3 (for taxanes), C-MYC, TOP2, P95, PTEN, E-Cad, HER2, PI3K or a combination thereof. For example, BCRP, ERCC1, MRP1, p95, PGP, RRM1, TLE3, TopoI, TopoII, TS, PTEN and E-cad can be assayed by IHC, HER2, cMYC and TOP2A can be assayed by FISH, and PI3K can be assayed by sequencing. The panels can be used to identify therapeutics for relapsed or refractive cancers.

In one embodiment, the breast cancer is a triple negative breast cancer. Triple negative breast cancer, which refers to cancers that are estrogen receptor (ER) negative, progesterone receptor (PR) negative, and human epidermal growth factor receptor 2 (Her-2) negative, comprise approximately 15% of all breast cancers and have an aggressive clinical course with high rates of local and systemic relapse. The clinical course reflects the biology of the tumor as well as the absence of conventional targets for treatment such as hormonal therapy for ER or PR positive patients and trastuzumab for Her-2 over-expressing tumors. Despite the availability of antimetabolites such as gemcitabine and platinum complex agents such as carboplatin, there is no accepted standard of care for ER negative breast cancer. In particular, triple negative metastatic breast cancer is refractory to standard treatments and is refractory to serum estrogen receptor modulator (SERM) chemotherapy.

DNA repair deficits can be a characteristic of triple negative cancers. Such cancers frequently harbor defects in DNA double-strand break repair through homologous recombination (HR), such as BRCA1 dysfunction (Rottenberg S, et.al. Proc Natl Acad Sci USA. 2008 Nov. 4; 105(44):17079-84). These tumors exhibit more DNA copy alterations and loss of heterozygosity than other breast cancers, features suggestive of genomic instability. Furthermore, sporadic triple negative tumors share phenotypic and cytogenetic features with familial BRCA1 associated cancer and correlate with BRCA1 cancers using microarray RNA expression data. BRCA1 mutant tumors are thought to be deficient in DNA repair, particularly homologous recombination, and these similarities may suggest that a similar DNA repair deficiency may play a role in triple negative tumors.

In some embodiments, the invention provides a method of determining a therapeutic treatment for an individual having a triple negative breast cancer breast cancer comprising: a) determining an expression level of a gene and/or a mutation in a gene from a biological sample of said individual, wherein said gene is selected from the group consisting of: AR, KRAS, BRCA1, PARP-1, SPARC, CK 5/6, CK14, CK17, TOP2A, PGP, MRP1, TS, ERCC1, BCRP, RRM1, TOPOI, TOPOII, TLE3; and b) identifying a therapeutic based on said mutation or wherein said gene exhibits a change in expression as compared to a reference. In some embodiments, AR, KRAS, BRCA1, PARP-1, SPARC, CK 5/6, CK14, CK17, TOP2A, PGP, MRP1, TS, ERCC1, BCRP, RRM1, TOPOI, TOPOII TLE3 are assayed using IHC. In some embodiments, KRAS is assayed by sequencing. The panel can be used to identifying therapeutics for relapsed or refractive cancers.

In some embodiments, the breast cancer comprises Ductal Carcinoma in Situ (DCIS). In one aspect, the invention provides a method for identifying a therapeutic for an individual with DCIS comprising: a) determining an expression level of a gene from a biological sample of said individual, wherein said gene is selected from the group consisting of: ER, PR HER2, Ki-67, P53, BCL2 and E-Cadherin; and b) identifying a therapeutic that said individual has not previously been treated for said condition, when said gene exhibits a change in expression as compared to a reference. The expression levels can be determined by, e.g., IHC, FISH, microarray, sequencing, real-time PCR or other methods as disclosed herein. A therapeutic can be chosen based on the expression of the gene or of a mutation thereof.

In an aspect, the invention provides a method for identifying a therapeutic for an individual having breast cancer comprising: (a) determining an expression level of a gene and/or a mutation in a gene from a biological sample of said individual, wherein said gene is selected from the group consisting of: SPARC, TOP2A, TOTO1, PGP, BCRP, MRP1, PTEN, TS, ERCC1, RRM1, MGMT, c-kit, PDGFR, AR, EGFR, KRAS, BRAF, p95 or PI3K; and (b) identifying a therapeutic for said individual when said gene exhibits a change in expression as compared to a reference. In some embodiments, the individual has refractive breast cancer or has relapsed. The cancer can be metastatic. The expression and/or the mutation can be determined using IIIC, FISII, microarray, sequencing, real-time PCR or other methods as disclosed herein.

In a related aspect, the invention provides a method of identifying a candidate treatment for a subject in need thereof by using molecular profiling of sets of known biomarkers. For example, the method can identify a chemotherapeutic agent for an individual with a cancer. The method comprises: obtaining a sample from the subject; performing an immunohistochemistry (IIIC) analysis on the sample to determine an IHC expression profile on one or more, e.g. 2, 3, 4, 5, 6, 7, 8, 9, 10 or more, of: AR, c-Kit, CAV-1, CK 5/6, CK14, CK17, ECAD, ER, Her2/Neu, Ki67, MRP1, P53, P95, PDGFR, PUP, PR, PTEN, SPARC (using a monoclonal and/or polyclonal antibody), TLE3, TOP2A and TS; performing a microarray analysis on the sample to determine a microarray expression profile on one or more, e.g. 2, 3, 4, 5, 6, 7, 8, 9, 10 or more, of: ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70; performing a fluorescent in-situ hybridization (FISH) analysis on the sample to determine a FISH mutation profile on at least HER2. If the cancer is a HER2+ breast cancer, the method further comprises: performing FISH analysis on the sample to determine a FISH mutation profile for cMYC and TOP2A; and performing DNA sequencing on the sample to determine a sequencing mutation profile on at least PI3K (PIK3CA). If the cancer is (ER+ or PR+) and HER2− breast cancer, the method further comprises: performing IHC analysis on the sample to determine an IIIC expression profile on one or more of Cyclin D1 and EGFR; and performing FISII analysis on the sample to determine a FISH mutation profile for cMYC. If the cancer comprises: 1) triple negative (i.e., ER−, PR− and HER2−) breast cancer, 2) HER2+ breast cancer, or 3) (ER+ or PR+) and HER2− breast cancer, and the cancer is fourth line, metastatic or beyond, or has the therapeutic history is not known, the method further comprises: performing IHC analysis on the sample to determine an IHC expression profile on one or more of BCRP, ERCC1, MGMT, RRM1 and TOPO1; and performing FISII analysis on the sample to determine a FISH mutation profile for EGFR. The molecular profiling according to the method is illustrated in FIGS. 42 and 43. Once the molecular profiling is performed, the method further comprises comparing the IHC expression profile, microarray expression profile, FISH mutation profile and sequencing mutation profile against a rules database, wherein the rules database comprises a mapping of treatments whose biological activity is known against diseased cells that: i) overexpress or underexpress one or more proteins included in the IHC expression profile; ii) overexpress or underexpress one or more genes included in the microarray expression profile; iii) have zero or more mutations in one or more genes included in the FISH mutation profile; and/or iv) have zero or more mutations in one or more genes included in the sequencing mutation profile; and identifying the treatment if the comparison against the rules database indicates that the treatment should have biological activity against the cancer; and the comparison against the rules database does not contraindicate the treatment for treating the cancer. In some embodiments, the IHC expression profiling is performed on at least 20%, 30%, 40%, 50%, 60%, 70%, 80%, 90%, or 95% of the gene products above. In some embodiments, the microarray expression profiling is performed on at least 20%, 30%, 40%, 50%, 60%, 70%, 80%, 90%, or 95% of the genes listed above. In some embodiments, IHC is performed on 100% of the gene products indicated above. The microarray expression profiling can also be performed on 100% of the genes indicated above. The molecular profiling steps can be performed in any order. In some embodiments, not all of the molecular profiling steps are performed. As a non-limiting example, microarray analysis is not performed if the sample quality does not meet a threshold value, as described herein. In some embodiments, the biological material is mRNA and the quality control test comprises a A260/A280 ratio and/or a Ct value of RT-PCR using a housekeeping gene, e.g., RPL13a. In embodiments, the mRNA does not pass the quality control test if the A260/A280 ratio <1.5 or the RPL13a Ct value is >30. In that case, microarray analysis may not be performed. Alternately, microarray results may be attenuated, e.g., given a lower priority as compared to the results of other molecular profiling techniques.

Prognostics

In another aspect, the invention provides a method of providing a prognosis for a cancer. The method comprises performing molecular profiling on the sample as described herein and providing a prognosis based on the molecular profiling results. Accordingly, molecular profiling can be used to simultaneously identify a candidate therapeutic and provide a prognosis. In an embodiment, the method for prognosing a cancer in an individual comprises: (a) determining a level of a gene or gene product and/or a mutation in a gene from a biological sample of said individual, wherein said gene is selected from the group of genes listed in Table 10; and (b) prognosing the cancer based whether the gene is up or down regulated in the cancer as compared to a control. Table 10 indicates whether the differential regulation of the gene, or gene product thereof, as compared to the control indicates a good prognosis or bad prognosis. In the table, presence and absence also refer to overexpression and underexpression, respectively, as compared to the control. Any appropriate control can be used. In embodiments, the control comprises a non-diseased sample from the individual or from another individual. The method can be applied to the various cancers described herein. For example, the cancer assessed can be a breast cancer. In some embodiments, the individual has refractive cancer or has relapsed. The cancer can be metastatic. The expression and/or the mutation can be determined using IHC, FISH, microarray, sequencing, real-time PCR or other molecular profiling methods as disclosed herein. In an embodiment, MC is used to determine the expression of the protein comprising the gene product. In another embodiment, DNA microarray analysis is used. The method can be performed using the same molecular profiling results as the theranostic methods of the invention. In this manner, the invention provides a method for analyzing a cancer to simultaneously identify a candidate therapeutic and provide a prognosis.

TABLE 10
Prognostic Markers

| Biomarker | Summary |
|---|---|
| Caveolin 1 | Presence of Cav-1 indicates good prognosis. |
| Caveolin 1 | Absence of Cav-1 indicates bad prognosis. |
| CK5/6 | Presence of CK5/6 indicates bad prognosis. |
| CK5/6 | Absence of CK5/6 indicates good prognosis. |
| CK14 | Presence of CK14 indicates bad prognosis. |
| CK14 | Absence of CK14 indicates good prognosis. |
| CK17 | Presence of CK17 indicates bad prognosis. |
| CK17 | Absence of CK17 indicates good prognosis. |
| C-kit | Presence of c-kit indicates bad prognosis. |
| C-kit | Absence of c-kit indicates good prognosis. |
| c-myc | Amplification of c-myc indicates bad prognosis. |
| c-myc | Non-amplification of c-myc indicates good prognosis. |
| Cyclin D1 | Presence of Cyclin D1 indicates bad prognosis. |
| Cyclin D1 | Absence of Cyclin D1 indicates good prognosis. |
| E-cadherin | Presence of E-cadherin indicates good prognosis. |
| E-cadherin | Absence of E-cadherin indicates bad prognosis. |
| EGFR | Presence of EGFR indicates bad prognosis. |
| EGFR | Absence of EGFR indicates good prognosis. |
| P53 | Presence of P53 indicates good prognosis. |
| P53 | Absence of P53 indicates bad prognosis. |
| PDGFR | Presence of PDGFR indicates bad prognosis. |
| PDGFR | Absence of PDGFR indicates good prognosis. |

### EXAMPLES

### Example 1

### IHC and Microarray Testing of Over 500 Patients

The data reflected in the table depicted in FIGS. 26A-H and FIGS. 27A-27H relates to 544 patients whose diseased tissue samples underwent MC testing (FIG. 26) and 540 patients whose diseased tissue samples underwent gene microarray testing (FIG. 27) in accordance with IHC and microarray testing as previously described above. The patients were all in advanced stages of disease.

The data show biomarker patterns or biomarker signature sets in a number of tumor types, diseased tissue types, or diseased cells including adipose, adrenal cortex, adrenal gland, adrenal gland-medulla, appendix, bladder, blood vessel, bone, bone cartilage, brain, breast, cartilage, cervix, colon, colon sigmoid, dendritic cells, skeletal muscle, endometrium, esophagus, fallopian tube, fibroblast, gallbladder, kidney, larynx, liver, lung, lymph node, melanocytes, mesothelial lining, myoepithelial cells, osteoblasts, ovary, pancreas, parotid, prostate, salivary gland, sinus tissue, skeletal muscle, skin, small intestine, smooth muscle, stomach, synovium, joint lining tissue, tendon, testis, thymus, thyroid, uterus, and uterus corpus.

In 99 individuals with advanced breast cancer, immunohistochemistry analysis of 20 gene expressed proteins (FIG. 26B) showed that the gene expressed proteins analyzed were overexpressed a total of 367 times and that 16.35% of that total overexpression was attributable to HSP90 overexpression followed by 12.53% of the overexpression being attributable to TOP2A overexpression and 11.17% of the overexpression being attributable to SPARC. In addition, 9.81% of the overexpression was attributable to androgen receptor overexpression, 9.54% of the overexpression was attributable to PDGFR overexpression, and 9.26% of the overexpression was attributable to c-kit overexpression.

Accordingly, a biomarker pattern or biomarker signature set can be identified for advanced stage breast cancer and a therapeutic agent or therapeutic protocol can be identified which is capable of interacting with the biomarker pattern or signature set.

Another biomarker pattern or biomarker signature set for advanced stage breast cancer is shown from the microarray data in the table represented by FIGS. 27A-H. For example, in 100 individuals with advanced breast cancer (FIG. 27B), gene microarray analysis of 64 genes showed that the genes analyzed exhibited a change in expression a total of 1,158 times and that 6.39% of that total change in expression was attributable to SSTR3 change in expression followed by 5.79% of the change in expression being attributable to VDR change in expression and 5.35% of the change in expression being attributable to BRCA2 change in expression. Accordingly, another biomarker pattern or biomarker signature set can be identified for advanced stage breast cancer and another therapeutic agent or therapeutic protocol can be identified which is capable of interacting with this biomarker pattern or signature set.

### Example 2

### IHC Testing of Over 1300 Patients

FIGS. 28A through 28O represent a table that shows the frequency of a significant change in expression of certain gene expressed proteins by tumor type, i.e. the number of times that a gene expressed protein was flagged as a target by tumor type as being significantly overexpressed by immunohistochemistry analysis. The table also identifies the total number of times an overexpression of any gene expressed protein occurred in a particular tumor type using immunohistochemistry.

The data reflected in the table depicted in FIGS. 28A through 28O relates to 1392 patients whose diseased tissue underwent IIIC testing in accordance with IIIC testing as previously described above. The patients were all in advanced stages of disease.

The data show biomarker patterns or biomarker signature sets in a number of tumor types, diseased tissue types, or diseased cells including accessory, sinuses, middle and inner ear, adrenal glands, appendix, hematopoietic system, bones and joints, spinal cord, breast, cerebellum, cervix uteri, connective and soft tissue, corpus uteri, esophagus, eye, nose, eyeball, fallopian tube, extrahepatic bile ducts, other mouth, intrahepatic bile ducts, kidney, appendix-colon, larynx, lip, liver, lung and bronchus, lymph nodes, cerebral, spinal, nasal cartilage, excl. retina, eye, nos, oropharynx, other endocrine glands, other female genital, ovary, pancreas, penis and scrotum, pituitary gland, pleura, prostate gland, rectum renal pelvis, ureter, peritonem, salivary gland, skin, small intestine, stomach, testis, thymus, thyroid gland, tongue, unknown, urinary bladder, uterus, nos, vagina & labia, and vulva, nos.

In 254 individuals with advanced breast cancer, immunohistochemistry analysis of 19 gene expressed proteins (FIG. 28C) showed that the gene expressed proteins analyzed were overexpressed a total of 767 times and that 13.43% of that total overexpression was attributable to SPARC overexpression followed by 12.26% of the overexpression being attributable to c-kit overexpression and 11.47% of the overexpression being attributable to EGFR. In addition, 11.34% of the overexpression was attributable to androgen receptor overexpression, 11.08% of the overexpression was attributable to HSP90 overexpression, and 10.43% of the overexpression was attributable to PDGFR overexpression. Accordingly, a biomarker pattern or biomarker signature set can be identified for advanced stage breast cancer and a therapeutic agent or therapeutic protocol can be identified which is capable of interacting with the biomarker pattern or signature set.

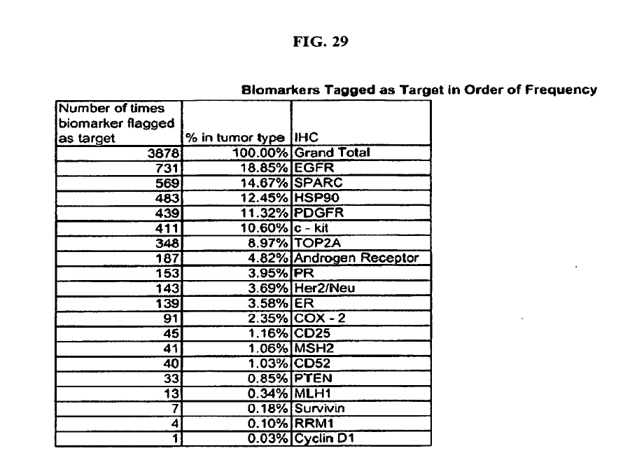

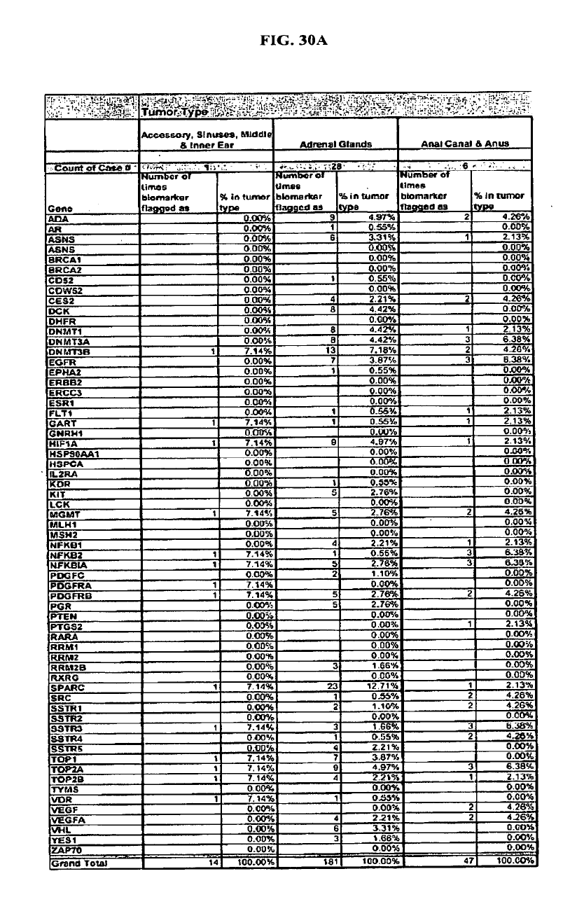

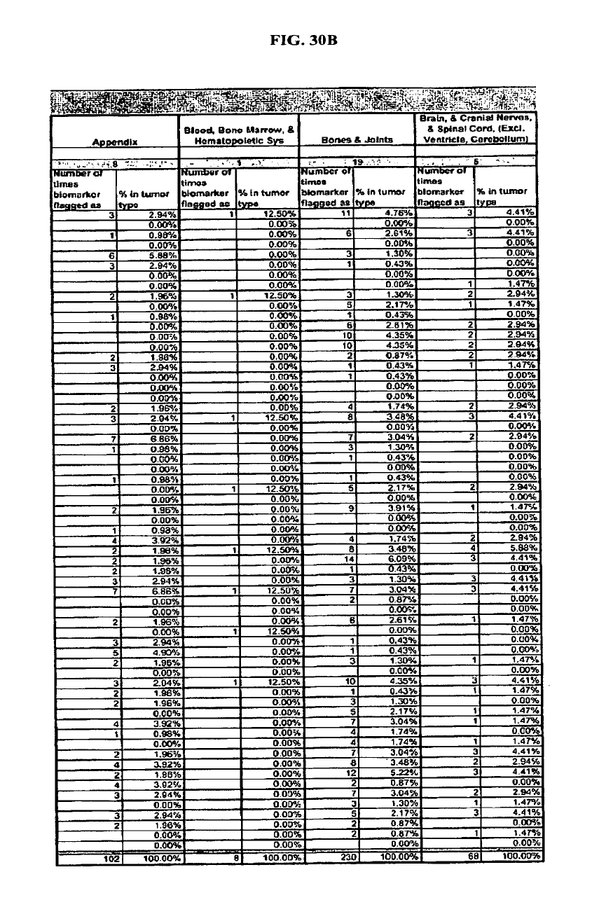

FIG. 29 depicts a table showing biomarkers (gene expressed proteins) tagged as targets in order of frequency in all tissues that were IHC tested. Immunohistochemistry of the 19 gene expressed proteins showed that the 19 gene expressed proteins were tagged 3878 times as targets in the various tissues tested and that EGFR was the gene expressed protein that was overexpressed the most frequently followed by SPARC.

### Example 3

### Microarray Testing of Over 300 Patients

FIGS. 30A through 30O represent a table that shows the frequency of a significant change in expression of certain genes by tumor type, i.e. the number of times that a gene was flagged as a target by tumor type as being significantly overexpressed or underexpressed by microarray analysis. The table also identifies the total number of times an overexpression or underexpression of any gene occurred in a particular tumor type using gene microarray analysis.

The data reflected in the table depicted in FIGS. 30A through 30O relates to 379 patients whose diseased tissue underwent gene microarray testing in accordance microarray testing as previously described above. The patients were all in advanced stages of disease. The data show biomarker patterns or biomarker signature sets in a number of tumor types, diseased tissue types, or diseased cells including accessory, sinuses, middle and inner ear, adrenal glands, anal canal and anus, appendix, blood, bone marrow & hematopoietic sys, bones and joints, brain & cranial nerves and spinal cord (excl. ventricle & cerebellum), breast, cerebellum, cervix uteri, connective & soft tissue, corpus uteri, esophagus, eye, nos, eyeball, fallopian tube, gallbladder 7 extrahepatic bile ducts, gum, floor of mouth & other mouth, intrahepatic bile ducts, kidney, large intestine (excl. appendix-colon), larynx, lip, liver, lung & bronchus, lymph nodes, meninges (cerebral, spinal), nasal cavity (including nasal cartilage), orbit & lacrimal gland (excl. retina, eye, nos), oropharynx, other endocrine glands, other fenale genital, ovary, pancreas, penis & scrotum, pituitary gland, pleura, prostate gland, rectum, renal pelvis & ureter, retroperitoneum & peritoneum, salivary gland, skin, small intestine, stomach, testis, thymus, thyroid gland, tongue, unknown, unspecified digestive organs, urinary bladder, uterus, nos, vagina & labia, and vulva, nos.

For example, in 168 individuals with advanced breast cancer (FIG. 30C), microarray analysis of 63 genes showed that the genes analyzed were either overexpressed or underexpressed a total of 1863 times and that 5.05% of that total change in expression was attributable to SSTR3 change in expression followed by 4.83% of the change in expression being attributable to NKFBIA change in expression and 4.62% of the change in expression being attributable to VDR. In addition, 4.35% of the change in expression was attributable to MGMT change in expression, 4.19% of the change in expression was attributable to ADA change in expression, and 3.97% of the change in expression was attributable to CES2 change in expression.

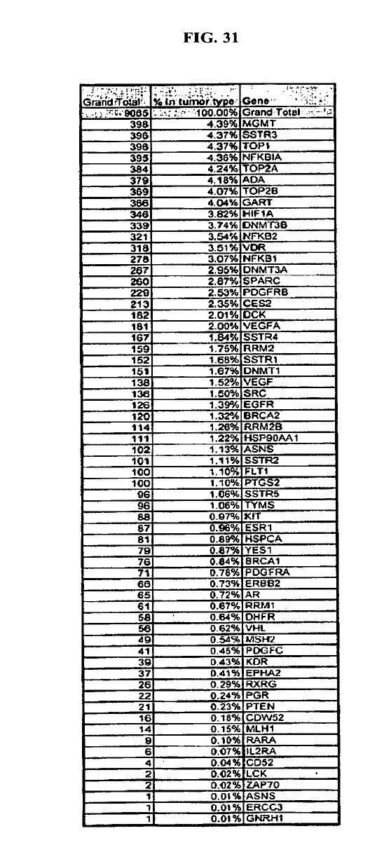

FIG. 31 depicts a table showing biomarkers as targets in order of frequency in all tissues that were tested.

### Example 4

### A Study Utilizing Molecular Profiling of Patients' Tumors to Find Targets and Select Treatments for Refractory Cancers

The primary objective was to compare progression free survival (PFS) using a treatment regimen selected by molecular profiling with the PFS for the most recent regimen the patient progressed on (e.g. patients are their own control) (FIG. 32). The molecular profiling approach was deemed of clinical benefit for the individual patient who had a PFS ratio (PFS on molecular profiling selected therapy/PFS on prior therapy) of ≧1.3.

The study was also performed to determine the frequency with which molecular profiling by IHC, FISH and microarray yielded a target against which there is a commercially available therapeutic agent and to determine response rate (RECIST) and percent of patients without progression or death at 4 months.

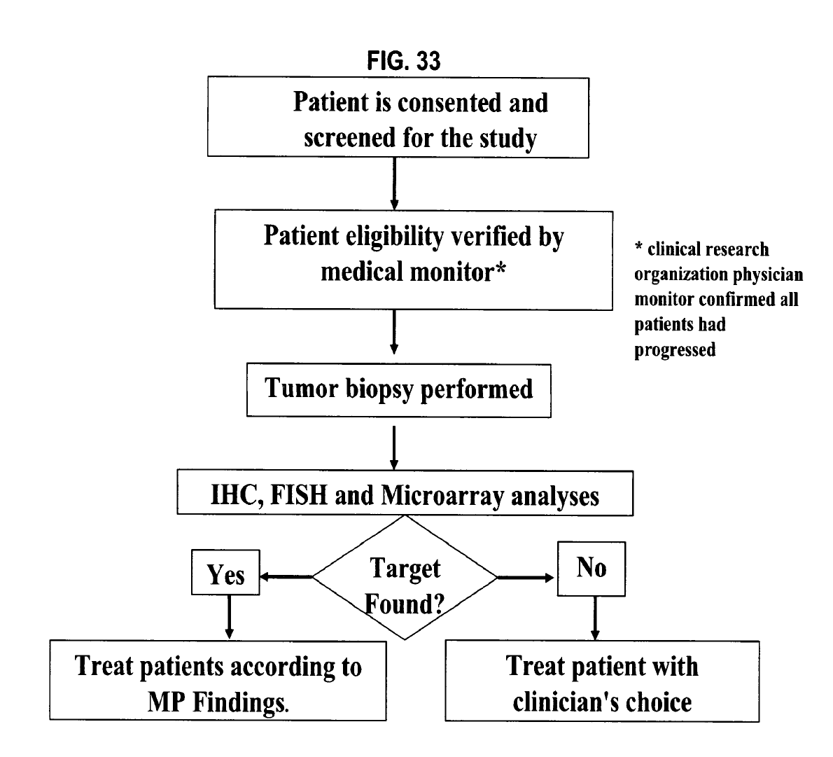

The study was conducted in 9 centers throughout the United States. An overview of the method is depicted in FIG. 33. As can be seen in FIG. 33, the patient was screened and consented for the study. Patient eligibility was verified by one of two physician monitors. The same physicians confirmed whether the patients had progressed on their prior therapy and how long that PFS (TTP) was. A tumor biopsy was then performed, as discussed below. The tumor was assayed using IHC, FISH (on paraffin-embedded material) and microarray (on fresh frozen tissue) analyses.

The results of the IHC/FISH and microarray were given to two study physicians who in general used the following algorithm in suggesting therapy to the physician caring for the patient: 1) IIIC/FISII and microarray indicated same target was first priority; 2) IHC positive result alone next priority; and 3) microarray positive result alone the last priority.

The patient's physician was informed of the suggested treatment and the patient was treated with the suggested agent(s) (package insert recommendations). The patient's disease status was assessed every 8 weeks and adverse effects were assessed by the NCI CTCAE version 3.0.

To be eligible for the study, the patient was required to: 1) provide informed consent and HIPAA authorization; 2) have any histologic type of metastatic cancer; 3) have progressed by RECIST criteria on at least 2 prior regimens for advanced disease; 4) be able to undergo a biopsy or surgical procedure to obtain tumor samples; 5) be ≧18 years, have a life expectancy >3 months, and an Eastern Cooperative Oncology Group (ECOG) Performance Status or 0-1; 6) have measurable or evaluable disease; 7) be refractory to last line of therapy (documented disease progression under last treatment; received ≧6 weeks of last treatment; discontinued last treatment for progression); 8) have adequate organ and bone marrow function; 9) have adequate methods of birth control; and 10) if CNS metastases then adequately controlled. The ECOG performance scale is described in Oken, M. M., Creech, R. H., Tormey, D. C., Horton, J., Davis, T. E., McFadden, E. T., Carbone, P. P.: Toxicity And Response Criteria Of The Eastern Cooperative Oncology Group. Am J Clin Oncol 5:649-655, 1982, which is incorporated by reference in its entirety. Before molecular profiling was performed, the principal investigator at the site caring for the patient must designate what they would treat the patient with if no molecular profiling results were available.

Methods

All biopsies were performed at local investigators' sites. For needle biopsies, 2-3 18 gauge needle core biopsies were performed. For DNA microarray (MA) analysis, tissue was immediately frozen and shipped on dry ice via FedEx to a central CLIA certified laboratory, Caris MPI in Phoenix, Ariz. For IHC, paraffin blocks were shipped on cold packs. IHC was considered positive for target if 2+ in ≧30% of cells. The MA was considered positive for a target if the difference in expression for a gene between tumor and control organ tissue was at a significance level of p≦0.001.

Ascertainment of the Time to Progression to Document the Progression-Free Survival Ratio

Time to progression under the last line of treatment was documented by imaging in 58 patients (88%). Among these 58 patients, documentation by imaging alone occurred in 49 patients (74%), and documentation by imaging with tumor markers occurred in nine patients (14%; ovarian cancer, n 3; colorectal, n 1; pancreas, n 1; prostate, n 3; breast, n 1). Patients with clinical proof of progression were accepted when the investigator reported the assessment of palpable and measurable lesions (i.e., inflammatory breast cancer, skin/subcutaneous nodules, or lymph nodes), which occurred in six patients (9%). One patient (2%) with prostate cancer was included with progression by tumor marker. In one patient (2%) with breast cancer, the progression was documented by increase of tumor marker and worsening of bone pain. The time to progression achieved with a treatment based on molecular profiling was documented by imaging in 44 patients (67%) and by clinical events detected between two scheduled tumor assessments in 20 patients. These clinical events were reported as serious adverse events related to disease progression (e.g., death, bleeding, bowel obstruction, hospitalization), and the dates of reporting were censored as progression of disease. The remaining two patients were censored at the date of last follow-up.

IHC/FISH

For IHC studies, the formalin fixed, paraffin embedded tumor samples had slices from these blocks submitted for IHC testing for the following proteins: EGFR, SPARC, C-kit, ER, PR, Androgen receptor, PGP, RRM1, TOPO1, BRCP1, MRP1, MGMT, PDGFR, DCK, ERCC1, Thymidylate synthase, Her2/neu and TOPO2A. IHCs for all proteins were not carried out on all patients' tumors.

Formalin-fixed paraffin-embedded patient tissue blocks were sectioned (4 μm thick) and mounted onto glass slides. After deparaffination and rehydration through a series of graded alcohols, pretreatment was performed as required to expose the targeted antigen.

Human epidermal growth factor receptor 2 (IIER2) and epidermal growth factor receptor (EGFR) were stained as specified by the vendor (DAKO, Denmark). All other antibodies were purchased from commercial sources and visualized with a DAB biotin-free polymer detection kit. Appropriate positive control tissue was used for each antibody. Negative control slides were stained by replacing the primary antibody with an appropriately matched isotype negative control reagent. All slides were counterstained with hematoxylin as the final step and cover slipped. Tissue microarray sections were analyzed by FISH for EGFR and HER-2/neu copy number per the manufacturer's instructions. FISH for HER-2/neu (was done with the PathVysion HER2 DNA Probe Kit (Abbott Molecular, Abbott Park, Ill.). FISH for EGFR was done with the LSI EGFR/CEP 7 Probe (Abbott Molecular).

All slides were evaluated semi-quantitatively by a first pathologist, who confirmed the original diagnosis as well as read each of the immunohistochemical stains using a light microscope. Some lineage immunohistochemical stains were performed to confirm the original diagnosis, as necessary. Staining intensity and extent of staining were determined; both positive, tumor-specific staining of tumor cells and highly positive (≧2+), pervasive (≧30%) tumor specific staining results were recorded. IHC was considered positive for target if staining was ≧2+ in ≧30% of cells. Rather than look for a positive signal without qualification, this approach raises the stringency of the cut point such that it would be a significant or more demonstrative positive. A higher positive is more likely to be associated with a therapy that would affect the time to progression. The cut point used (i.e., staining was ≧2+ in ≧30% of cells) is similar to some cut points used in breast cancer for HER2/neu. When IHC cut points were compared with evidence from the tissue of origin of the cancer, the cut points were equal to or higher (more stringent) than the evidence cut points. A standard 10% quality control was performed by a second pathologist.

Microarray

Tumor samples obtained for microarray were snap frozen within 30 minutes of resection and transmitted to Caris-MPI on dry ice. The frozen tumor fragments were placed on a 0.5 mL aliquot of frozen 0.5M guanidine isothiocyanate solution in a glass tube, and simultaneously thawed and homogenized with a Covaris S2 focused acoustic wave homogenizer (Covaris, Woburn, Mass.). A 0.5 mL aliquot of TriZol was added, mixed and the solution was heated to 65° C. for 5 minutes then cooled on ice and phase separated by the addition of chloroform followed by centrifugation. An equal volume of 70% ethanol was added to the aqueous phase and the mixture was chromatographed on a Qiagen RNeasy column (Qiagen, Germantown, Md.). RNA was specifically bound and then eluted. The RNA was tested for integrity by assessing the ratio of 28S to 18S ribosomal RNA on an Agilent BioAnalyzer (Agilent, Santa Clara, Calif.). Two to five micrograms of tumor RNA and two to five micrograms of RNA from a sample of a normal tissue representative of the tumor's tissue of origin were separately converted to cDNA and then labeled during T7 polymerase amplification with contrasting fluor tagged (Cy3, Cy5) cytidine triphosphate. The labeled tumor and its tissue of origin reference were hybridized to an Agilent H1Av2 60-mer olio array chip with 17,085 unique probes.

The arrays contain probes for 50 genes for which there is a possible therapeutic agent that would potentially interact with that gene (with either high expression or low expression). Those 50 genes included: ADA, AR, ASNA, BCL2, BRCA2, CD33, CDW52, CES2, DNMT1, EGFR, ERBB2, ERCC3, ESR1, FOLR2, GART, GSTP1, HDAC1, HIF1A, HSPCA, IL2RA, KIT, MLH1, MS4A1, MASH2, NFKB2, NFKBIA, OGFR, PDGFC, PDGFRA, PDGFRB, PGR, POLA, PTEN, PTGS2, RAF1, RARA, RXRB, SPARC, SSTR1, TK1, TNF, TOP1, TOP2A, TOP2B, TXNRD1, TYMS, VDR, VEGF, VHL, and ZAP70.

The chips were hybridized from 16 to 18 hours at 60° C. and then washed to remove non-stringently hybridized probe and scanned on an Agilent Microarray Scanner. Fluorescent intensity data were extracted, normalized, and analyzed using Agilent Feature Extraction Software. Gene expression was judged to be different from its reference based on an estimate of the significance of the extent of change, which was estimated using an error model that takes into account the levels of signal to noise for each channel, and uses a large number of positive and negative controls replicated on the chip to condition the estimate. Expression changes at the level of p≦0.001 were considered as significantly different.

Statistical Considerations

The protocol called for a planned 92 patients to be enrolled of which an estimated 64 patients would be treated with therapy assigned by molecular profiling. The other 28 patients were projected to not have molecular profiling results available because of (a) inability to biopsy the patient; (b) no target identified by the molecular profiling; or (c) deteriorating performance status. Sixty four patients were required to receive molecular profiling treatment in order to reject the null hypothesis (Ho) that: ≦15% of patients would have a PFS ratio of ≧1.3 (e.g. a non-promising outcome).

Treatment Selection

Treatment for the patients based on molecular profiling results was selected using the following algorithm: 1) IIIC/FISII and microarray indicates same target; 2) IIIC positive result alone; 3) microarray positive result alone. The patient's physician was informed of suggested treatment and the patient was treated based on package insert recommendations. Disease status was assessed every 8 weeks. Adverse effects were assessed by NCI CTCAE version 3.0.

The targets and associated drugs are listed in Table 11.

TABLE 11
Pairings of Targets and Drugs

| Potential | Agents Suggested as |
|---|---|
| Target | Interacting With the Target |
| IHC | |
| EGFR | Cetuximab, erlotinib, gefitinib |
| SPARC | Nanoparticle albumin-bound paclitaxel |
| c-KIT | Imatinib, sunitinib, sorafenib |
| ER | Tamoxifen, aromatase inhibitors, |
| | toremifene, progestational agent |
| PR | Progestational agents, tamoxifen, aromatase |
| | inhibitor, goserelin |
| Androgen | Flutamide, abarelix, bicalutamide, |
| receptor | leuprolide, goserelin |
| PGP | Avoid natural products, doxorubicin, etoposide, |
| | docetaxel, vinorelbine |
| HER2/NEU | Trastuzumab |
| PDGFR | Sunitinib, imatinib, sorafenib |
| CD52 | Alemtuzumab |
| CD25 | Denileukin diftitox |
| HSP90 | Geldanamycin, CNF2024 |
| TOP2A | Doxorubicin, epirubicin, ctoposidc |
| Microarray | |
| ADA | Pentostatin, cytarabine |
| AR | Flutamide, abarelix, bicalutamide, |
| | leuprolide, goserelin |
| ASNA | Asparaginase |
| BCL2 | Oblimersen sodium^{†} |
| BRCA2 | Mitomycin |
| CD33 | Gemtuzumab ozogamicin |
| CDW52 | Alemtuzumab |
| CES-2 | Irinotecan |
| DCK | Gemcitabine |
| DNMT1 | Azacitidine, decitabine |
| EGFR | Cetuximab, erlotinib, gefitinib |
| ERBB2 | Trastuzumab |
| ERCC1 | Cisplatin, carboplatin, oxaliplatin |
| ESR1 | Tamoxifen, aromatase inhibitors, toremifene, |
| | progestational agent |
| FOLR2 | Methotrexate, pemetrexed |
| GART | Pemetrexed |
| GSTP1 | Platinum |
| HDAC1 | Vorinostat |
| HIF1α | Bevacizumab, sunitinib, sorafenib |
| HSPCA | Geldanamycin, CNF2024 |
| IL2RA | Aldesleukin |
| KIT | Imatinib, sunitinib, sorafenib |
| MLH-1 | Gemcitabine, oxaliplatin |
| MSH1 | Gemcitabine |
| MSH2 | Gemcitabine, oxaliplatin |
| NFKB2 | Bortezomib |
| NFKB1 | Bortezomib |
| OGFR | Opioid growth factor |
| PDGFC | Sunitinib, imatinib, sorafenib |
| PDGFRA | Sunitinib, imatinib, sorafenib |
| PDGFRB | Sunitinib, imatinib, sorafenib |
| PGR | Progestational agents, tamoxifen, aromatase |
| | inhibitors, goserelin |
| POLA | Cytarabine |
| PTEN | Rapamycin (if low) |
| PTGS2 | Celecoxib |
| RAF1 | Sorafenib |
| RARA | Bexarotene, all-trans-retinoic acid |
| RXRB | Bexarotene |
| SPARC | Nanoparticle albumin-bound paclitaxel |
| SSTR1 | Octreotide |
| TK1 | Capecitabine |
| TNF | Infliximab |
| TOP1 | lrinotecan, topotecan |
| TOP2A | Doxorubicin, etoposide, mitoxantrone |
| TOP2B | Doxorubicin, etoposide, mitoxantrone |
| TXNRD1 | Px12 |
| TYMS | Fluorouracil, capecitabine |
| VDR | Calcitriol |
| VEGF | Bevacizumab, sunitinib, sorafenib |
| VHL | Bevacizumab, sunitinib, sorafenib |
| ZAP70 | Geldanamycin, CNP2024 |

Results

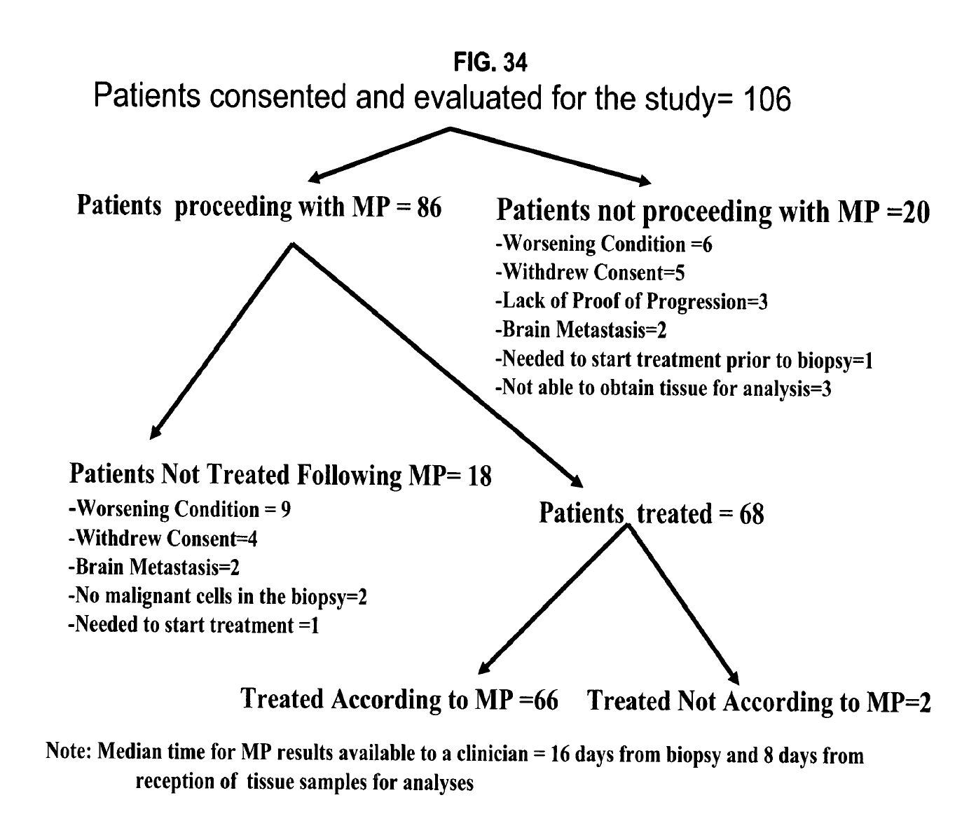

The distribution of the patients is diagrammed in FIG. 34 and the characteristics of the patients shown in Tables 12 and 13. As can be seen in FIG. 34, 106 patients were consented and evaluated. There were 20 patients who did not proceed with molecular profiling for the reasons outlined in FIG. 34 (mainly worsening condition or withdrawing their consent or they did not want any additional therapy). There were 18 patients who were not treated following molecular profiling (mainly due to worsening condition or withdrawing consent because they did not want additional therapy). There were 68 patients treated, with 66 of them treated according to molecular profiling results and 2 not treated according to molecular profiling results. One of the two was treated with another agent because the clinician caring for the patient felt a sense of urgency to treat and the other was treated with another agent because the insurance company would not cover the molecular profiling suggested treatment.

The median time for molecular profiling results being made accessible to a clinician was 16 days from biopsy (range 8 to 30 days) and a median of 8 days (range 0 to 23 days) from receipt of the tissue sample for analysis. Some modest delays were caused by the local teams not sending the patients' blocks immediately (due to their need for a pathology workup of the specimen). Patient tumors were sent from 9 sites throughout the United States including: Greenville, S.C.; Tyler, Tex.; Beverly Hills, Calif.; Huntsville, Ala.; Indianapolis, Ind.; San Antonio, Tex.; Scottsdale, Ariz. and Los Angeles, Calif.

Table 12 details the characteristics of the 66 patients who had molecular profiling performed on their tumors and who had treatment according to the molecular profiling results. As seen in Table 8, of the 66 patients the majority were female, with a median age of 60 (range 27-75). The number of prior treatment regimens was 2-4 in 53% of patients and 5-13 in 38% of patients. There were 6 patients (9%), who had only 1 prior therapy because no approved active 2^{nd} line therapy was available. Twenty patients had progressed on prior phase I therapies. The majority of patients had an ECOG performance status of 1.

TABLE 12
Patient Characteristics (n = 66)

| Characteristic | n | % |
|---|---|---|
| Gender | | |
| Female | 43 | 65 |
| Male | 23 | 35 |
| Age | | |
| Median (range) | 60 | (27-75) |
| Number of Prior Treatments | | |
| 2-4* | 35 | 53 |
| 5-13 | 25 | 38 |
| ECOG | | |
| 0 | 18 | 27 |
| 1 | 48 | 73 |

*Note:
6 patients (9%) had 1 prior

As seen in Table 13, tumor types in the 66 patients included breast cancer 18 (27%), colorectal 11 (17%), ovarian 5 (8%), and 32 patients (48%) were in the miscellaneous categories. Many patients had the more rare types of cancers.

TABLE 13
Patient Tumor Types (n = 66)

| Tumor Type | n | % |
|---|---|---|
| Breast | 18 | 27 |
| Colorectal | 11 | 17 |
| Ovarian | 5 | 8 |
| Miscellaneous | 32 | 48 |
| Prostate | 4 | 6 |
| Lung | 3 | 5 |
| Melanoma | 2 | 3 |
| Small cell (esopha/retroperit) | 2 | 3 |
| Cholangiocarcinoma | 2 | 3 |
| Mesothelioma | 2 | 3 |
| H&N (SCC) | 2 | 3 |
| Pancreas | 2 | 3 |
| Pancreas neuroendocrine | 1 | 1.5 |
| Unknown (SCC) | 1 | 1.5 |
| Gastric | 1 | 1.5 |
| Peritoneal pseudomyxoma | 1 | 1.5 |
| Anal Canal (SCC) | 1 | 1.5 |
| Vagina (SCC) | 1 | 1.5 |
| Cervis | 1 | 1.5 |
| Renal | 1 | 1.5 |
| Eccrine seat adenocarinoma | 1 | 1.5 |
| Salivary gland adenocarinoma | 1 | 1.5 |
| Soft tissue sarcoma (uterine) | 1 | 1.5 |
| GIST (Gastric) | 1 | 1.5 |
| Thyroid-Anaplastic | 1 | 1.5 |

Primary Endpoint: PFS Ratio ≧1.3

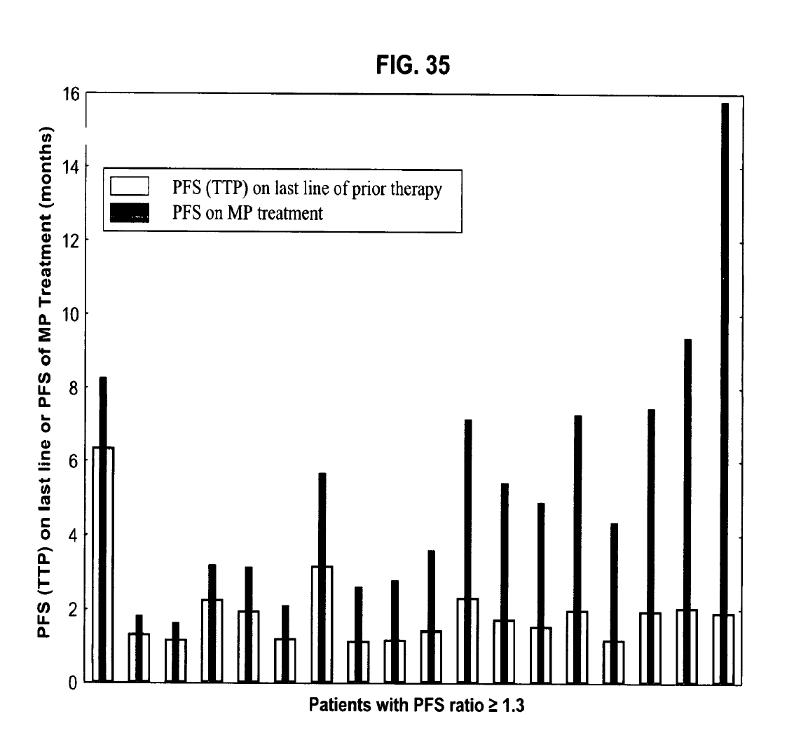

As far as the primary endpoint for the study is concerned (PFS ratio of ≧1.3), in the 66 patients treated according to molecular profiling results, the number of patients with PFS ratio greater or equal to 1.3 was 18 out of the 66 or 27%, 95% CI 17-38% one-sided, one-sample non parametric test p=0.007. The null hypothesis was that ≦15% of this patient population would have a PFS ratio of ≧1.3. Therefore, the null hypothesis is rejected and our conclusion is that this molecular profiling approach is beneficial. FIG. 35 details the comparison of PFS on molecular profiling therapy (the bar) versus PFS (TTP) on the patient's last prior therapy (the boxes) for the 18 patients. The median PFS ratio is 2.9 (range 1.3-8.15).

If the primary endpoint is examined, as shown in Table 14, a PFS ratio ≧1.3 was achieved in 8/18 (44%) of patients with breast cancer, 4/11 (36%) patients with colorectal cancer, 1/5 (20%) of patients with ovarian cancer and 5/32 (16%) patients in the miscellaneous tumor types (note that miscellaneous tumor types with PFS ratio ≧1.3 included: lung 1/3, cholangiocarcinoma 1/3, mesothelioma 1/2, eccrine sweat gland tumor 1/1, and GIST (gastric) 1/1).

TABLE 14
Primary Endpoint-PFS Ratio ≧ 1.3 By Tumor Type

| | | Number | |
|---|---|---|---|
| | Total | with PFS | |
| Tumor Type | Treated | Ratio ≧ 1.3 | % |
| Breast | 18 | 8 | 44 |
| Colorectal | 11 | 4 | 36 |
| Ovarian | 5 | 1 | 20 |
| Miscellaneous* | 32 | 5 | 16 |
| Total | 66 | 18 | 27 |

*lung 1/3, cholangiocarcinoma ½, mesothelioma ½, eccrine sweat 1/1, GIST (gastric) 1/1

The treatment that the 18 patients with the PFS ≧1.3 received based on profiling is detailed in Table 15. As can be seen in that table for breast cancer patients, the treatment ranged from diethylstibesterol to nab paclitaxel+ gemcitabine to doxorubicin. Treatments for patients with other tumor types are also detailed in Table 15. The table further shows a comparison of the drugs that the responding patients received versus the drugs that would have been suggested without molecular profiling and indicates which targets were used to suggest the therapies. Overall, 14 were treated with combinations and 4 were treated with single agents.

TABLE 15
Targets Noted in Patients’ Tumors, Treatment Suggested on the Basis of These Results,
and Treatment Investigator Would Use if No Target Was Identified (in patients with PFS ratio ≧1.3)

| | | | Treatment the |
|---|---|---|---|
| | | Treatment Suggested | Investigator Would |
| | Targets Used to | on Basis of Patient's | Have Used if No |
| Location of Primary | Suggest Treatment | Tumor Molecular | Results From |
| Tumor | and Method Used | Profiling | Molecular Profiling |
| Breast | ESR1: I; ESR1: M | DES 5 mg TID | Investigational |
| Cholangiocarcinoma | EGFR: I; TOP1: M | CPT-11 350 mg/m² | Investigational |
| | | every 3 weeks; | |
| | | cctuximab 400 mg/m² | |
| | | day 1,250 mg/m² every | |
| | | week | |
| Breast | SPARC: I; SPARC, | NAB paclitaxel 260 | Docetaxel, trastuzumab |
| | ERBB2: M | mg/m² every 3 weeks; | |
| | | trastuzumab 6 mg/kg | |
| | | every 3 weeks | |
| Eccrine sweat gland | c-KIT: I; c-KIT: M | Sunitinib 50 mg/d, 4 | Best supportive care |
| (right forearm) | | weeks on/2 weeks off | |
| Ovary | HER2/NEU, ER: I; | Lapatinib 1,250 mg PO | Bevacizumab |
| | HER2/NEU: M | days 1-21; tamoxifen 20 | |
| | | mg PO | |
| Colon/rectum | PDGFR, c-KIT: I I; | CPT-11 70 mg/m² | Cetuximab |
| | PDGFR, TOP1: M | weekly for 4 weeks on/2 | |
| | | weeks off; sorafenib | |
| | | 400 mg BID | |
| Breast | SPARC: I; DCK: M | NAB paclitaxel 90 | Mitomycin |
| | | mg/m² every 3 weeks; | |
| | | gcmcitabinc 750 mg/m² | |
| | | days 1, 8, 15, every 3 | |
| | | weeks | |
| Breast | ER: I; ER, TYMS: M | Letrozole 2.5 mg daily; | Capecitabine |
| | | capecitabine 1,250 | |
| | | mg/m² BID, 2 weeks | |
| | | on/1 week off | |
| Malignant mesothelioma | MLH1, MLH2: I; | Gemcitabine 1,000 | Gemcitabine |
| | RRM2B, RRM1, RRM2, | mg/m² days 1 and 8, | |
| | TOP2B: M | every 3 weeks; | |
| | | etoposide 50 mg/m² | |
| | | 3 days every 3 weeks | |
| Breast | MSH2 | Oxaliplatin 85 mg/m² | Investigational |
| | | every 2 weeks; | |
| | | fluorouracil (5FU) | |
| | | 1,200 mg/m² days 1 and | |
| | | 2, every 2 weeks; | |
| | | trastuzumab 4 mg/kg | |
| | | day 1, 2 mg/kg every | |
| | | week | |
| Non-small-cell lung | EGFR: I; EGFR | Cetuximab 400 mg/m² | Vinorelbine |
| cancer | | day 1,250 mg/m² every | |
| | | week; CPT-11 125 | |
| | | mg/m² weekly for 4 | |
| | | weeks on/2 weeks off | |
| Colon/rectum | MGMT | Temozolomide 150 | Capecitabine |
| | | mg/m² for 5 days every | |
| | | 4 weeks; bevacizumab 5 | |
| | | mg/kg every 2 weeks | |
| Colon/rectum | PDGFR, c-KIT: I; | Mitomycin 10 mg once | Capecitabine |
| | PDGFR: KDR, HIF1A, | every 4-6 weeks; | |
| | BRCA2: M | sunitinib 37.5 mg/d, 4 | |
| | | weeks on/2 weeks off | |
| Breast | DCK, DHFR: M | Gemcitabine 1,000 | Best supportive care |
| | | mg/m² days 1 and 8 | |
| | | every 3 weeks; | |
| | | pemetrexed 500 mg/m² | |
| | | days 1 and 8, every 3 | |
| | | weeks | |
| Breast | TOP2A: I; TOP2A: M | Doxorubicin 50 mg/m² | Vinorelbine |
| | | every 3 weeks | |
| Colon/rectum | MGMT, VEGFA, | Temozolomide 150 | Panitumumab |
| | H1F1A: M | mg/m² for 5 days every | |
| | | 4 weeks; sorafenib 400 | |
| | | mg BID | |
| Breast | ESR1, PR: I; ESRI, PR: | Exemestane 25 mg | Doxorubicin liposomal |
| | M | every day | |
| GIST (stomach) | EGFR: T; EGFR, | Gemcitabine 1,000 | None |
| | RRM2: M | mg/m² days 1, 8, and 15 | |
| | | every 4 weeks; | |
| | | cetuximab 400 mg/m² | |
| | | day 1,250 mg/m² every | |
| | | week | |

*Abbreviations used in Table 15: I, immunohistochemistry; M, microarray; DES, diethylstilbestrol; CPT-11, irinotecan; TID, three times a day; NAB, nanoparticle albumin bound; PO, orally; BID, twice a day; GIST, GI stromal tumor.

Secondary Endpoints

The results for the secondary endpoint for this study are as follows. The frequency with which molecular profiling of a patients' tumor yielded a target in the 86 patients where molecular profiling was attempted was 84/86 (98%). Broken down by methodology, 83/86 (97%) yielded a target by IHC/FISH and 81/86 (94%) yielding a target by microarray. RNA was tested for integrity by assessing the ratio of 28S to 18S ribosomal RNA on an Agilent BioAnalyzer. 83/86 (97%) specimens had ratios of 1 or greater and gave high intra-chip reproducibility ratios. This demonstrates that very good collection and shipment of patients' specimens throughout the United States and excellent technical results can be obtained.

By RECIST criteria in 66 patients, there was 1 complete response and 5 partial responses for an overall response rate of 10% (one CR in a patient with breast cancer and PRs in breast, ovarian, colorectal and NSCL cancer patients). Patients without progression at 4 months included 14 out of 66 or 21%.

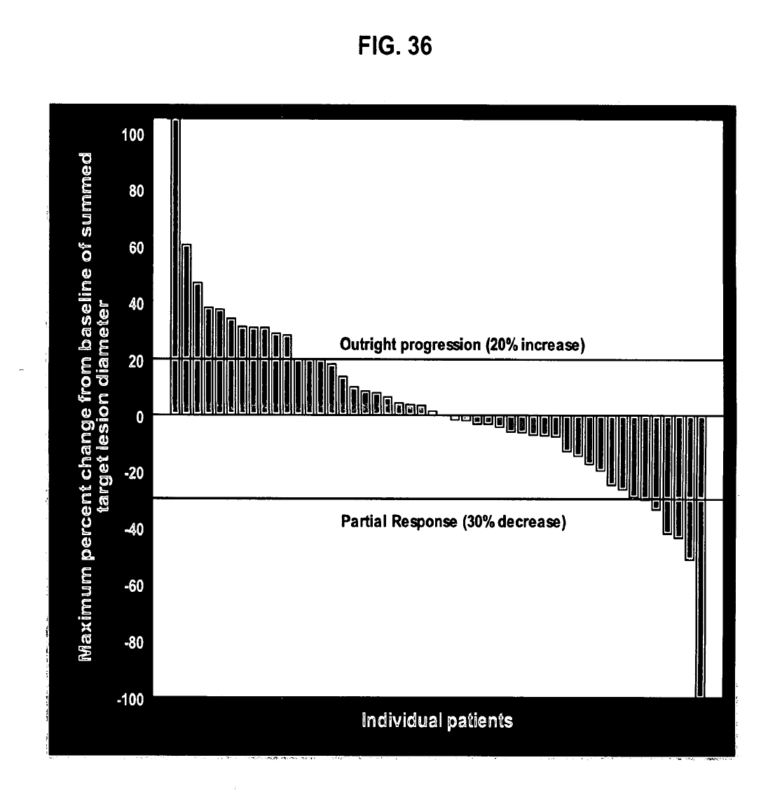

In an exploratory analysis, a waterfall plot for all patients for maximum % change of the summed diameters of target lesions with respect to baseline diameters was generated. The patients who had progression and the patients who had some shrinkage of their tumor sometime during their course along with those partial responses by RECIST criteria is demonstrated in FIG. 36. There is some shrinkage of patient's tumors in over 47% of the patients (where 2 or more evaluations were completed).

Other Analyses-Safety

As far as safety analyses there were no treatment related deaths. There were nine treatment related serious adverse events including anemia (2 patients), neutropenia (2 patients), dehydration (1 patient), pancreatitis (1 patient), nausea (1 patient), vomiting (1 patient), and febrile neutropenia (1 patient). Only one patient (1.5%) was discontinued due to a treatment related adverse event of grade 2 fatigue.

Other Analyses-Relationship Between What the Clinician Caring for the Patient would have Selected Versus What the Molecular Profiling Selected

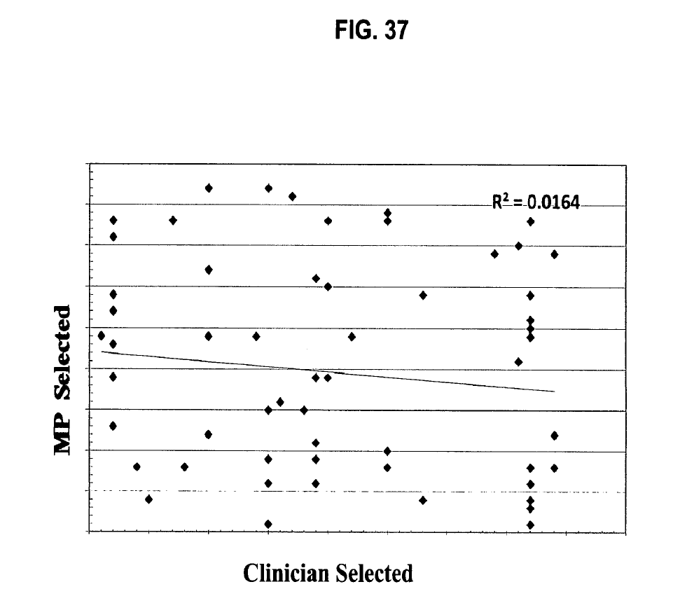

The relationship between what the clinician selected to treat the patient before knowing what molecular profiling results suggested for treatment was also examined. As detailed in FIG. 37, there is no pattern between the two. More specifically, no matches for the 18 patients with PFS ratio ≧1.3 were noted.

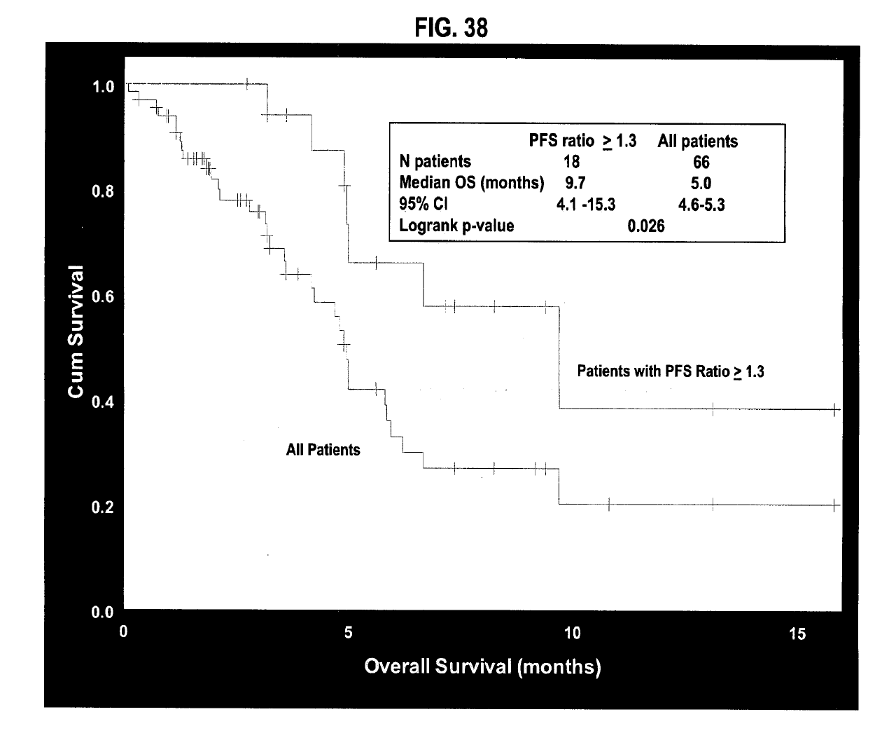

The overall survival for the 18 patients with a PFS ratio of ≧1.3 versus all 66 patients is shown in FIG. 38. This exploratory analysis was done to help determine if the PFS ratio had some clinical relevance. The overall survival for the 18 patients with the PFS ratio of ≧1.3 is 9.7 months versus 5 months for the whole population—log rank 0.026. This exploratory analysis indicates that the PFS ratio is correlated with the clinical parameter of survival.

### CONCLUSIONS

This prospective multi-center pilot study demonstrates: (a) the feasibility of measuring molecular targets in patients' tumors from 9 different centers across the US with good quality and sufficient tumor collection—and treat patients based on those results; (b) this molecular profiling approach gave a longer PFS for patients on a molecular profiling suggested regimen than on the regimen they had just progressed on for 27% of the patients (confidence interval 17-38%) p=0.007; and (c) this is a promising result demonstrating use and benefits of molecular profiling

The results also demonstrate that patients with refractory cancer can commonly have simple targets (such as ER) for which therapies are available and can be beneficial to them. Molecular profiling for patients who have exhausted other therapies and who are perhaps candidates for phase I or II trials could have this molecular profiling performed.

### Example 5

### Molecular Profilins System

Molecular profiling is performed to determine a treatment for a disease, typically a cancer. Using a molecular profiling approach, molecular characteristics of the disease itself are assessed to determine a candidate treatment. Thus, this approach provides the ability to select treatments without regard to the anatomical origin of the diseased tissue, or other “one-size-fits-all” approaches that do not take into account personalized characteristics of a particular patient's affliction. The profiling comprises determining gene and gene product expression levels, gene copy number and mutation analysis. Treatments are identified that are indicated to be effective against diseased cells that overexpress certain genes or gene products, underexpress certain genes or gene products, carry certain chromosomal aberrations or mutations in certain genes, or any other measureable cellular alterations as compared to non-diseased cells. Because molecular profiling is not limited to choosing amongst therapeutics intended to treat specific diseases, the system has the power to take advantage of any useful technique to measure any biological characteristic that can be linked to a therapeutic efficacy. The end result allows caregivers to expand the range of therapies available to treat patients, thereby providing the potential for longer life span and/or quality of life than traditional “one-size-fits-all” approaches to selecting treatment regimens.

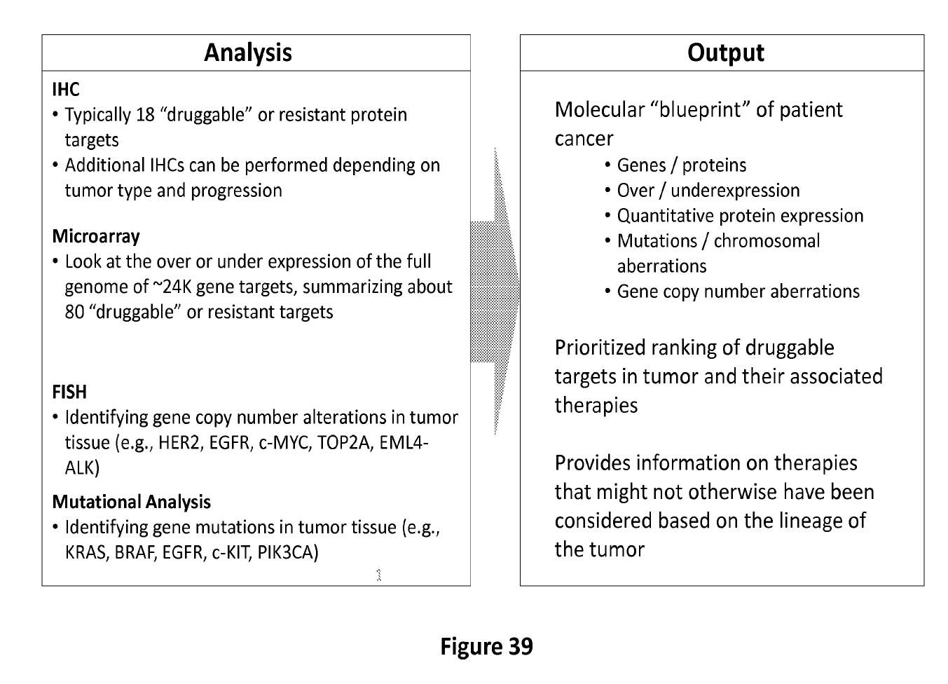

A molecular profiling system has several individual components to measure expression levels, chromosomal aberrations and mutations. The components are shown in FIG. 39. These include immunohistochemistry assays (IHC) on formalin fixed paraffin embedded (FFPE) cancer tissue. To perform IHC on a sample, a paraffin embedded block with a large section of tumor (at least 20% viable neoplasm) from the procedure which is preferred. For any tumor, IHC is run for 18 target genes comprising druggable or drug resistant targets. IHC can be performed on additional genes depending on disease characteristics, e.g., tumor origin and progression. In addition to IIIC, gene expression arrays, such as the Agilent 44K chip (Agilent Technologies, Inc., Santa Clara, Calif.). This system is capable of determining the relative expression level of roughly 44,000 different sequences through RT-PCR from RNA extracted from fresh frozen tissue. The expression of 80 druggable or drug resistant targets is examined in further detail. Because of the practicalities involved in obtaining fresh frozen tissue, only a portion of samples with sufficient quantity and quality of mRNA are analyzed using microarray analysis. The system also assesses gene copy number and/or other chromosomal abnormalities for a number of genes using FISH (fluorescence in situ hybridization). Finally, mutation analysis is done by DNA sequencing for a several specific mutations. All of this data is stored for each patient case. Microarray results MC, FISII and DNA sequencing analysis for a number of genes that have been shown to impact therapeutic options are used to generate a final patient report. The report can include a prioritized list of druggable targets and their associated therapies. The report is explained by a practicing oncologist. Once the data are reported, the final decisions rest with the treating physician. Based on this approach, the treating physician has information on therapies that might not otherwise have been considered based on the lineage of the disease.

### Example 6

### Illumina Expression Analysis

The Illumina Whole Genome DASL assay (Illumina Inc., San Diego, Calif.) offers a method to simultaneously profile over 24,000 transcripts from minimal RNA input, from both fresh frozen (FF) and formalin-fixed paraffin embedded (FFPE) tissue sources, in a high throughput fashion. The analysis makes use of the Whole-Genome DASL Assay with UDG (IIlumina, cat#DA-903-1024/DA-903-1096), the Illumina IIybridization Oven, and the Illumina iScan System.

A small piece (0.25 gm-0.5 gm) of tumor or 4-5 cores flash-frozen within 30 minutes of extraction from the patient is preferred to preserve the RNA. This tissue is preferably preservative-free (e.g., no exposure to alcohol) and remains frozen (e.g., either in a −80° freezer or on dry ice once frozen). If fresh tissue is not available, one paraffin block (40% Tumor) or 45 unstained slides can be used. The sample can be treated to preserve the RNA, e.g., using RNAlater® RNA stabilization solution according to the manufacturer's instructions (Applied Biosystems/Ambion, Austin, Tex.). The RNA preservative stabilization solution is an aqueous tissue storage reagent that rapidly permeates most tissues to stabilize and protect RNA in fresh specimens. Samples in RNA Preservative solution can be stored for periods of time that may otherwise render RNA unusable for molecular profile assays.

The Whole Genome DASL assay is performed following the manufacturer's instructions. Total RNA isolated from either FF or FFPE sources is converted to cDNA using biotinylated oligo(dT) and random nonamer primers. The use of both oligo(dT) and random nonamer primers helps ensure cDNA synthesis of degraded RNA fragments, such as those obtained from FFPE tissue. The biotinylated cDNA is then annealed to the DASL Assay Pool (DAP) probe groups. Probe groups contain oligonucleotides specifically designed to interrogate each target sequence in the transcripts. The probes span around 50 bases, allowing for the profiling of partially degraded RNA.

The assay probe set consists of an upstream oligonucleotide containing a gene specific sequence and a universal PCR primer sequence (P1) at the 5′ end, and a downstream oligonucleotide containing a gene specific sequence and a universal PCR primer sequence (P2) at the 3′ end. The upstream oligonucleotide hybridizes to the targeted cDNA site, and then extends and ligates to its corresponding downstream oligonucleotide to create a PCR template that can be amplified with universal PCR primers according to the manufacturer's instructions.

The resulting PCR products are hybridized to the HumanRef-8 Expression BeadChip to determine the presence or absence of specific genes. The HumanRef-8 BeadChip features up-to-date content covering >24,000 annotated transcripts derived from the National Center for Biotechnology Information Reference Sequence (RefSeq) database (Build 36.2, Release 22). For details see Tables 16 and 17.

TABLE 16
HumanRef-8 Expression Array

| Characteristic | Number |
|---|---|
| Transcripts | 24,526 |
| Genes | 18,401 |
| Probe Beads | ~1,000,000 |
| Probe Beads/Transcript | ~41 |
| Control Probes | ~850 |
| Probes for 50-base site on transcript | Two 25-mers |

TABLE 17
RefSeq* Content of the HumanRef-8 BeadChip

| Probes | Description | Number |
|---|---|---|
| NM | Coding transcripts, well established annotations | 23,811 |
| XM | Coding transcripts, provisional annotations | 426 |
| NR | Non-coding transcripts, well established annotations | 263 |
| XR | Non-coding transcripts, provisional annotations | 26 |
| Total | | 24,526 |

*Build 36.2, Release 22

After hybridization, HumanRef-8 Expression BeadChips are scanned using the iScan system. This system incorporates high-performance lasers, optics, and detection systems for rapid, quantitative scanning. The system offers a high signal-to-noise ratio, high sensitivity, low limit of detection, and broad dynamic range, leading to exceptional data quality.

Whole genome gene expression analysis using DASL chemistry microarrays allows for an estimate of whether a particular gene is producing inure or less mRNA in the tumor than in the cell type from which the tumor was derived. Based on the activity, greater or lesser, of a given gene, may increase the likelihood that a tumor will respond to a particular therapeutic depending on the type of cancer being treated. The differential gene expression of a subject's tumor when compared to normal tissue can provide a useful diagnostic tool for helping an oncologist determine the appropriate treatment route.

The DASL chemistry addresses the limitation of working with degraded FFPE RNA by deviating from the traditional direct hybridization microarray methodologies. However, there is much variability in fixation methods of FFPE tissue, which can lead to higher levels of RNA degradation. The DASL assay can be used for partially degraded RNAs, but not for entirely degraded RNAs. To qualify RNA samples prior to DASL assay analysis, RNA quality is checked using a real-time qPCR method where the highly expressed ribosomal protein gene, RPL13a, is amplified using SYBR green chemistry. If a sample has a cycle threshold value ≦29, then the sample is considered to be intact enough to proceed with the DASL chemistry. See Biotinylated cDNA Pre-Qualification, Illumina, Inc.; Abramovitz, M., et al., Optimization of RNA extraction from FFPE tissues for expression profiling in the DASL assay. Biotechniques, 2008. 44(3): p. 417-23. Any sample that has an A260/A280 ratio <1.5, or a RPL13a Ct value >30 is considered too degraded or too heavily modified to be processed using the Whole Genome DASL gene expression chemistry. See Abramovitz.

Prior to hybridization on the HumanRef-8 Expression BeadChip, the sample is precipitated. The sample precipitate will be in the form of a blue pellet. If the blue pellet is not visible for that sample, the sample must be re-processed prior to hybridization on the BeadChip.

Although the Whole Genome DASL assay examines the expression of thousands of genes, expression of only the genes of interest need be analyzed.

In order to standardize the reporting of patient data using the Illumina Whole Genome DASL technology, the algorithm below is used. The data is obtained using the Genome Studios Software v2009.1 (Gene Expression Module version 1.1.1).

Step 1: The detection p-values determined by the Genome Studios software must be less than 0.01. This value is determined by examining the variability of the signals generated by the duplicate copies of the same probe for a particular gene in relation to the variability observed in the negative control probes present on the array. If the detection p-value for either the control or the patient sample is greater than 0.01 for a particular gene the expression for that gene is reported out as “Indeterminate.” A cut-off of 0.01 was selected as it indicates that there is less than a one percent chance that the data would be observed given that the null hypothesis of no change in expression is true. The p-value can be corrected for multiple comparisons.

Step 2: The p-value of the differential expression must be less than 0.001. This p-value is determined by using the following equation: 1/(10̂(D/(10*SIGN(PS-CS)))). In this equation “D” represents the differential expression score that is generated by the Genome Studios. The “PS” and “CS” represents the relative fluorescence units (RFU) obtained on the array of a particular gene for the patient sample (PS) and control sample (CS) respectively. The “SIGN” function converts the sign of the value generated by subtracting the CS RFU from the PS RFU into a numerical value. If PS minus CS is >0 a value of 1 will be generated. If PS minus CS is <0 a value of −1 will be generated. If PS equals CS then a value of 0 will be generated. If the differential expression p-value is greater than 0.001 for any particular gene the expression for that gene is reported out as “No Change.” A cut off of 0.001 was chosen because genes passing this threshold can be validated as differentially expressed by alternative methods approximately 95% of the time.

Step 3: If the expression ratio is less than 0.66 for a particular gene, the expression for that gene will be reported out as “Underexpressed.” If the expression ratio is greater than 1.5, the expression for that gene will be reported out as “Overexpressed.” If the expression ratio is between 0.66 and 1.5 the expression for a particular gene will be reported out as “No Change.” The expression ratio is determined by obtained by dividing the RFUs for a gene from the patient sample by the RFUs for the same gene from the control sample (PS/CS). “No Change” indicates that there is no difference in expression for this gene between tumor and control tissues at a significance level of p≦=0.001. A significance level of p≦=0.001 was chosen since genes passing this threshold can be validated as differentially expressed by alternative methods approximately 95% of the time.

“Not Informative (NI)” indicates that the data obtained for either the patient sample or the control sample were not of high enough quality to confidently make a call on the expression level of that particular RNA transcript.

Step 4: In some where FFPE samples only are used, all genes that are identified as “Under expressed”, using the above algorithm, will be reported out as “Indeterminate.” This is due to the degraded nature of the RNA obtained from FFPE samples and as such, it may not be possible to determine whether or not the reduced RFUs for a gene in the patient sample relative to the control sample is due to the reduced presence of that particular RNA or if the RNA is highly degraded and impeding the detection of that particular RNA transcript. With improved technologies, some or all genes as “Underexpressed” with FFPE samples are reported.

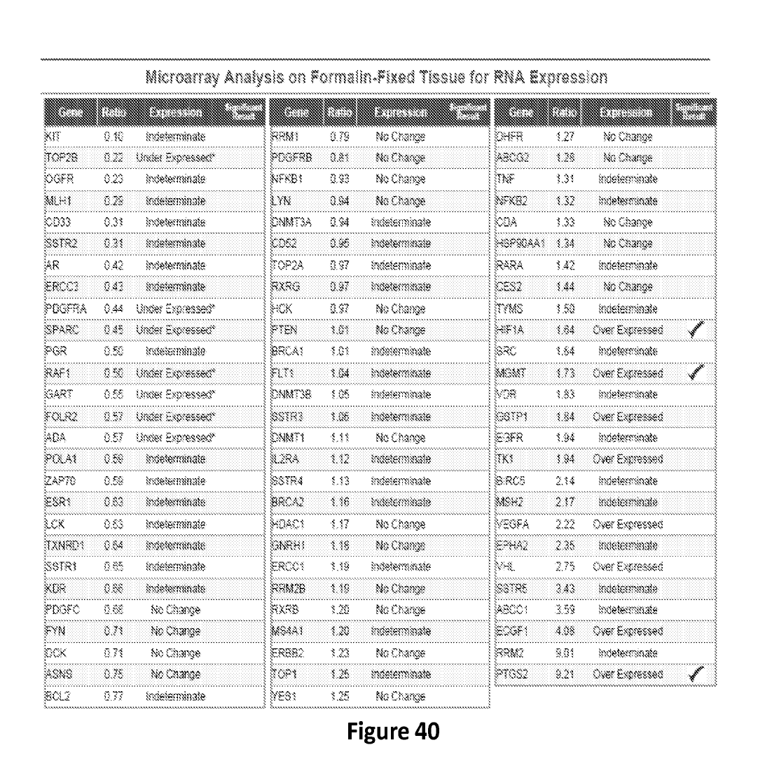

FIG. 40 shows results obtained from microarray profiling of an FFPE sample. Total RNA was extracted from tumor tissue and was converted to cDNA. The cDNA sample was then subjected to a whole genome (24K) microarray analysis using Illumina cDNA-mediated annealing, selection, extension and ligation (DASL) process. The expression of a subset of 80 genes was then compared to a tissue specific normal control and the relative expression ratios of these 80 target genes indicated in the figure was determined as well as the statistical significance of the differential expression.

### Example 7

### Molecular Profiling System and Report

A system has several individual components including a gene expression array using the Illumina Whole Genome DASL Assay as described in Example 6. In addition to this gene expression array, the system also performs a subset of immunohistochemistry assays on formalin fixed paraffin embedded (FFPE) cancer tissue. Gene copy number is determined for a number of genes via FISH (fluorescence in situ hybridization) and mutation analysis is done by DNA sequencing for a several specific mutations. All of this data is stored for each patient case. Data is reported from the microarray, IHC, FISH and DNA sequencing analysis. All laboratory experiments are performed according to Standard Operating Procedures (SOPs).

DNA for mutation analysis is extracted from formalin-fixed paraffin-embedded (FFPE) tissues after macrodissection of the fixed slides in an area that % tumor nuclei ≧10% as determined by a pathologist. Extracted DNA is only used for mutation analysis if % tumor nuclei ≧10%. DNA is extracted using the QIAamp DNA FFPE Tissue kit according to the manufacturer's instructions (QIAGEN Inc., Valencia, Calif.). DNA can also be extracted using the QuickExtract™ FFPE DNA Extraction Kit according to the manufacturer's instructions (Epicentre Biotechnologies, Madison, Wis.). The BRAF Mutector I BRAF Kit (TrimGen, cat#MH1001-04) is used to detect BRAF mutations (TrimGen Corporation, Sparks, Md.). The DxS KRAS Mutation Test Kit (DxS, #KR-03) is used to detect KRAS mutations (QIAGEN Inc., Valencia, Calif.). BRAF and KRAS sequencing of amplified DNA is performed using Applied Biosystems' BigDye® Terminator V1.1 chemistry (Life Technologies Corporation, Carlsbad, Calif.).

IHC is performed according to standard protocols. IHC detection systems vary by marker and include Dako's Autostainer Plus (Dako North America, Inc., Carpinteria, Calif.), Ventana Medical Systems Benchmark® XT (Ventana Medical Systems, Tucson, Ariz.), and the Leica/Vision Biosystems Bond System (Leica Microsystems Inc., Bannockburn, Ill.). All systems are operated according to the manufacturers' instructions. American Society of Clinical Oncology (ASCO) and College of American Pathologist (CAP) standards are followed for ER, PR, and HER2 testing. ER, PR and HER2 as well as Ki-67, p53, and E-cad IIICs analyzed by the ACIS® (Automated Cellular Imaging System). The ACIS system comprises a microscope that scans the slides and constructs an image of the entire tissue section. Ten areas of tumor are analyzed for percentage positive cells and staining intensity within the selected fields.

FISH is performed on formalin-fixed paraffin-embedded (FFPE) tissue. FFPE tissue slides for FISH must be Hematoxylin and Eosin (H&E) stained and given to a pathologist for evaluation. Pathologists will mark areas of tumor for FISH analysis. The pathologist report shows whether tumor is present and sufficient enough to perform a complete analysis. FISH is performed using the Abbott Molecular VP2000 according to the manufacturer's instructions (Abbott Laboratories, Des Plaines, Iowa).

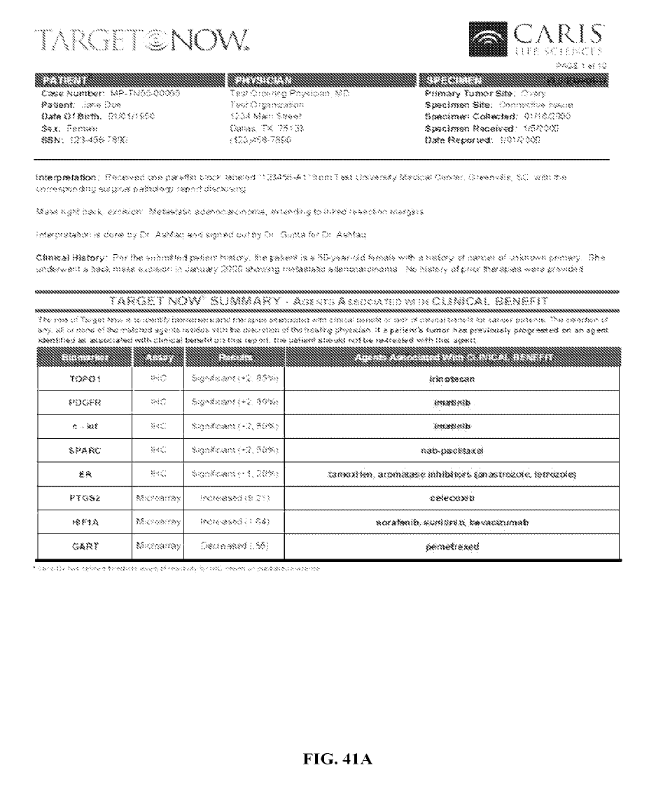

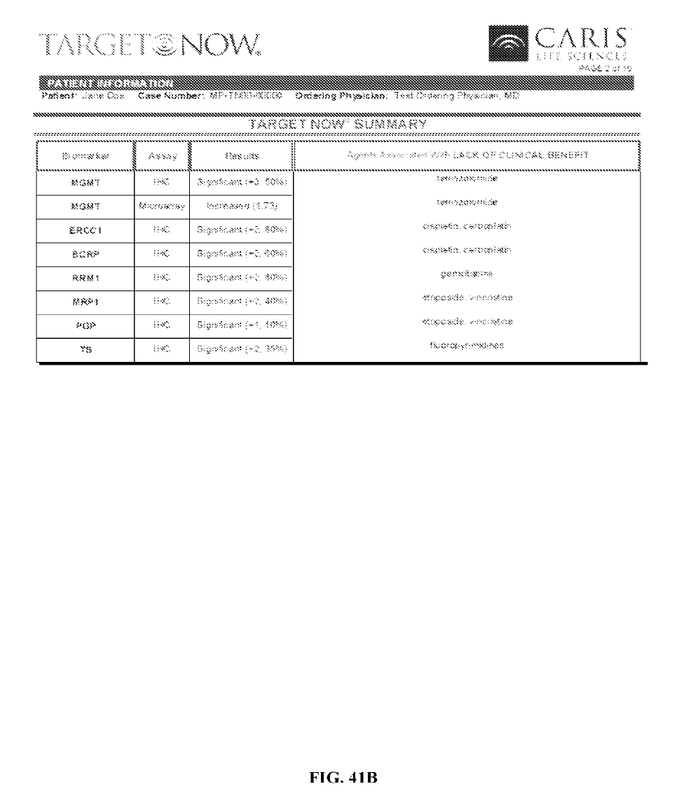

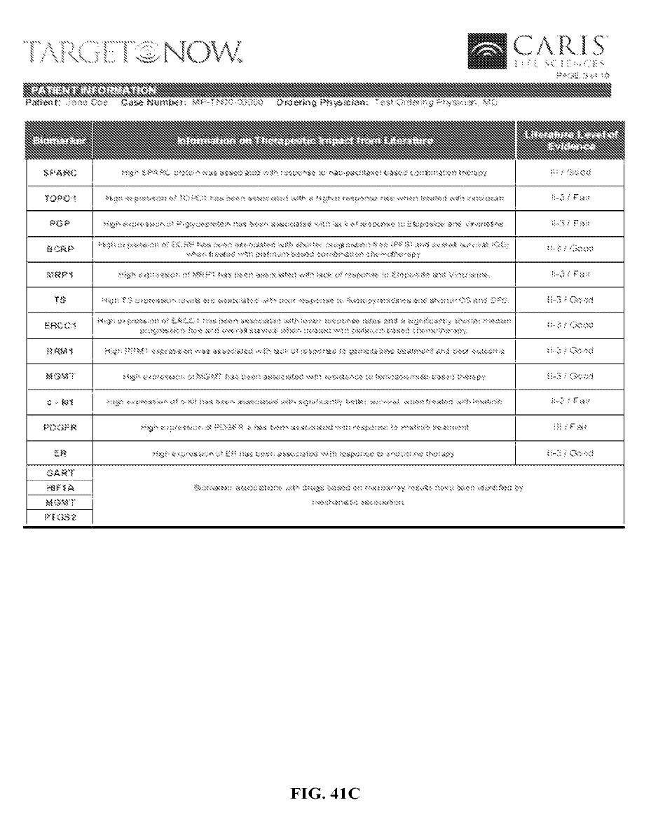

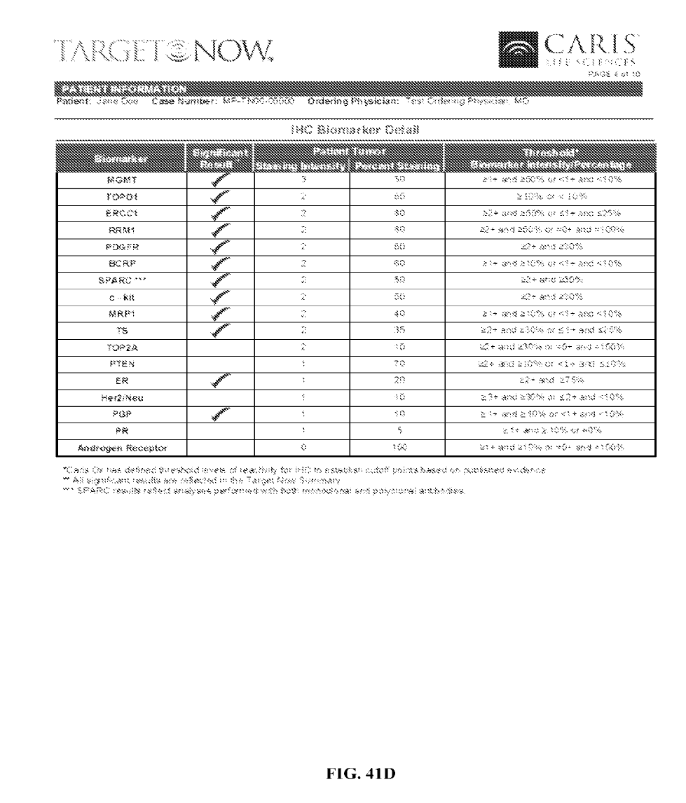

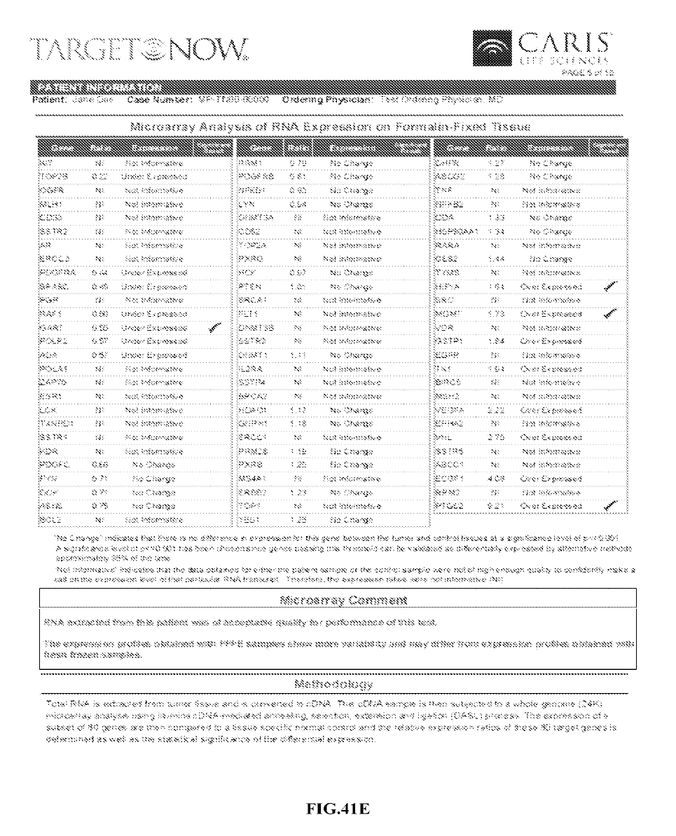

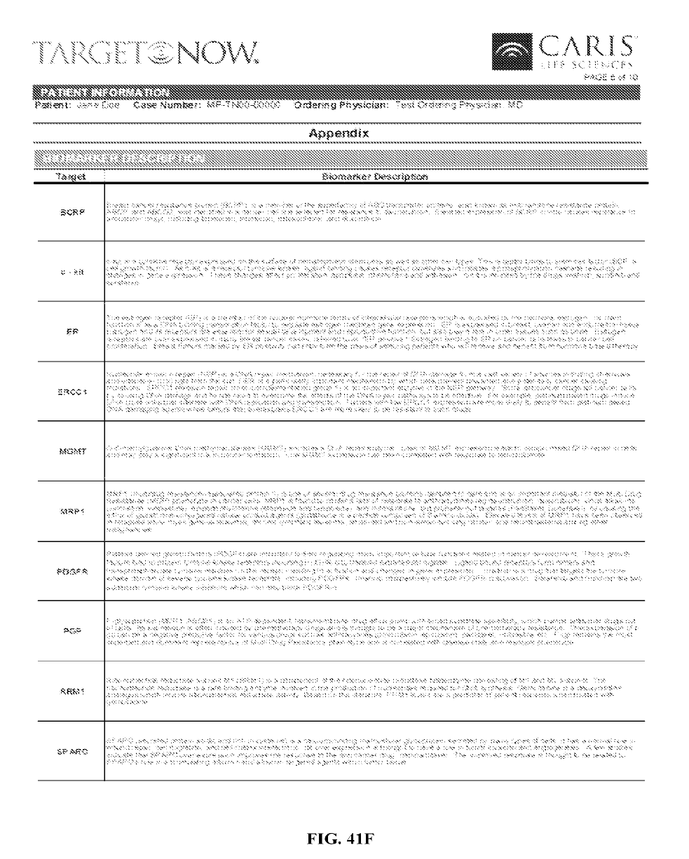

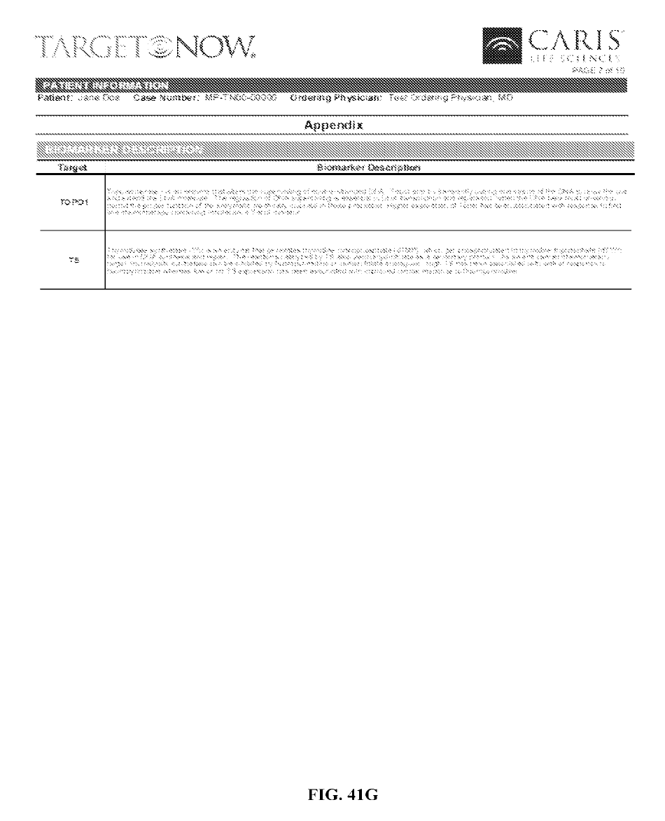

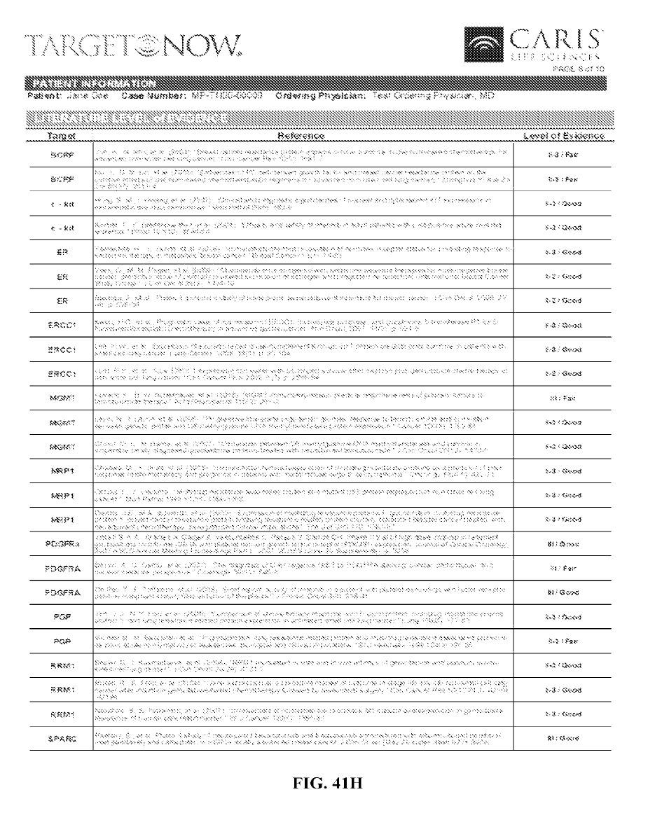

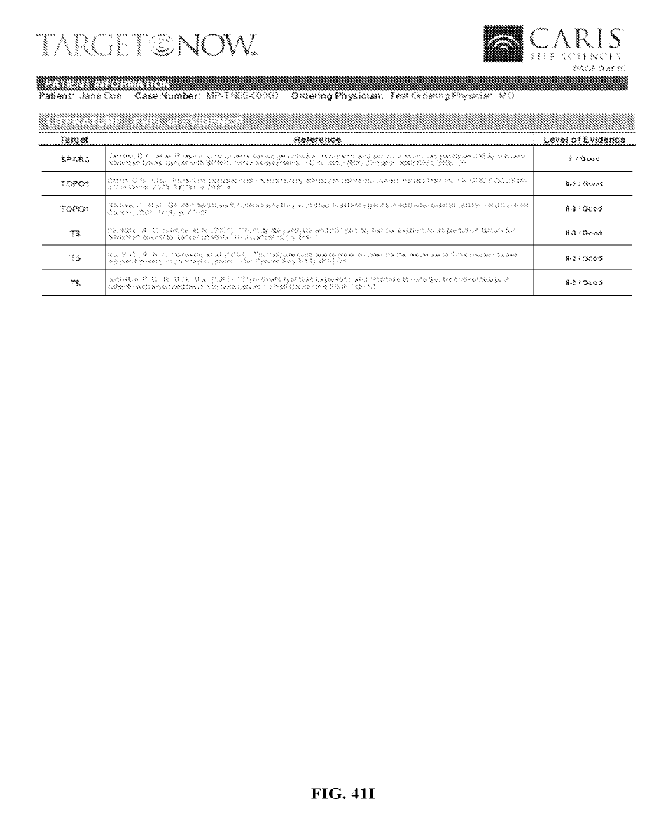

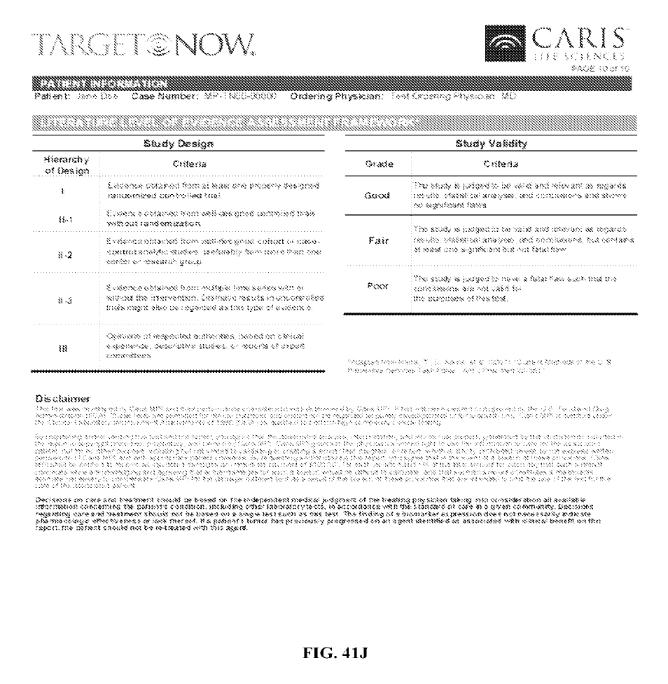

A report generated by the system in shown in FIGS. 41A-41J. FIG. 41A shows that the patient had a primary tumor in the ovary. A paraffin block sample was used. FIGS. 41A-41B illustrate a Summary listing of biomarkers identified as differentially expressed by microarray or IHC analysis. Treatment options corresponding to each differentially expressed biomarker is presented. The subject's caregiver (e.g., physician) can decide which candidate treatments to apply. FIG. 41C presents a table of literature evidence linking the candidate treatments to the biomarkers. FIG. 41D presents the results of IHC analysis and FIG. 41E presents the results of microarray analysis. FIGS. 41F-41G present a summary description of the differentially expressed biomarkers. FIGS. 41H-41I present a summary description of literature supporting the candidate therapeutics linked to the differentially expressed biomarkers with a rating for the level of evidence attached to each publication. FIG. 41J presents a chart explaining the codes for level of evidence.

### Example 8

### Workflow for Identifying a Therapeutic Agent for Breast Cancer

FIG. 42 illustrates a diagram that outlines a workflow for identifying a therapeutic agent by analyzing a sample from an individual with breast cancer (421). The sample is cut into a number of slides (422) and stained with hematoxylin and eosin (H&E) (423). The stained slides are read by a pathologist (424) to determine what panel of markers to test, e.g., whether to analyze the sample using a complete biomarker panel analysis or a tumor-specific biomarker panel analysis, e.g., for breast cancer sample analysis (425). The pathologist also identifies sections (426) for DNA microarray analysis (427), FISH analysis, e.g., to measure IIER2 expression (428), or mutational analysis via sequencing (429). DNA microarray analysis can be performed on a whole genome scale, with focus on genes that are informative for therapeutic treatment options, including at least ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. IHC is run on selected sections to analyze expression of biomarkers including AR, c-kit, CAV-1, CK 5/6, CK14, CK17, ECAD, ER, Her2/Neu, Ki67, MRP1, P53, PDGFR, PGP, PR, PTEN, SPARC, TLE3 and TS (4210). Each marker can be analyzed using a single or multiple antibodies for IHC detection. For example, SPARC is detected using an anti-SPARC monoclonal antibody (referred to herein as SPARC MC, SPARC Mono, SPARC m or the like), and an anti-SPARC polyclonal antibody (referred to herein as SPARC PC, SPARC Poly, SPARC p or the like), Given the results of the previous analysis, the sample is further analyzed with relevant marker panels (4211). The sample is classified as HER2+(4212), Triple Negative (4216), or ER/PR+, HER2− (4218). Further analysis depends on whether prior analysis determined that the sample should undergo “complete” biomarker panel analysis or a “tumor-specific” biomarker panel analysis. Tumor-specific analysis is performed for any cancer with a primary diagnosis, or first line, second line or third line therapy. Complete biomarker analysis is indicated for cancers that are fourth line, metastatic or beyond. Complete is also performed if the therapeutic history of the cancer is unknown (and thus becomes the default). In this manner, unnecessary testing can be avoided. HER2+(4212) samples are further analyzed by FISH for CMYC and TOP2A (4213), by IHC for p95 for tumor-specific analysis or for BCRP, ERCC1, MGMT, P95, RRM1, TOP2A and TOPOI for complete analysis (4214), and by sequencing for mutation analysis of PIK3CA (4215). Triple negative (4216) samples are analyzed by IIIC for p95 for tumor-specific analysis or for BCRP, ERCC1, MGMT, P95, RRM1, TOP2A and TOPO1 for complete analysis (4217). ER/PR+, HER2− (4218) samples are further analyzed by FISH for CMYC (4219), by IHC for p95 for tumor-specific analysis or for BCRP, ERCC1, MGMT, P95, RRM1, TOP2A and TOPO1 for complete analysis (4220). The results of the analysis are used to identify a therapeutic for the individual. The workflow can be generalized for the analysis of other diseases and tumor types.

FIGS. 43A-B illustrate a biomarker centric view of the workflow described above. In FIG. 43A, initial IHC and FISH results on the indicated biomarkers is used to characterize the cancer as HER2+, Triple Negative, or ER/PR+, HER2−. The characterization guides the additional IHC, FISH and sequencing analysis that is performed. “DNA MA” indicates that a DNA microarray is performed on all samples that meet the quality threshold as described herein. DNA microarray analysis can be performed on a whole genome scale, with focus on genes that are informative for therapeutic treatment options, including at least ABCC1, ABCG2, ADA, AR, ASNS, BCL2, BIRC5, BRCA1, BRCA2, CD33, CD52, CDA, CES2, DCK, DIIFR, DNMT1, DNMT3A, DNMT3B, ECGF1, EGFR, EPIIA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. IHC is run on selected sections to analyze expression of biomarkers including AR, c-kit, CAV-1, CK 5/6, CK14, CK17, ECAD, ER, Her2/Neu, Ki67, MRP1, P53, PDGFR, PGP, PR, PTEN, SPARC, TLE3 and TS. FIG. 43B outline shows the criteria used to perform additional assays. Tumor-specific analysis is used in the case of cancer with a primary diagnosis, or first line, second line or third line therapy. Complete biomarker analysis is indicated for cancers that are fourth line, metastatic or beyond.

Table 18 indicates prognostic markers in the breast cancer profiling. The markers used in the profiling can be used for theranostic (e.g., to guide selection of a candidate therapeutic) and prognostic purposes. “Y” in the “Prognostic?” column indicates that the marker can indicate a prognosis. Further details are described herein.

TABLE 18
Prognostic Breast Cancer Profiling

| | | | | Triple | ER/PR+/ |
|---|---|---|---|---|---|
| | | | HER2+ | Neg | HER2- |
| Biomarker | Method | Prognostic? | Profile | Profile | Profile |
| AR | IHC | | Y | Y | Y |
| Caveolin-1 | IHC | Y | Y | Y | Y |
| CK 14 | IHC | Y | Y | Y | Y |
| CK 17 | IHC | Y | Y | Y | Y |
| CK 5/6 | IHC | Y | Y | Y | Y |
| c-Kit | IHC | Y | Y | Y | Y |
| cMYC | FISH | Y | Y | | Y |
| Cyclin D1 | IHC | Y | | | Y |
| ECAD | IHC | Y | Y | Y | Y |
| EGFR | IHC | Y | | | Y |
| ER (ESR1) | IHC | | Y | Y | Y |
| HER2 | IHC/ | | Y | Y | Y |
| (ERBB2) | FISH | | | | |
| Ki67 | IHC | | Y | Y | Y |
| MRP1 | IHC | | Y | Y | Y |
| (ABCC1) | | | | | |
| P53 | IHC | Y | Y | Y | Y |
| P95 | IHC | | Y | Y | Y |
| PDGFR | IHC | Y | Y | Y | Y |
| PGP (ABCB1) | IHC | | Y | Y | Y |
| PI3K | SEQ | | Y | | |
| PR | IHC | | Y | Y | Y |
| PTEN | IHC | | Y | Y | Y |
| SPARC | IHC | | Y | Y | Y |
| TLE3 | IHC | | Y | Y | Y |
| TOP2A | FISH | | Y | | |
| TOP2A | IHC | | Y | Y | Y |
| TS (TYMS) | IHC | | Y | Y | Y |

Table 19 provides illustrative candidate treatments corresponding to the molecular profiling described in this Example. In the table, a positive result for the indicated biomarker using the indicated technique guides selection of the corresponding therapeutic agent, or that of a related agent.

TABLE 19
Illustrative Drug-biomarker Associations

| Drug | Method | Biomarker(s) |
|---|---|---|
| 5-fluorouracil | DNA Microarray | TYMS |
| | IHC | TS |
| aminoglutethimide | DNA Microarray | ESR1, PR |
| | IHC | ER, PR |
| anastrozole | DNA Microarray | ESR1, PR |
| | IHC | ER, PR |
| capecitabine | DNA Microarray | TYMS |
| | IHC | TS |
| doxorubicin | DNA Microarray | ABCB1, TOP2A |
| | FISH | HER2, TOP2A |
| | IHC | PGP, TOP2A |
| epirubicin | DNA Microarray | ABCB1, TOP2A |
| | FISH | HER2, TOP2A |
| | IHC | PGP, TOP2A |
| exemestane | DNA Microarray | ESR1, PR |
| | IHC | ER, PR |
| fulvestrant | DNA Microarray | ESR1, PR |
| | IHC | ER, Ki67, PR |
| gonadorelin | DNA Microarray | PR |
| goserelin | DNA Microarray | PR |
| irinotecan | IHC | TOPO1 |
| lapatinib | FISH | HER2 |
| | IHC | HER2 |
| letrozole | DNA Microarray | ESR1, PR |
| | IHC | ER, PR |
| leuprolide | DNA Microarray | PR |
| liposomal- | DNA Microarray | ABCB1, TOP2A |
| doxorubicin | FISH | HER2, TOP2A |
| | IHC | PGP, TOP2A |
| medroxyprogesterone | DNA Microarray | ESR1, PR |
| | IHC | ER, PR |
| megestrol acetate | DNA Microarray | ESR1, PR |
| | IHC | ER, PR |
| methotrexate | DNA Microarray | ABCC1, DHFR |
| | IHC | MRP1 |
| nab-paclitaxel | DNA Microarray | SPARC |
| | IHC | SPARC mono, SPARC poly |
| pemetrexed | DNA Microarray | DHFR, GART, TYMS |
| | IHC | TS |
| tamoxifen | DNA Microarray | ESR1, PR |
| | IHC | ER, Ki67, PR |
| taxanes | IHC | TLE3 |
| toremifene | DNA Microarray | ESR1, PR |
| | IHC | ER, Ki67, PR |
| trastuzumab | FISH | HER2 |
| | IHC | HER2, P95, PTEN |
| | Mutation | PIK3CA |
| | (sequence analysis) | |

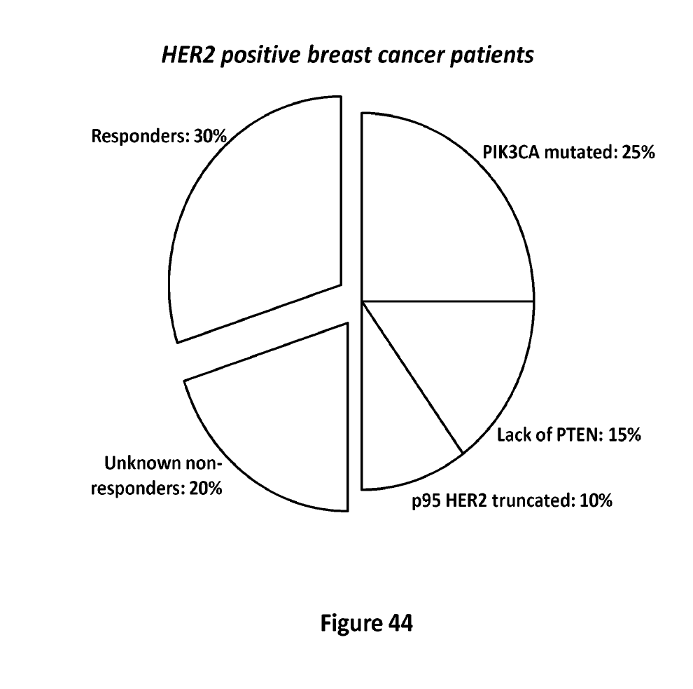

An illustrative benefit of the molecular profiling approach is illustrated in FIG. 44. For every 100 HER2+ patients, only about 30 (30%) will be Responders to treatment with trastuzumab. Molecular profiling according to the Example identifies 50 (50%) out of the 70 patients (70%) not likely to respond, e.g., because of PIK3CA mutations (25%), lack of PTEN (15%) or a p95 HER2 truncation (10%). HER2 spans the cell membrane and trastuzumab binds the external portion of the protein. However, most HER2 tests, including the FDA approved tests available from Dako (Dako North America, Inc., Carpinteria, Calif.) and Ventana (Ventana Medical Systems, Inc., Tucson, Ariz.), target the internal domain of HER2. Profiling according to the invention uses two antibodies for HER2: one with affinity to the internal domain, another with affinity to both the internal and external domains. If the latter antibody is negative but the tests targeting the internal domain are positive (e.g., the FDA approved tests), then HER2 is “p95 truncated” and trastuzumab will not be effective. By identifying patients unlikely to respond, efficacy of trastuzumab for a selected population can be increased from 30% to 60%. Furthermore, the molecular profiling methods of the invention can identify candidate treatments that are more likely to be effective in the trastuzumab non-responders.

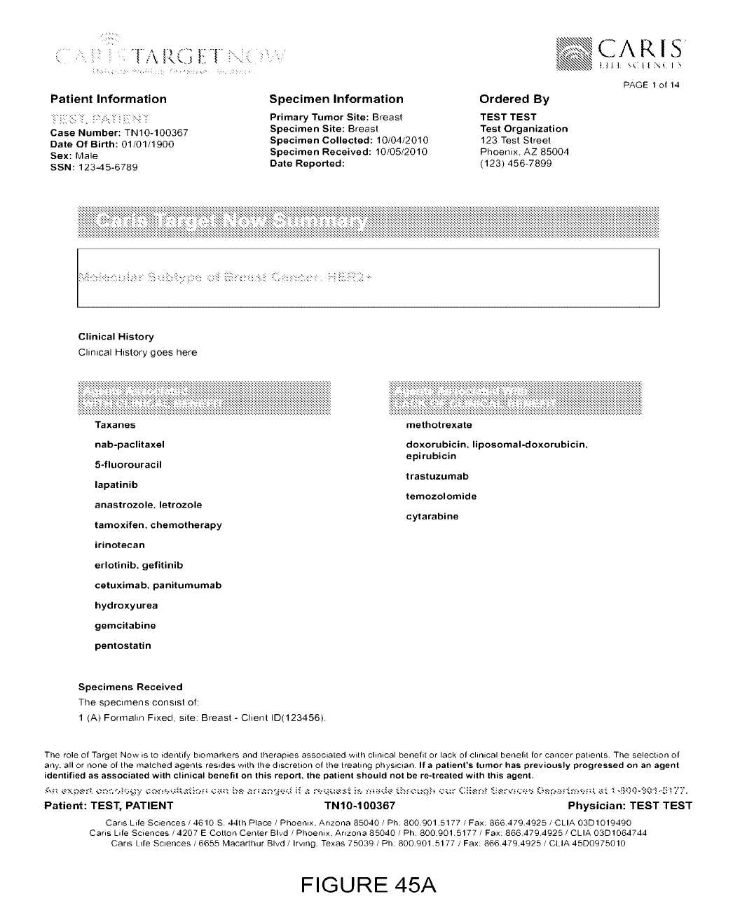

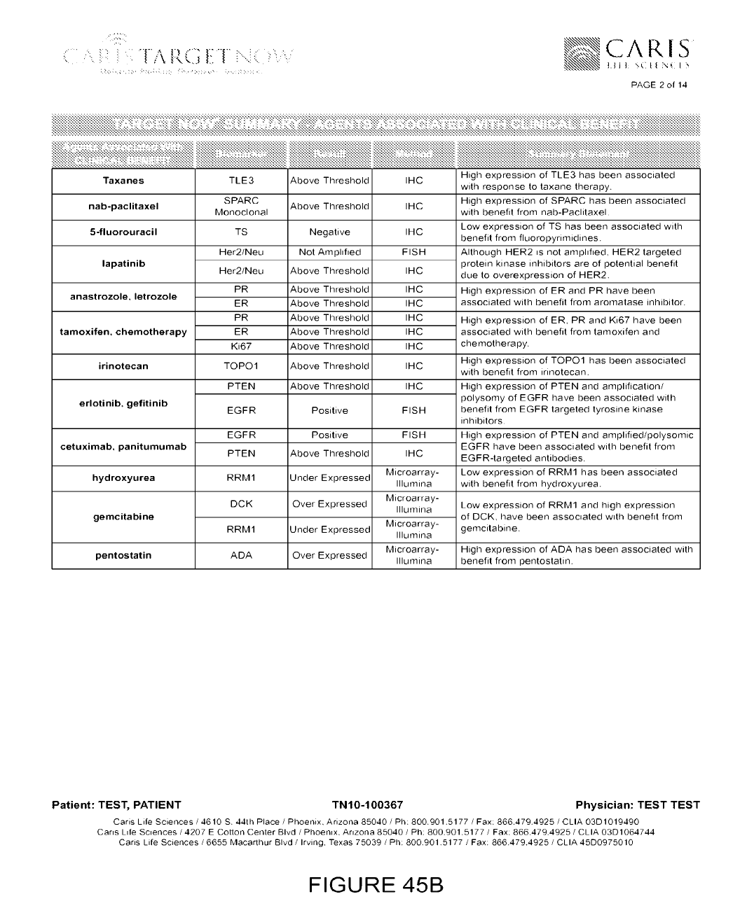

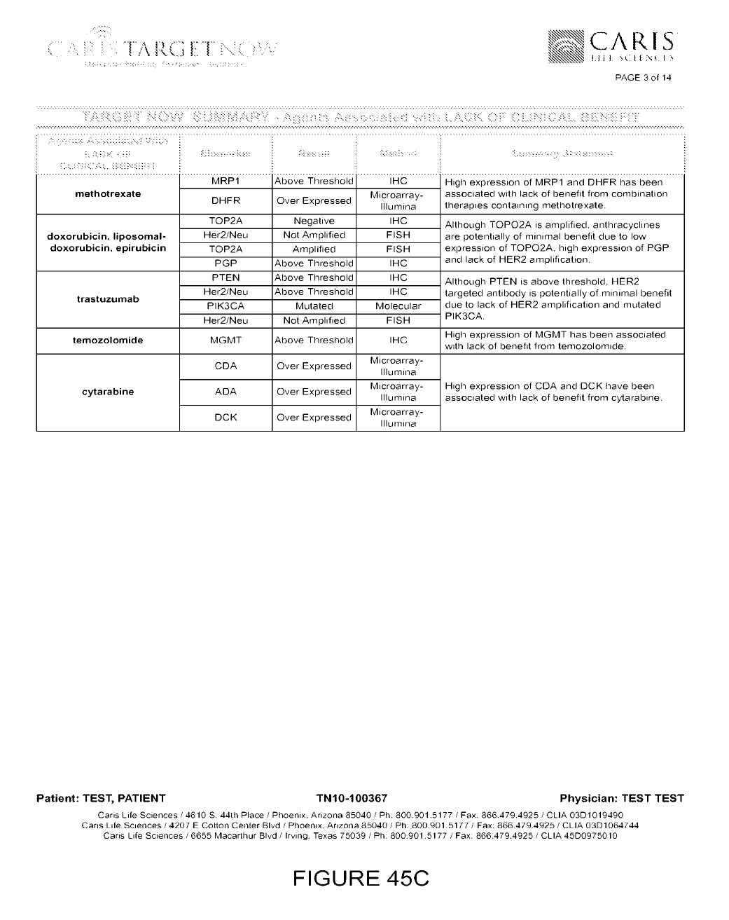

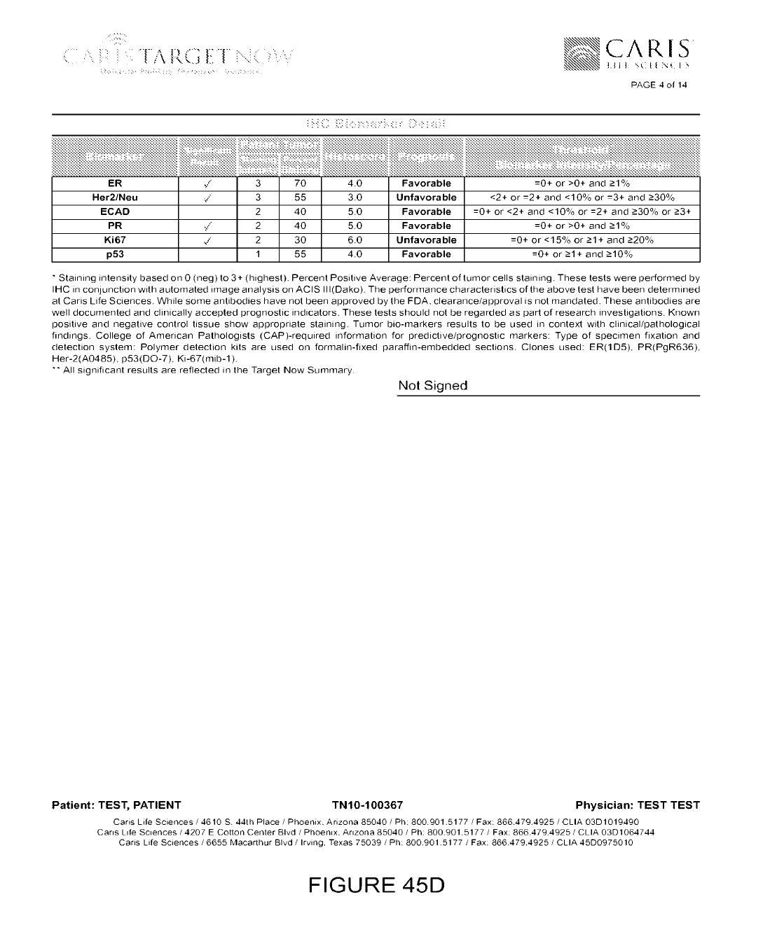

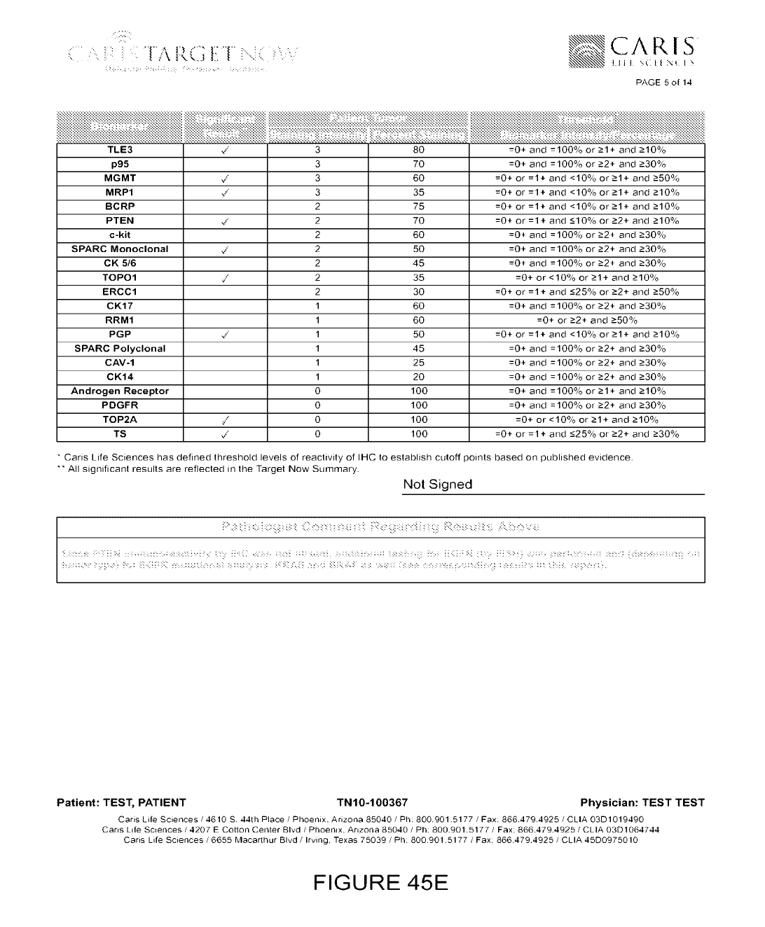

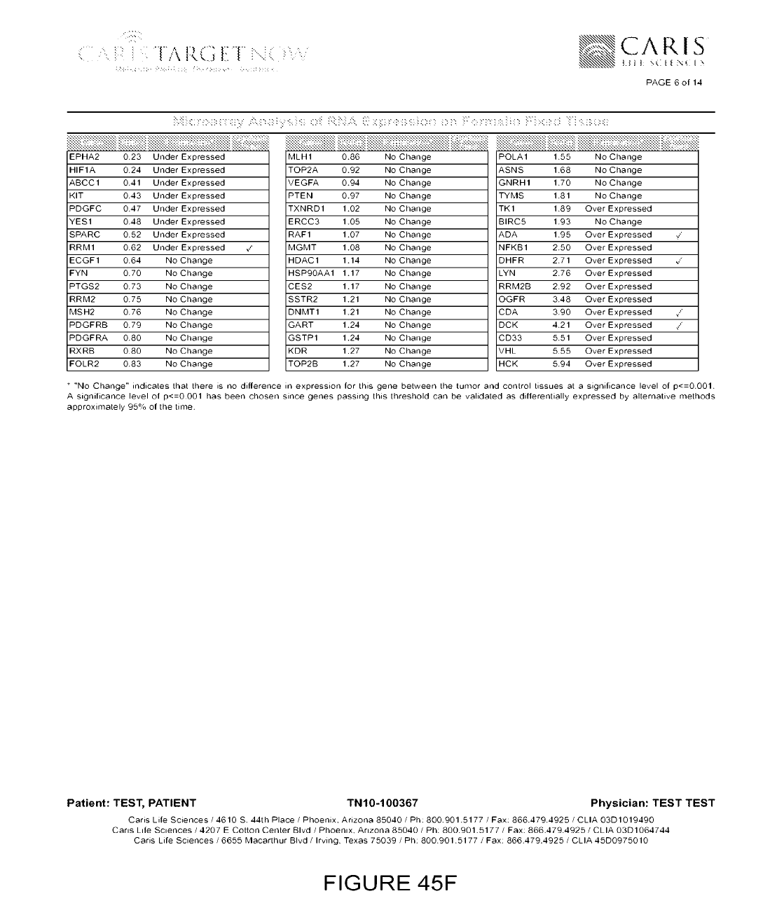

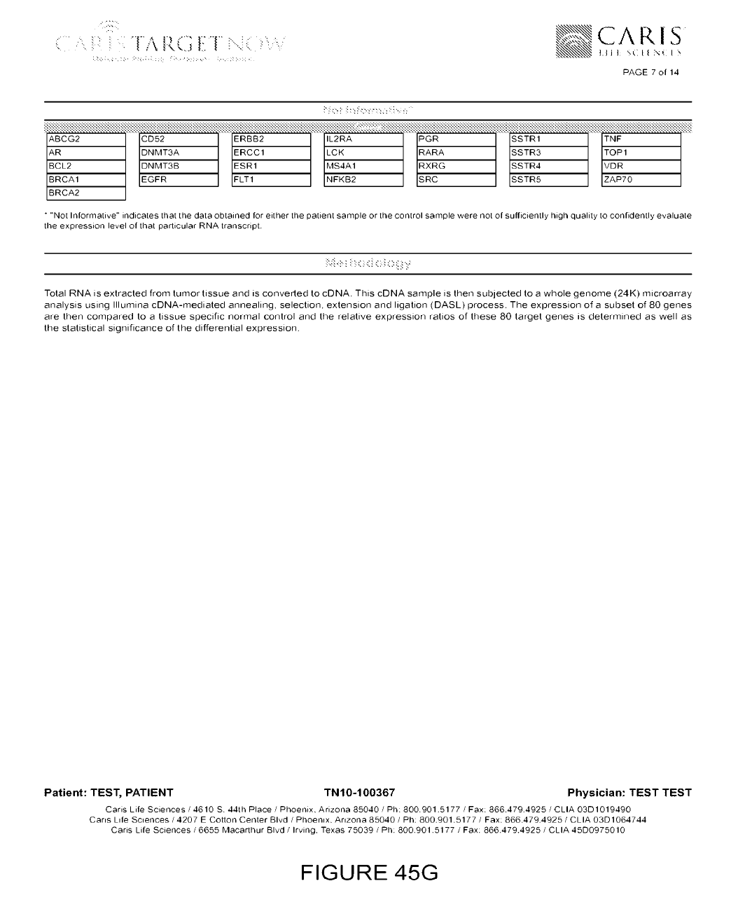

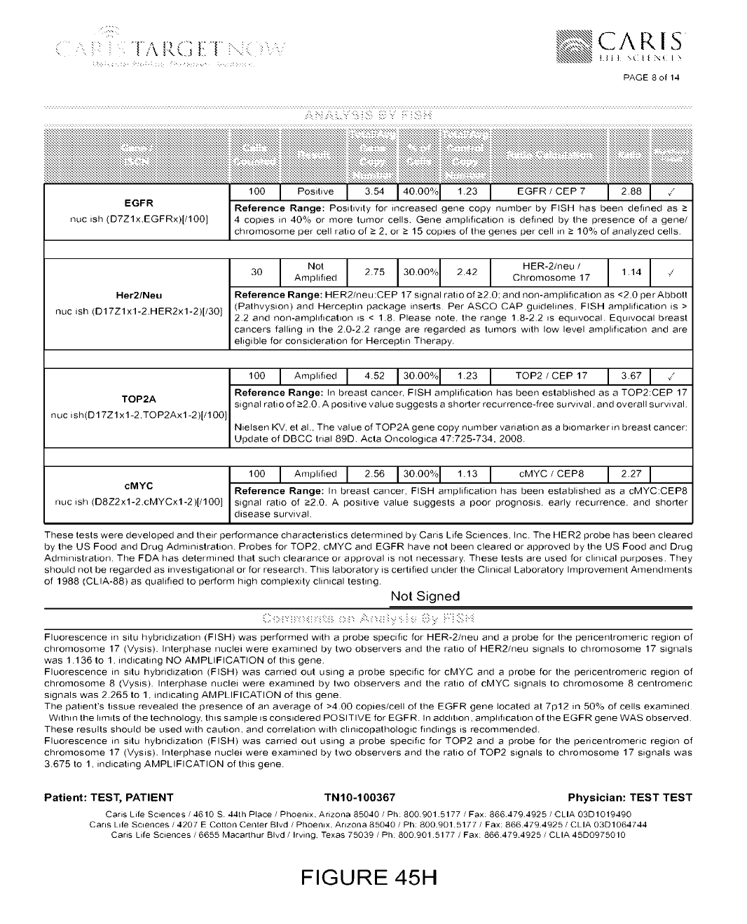

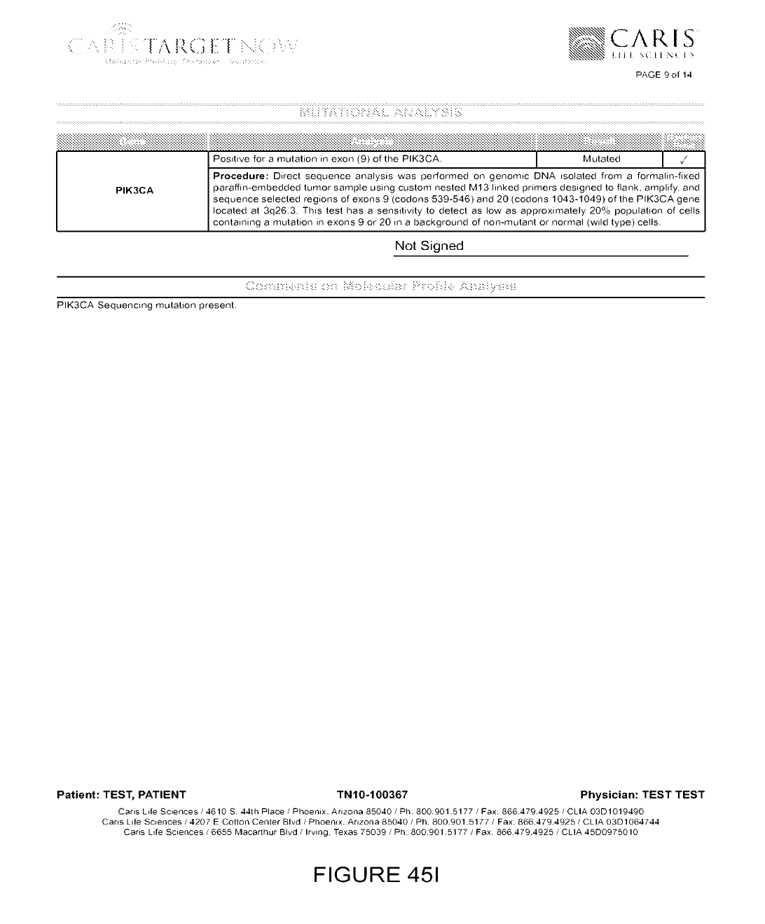

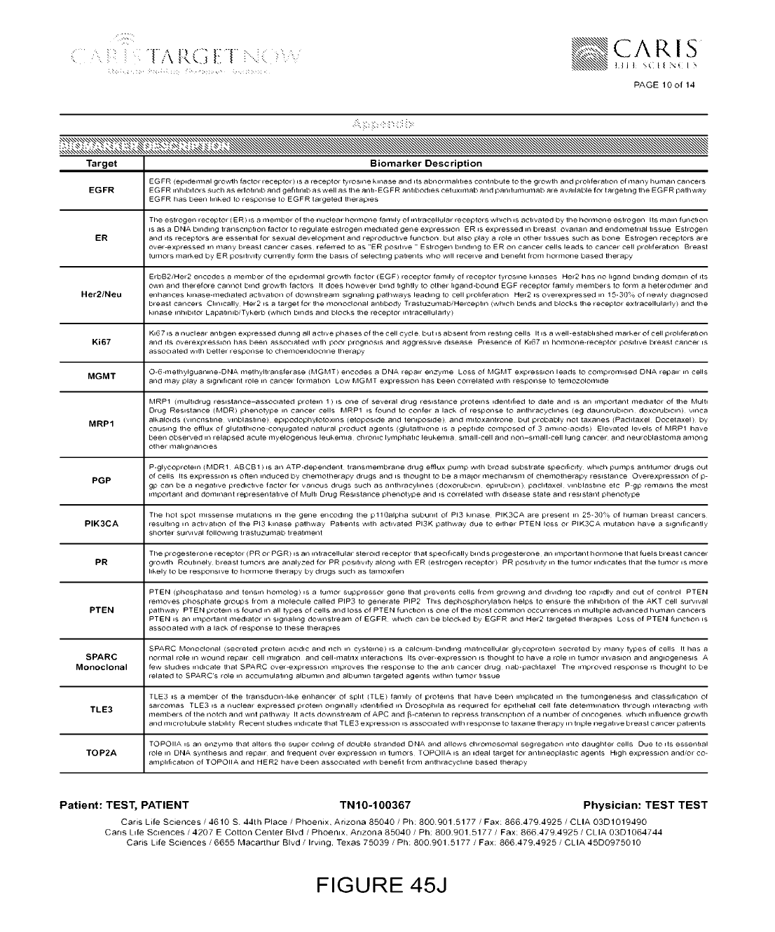

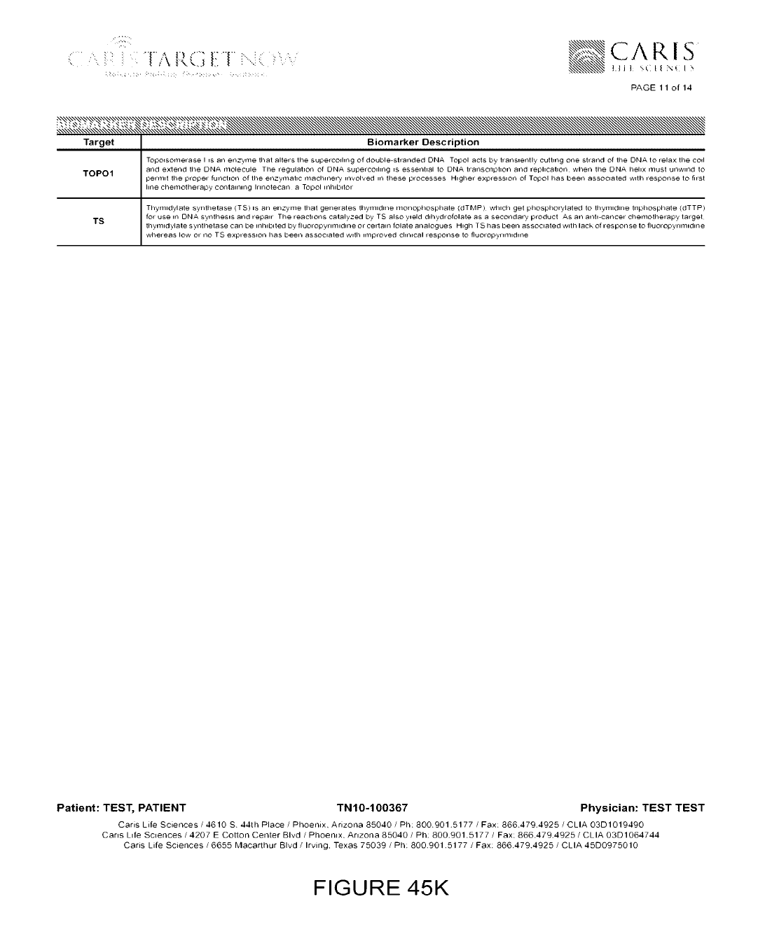

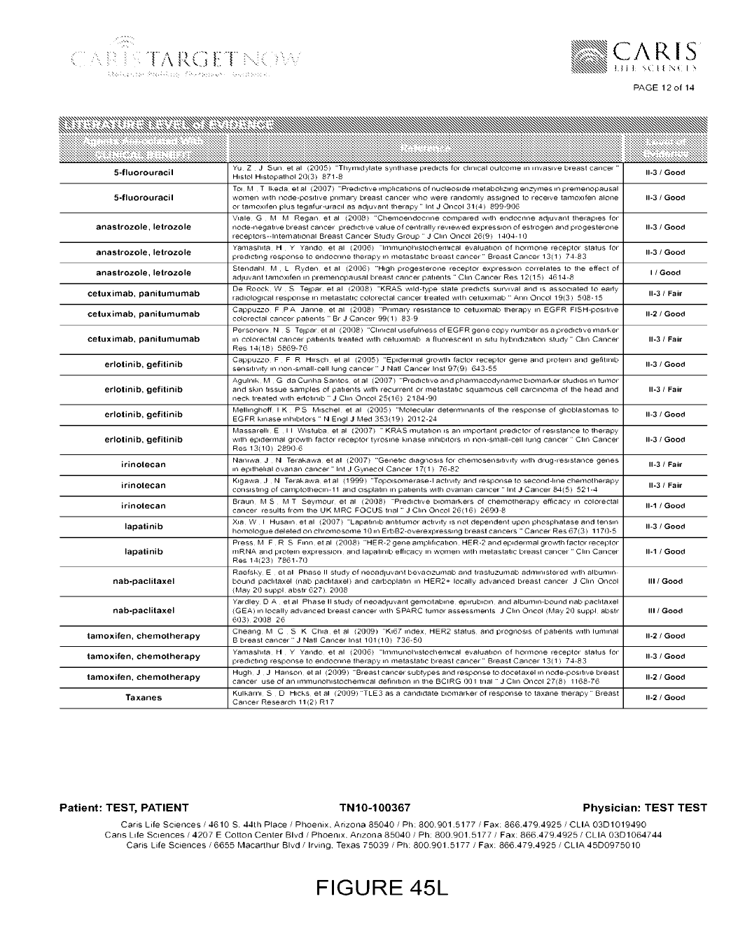

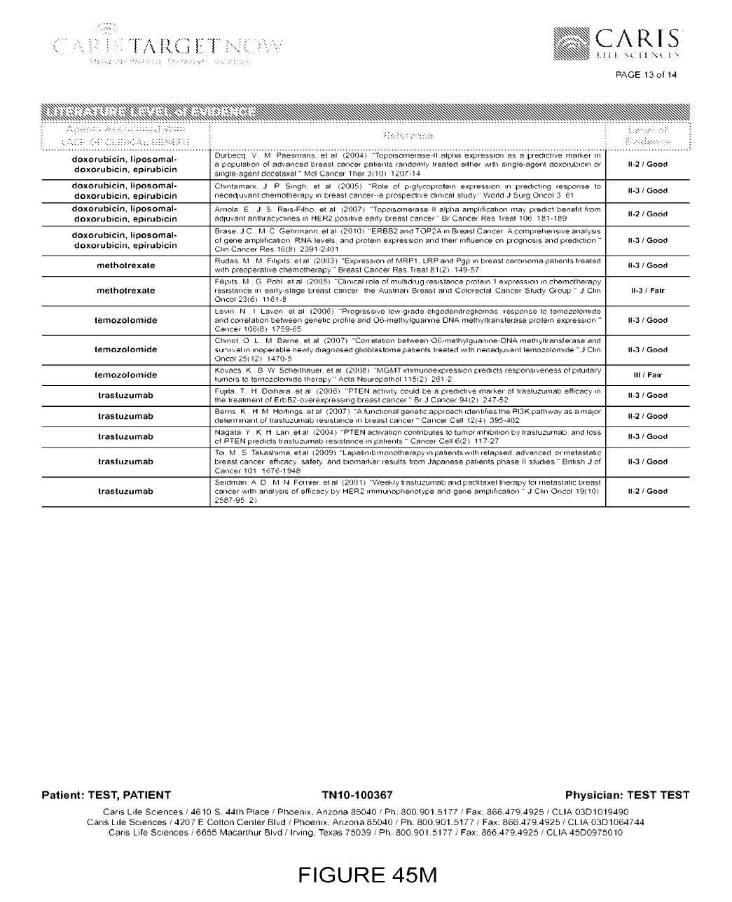

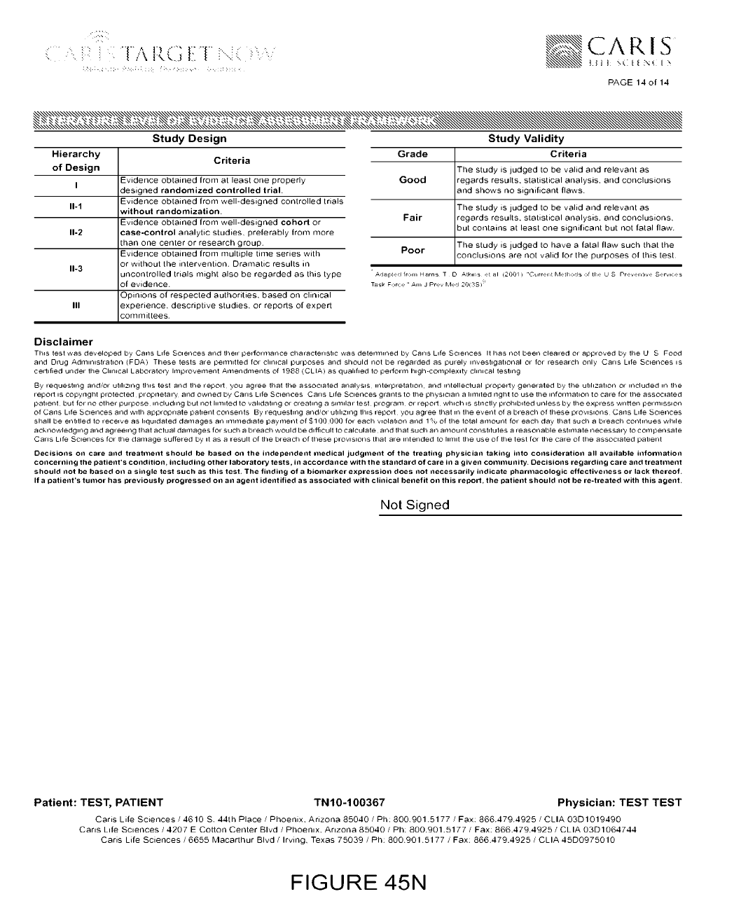

An illustrative report generated by the system in shown in FIGS. 45A-45N. FIG. 45A shows that the patient had a primary tumor in the breast determined to be HER2+, and provides a Summary of candidate therapeutic agents associated with beneficial or not for treating the tumor based on molecular profiling results. FIG. 45B illustrates a more detailed Summary listing for each agent associated with benefit, including the informative biomarkers and experimental methods used to assess those biomarkers. FIG. 45C illustrates a more detailed Summary listing for each agent associated with lack of benefit, including the informative biomarkers and experimental methods used to assess those biomarkers. FIG. 45D and FIG. 45E present the results of IHC analysis. FIG. 45F and FIG. 45G present the results of DNA microarray analysis, wherein results for informative biomarkers are shown in FIG. 45F whereas the non-informative biomarkers are shown in FIG. 45G. “Non-informative” indicates that the data obtained for the patient sample or control sample were not of sufficiently high quality to confidently evaluate the expression level of those RNA transcripts. FISH analysis is presented in FIG. 45H and mutational analysis is presented in FIG. 45I. Mutational analysis included direct sequence analysis of exon 9 of PIK3CA. FIG. 45J and FIG. 45K present a summary description of the relevant biomarkers. FIG. 45L and FIG. 45M present a summary description of literature supporting the candidate therapeutics linked to the informative biomarkers with a rating for the level of evidence attached to each publication. FIG. 45N is a chart depicting the codes for level of evidence.

### Example 9

### Biomarker and Drug-Centric Molecular Profiling

FIG. 46 illustrates a diagram showing a biomarker centric (FIG. 46A) and therapeutic centric (FIG. 46B) approach to identifying a therapeutic agent. Mutational analysis is performed on the markers with symbols in italics. This typically comprises a sequencing approach (e.g., Sanger sequencing or pyrosequencing) or an amplification approach (e.g., real time PCR). ISH, e.g., FISH, is performed on the markers whose symbols are underlined. The remaining markers are analyzed by IHC. DNA microarrays are performed on all samples with RNA of sufficient quality. In the biomarker-centric approach of FIG. 46A, the panel of markers that are run on a sample to identify a candidate therapeutic can depend on the origin of the tumor. Each circle surrounds the markers that are analyzed for a cancer of the indicated origin. Markers analyzed for breast cancers include FISH for cMYC and HER2, mutational analysis for PIK3CA, and IHC for P53, Ki67, p95, CK 14, CK 5/6, Cyclin D1, CAV-1, CK17, EGFR, ECAD, c-kit, MGMT, PDGFR, AR, MPR1, SPARC, PTEN, TOP2A, TS, PR, ER, PGP, HER2 and TLE3. Markers analyzed for ovarian cancers include FISH for IIER2, and MC for TOP2A, TS, PR, ER, PGP, IIER2, TLE3, BRCA1, BRCA2, IGFRBP3, IGFRBP4, IGFRBP5, TOPO1, ERCC1 and RRM1. Markers analyzed for colorectal cancers include sequencing for BRAF and KRAS, and IHC for TOP2A, TS, PTEN and COX2. Markers analyzed for lung cancers include FISH for EGFR, EML4-ALK fusion and MET, sequencing for EGFR, BRAF and KRAS, and IIIC for TOP2A, PTEN, COX2, TOPO1, ERCC1, RRM1, MPR1, SPARC, BCRP, β-III tubulin, IGFR1 and cMET. Analysis according to the “complete” (e.g., non-origin based) approach include FISH for EGFR and HER2, sequencing for EGFR, c-kit, BRAF and KRAS, and IHC for TOP2A, PTEN, TS, COX2, TOPO1, ERCC1, RRM1, MPR1, SPARC, BCRP, c-kit, MGMT, PDGFR, AR, PR, ER, PGP, and HER2. Additional markers that can be incorporated into biomarker-centric profiles are presented in Table 20.

TABLE 20
Biomarker-centric Profiles

| Biomarker | Gene | IHC | FISH | Mutation | DNA MA | Profile |
|---|---|---|---|---|---|---|
| c-Met | MET | ✓ | | | | Lung |
| EML | EML4, | ✓ | ✓ | | | Lung |
| 4-ALK | ALK | | | | | |
| Fusion | | | | | | |
| hENT-1 | SLC29A1 | ✓ | | | | Ovarian |
| IGFRBP | IGFRBP3, | ✓ | | | | Ovarian |
| | IGFRBP4, | | | | | |
| | IGFRBP5 | | | | | |
| IGF-1R | IGF1R | ✓ | ✓ | | ✓ | Ovarian, |
| | | | | | | Lung |
| MMR | MLH1, | ✓ | | | | Colorectal |
| | MSH2, | | | | | |
| | MSH5 | | | | | |
| p16 | CDKN2A | ✓ | | | | Colorectal |
| p21 | CDKN1A | ✓ | | | | |
| p27 | CDKN1B | ✓ | | | | |
| PARP-1 | PARP1 | ✓ | | | ✓ | Ovarian |
| PI3K | PIK3CA | ✓ | | ✓ | | Breast, |
| | | | | | | Ovarian, |
| | | | | | | Colon |
| TLE3 | TLE3 | ✓ | | | | Breast |
| | | | | | | Ovarian |

In the therapeutic-centric approach of FIG. 46B, the “complete” panel is performed to assess all markers without regard to cancer origin. The panel includes all markers listed for the biomarker centric panel.

### Example 10

### Molecular Profiling for Hormone Receptor Positive, HER-2 Negative Breast Cancer Types

Approximately 42% to 59% of breast cancers are of the hormone receptor positive A subtype, 6% to 19% are hormone receptor positive B. (Komen Foundation. Molecular Subtypes of Breast Cancer. ww5.komen.org/content.aspx?id=5372 Last accessed May 17, 2010). Hormone receptor positive A tumors tend to have the best prognosis, with high survival rates and low recurrence rates. Hormone receptor positive B patients have a lower survival rate compared with hormone receptor positive A patients.

Molecular profiling can help determine the status of a subject's hormone receptor positive, HER-2 negative breast cancer and to deliver an evidence-based report with individualized therapeutic guidance. Biomarker data derived from the tests listed in Table 21 can be used to make informed treatment decisions for hormone receptor positive, HER-2 negative cancer patients, including without limitation those who are metastatic and have completed 3^{rd} line therapy, or are metastatic and their HER-2 status has changed, or who have unique circumstances that create questions for their therapeutic management, or have exhausted standard of care therapies.

Examples of drug therapies that may be associated with clinical benefit or lack of clinical benefit based on biomarker status include Monoclonal Antibody (trastuzumab), Protein Kinase Inhibitor (lapatinib), Anthracyclines (doxorubicin, liposomal doxorubicin, epirubicin), Taxanes (paclitaxel, docetaxel, nab-paclitaxel), Platinum Analogs (carboplatin, cisplatin), Anti-Neoplastic Agent (gemcitabine), Camptothecin (irinotecan), Anti-Estrogen Therapy (fulvestrant), Armatase Inhibitors (anastrozole, exemestane, letrozole), Pyrimidine Analogues (capecitabine, 5-fluorouracil), Vinca Alkaloids (vinblastine, vinorelbine), Gonatropin Releasing Hormone Analogues (goserelin, leuprolide), Anti-Androgens (bicalutamide, flutamide, goserelin), Folic Acid Analogue (methotrexate), Selective Estrogen Receptor Modulators (tamoxifen, toremifene).

TABLE 21
Molecular Profiling for Hormone ReceptorPositive and
HER2 Negative Breast Cancer: Biomarkers Assessed

| Third line metastatic or prior | | Fourth line metastatic or beyond | | |
|---|---|---|---|---|
| IHC | | IHC | | |
| CAV-1 | P53 | AR | HER2 | PTEN |
| c-KIT | P95 | BCRP | Ki67 | RRM1 |
| CYCLIN D1 | PR | CAV-1 | MGMT | SPARC |
| EGFR | PDGFR | CYCLIN D1 | MRP1 | Mono |
| ER | PGP | c-KIT | P53 | SPARC |
| HER2 | PTEN | EGFR | P95 | Poly |
| Ki67 | TS | ER | PDGFR | TOPO1 |
| | | ERCC1 | PGP | TOP2A |
| | | | PR | TS |

| FISH | | FISH | |
|---|---|---|---|
| HER2 | cMYC | HER2 | cMYC |

| Mutation Analysis | Mutation Analysis |
|---|---|
| NA | NA |
| DNA Microarray | DNA Microarray |
| Whole genome expression array | Whole genome expression array |

### Example 11

### Molecular Profiling for HER-2 Positive Breast Cancer

Breast cancer is the second most frequently diagnosed cancer in women. (American Cancer Society. (2009). Cancer Facts & FIGS. 2009. Atlanta: American Cancer Society. p. 9-11.) Approximately 25% of breast cancers overexpress HER-2. These tumors tend to grow faster and are generally more likely to recur than tumors that do not overproduce HER-2. (National Cancer Institute. Breast Cancer Treatment (PDQ®), available at www. cancer.gov/cancertopics/pdq/treatment/breast/HealthProfessionallpage8) A challenge for treating physicians is properly selecting the order of available treatment agents when the patient progresses beyond standard of care.

Molecular profiling can help determine the status of a subject's HER-2 positive breast cancer and to deliver an evidence-based report with individualized therapeutic guidance. Biomarker data derived from the tests listed in Table 22 can be used to make informed treatment decisions for HER-2 positive breast cancer patients, including without limitation those who have progressed on trastuzumab, or are metastatic and have completed 3^{rd} line therapy, or are metastatic and their HER-2 status has changed, or have unique circumstances that create questions for their therapeutic management, or have exhausted standard of care therapies.

Examples of drug therapies that may be associated with clinical benefit or lack of clinical benefit based on biomarker status include Monoclonal Antibody (trastuzumab), Protein Kinase Inhibitor (lapatinib), Anthracyclines (doxorubicin, liposomal doxorubicin, epirubicin), Taxanes (paclitaxel, docetaxel, nab-paclitaxel), Platinum Analogs (carboplatin, cisplatin), Anti-Neoplastic Agent (gemcitabine), Camptothecin (irinotecan), Anti-Estrogen Therapy (fulvestrant), Armatase Inhibitors (anastrozole, exemestane, letrozole), Pyrimidine Analogues (capecitabine, 5-fluorouracil), Vinca Alkaloids (vinblastine, vinorelbine), Gonatropin Releasing Hormone Analogues (goserelin, leuprolide), Anti-Androgens (bicalutamide, flutamide, goserelin), Folic Acid Analogue (methotrexate), Selective Estrogen Receptor Modulators (tamoxifen, toremifene).

TABLE 22
Molecular Profiling for HER2 Positive Breast Cancer: Biomarkers Assessed

| Third line metastatic or prior | | Fourth line metastatic or beyond | | | |
|---|---|---|---|---|---|
| IHC | | IHC | | | |
| E-cadherin | P95 | AR | HER2 | PDGFR | SPARC Mono |
| ER | PGP | BCRP | Ki67 | PGP | SPARC Poly |
| HER2 | PR | c-KIT | MGMT | PR | TLE3 |
| Ki67 | PTEN | E-cadherin | MRP1 | PTEN | TOPO1 |
| MRP1 | TLE3 | ER | P53 | RRM1 | TOP2A |
| P53 | TS | ERCC1 | P95 | | TS |

| FISH | | FISH | |
|---|---|---|---|
| HER2 | cMYC | HER2 | cMYC |
| | TOP2A | | TOP2A |

| Mutation Analysis | Mutation Analysis |
|---|---|
| PIK3CA | PIK3CA |
| DNA Microarray | DNA Microarray |
| Whole genome expression array | Whole genome expression array |

### Example 12

### Molecular Profiling for Triple-Negative Breast Cancer

Approximately 10% to 15% of breast cancers are known to be “triple-receptor-negative.” (Dawood S, Broglio K, Esteva F J, Yang W, Kau S W, Islam R, Albarracin. C, Yu T K, Green M, Hortobagyi G N, Gonzalez-Angulo A M. Survival among women with triple receptor-negative breast cancer and brain metastases. Ann Oncol. 2009 April; 20(4):621-7. Epub 2009 Jan. 15.) Patients with triple negative breast cancer are more likely to relapse during the first 3 years following therapy. (Bauer K R, Brown M, Cress R D, Parise C A, Caggiano V. Descriptive analysis of estrogen receptor (ER)-negative, progesterone receptor (PR)-negative, and HER2-negative invasive breast cancer, the so-called triple-negative phenotype: a population-based study from the California cancer Registry. Cancer. 2007 May 1; 109(9):1721-8.) The relative survival for all women with triple-negative tumors is 77% at 5 years, compared with 93% for other breast cancers. (Bauer K R, Brown M, Cress R D, Parise C A, Caggiano V. Descriptive analysis of estrogen receptor (ER)-negative, progesterone receptor (PR)-negative, and HER2-negative invasive breast cancer, the so-called triple-negative phenotype: a population-based study from the California cancer Registry. Cancer. 2007 May 1; 109(9):1721-8.)

Molecular profiling can help determine the status of a subject's triple-negative breast cancer and to deliver an evidence-based report with individualized therapeutic guidance. Biomarker data derived from the tests listed in Table 23 can be used to make informed treatment decisions for triple-negative breast cancer patients, including without limitation those who are basal type and/or triple negative, or are metastatic and have completed 3^{rd} line therapy, or have unique circumstances that create questions for their therapeutic management, or have exhausted standard of care therapies.

Examples of drug therapies that may be associated with clinical benefit or lack of clinical benefit based on biomarker status include Anthracyclines (doxorubicin, liposomal doxorubicin, epirubicin), Taxanes (paclitaxel, docetaxel, nab-paclitaxel), Platinum Analogs (carboplatin, cisplatin), Anti-Neoplastic Agent (gemcitabine), Camptothecin (irinotecan), Pyrimidine Analogues (capecitabine, 5-fluorouracil), Vinca Alkaloids (vinblastine, vinorelbine), Gonatropin Releasing Hormone Analogues (goserelin, leuprolide), Anti-Androgens (bicalutamide, flutamide, goserelin).

TABLE 23
Molecular Profiling for Triple-Negative
Breast Cancer: Biomarkers Assessed

| Third line metastatic or prior | | Fourth line metastatic or beyond | | |
|---|---|---|---|---|
| IHC | | IHC | | |
| AR | Ki67 | AR | Ki67 | RRM1 |
| CK 5/6 | MRP1 | BCRP | MGMT | SPARC Mono |
| CK 14 | P53 | CK 5/6 | MRP1 | SPARC Poly |
| CK 17 | P95 | CK 14 | P53 | TLE3 |
| ER | PGP | CK 17 | P95 | TOPO1 |
| HER2 | PR | c-KIT | PDGFR | TOP2A |
| | SPARC Mono | ER | PGP | TS |
| | SPARC Poly | ERCC1 | PR | |
| | TS | HER2 | PTEN | |

| FISH | FISH |
|---|---|
| HER2 | HER2 |
| Mutation Analysis | Mutation Analysis |
| NA | NA |
| DNA Microarray | DNA Microarray |
| Whole genome expression array | Whole genome expression array |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

LENGTHY TABLES
The patent application contains a lengthy table section. A copy of the table is available in electronic form from the USPTO web site (http://seqdata.uspto.gov/?pageRequest=docDetail&DocID=US20160186266A1). An electronic copy of the table will also be available from the USPTO upon request and payment of the fee set forth in 37 CFR 1.19(b)(3).

1-2. (canceled)

3. A method of generating a report comprising a list of candidate treatments for a subject with a breast cancer, comprising:
a. performing immunohistochemistry (IHC) on a sample from the subject for a panel of proteins, wherein the panel of proteins comprises HER2, ER, PR, P53 and Ki67;
b. performing gene expression analysis on the sample for a panel of genes, wherein the panel of genes comprises ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IL2RA, KDR, KIT, LCK, LYN, MGMT, MLH1, MS4A1, MSH2, NFKB1, NFKB2, OGFR, PDGFC, PDGFRA, PDGFRB, PGR, POLA1, PTEN, PTGS2, RAF1, RARA, RRM1, RRM2, RRM2B, RXRB, RXRG, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70;
c. performing fluorescent in-situ hybridization (FISH) on the sample for one or more genes, wherein the one or more genes comprises HER2;
d. performing additional analysis on the sample dependent on the HER2, ER and PR receptor status of the breast cancer, wherein if the breast cancer is HER2 positive (HER2+), then the panel of proteins assessed in step a) further comprises AR, C-Kit, MRP1, PDGFR, PGP, PTEN, SPARC, TOP2A, TS, CAV1, CK14, CK17, CK5/6, ECAD, P95, and TLE3; the one or more genes assessed in step c) further comprises cMYC and TOP2A; and sequence analysis is performed on the sample to detect mutations in PIK3CA;
else if the breast cancer is HER2 negative (HER2−) and positive for either ER (ER+) or PR (PR+), then the panel of proteins assessed in step a) further comprises AR, C-Kit, MRP1, PDGFR, PGP, PTEN, SPARC, TOP2A, TS, CAV-1, CK14, CK17, CK 5/6, CYCLIN D1, ECAD, EGFR, P95, and TLE3; and the one or more genes assessed in step c) further comprises cMYC; or
else if the breast cancer is triple negative (HER2−, ER− and PR−), then the panel of proteins assessed in step a) further comprises AR, C-Kit, MRP1, PDGFR, PGP, PTEN, SPARC, TS, TOP2A, CAV1, CK14, CK17, CK5/6, ECAD, P95, and TLE3;
e. identifying one or more treatments having potential benefit for treating the subject's breast cancer based on the results of the IHC, gene expression, FISH and sequence analysis performed in steps a)-d); and
f. generating a report comprising the results of the IHC, gene expression, FISH and sequence analysis in performed steps a)-d), and further comprising a list of the one or more treatments identified in step e).

4. The method of claim 3, wherein identifying the one or more treatments having potential benefit for treating the subject's breast cancer in step e) comprises:
i. correlating the results of the IHC, gene expression, FISH and sequence analysis performed in steps a)-d) with a rules database, wherein the rules database comprises a mapping of treatments whose biological activity has been assessed against cancer cells that amplify, overexpress, underexpress, and/or have mutations in one or more genes or gene products assessed by the IHC, gene expression, FISH and sequence analysis performed in steps a)-d); and
ii. identifying the one or more treatments based on the correlating in (i).

5. The method of claim 4, wherein the rules database comprises one or more of rules listed in Table 3 and/or Table 4.

6. The method of claim 4, wherein the mapping of treatments contained within the rules database are based on a predicted efficacy of various treatments particular for a target gene or gene product.

7. The method of claim 3, wherein the sample comprises formalin-fixed paraffin-embedded (FFPE) tissue, fresh frozen (FF) tissue, or tissue comprised in a solution that preserves nucleic acid or protein molecules.

8.-15. (canceled)

16. The method of claim 3, wherein the gene expression analysis comprises using polymerase chain reaction (PCR), real-time PCR (qPCR; RT-PCR), next generation sequencing, a low density microarray, an expression microarray, a comparative genomic hybridization (CGH) microarray, a single nucleotide polymorphism (SNP) microarray, a proteomic array, an antibody array, or a combination thereof.

17. The method of claim 3, wherein the the panel of proteins assessed in step a) further comprises BCRP, ERCC1, MGMT, RRM1 and TOPO1; and wherein the one or more genes assessed in step c) further comprises EGFR.

18. (canceled)

19. The method of claim 3, wherein the panel of proteins assessed in step a) or the one or more genes assessed in step c) further comprises one or more of hENT1, cMet, P21, PARP-1, TLE3 and IGF1R.

20. (canceled)

21. The method of claim 3, wherein the panels of genes or gene products assessed by the IHC, gene expression, FISH and sequence analysis performed in steps a)-d) further comprises one or more of ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKN1A, CDKN1B, CDK2, CDW52, CES2, CK 14, CK 17, CK 5/6, c-KIT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, GART, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, LYN, MET, MGMT, MLH1, MMR, MRP1, MS4A1, MSH2, MSH5, Myc, NFKB1, NFKB2, NFKBIA, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, RAF1, RARA, RRM1, RRM2, RRM2B, RXRB, RXRG, SPARC, SRC, SSTR1, SSTR2, SSTR3, SSTR4, SSTR5, Survivin, TK1, TLE3, TNF, TOP1, TOP2A, TOP2B, TS, TXN, TXNRD1, TYMS, VDR, VEGF, VEGFA, VEGFC, VHL, YES1, and ZAP70.

22-27. (canceled)

28. The method of claim 3, wherein a prioritized list of candidate treatments is identified.

29. The method of claim 28, wherein prioritizing comprises ordering the treatments from higher priority to lower priority according to obtaining usable profiling results for a gene or its gene products using: 1) gene expression analysis and either IHC or FISH analysis; 2) IHC analysis but not gene expression analysis; and 3) gene expression analysis but not IHC analysis.

30. The method of claim 3, wherein the list of candidate treatments comprises one or more therapeutic agents.

31-33. (canceled)

34. The method of claim 30, wherein the one or more therapeutic agents comprise one or more of 5-fluorouracil, abarelix, alemtuzumab, aminoglutethimide, anastrozole, asparaginase, aspirin, ATRA, azacitidine, bevacizumab, bexarotene, bicalutamide, calcitriol, capecitabine, carboplatin, celecoxib, cetuximab, chemotherapy, cholecalciferol, cisplatin, cytarabine, dasatinib, daunorubicin, decitabine, doxorubicin, epirubicin, erlotinib, etoposide, exemestane, flutamide, fulvestrant, gefitinib, gemcitabine, gonadorelin, goserelin, hydroxyurea, imatinib, irinotecan, lapatinib, letrozole, leuprolide, liposomal-doxorubicin, medroxyprogesterone, megestrol, megestrol acetate, methotrexate, mitomycin, nab-paclitaxel, octreotide, oxaliplatin, paclitaxel, panitumumab, pegaspargase, pemetrexed, pentostatin, sorafenib, sunitinib, tamoxifen, Taxanes, temozolomide, toremifene, trastuzumab, VBMCP, and vincristine.

35. The method of claim 3, wherein the subject has been previously treated with one or more of the candidate treatments.

36. The method of claim 3, wherein the subject has not previously been treated with one or more of the candidate treatments.

37. The method of claim 3, wherein the cancer comprises a metastatic cancer.

38. The method of claim 3, wherein the cancer comprises a recurrent cancer.

39. The method of claim 3, wherein the cancer is refractory to a prior treatment.

40. The method of claim 39, wherein the prior treatment comprises the standard of care for the cancer.

41-54. (canceled)

55. The method of claim 3, further comprising determining a prognosis for the cancer based on the results of the IHC, gene expression, FISH and sequence analysis performed in steps a)-d).

56. The method of claim 55, wherein the prognosis is based on the analysis of one or more of the biomarkers in Table 6.

57-82. (canceled)

83. The method of claim 4, wherein the rules database comprises the rules listed in Table 4.
